SEQUENCE LISTING (2 sequences)

SEQ ID NO: 1 - protein, 670 aa, Mus musculus
MGKSEKEVATHGVRCFSKIKAFLLALTCAYVSKSLSGTYMNSMLTQIERQFGIPTSVVGLINGSFEIGNLLLIIFVSYFGTKLHRPIMIGVGCAVMGLGCFLISIPHFLMGRYEYETTILPTSNLSSNSFVCTENRTQTLKPTQDPTECVKEMKSLMWIYVLVGNIIRGMGETPIMPLGISYIEDFAKSENSPLYIGILETGMTIGPLIGLLLGSSCANIYVDTGSVNTDDLTITPTDTRWVGAWWIGFLVCAGVNILTSIPFFFFPKTLLKEGLQDNGDGTENAKEEKHREKIKEENRGITKDFFLFMKSLSCNPIYMIFILISVIQVNAFINSFTFMPKYLEQQYGKSTAEIVFLMGLYMLPPICLGYLIGGLIMKKFKITVKKAAYIGFWLSLTEYLLSFVSYIMTCDNFPVAGLTTSYEGVQHPLYVENNVLADCNTKCSCLTNTWDPVCGDNGLSYMSACLAGCEKSVGTGTNMVFQNCSCIQSSGNASAVLGLCDKGPECANKLQYFLIISIIGCFIFSLGAIPGYMVLLRCMKSEEKSLGVGLHTFCMRILGGIPAPIYFGALIDRTCLHWGTLKCGEPGACRMYDINSFRRIYLGLPAALRGASFLPALFILILMRKFQFPGDIDSSDTDPAEMKLTAKESKCTNVHRSPTMQNDGERKTKL

SEQ ID NO: 2 - protein, 670 aa, Homo sapiens
MGETEKRIETHRIRCLSKLKMFLLAITCAFVSKTLSGSYMNSMLTQIERQFNIPTSLVGFINGSFEIGNLLLIIFVSYFGTKLHRPIMIGIGCVVMGLGCFLKSLPHFLMNQYEYESTVSVSGNLSSNSFLCMENGTQILRPTQDPSECTKEVKSLMWVYVLVGNIVRGMGETPILPLGISYIEDFAKFENSPLYIGLVETGAIIGPLIGLLLASFCANVYVDTGFVNTDDLIITPTDTRWVGAWWFGFLICAGVNVLTAIPFFFLPNTLPKEGLETNADIIKNENEDKQKEEVKKEKYGITKDFLPFMKSLSCNPIYMLFILVSVIQFNAFVNMISFMPKYLEQQYGISSSDAIFLMGIYNLPPICIGYIIGGLIMKKFKITVKQAAHIGCWLSLLEYLLYFLSFLMTCENSSVVGINTSYEGIPQDLYVENDIFADCNVDCNCPSKIWDPVCGNNGLSYLSACLAGCETSIGTGINMVFQNCSCIQTSGNSSAVLGLCDKGPDCSLMLQYFLILSAMSSFIYSLAAIPGYMVLLRCMKSEEKSLGVGLHTFCTRVFAGIPAPIYFGALMDSTCLHWGTLKCGESGACRIYDSTTFRYIYLGLPAALRGSSFVPALIILILLRKCHLPGENASSGTELIETKVKGKENECKDIYQKSTVLKDDELKTKL

(19) United States
(12) Patent Application Publication
(10) Pub. No.: US 2026/0272895 A1
(43) Pub. Date: Sep. 17, 2026

(54) METHODS AND KITS FOR DELIVERY OF COMPOUNDS INTO CELLS

(71) Applicant: Yale University, New Haven, CT (US)

(72) Inventors: Jaime Grutzendler, New Haven, CT (US); Roshan Gunasekara, New Haven, CT (US); Lejie Zhang, New Haven, CT (US)

(21) Appl. No.: 19/469,013

(22) PCT Filed: Mar. 27, 2024
(86) PCT No.: PCT/US2024/021731

§ 371 Date: Sep. 25, 2025

Related U.S. Application Data
(60) Provisional application No. 63/492,447, filed on Mar. 27, 2023.

(51) Int. Cl. A61K 31/4375; A61K 31/352; A61K 31/4178; A61K 31/4196; A61K 31/5377; A61K 31/69; A61K 38/17; A61K 47/54; A61K 47/55

(52) U.S. Cl. CPC A61K 31/4375; A61K 31/352; A61K 31/4178; A61K 31/4196; A61K 31/5377; A61K 31/69; A61K 38/1709; A61K 47/545; A61K 47/55

(57) ABSTRACT

Disclosed herein are methods of delivering a compound into a cell. In some embodiments, the method includes expressing in the cell a protein that confers a receptiveness for a compound herein, as well as contacting the cell with the compound herein. Also disclosed herein are kit for performing the method. In certain embodiments, the method and the kit are method and kit for performing gene therapy in a subject.

### CROSS-REFERENCE TO RELATED APPLICATIONS

This application claims priority under 35 U.S.C. § 119(e) to U.S. Provisional Patent Application No. 63/492,447, filed Mar. 27, 2023, which application is hereby incorporated herein by reference in its entirety.

### STATEMENT REGARDING FEDERALLY SPONSORED RESEARCH OR DEVELOPMENT

This invention was made with government support under NS128953 and NS115544 awarded by the National Institutes of Health. The government has certain rights in the invention.

### SEQUENCE LISTING

The XML file named “047162-7454WO1—Sequence Listing.xml” created on Mar. 27, 2024, comprising 4.2 Kbytes, is hereby incorporated herein by reference in its entirety.

### BACKGROUND

A significant challenge for developing effective treatments is that therapeutic manipulation of signaling pathways often have both beneficial and detrimental effects in a cell-type specific manner.

To address this challenge, strategies have been developed to improve target selectivity. Such strategies include the use of antibody-drug conjugates to target specific membrane neoantigens, especially in the cancer field, and strategies to broadly restrict therapeutics to the brain by using binary pharmacology. However, there is no systematic approach to target intracellular pathways in a cell-type specific manner.

As such, there is a need for systematic approach to deliver treatment in a cell-type specific manner. The present invention addresses this need.

### BRIEF SUMMARY

In one aspect, the disclosure provides a method of delivering a compound into a cell. In certain embodiments, the method comprises:
expressing Slco1a4 or SLCO1A2 in the cell; and
contacting with the cell a compound of formula (I), or a salt, solvate, tautomer, geometric isomer, or isotopologue thereof, and any combinations thereof:

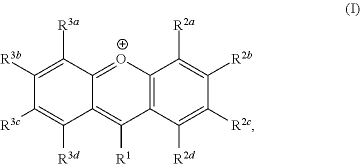

wherein:
R¹ is

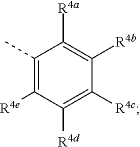

R^{2a}, R^{2b}, R^{2c} and R^{2d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B},
   wherein two vicinal substituents selected from the group consisting of R^{2a}, R^{2b}, R^{2c}, and R^{2d} can combine with the atoms to which they are bound to form an optionally substituted C₆-C₁₀ aryl or optionally substituted C₂-C₁₀ heteroaryl, and
   wherein no more than three of R^{2a}, R^{2b}, R^{2c}, and R^{2d} are H;
R^{3a}, R^{3b}, R^{3c}, R^{3d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D},
   wherein two vicinal substituents selected from the group consisting of R^{3a}, R^{3b}, R^{3c}, and R^{3d} can combine with the atoms to which they are bound to form an optionally substituted C₆-C₁₀ aryl or optionally substituted C₂-C₁₀ heteroaryl, and
   wherein no more than three of R^{3a}, R^{3b}, R^{3c}, and R^{3d} are H:
R^{4a}, R^{4b}, R^{4c}, R^{4d}, and R^{4e} are each independently selected from the group consisting of H, optionally substituted C₁-C₆ alkyl, CN, NO₂, S(═O)₂OH, and S(═O)₂R⁵,
   wherein at least one of R^{4a}, R^{4b}, R^{4c}, R^{4d}, and R^{4e} is S(═O)₂OH, and
   wherein at least one of R^{4a}, R^{4b}, R^{4c}, R^{4d}, and R^{4e} is S(═O)₂R⁵;
R⁵ is at least one selected from the group consisting of H, OH, halogen, and

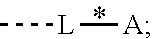

each occurrence of L is independently selected from the group consisting of a bond and a linker;
each occurrence of A is H, optionally substituted C₁-C₆ alkyl, an imaging agent, a polymeric macromolecule, or a therapeutic agent;
R^{A} and R^{B}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{A} and R^{B} can independently with one of R^{2a}, R^{2b}, R^{2c} and R^{2d} to form an optionally substituted C₅-C₈ heterocycloalkyl or C₅-C₈ heterocycloalkenyl; and
R^{C} and R^{D}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{C} and R^{D} can independently with one of R^{3a}, R^{3b}, R^{3c} and R^{3d} to form an optionally substituted C₅C₈ heterocycloalkyl or C₅-C₈ heterocycloalkenyl.

In another aspect, the disclosure provides a kit for delivering a compound into a cell. In certain embodiments, the kit comprises:
a Slco1a4 protein, a SLCO1A2 protein, or a nucleic acid encoding the same; and
a compound of formula (I), or a salt, solvate, tautomer, geometric isomer, or isotopologue thereof, and any combinations thereof:

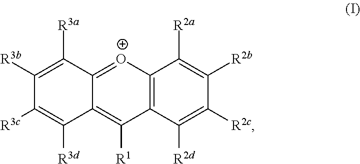

wherein:
R¹ is

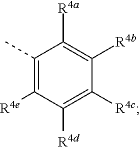

R^{2a}, R^{2b}, R^{2c}, and R^{2d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B},
   wherein two vicinal substituents selected from the group consisting of R^{2a}, R^{2b}, R^{2c}, and R^{2d} can combine with the atoms to which they are bound to form an optionally substituted C₆-C₁₀ aryl or optionally substituted C₂-C₁₀ heteroaryl, and
   wherein no more than three of R^{2a}, R^{2b}, R^{2c}, and R^{2d} are H;
R^{3a}, R^{3b}, R^{3c}, R^{3d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D},
   wherein two vicinal substituents selected from the group consisting of R^{3a}, R^{3b}, R^{3c}, and R^{3d} can combine with the atoms to which they are bound to form an optionally substituted C₆-C₁₀ aryl or optionally substituted C₂-C₁₀ heteroaryl, and
   wherein no more than three of R^{3a}, R^{3b}, R^{3c}, and R^{3d} are H; R^{4a}, R^{4b}, R^{4c}, R^{4d}, and R^{4e} are each independently selected from the group consisting of H, optionally substituted C₁-C₆ alkyl, CN, NO₂, S(═O)₂OH, and S(═O)₂R⁵,
   wherein at least one of R^{4a}, R^{4b}, R^{4c}, R^{4d}, and R^{4e} is S(═O)₂OH, and
   wherein at least one of R^{4a}, R^{4b}, R^{4c}, R^{4d}, and R^{4e} is S(═O)₂R⁵;
R⁵ is at least one selected from the group consisting of H, OH, halogen, and

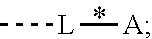

each occurrence of L is independently selected from the group consisting of a bond and a linker;
each occurrence of A is H, optionally substituted C₁-C₆ alkyl, an imaging agent, a polymeric macromolecule, or a therapeutic agent;
R^{A} and R^{B}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{A} and R^{B} can independently with one of R^{2a}, R^{2b}, R^{2c}, and R^{2d} to form an optionally substituted C₅-C₈ heterocycloalkyl or C₅-C₈ heterocycloalkenyl; and
R^{C} and R^{D}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
wherein each of R^{C} and R^{D} can independently with one of R^{3a}, R^{3b}, R^{3c} and R^{3d} to form an optionally substituted C₅-C₈ heterocycloalkyl or C₅-C₈ heterocycloalkenyl.

### BRIEF DESCRIPTION OF THE FIGURES

The drawings illustrate generally, by way of example, but not by way of limitation, various embodiments of the present application.

FIGS. 1A-1B illustrate that the compounds of the present disclosure readily cross the blood brain barrier despite their hydrophilic nature. FIGS. 1A-1B show 2-photon imaging of the mouse brain cortex following intravenous administration of compound 1 of the present disclosure. Blue arrows point to the interstitial space where there is a marked increase in fluorescence comparing 10 min (FIG. TA) and 130 min (FIG. 1B) post injection images. Below each image there is a fluorescence intensity plot along a line profile (blue line) across capillary vessels and adjacent interstitial space.

FIGS. 2A-2B show in vivo 2-photon imaging at 10 min (FIG. 2A), and 130 min (FIG. 2B) post administration of compound 1. The white arrow in FIG. 2A points to compound 1 within the brain capillary lumen at 10 minutes IV post injection. At 130 mins FIG. 2B there is a marked uptake of the compound into the capillary endothelial wall (blue arrow). A brightly labeled endothelial cell body is also seen (blue arrowhead).

FIG. 3 depicts that 2-hours post intravenous compound injection a significant portion of the compound (i.e., compound 1) is located in the brain interstitial space. The lack of cell body labeling (blue arrowhead pointing to black circular cell bodies on the red background) indicates that the compound does not enter non-endothelial cells.

FIG. 4 illustrates the highly specific endothelial labeling with the compounds of the present disclosure. FIG. 4 depicts direct topical application of compound 1 to the brain surface through a craniotomy in a live mouse. Highly specific labeling of endothelial cells within capillaries occurs 20 minutes after application of the compound.

FIG. 5 illustrates that certain compounds of the present disclosure are transported via vesicular transcytosis. Using a lower concentration of compound 1 at 1.6 mg of compound 1 in 120 μL of PBS, at around 2 hours post injection, a bright punctate labeling becomes apparent in the endothelium.

FIGS. 6A-6C illustrate topical brain application of a conjugate of compound 1 according to the disclosure with the cancer drug methotrexate (compound 52a/52b). The uptake of the compound into endothelial cells of small capillaries (FIGS. 6A-6B), and arterioles (FIG. 6C) in the brain is very strong.

FIGS. 7A-7B depict labeling of endothelial cells in vivo with fluorinated (FIG. 7A) and iodinated (FIG. 7B) compounds.

FIG. 8A provides a schematic representation of an exemplary combinatorial library synthesis described in the present disclosure. FIG. 8B depicts exemplary backbones and/or scaffolds for the small molecule fluorophores. FIG. 8C provides a schematic representation of exemplary small molecule fluorophore library screening methodology as described in the present disclosure. FIG. 8D depicts in vivo two-photon images captured from validated small molecules that are taken up by pericytes, astrocytes, neuronal soma, endothelium, and axons. FIG. 8E depicts exemplary compounds of the present disclosure which are specific for endothelial uptake in vivo. FIG. 8F depicts topical administration of Endo-Red compound in Tie2GFP transgenic mice as revealed by in vivo two-photon images. FIG. 8G depicts exemplary compounds of the present disclosure comprising fluorine (i.e., compound 2) and iodine (i.e., compound 4) substitution (i.e., substituted Endo-Red compounds). FIG. 8H depicts exemplary compounds of the present disclosure (i.e., compounds 2 and 4) selectively labeling endothelial cells in vivo as revealed by two-photon imaging microscope. FIG. 8I depicts ex vivo confocal images of retina labeling with exemplary compounds of the present disclosure (i.e., compounds 2 and 4).

FIG. 9A depicts uptake of an exemplary Endo-Red compound by organic anion transporting polypeptides Slco1a4 (mice) in HEK293 cells and Single Cell Ribonucleic acid (RNA) sequence Database results for Slco1a4 (mice). FIG. 9B depicts an exemplary Endo-Red compound by organic anion transporting polypeptides SLCO1A2 (human ortholog) in HEK293 cells and Single Cell Ribonucleic acid (RNA) sequence Database results for SLCO1A2 (human ortholog). FIG. 9C depicts in vivo two-photon images captured from (a) wild type and (b) Slco1a4 transporter knockout mice after 5 μM topical administration of an exemplary Endo-Red compound. FIG. 9D depicts exemplary Endo-Red compound uptake into (a) SLCO1A2 and (b) SLCO1A2 E172D mutated transporter as revealed by in vivo two-photon images. FIG. 9E provides further confirmation that SLCO1A2 E172D mutation reduces the Endo-Red uptake in HEK293.

FIG. 10A depicts exemplary Endo-Red family compounds conjugated to colchicine (i.e., compounds 47 and 48). FIG. 10B provides in vivo two-photon images captured from 10 μM topical administration of an exemplary colchicine-conjugated compound of the present disclosure. FIG. 10C depicts an exemplary Endo-Red family compound conjugated to deferoxamine (i.e., compound 49). FIG. 10D depicts topical administration of an exemplary deferoxamine-conjugated compound of the present disclosure in WT mice as revealed by in vivo two-photon images. FIG. TOE depicts an exemplary Endo-Red family compound conjugated to taxol (i.e., compound 51). FIG. 10F depicts topical administration of an exemplary taxol-conjugated compound of the present disclosure in WT mice as revealed by in vivo two-photon images. FIG. 10G depicts an exemplary synthesis of an Endo-Red family compound conjugated to JAK inhibitor tofacitinib (i.e., compound 53). FIG. 10H shows that the exemplary tofacitinib-conjugate is taken up by organic anion transporting polypeptides (a) Slco1a4 (mice)/(b) SLCO1A2 (human ortholog) in HEK293 cells.

FIG. 11A depicts alpha tubulin staining of 30 μM (a) colchicine, (b) colchicine-conjugate, and (c) Endo-Red in NIH3T3 cells. The data shows equal disruption of the tubulin structure by both colchicine and colchicine-conjugate compounds. FIG. 11B depicts alpha tubulin staining of 100 μM (a) colchicine, (b) colchicine-conjugate, and (c) Endo-Red in NIH3T3 cells. The data shows equal disruption of the tubulin structure by both colchicine and colchicine-conjugate compounds. FIG. 11C depicts phalloidin in NIH3T3 cells. The data shows equal disruption of the tubulin structure by both colchicine and colchicine-conjugate compounds. FIG. 11D depicts phalloidin staining of 100 μM (a) colchicine, (b) colchicine-conjugate, and (c) Endo-Red in NIH3T3 cells. The data shows disruption of the tubulin structure by colchicine and mild effect by the colchicine-conjugate.

FIGS. 12A-12C depict intraperitoneal administration of ~80 μM (FIG. 12A) colchicine-conjugate, (FIG. 12B) colchicine, and (FIG. 12C) Endo-Red family compounds to P12 pups for 10 consecutive days.

FIG. 13A depicts in vivo two-photon images captured from (a) Slco1a4-GFP and (b) SLCO1A2-GFP transporter overexpressed neurons after topical application of Endo-Red compound. FIG. 13B depicts confocal histology images of labeling endothelial cells and neurons by Endo-Red compound in mice with SLCO1A2-GFP overexpressed in neurons.

FIGS. 14A-14F: Intravital imaging-based screening of combinatorial fluorophore library uncovers molecules that specifically target brain and retina endothelium. FIG. 14A: Flow chart describing strategy for imaging-based fluorophore library screening in the live mouse brain. FIG. 14B: chemical structures of endothelial-specific compounds (eEDiTS). FIG. 14C: Intravital in vivo two-photon images captured from the cortex of wild type mice labeled with eEDiTS (50 μM), showing labeling of endothelial processes (arrow) and cell bodies (arrowhead), while no other cell types are labeled. Scale bars 20 μm. FIG. 14D: Confocal images of a mouse retina explant following intravitreal injection of eEDiTS (50 μM), showing selective labeling of endothelial processes (arrow) and cell bodies (arrowhead). Scale bars 20 μm. FIG. 14E: In vivo two-photon images obtained after topical cortical administration of eEDiTS (50 μM) in Tie2-GFP endothelial reporter mice demonstrates precise colocalization between eEDiTS and GFP labeling in endothelial processes (arrow) and cell bodies (arrowhead). Scale bars, 20 μm (upper panels) and 5 μm (lower panels). FIG. 14F: In vivo two-photon imaging after intravenous administration of eEDiTS (0.1 mM) in Tie2-GFP mice demonstrates flowing intravascular compound (asterisks) as well as precise colocalization between eEDiTS and GFP labeling primarily at endothelial cell bodies (arrowhead). Scale bars, 20 μm (upper panels) and 5 μm (lower panels).

FIGS. 15A-15C: Intracellular eEDiTS uptake is mediated by the solute carriers Slco1a4/SLCO1A2. FIG. 15A: Confocal images of HEK293 cells transfected with either Slco1a4, SLCO1A2, or GFP control following administration of eEDiTS (10 μM) showing robust and specific eEDiTS uptake in Slco1a4 or SLCO1A2 transfected cells. The Slco1a4 and SLCO1A2 proteins were fused to a FLAG Tag for visualization. Scale bars, 10 μm. FIG. 15B: In vivo two-photon brain images after topical cortical administration of eEDiTS (50 μM) in Slco1a4 knockout mice (Oatp1a/1b cluster knockout) (left) compared to wildtype (right) mice demonstrates a complete elimination of eEDiTS uptake in endothelial processes (arrow) and cell bodies (arrowhead). Scale bar, 20 μm. FIG. 15C: In vivo two-photon brain images after intravenous administration of eEDiTS (0.1 mM) in mice lacking Slco1a4 demonstrates no eEDiTS uptake in either endothelial processes (arrow) or cell bodies (arrowhead) as compared to wildtype mice (upper panels) while one can still visualize the fluorescence of flowing intravascular eEDiTS (bottom panels, asterisks). Scale bar, 20 μm.

FIGS. 16A-16E: Conjugation of eEDiTS to a pharmacological agent does not disrupt its membrane transport cellular uptake specificity. FIG. 16A: Synthetic route for conjugating eEDiTS to Colchicine. FIG. 16B: Confocal images of HEK293 cells transfected with either Slco1a4, SLCO1A2, or GFP control following administration of Colchicine-eEDiTS (10 μM) showing robust and selective uptake in Slco1a4 and SLCO1A2 transfected cells. Scale bars, m. FIG. 16C: Intravital in vivo two-photon images captured from the cortex of wild type mice labeled topically with Colchicine-eEDiTS (50 μM), showing labeling of endothelial processes (arrow) and cell bodies (arrowhead). Scale bars, 20 μm. FIG. 16D: In vivo two-photon images after topical cortical administration of Colchicine-eEDiTS (50 μM) in Tie2-GFP mice showing a precise colocalization between Colchicine-eEDiTS and GFP labeling of endothelial processes (arrow) and cell bodies (arrowhead). Scale bars, 20 μm (upper panels) and 5 μm (lower panels). FIG. 16E: In vivo two-photon brain images after topical cortical administration of Colchicine-eEDiTS (50 μM) in Slco1a4 KO (bottom panel) and wildtype mice (top panel) demonstrates a complete elimination of Colchicine-eEDiTS uptake in both endothelial processes (arrow) and cell bodies (arrowhead). Scale bar, 20 μm.

FIGS. 17A-17C: Colchicine-eEDiTS conjugate retains its pharmacological properties. FIG. 17A: Confocal images of α-tubulin immunofluorescence (green) in NIH3T3 cells treated with either vehicle, eEDiTS, Colchicine, or Colchicine-eEDiTS. The normal filamentous pattern of tubulin staining in vehicle and eEDiTS treated cells is replaced by a diffuse labeling pattern with Colchicine and Colchicine-eEDiTS treatments (arrowheads) due to a dose-dependent microtubule inhibitory effect. Notice that Colchicine and Colchicine-eEDiTS treatments were also associated with changes in cell morphology and presence of multinucleated cells. Scale bars, 20 μm. FIG. 17B: Quantification of the degree of multinucleated cells at various compound concentrations. FIG. 17C: Fluorescence-based cell free tubulin polymerization assay comparing the effects of eEDiTS, Colchicine-eEDiTS. Colchicine, and Vehicle (3 μM) (N=3 replicates per group).

FIGS. 18A-18C: Colchicine-eEDiTS conjugate demonstrates markedly reduced local and systemic toxicity. FIG. 18A: local administration of Colchicine, Colchicine-eEDiTS or eEDiTS (intradermal injections on the right lower back quadrant) following fur shaving demonstrates their differential effects on fur regrowth at various time points and drug concentrations. FIG. 18B: changes in grayscale intensity between drug injected and un-injected back quadrants (see methods) were plotted to depict rates of fur regrowth as a result of drug treatments. FIG. 18C: Plots showing the daily measures of body weight in 3-week-old mice that were injected daily intraperitoneally for 5 consecutive days with either Colchicine. Colchicine-eEDiTS or eEDiTS (2.5 μmol/gram) (N=3 mice per treatment group).

FIGS. 19A-19C: AAV-mediated gene therapy introducing SLCO1A2 in neurons leads to robust uptake of eEDiTS. FIG. 19A: Schematic diagram depicting subarachnoid infusion of AAV8-CAG-SLCO1A2 GFP or AAV8-CAG-GFP in P1 mice to predominantly infect cortical neurons. FIG. 19B: Intravital in vivo two-photon images captured from the cortex of wild type mice 3 weeks after AAV injections predominantly shows layer II GFP neuronal labeling (green). Topical administration of eEDITS (50 μM), demonstrates robust uptake by cells expressing SLCO1A2 (left top panel) but not GFP control (right top panel). Notice the loss of endothelial uptake with expression of SLCO1A2, likely due to competition for compound uptake. Fluorescent intensity colocalization profiles are shown at the bottom graphs. FIG. 19C: Intravital in vivo two-photon images captured from the cortex. Topical administration of Colchicine-eEDITS (50 μM), demonstrates robust uptake by cells expressing SLCO1A2 (left top panel) but not GFP control (right top panel). Fluorescent intensity colocalization profiles are shown at the bottom graphs. Scale bars for B and C, 10 μm.

FIGS. 20A-20C: Intravital imaging-based screening of combinatorial fluorophore library uncovers molecules that specifically target various brain cell types. FIG. 20A: in vivo two-photon images captured from the cortex of wild type mice labeled with nEDiTS (50 μM), showing robust labeling of layer II cortical neurons. FIG. 20B: in vivo images in the cortex of wild type mice labeled with pEDiTS (50 μM), showing robust labeling of pericyte cell bodies and perivascular processes. FIG. 20C: in vivo images in the cortex of wild type mice labeled with aEDiTS (50 μM), showing robust labeling of astrocyte cell bodies and perivascular end-feet. Scale bars 20 μm.

FIG. 21A: schematic of experimental procedure. FIG. 21B: Evaluation of Intracellular eEDiTS uptake in HEK293 cells following transfection of various solute carriers. Notice the markedly increased uptake following administration of eEDiTS (10 μM) by cells expressing Slco1a4 compared to those expressing other transporters. Scale bars, 20 μm.

FIGS. 22A-22C: Slcoa1a4 is expressed in intraparenchymal blood vessels and is absent from pial arteries and penetrating arterioles. FIG. 22A: Imaging of a tangential section including the pial surface of the mouse brain in Tie2-GFP reporter mice after immunofluorescence staining with an antibody against Slco1a4. FIG. 22B: pial arterioles (1) do not display Slco1a4 labeling. FIG. 22C: Penetrating arterioles (2) do not display Slco1a4 labeling, while smaller intraparenchymal blood vessels extensively express Slco1a4. Note that not all vessels are GFP labeled in this transgenic reporter line. Scale Bars 20 μm.

FIG. 23A: Synthesis route for N-Boc-ethylenediamine-eEDiTS (643 Da). FIG. 23B: in vivo two-photon images of the mouse cortex following N-Boc-ethylenediamine-eEDiTS administration demonstrates robust endothelial cell body uptake (arrows). FIG. 23C: high zoom images shows both endothelial cell body uptake (arrowheads) and dimmer endothelial process labeling (arrows). Scale Bar 20 μm.

FIG. 24A: Synthesis route for conjugation of the iron chelator Deferoxamine with eEDiTS to obtain a large hydrophilic compound Deferoxamine-eEDiTS (1044 Da). FIG. 24B: in vivo two-photon images of the mouse cortex following Deferoxamine-eEDiTS administration demonstrates robust endothelial cell body uptake (arrows). FIG. 24C: Deferoxamine-eEDiTS preloaded with FeCh (1100 Da) retains the uptake properties and shows an even more robust labeling of endothelial cell bodies and processes. Scale Bar 20 μm.

FIGS. 25A-25F: Biodistribution of eEDiTS in various organs following intravenous administration. FIG. 25A: schematic depicting eEDiTS pharmacokinetics study. FIGS. 25B-25F: eEDiTS was injected intravenously (70 μL of 0.1 mM solution); 2.5 hours later mice were perfused, and tissues of various organs were imaged with confocal microscopy. Notably heart (FIG. 25B), spleen (FIG. 25C), and skeletal muscle (FIG. 25D) showed negligible parenchymal or endothelial uptake. In contrast both liver (FIG. 25E) and kidney (FIG. 25F) show robust cellular uptake in tubular epithelium and hepatocytes respectively, but not in glomerular or liver vasculature. Scale bars, 20 μm.

FIGS. 26A-26C: In utero electroporation of either Slco1a4 or SLCO1A2 constructs to transfect cortical neurons leads to robust uptake of eEDiTS in neurons. FIG. 26A: diagram depicting the in utero electroporation (IUE) procedure. FIGS. 26B-26C: in vivo two-photon images captured from the cortex of wild type mice 4 weeks after IUE shows robust eEDiTS uptake in neurons following topical cortical administration (50 μM) in both Slco1a4 (FIG. 26B) and SLCO1A2 (FIG. 26C) transfected mice. Note that the normally observed endothelial labeling in wildtype mice disappears in regions with robust neuronal uptake, presumably due to the overwhelming competitive uptake by the neurons. Scale bars, 10 μm.

FIGS. 27A-27C: In vivo time lapse imaging demonstrates blood-brain barrier crossing by eEDiTS: FIG. 27A: eEDiTS was injected intravenously (70 μL of 0.1 mM solution) and imaged through a cranial window overtime. Fluorescence intensities in multiple regions of interest per field of view in areas outside of blood vessels (dotted circles) were quantified and averaged. FIG. 27B: average fluorescence intensity plotted over time. (n=20 fields of view in each of 2 mice). FIG. 27C: intravenous administration of eEDiTs in a mouse that underwent in utero electroporation with SLCO1A2 construct to transfect cortical neurons. Fixed tissue imaging of mouse cortex shows robust labeling of neurons due to efficient BBB crossing of eEDiTs. Scale bars, 20 μm.

FIGS. 28A-28B: Demonstration that iodination or fluorination of eEDiTS does not disrupt its transport properties or endothelial specificity. FIG. 28A: chemical structures of fluorinated (Compound 43) and iodinated (compound 54) compounds. FIG. 28B: in vivo images following topical cortical administration (50 μM) showing robust endothelial specific uptake of both compound 43 and compound 54. Scale bars, 20 μm.

FIGS. 29A-29D: Fluorescent excitation and emission spectra of eEDiTS and Colchicine-eEDiTS. Fluorescent excitation and emission spectrum measured using 50 μM solution in deionized water of eEDiTS compounds 1 (FIG. 29A), 43 (FIG. 29B), and 46 (FIG. 29C), and colchicine-eEDiTS compound 47 (FIG. 20D).

FIGS. 30A-30F: illustrate the highly specific in vivo endothelial labeling with the compounds 56 (FIG. 30A), 57 (FIG. 30B), 58 (FIG. 30C), 59 (FIG. 30D), 60 (FIG. 30E), and 61 (FIG. 30F).

FIGS. 31A-31F: ¹H-NMR and ¹³C-NMR spectra for compounds 56 (FIG. 31A), 57 (FIG. 311B), 58 (FIG. 31C), 59 (FIG. 31D), 60 (FIG. 31E), and 61 (FIG. 31F).

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting: information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The study described herein (“the present study”) developed a series of organic compounds (also referred to as “the compounds herein”) that can enter endothelial cells in a cell-type specific manner. Notably, bifunctional conjugates including both the compounds herein and pharmaceutical compounds, were confirmed to retain both the cell-type specificity of the compounds herein, and the native pharmacological properties of the pharmaceutical compounds.

The present study identified the cell surface proteins responsible for intaking the series of organic compounds. In mice, the protein is solute carrier organic anion transporter family, member 1a4 (Slco1a4). In humans, the protein is solute carrier organic anion transporter family member 1A2 (SLCO1A2).

Without wishing to be bound by theories, by introducing the mouse Slco1a4 protein and/or human SLCO1A2 protein into a cell that does not naturally expressing these transporter proteins, the cells become receptive to the compounds herein and the pharmaceutical compound conjugates thereof, thus allowing specific targeting to these cells with both the compounds herein, and the bifunctional conjugates.

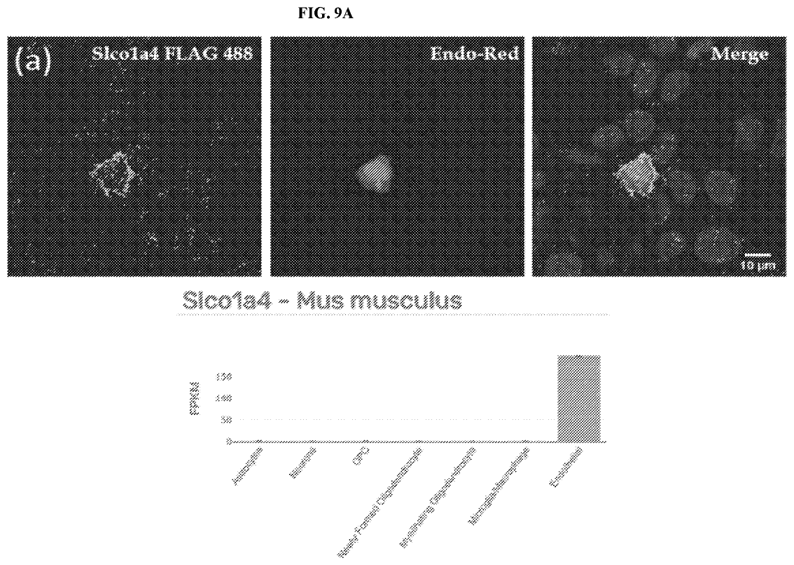

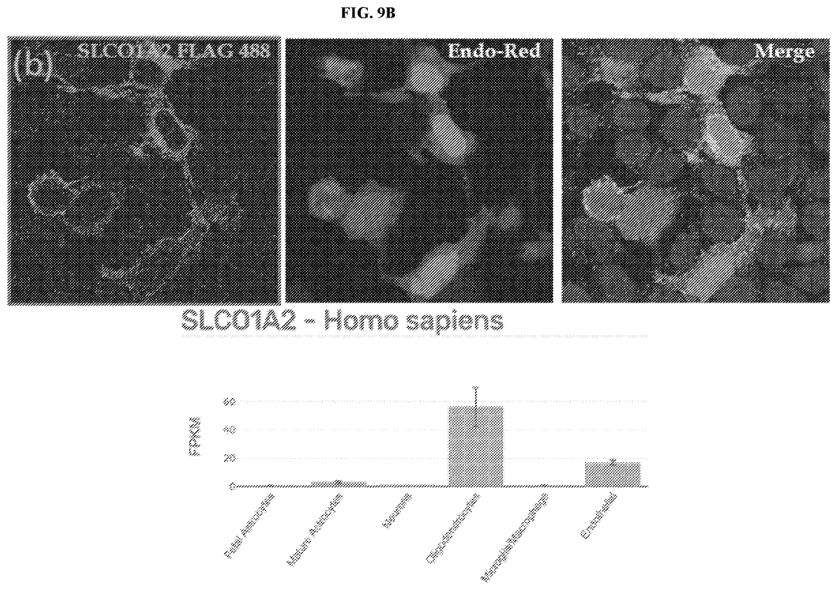

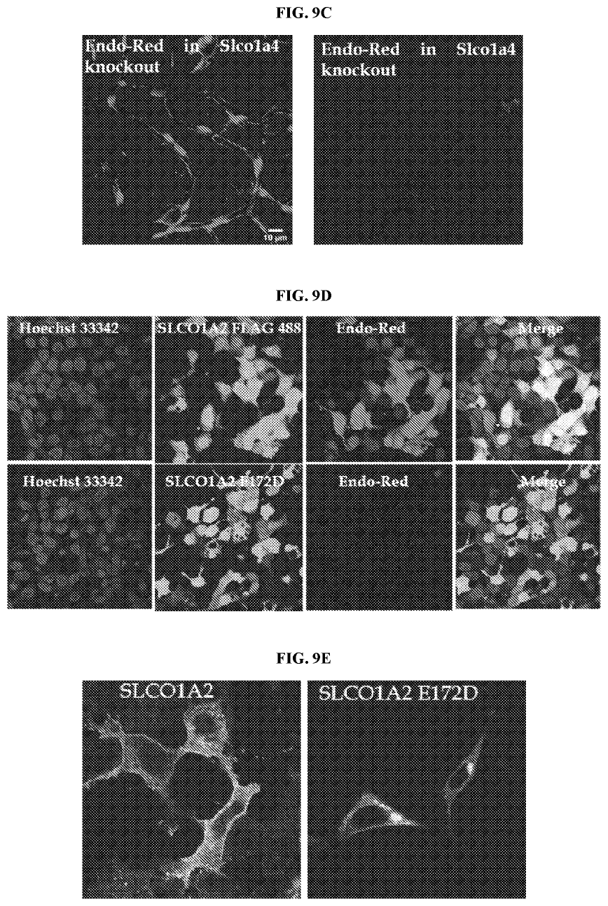

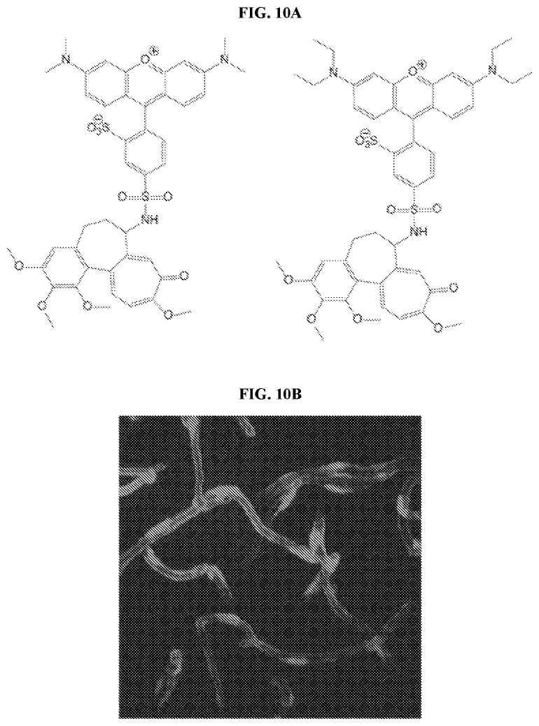

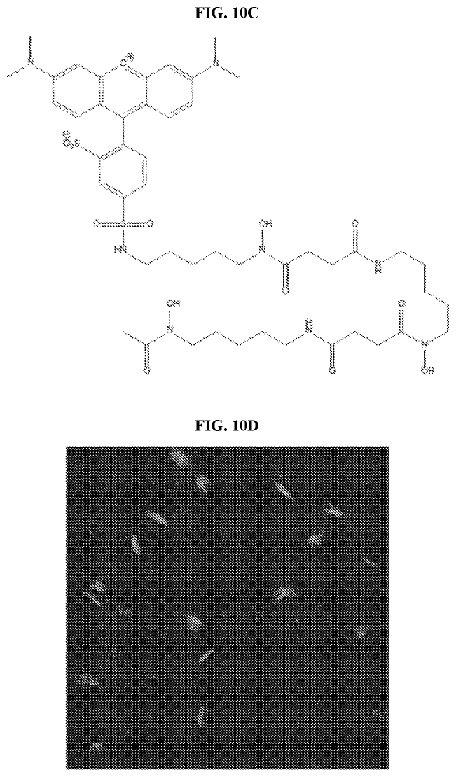

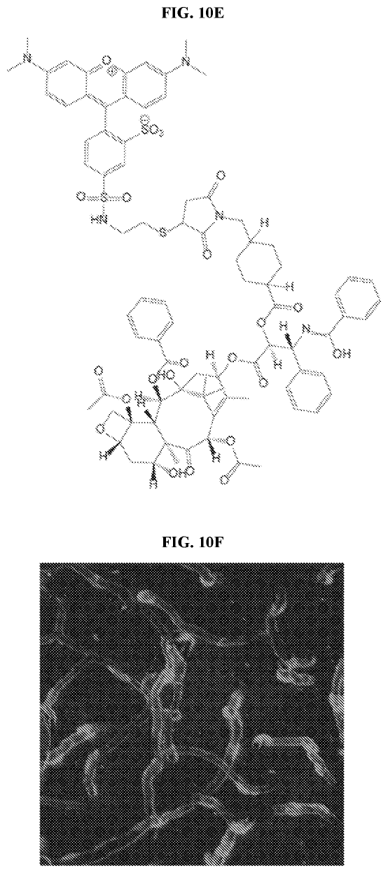

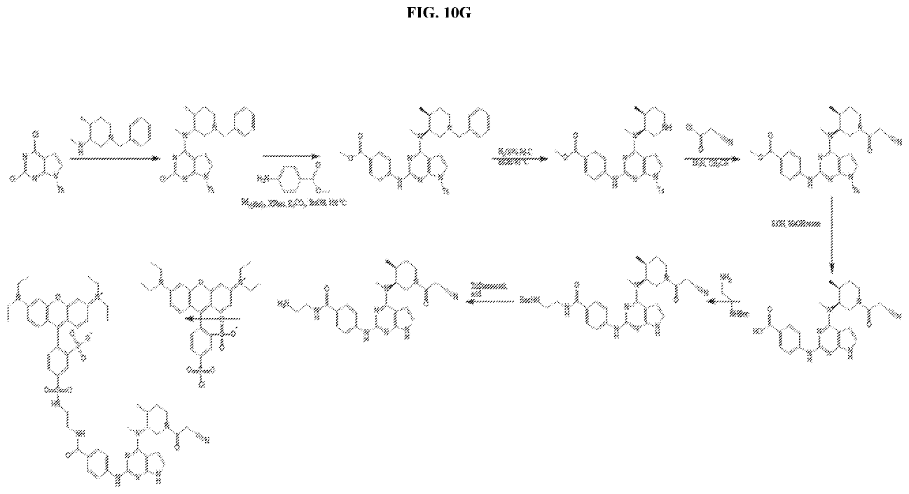

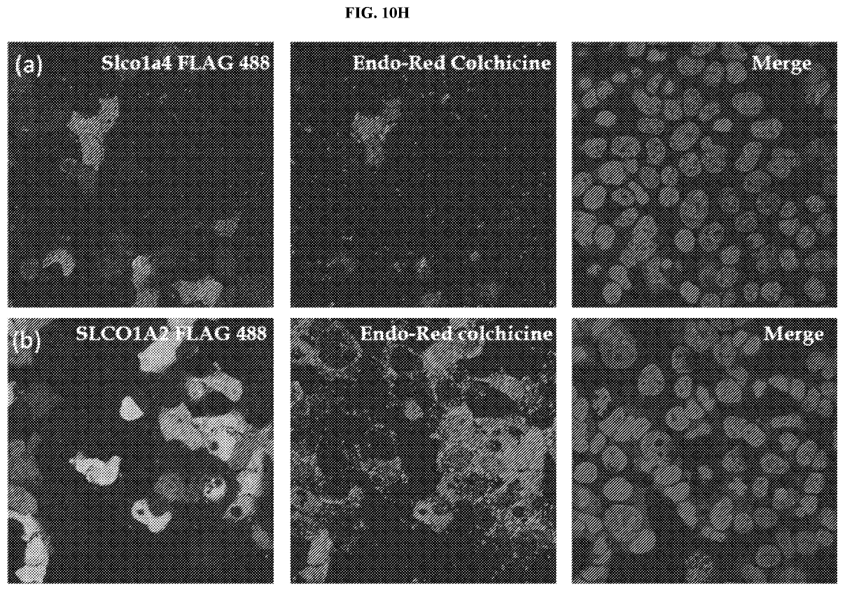

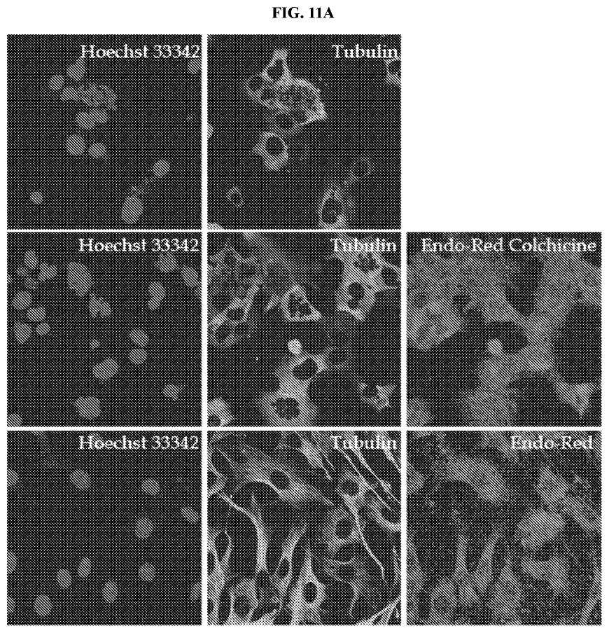

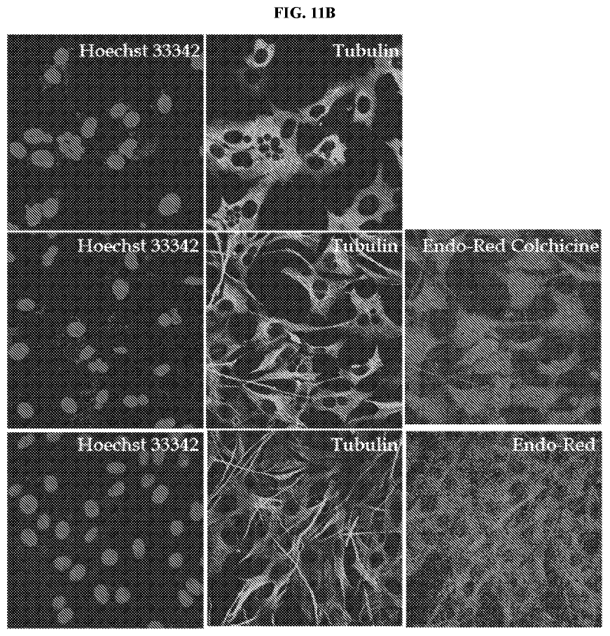

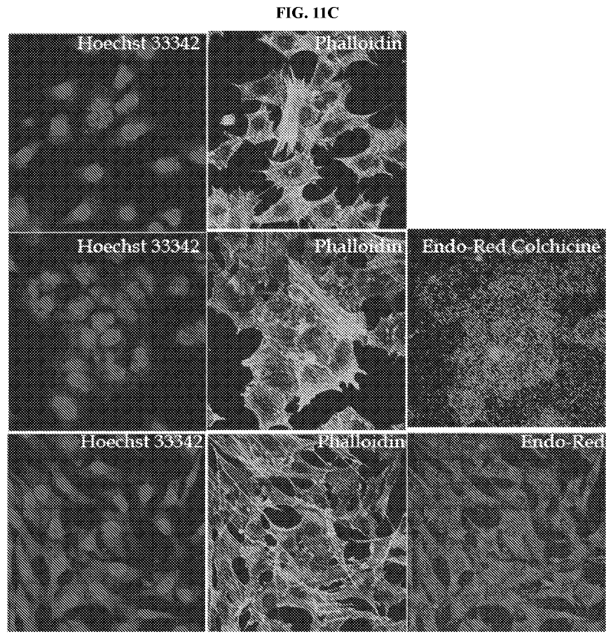

Indeed, the present study demonstrated that cells that do not expressing Slco1a4 or SLCO1A2 are not receptive to either the compounds herein or drug conjugates thereof. However, the introduction of exogenous Slco1a4 or SLCO1A2 in the same cells was able to alter the receptiveness of the cell, and allowed the specific entry of both the compounds herein and the drug conjugates thereof. (see e.g., FIGS. 9A-9F, 10H and 13A-13B).

Accordingly, in some aspects, the present study is directed to a method of delivering a compound into a cell. In some aspects, the present study is directed to a kit for delivering a compound into a cell. In some embodiments, the method and the kit are method and kit for performing gene therapy in a subject.

### Definitions

The term “about” as used herein can allow for a degree of variability in a value or range, for example, within 10%, within 5%, or within 1% of a stated value or of a stated limit of a range, and includes the exact stated value or range.

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CHCH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “cycloalkylene” or “cycloalkylenyl” as used herein refers to a bivalent saturated cycloalkyl radical

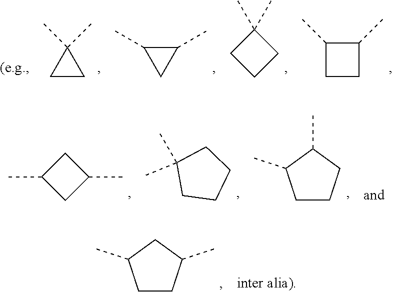

In certain embodiments, the term may be regarded as a product of removal of two hydrogen atoms from the corresponding cycloalkane (e.g., cyclobutyl) by removal of two hydrogen atoms from the same

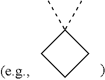

different

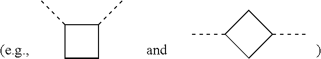

carbon atoms.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “ameliorated” if the severity of a symptom of the disease or disorder, the frequency with which such a symptom is experienced by a patient, or both, is reduced.

As used herein, the terms “effective amount.” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

In particular, in the case of a RNA (e.g., mRNA) and DNA, an “effective amount” or “therapeutically effective amount” of a therapeutic nucleic acid as relating to a RNA or DNA is an amount sufficient to produce the desired effect, e.g., mRNA-directed expression of an amount of a protein that causes a desirable biological effect in the organism within which the protein is expressed. For example, in some embodiments, the expressed protein is an active form of a protein that is normally expressed in a cell type within the body, and the therapeutically effective amount of the mRNA is an amount that produces an amount of the encoded protein that is at least 50% (e.g., at least 60%, or at least 70%, or at least 80%, or at least 90%) of the amount of the protein that is normally expressed in the cell type of a healthy individual. For example, in some embodiments, the expressed protein is a protein that is normally expressed in a cell type within the body, and the therapeutically effective amount of the mRNA is an amount that produces a similar level of expression as observed in a healthy individual in an individual with aberrant expression of the protein (i.e., protein deficient individual). Suitable assays for measuring the expression of an mRNA or protein include, but are not limited to dot blots, Northern blots, in situ hybridization, ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

The term “gene” refers to a nucleic acid (e.g., DNA or RNA) sequence that comprises partial length or entire length coding sequences necessary for the production of a polypeptide or precursor polypeptide.

The term “gene product,” as used herein, refers to a product of a gene such as a RNA transcript or a polypeptide.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heteroalkylenyl” or “heteroalkylene” as used herein refers to a bivalent heteroalkyl radical (e.g., —NH—CH₂CH₂—NH—). In certain embodiments, the term may be regarded as a divalent radical formed by the removal of two hydrogen atoms from one or more atoms of a heteroalkyl moiety, wherein the hydrogen atoms may be removed from the same or different atoms, and wherein the atoms may be carbon or a heteroatom.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl, C₂-C₃ heterocycloalkyl, C₂-C₄ heterocycloalkyl, C₂-C₈ heterocycloalkyl, C₂-C₆ heterocycloalkyl, C₂-C₇ heterocycloalkyl, C₂-C₈ heterocycloalkyl, C₂-C₉ heterocycloalkyl, C₂-C₁₀ heterocycloalkyl, C₂-C₁₁ heterocycloalkyl, and the like, up to and including a C₂₋₁₄5 heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocycloalkylene” or “heterocycloalkylenyl” as used herein refers to a bivalent saturated cycloalkyl radical

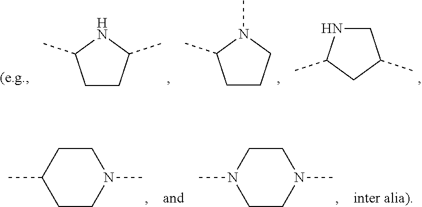

In certain embodiments, the term may be regarded as a product of removal of two hydrogen atoms from the corresponding heterocycloalkane (e.g., piperidine) by removal of two hydrogen atoms from the same

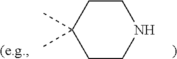

different

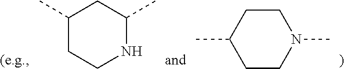

carbon atom(s) and/or heteroatom(s).

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

As used herein, the term “host cell” relates to a vertebrate cell. Preferably, the cell is a mammalian cell, more preferably, a mouse, rat, cat, dog, hamster, guinea pig, sheep, goat, pig, cattle, or horse cell. Still more preferably, the host cell is a primate cell. Most preferably, the host cell is a human cell. Preferably, the host cell comprises at least one therapeutic polynucleotide. Preferably, the host cell is a cell with a doubling time of more than 30 days, more preferably more than 90 days, even more preferably more than 180 days. Most preferably, said cell is a non-regenerating cell of a subject, preferably a pancreas cell, a lung cell, a heart cell, or a nerve cell, preferably of the central nervous system. Preferably, the host cell comprises a therapeutic polynucleotide.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

In general, when referring to “identity,” “homology,” or “similarity” between two different sequences, “identity,” “homology,” or “similarity” is that of an “aligned” sequence. Determined in relation to. An “aligned” sequence or “alignment” refers to a plurality of nucleic acid or protein (amino acid) sequences that often contain corrections for missing or additional bases or amino acids compared to the reference sequence.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “linker” as used herein refers to a divalent chemical moiety comprising a covalent bond or a chain of atoms that covalently conjugates one compound with another. Linkers include one or more divalent radicals, which may be arranged in any order, including but not limited to alkynylenyl, alkenylenyl, heteroalkylenyl (e.g., repeating alkyloxy units such as polyethyleneoxy (PEG) and polymethyleneoxy; and/or alkylamino units such as polyethyleneamino), heteroalkenylenyl, cycloalkylenyl, heterocycloalkylenyl, arylenyl, heteroarylenyl moieties, or one or more —O—, —NH—, —NCH₃—, —C(═O)—, —C(═O)O—, —OC(═O)—, —OC(═O)—, —NHC(═O)—, —C(═O)NH— moieties, and the like, or diacid ester and/or diamide moieties (e.g., succinate, succinimide, diglycolate, malonate, and/or caproamide). In certain embodiments, the linker comprises a linear arrangement of 1 to 100 or more atoms, including about 1 to about 75 atoms, 1 to about 50 atoms, 1 to about 25 atoms, or about 1 to 10 atoms. In certain embodiments, the linker comprises a polyethylene glycol linker containing from 1 to 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5 ethylene glycol units which may be further linked through amide groups, amino acids or other moieties compatible with polyethylene glycol groups. In certain embodiments, the link is cleavable and/or labile. In certain embodiments, the linker is non-cleavable and/or non-labile. Non-limiting examples of cleavable linkers include disulfides, hydrazones, peptides, or thioethers.

The term “local delivery,” as used herein, refers to delivery of an active agent or therapeutic agent such as a messenger RNA directly to a target site within an organism. For example, an agent can be locally delivered by direct injection into a disease site such as a tumor or other target site such as a site of inflammation or a target organ such as the liver, heart, pancreas, kidney, and the like.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (Pl, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mal. Cell. Probes, 8:91-98 (1994)).

As used herein, the term “nucleic acid” includes any oligonucleotide or polynucleotide, with fragments containing up to 60 nucleotides generally termed oligonucleotides, and longer fragments termed polynucleotides. In particular embodiments, oligonucleotides of the disclosure are from about 15 to about 60 nucleotides in length. Nucleic acid may be administered alone in the lipid particles of the disclosure, or in combination (e.g., co-administered) with lipid particles of the disclosure comprising peptides, polypeptides, or small molecules such as conventional drugs. In other embodiments, the nucleic acid may be administered in a viral vector.

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

These control sequences are “operably linked” coding sequence. As used herein, the term “operably linked” refers to an expression control sequence that is close to a gene of interest and an expression control that acts trans or distantly to control the gene of interest. Refers to both with an array.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate: agar: buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985. Easton, PA), which is incorporated herein by reference.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “therapeutic polynucleotide” is used in a broad sense and relates to any polynucleotide introduced into a cell or a subject, preferably into an isolated cell, for the purpose of ameliorating a disease or disorder or the symptoms accompanied therewith and/or for retaining health for at least a certain period of time. Accordingly, the term includes polynucleotides introduced into a cell or subject to induce a change in genome structure, gene expression and/or metabolism of said cell, including, e.g., preferably, introducing a gene therapy vector, as well as introducing a vector into somatic cells to induce formation of stem cells, or into stem cells, in particular pluripotent stem cells, to induce or enhance proliferation and/or differentiation. In certain embodiments, the therapeutic polynucleotide comprises DNA. In certain embodiments, the therapeutic polynucleotide comprises RNA.

Preferably, the therapeutic polynucleotide is a polynucleotide administered within the last 50 years, preferably the last 10 years, more preferably the last 5 years before the method of the present invention is applied. Preferably, the therapeutic polynucleotide is a polynucleotide comprising a viral sequence, more preferably a sequence from a virus for which integration into a host genome is a part of the life cycle or is known to occur at a medically relevant frequency. Thus, preferably, the therapeutic polynucleotide is a sequence of a retrovirus, of an adenovirus, adeno-associated virus, or the like. More preferably, the therapeutic polynucleotide is a polynucleotide comprising a vector sequence known to be maintained extrachromosomally, preferably as an episome. Preferably, a “sequence of a virus” is an incomplete genome of a virus or a variant thereof, e.g., preferably, a sequence comprising viral terminal repeats as the only viral sequences. Accordingly, preferably, the therapeutic polynucleotide is a non-naturally occurring polynucleotide, i.e. preferably, is an artificial polynucleotide. Thus, preferably, the therapeutic polynucleotide is a recombinant polynucleotide. More preferably, the therapeutic polynucleotide is a polynucleotide comprising nucleic acid sequences originating from at least one, more preferably of at least two species different from the species of said host cell. Preferably, the therapeutic polynucleotide comprises at least 25 nucleotides of heterologous sequence, more preferably at least 50 nucleotides, still more preferably at least 100, most preferably at least 250 nucleotides of heterologous sequence, wherein the term “heterologous polynucleotide” is understood by the skilled person and relates to a polynucleotide the nucleic acid sequence of which is derived from a species non-identical to the species of the host cell carrying said polynucleotide. Preferably, the therapeutic polynucleotide is not integrated into the genome of the host cell, more preferably is present in the host cell as an episome and/or the therapeutic polynucleotide is integrated into the genome of the host cell; i.e., preferably, the therapeutic polynucleotide is covalently linked to a chromosome of said host cell, preferably is contiguous with a chromosome of said host cell.

The terms “treat,” “treating” and “treatment,” as used herein, means reducing the frequency or severity with which symptoms of a disease or condition are experienced by a subject by virtue of administering an agent or compound to the subject.

Method of Delivering Compounds into Cells

As described elsewhere herein, the present study developed a series of organic compounds (also referred to as “the compounds herein”) that can enter, for example, endothelial cells in a cell-type specific manner. Bifunctional conjugates including small molecule pharmaceutical compounds and the compounds herein were confirmed to maintain both the native pharmacological properties of the pharmaceutical compounds and the cell-type specific delivery of the compounds herein.

This cell-type specific receptiveness to the compounds herein and drug-conjugates thereof was found to be caused by the cell-type specific expression of a cell surface transport protein in endothelial cells: Slco1a4 in mice and SLCO1A2 in humans.

Notably, the introduction of either Slco1a4 or SLCO1A2 into cell-types that do not naturally express either of these proteins was confirmed to confer receptiveness to both the compounds herein and the drug-conjugates thereof. This confirms that cells, such as cells in a subject can be genetically engineered to become receptive to the compounds herein or the drug-conjugates thereof, which would allow the specific targeting of the engineered cells with the compounds or drug-conjugates herein.

Accordingly, in some aspects, the present study is directed to a method of delivering a compound into a cell. In some embodiment, the method is a gene therapy method.

In some embodiments, the method includes expressing Slco1a4 or SLCO1A2 protein in a cell, and contacting the cell with a compound or a drug-conjugate herein.

In some embodiments, Slco1a4 is the polypeptide having the sequence set forth in SEQ ID NO:1 below. In some embodiments, Slco1a4 includes all the variants, such as splicing variants, of the polypeptide having the sequence set forth in SEQ ID NO:1. In some embodiments, Slco1a4 includes all the mRNA molecules encoding the polypeptides of this paragraph. In some embodiments, Slco1a4 includes the gene encoding the polypeptides and the mRNA molecules of this paragraph, as well as all the genes that occupies the same allele in the mouse genome.

Solute carrier organic anion transporter family
member 1A4 (Slco1a4)[Mus musculus],
NP_001342506.1
(SEQ ID NO: 1)
MGKSEKEVATHGVRCFSKIKAFLLALTCAYVSKSLSGTYMNSMLTQIERQ
FGIPTSVVGLINGSFEIGNLLLIIFVSYFGTKLHRPIMIGVGCAVMGLGC
FLISIPHFLMGRYEYETTILPTSNLSSNSFVCTENRTQTLKPTQDPTECV
KEMKSLMWIYVLVGNIIRGMGETPIMPLGISYIEDFAKSENSPLYIGILE
TGMTIGPLIGLLLGSSCANIYVDTGSVNTDDLTITPTDTRWVGAWWIGFL
VCAGVNILTSIPFFFFPKTLLKEGLQDNGDGTENAKEEKHREKIKEENRG
ITKDFFLFMKSLSCNPIYMIFILISVIQVNAFINSFTFMPKYLEQQYGKS
TAEIVFLMGLYMLPPICLGYLIGGLIMKKFKITVKKAAYIGFWLSLTEYL
LSFVSYIMTCDNFPVAGLTTSYEGVQHPLYVENNVLADCNTKCSCLTNTW
DPVCGDNGLSYMSACLAGCEKSVGTGTNMVFQNCSCIQSSGNASAVLGLC
DKGPECANKLQYFLIISIIGCFIFSLGAIPGYMVLLRCMKSEEKSLGVGL
HTFCMRILGGIPAPIYFGALIDRTCLHWGTLKCGEPGACRMYDINSFRRI
YLGLPAALRGASFLPALFILILMRKFQFPGDIDSSDTDPAEMKLTAKESK
CTNVHRSPTMQNDGERKTKL

In some embodiments, SLCO1A2 is the polypeptide having the sequence set forth in SEQ ID NO:2 below. In some embodiments, SLCO1A2 includes all the variants, such as splicing variants, of the polypeptide having the sequence set forth in SEQ ID NO:2. In some embodiments, SLCO1A2 includes all the mRNA molecules encoding the polypeptides of this paragraph. In some embodiments, SLCO1 A2 includes the gene encoding the polypeptides and the mRNA molecules of this paragraph, as well as all the genes that occupies the same allele in the human genome.

Solute carrier organic anion transporter family
member 1A2 isoform 1 (SLCO1A2, isoform 1) [Homo
sapiens]
(SEQ ID NO: 2)
MGETEKRIETHRIRCLSKLKMFLLAITCAFVSKTLSGSYMNSMLTQIERQ
FNIPTSLVGFINGSFEIGNLLLIIFVSYFGTKLHRPIMIGIGCVVMGLGC
FLKSLPHFLMNQYEYESTVSVSGNLSSNSFLCMENGTQILRPTQDPSECT
KEVKSLMWVYVLVGNIVRGMGETPILPLGISYIEDFAKFENSPLYIGLVE
TGAIIGPLIGLLLASFCANVYVDTGFVNTDDLIITPTDTRWVGAWWFGFL
ICAGVNVLTAIPFFFLPNTLPKEGLETNADIIKNENEDKQKEEVKKEKYG
ITKDFLPFMKSLSCNPIYMLFILVSVIQFNAFVNMISFMPKYLEQQYGIS
SSDAIFLMGIYNLPPICIGYIIGGLIMKKFKITVKQAAHIGCWLSLLEYL
LYFLSFLMTCENSSVVGINTSYEGIPQDLYVENDIFADCNVDCNCPSKIW
DPVCGNNGLSYLSACLAGCETSIGTGINMVFQNCSCIQTSGNSSAVLGLC
DKGPDCSLMLQYFLILSAMSSFIYSLAAIPGYMVLLRCMKSEEKSLGVGL
HTFCTRVFAGIPAPIYFGALMDSTCLHWGTLKCGESGACRIYDSTTFRYI
YLGLPAALRGSSFVPALIILILLRKCHLPGENASSGTELIETKVKGKENE
CKDIYQKSTVLKDDELKTKL

### Compounds and Drug Conjugates

In one aspect, the present disclosure provides certain drug conjugates.

In one aspect, the present disclosure provides a compound of formula (I), or a salt, solvate, tautomer, geometric isomer, or isotopologue thereof, and any combinations thereof:

wherein:

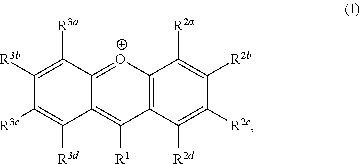

R¹ is

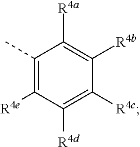

R^{2a}, R^{2b}, R^{2c}, and R^{2d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B},
   wherein two vicinal substituents selected from the group consisting of R^{2a}, R^{2b}, R^{2c}, and R^{2d} can combine with the atoms to which they are bound to form an optionally substituted C₆-C₁₀ aryl or optionally substituted C₂-C₁₀ heteroaryl, and
   wherein no more than three of R^{2a}, R^{2b}, R^{2c}, and R^{2d} are H;
R^{3a}, R^{3b}, R^{3c}, R^{3d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D},
   wherein two vicinal substituents selected from the group consisting of R^{3a}, R^{3b}, R^{3c}, and R^{3d} can combine with the atoms to which they are bound to form an optionally substituted C₆-C₁₀ aryl or optionally substituted C₂-C₁₀ heteroaryl, and
   wherein no more than three of R^{3a}, R^{3b}, R^{3c}, and R^{3d} are H;
R^{4a}, R^{4b}, R^{4c}, R^{4d}, and R^{4e} are each independently selected from the group consisting of H, optionally substituted C₁-C₆ alkyl, CN, NO₂, S(═O)₂OH, and S(═O)₂R⁵,
   wherein at least one of R^{4a}, R^{4b}, R^{4c}, R^{4d}, and R^{4e} is S(═O)₂OH, and
   wherein at least one of R^{4a}, R^{4b}, R^{4c}, R^{4d}, and R^{4e} is S(═O)₂R⁵;
R⁵ is at least one selected from the group consisting of H, OH, halogen, and

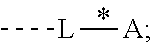

each occurrence of L is independently selected from the group consisting of a bond and a linker;
each occurrence of A is independently H, optionally substituted C₁-C₆ alkyl, an imaging agent, a polymeric macromolecule, or a therapeutic agent;
R^{A} and R^{B}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{A} and R^{B} can independently with one of R^{2a}, R^{2b}, R^{2c}, and R^{2d} to form an optionally substituted C₅-C₈ heterocycloalkyl or C₅-C₈ heterocycloalkenyl; and
R^{C} and R^{D}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{C} and R^{D} can independently with one of R^{3a}, R^{3b}, R^{3c}, and R^{3d} to form an optionally substituted C₅-C₈ heterocycloalkyl or C₅-C₈ heterocycloalkenyl.

In certain embodiments, R^{2a} is H. In certain embodiments, R^{2a} is F. In certain embodiments, R^{2a} is Cl. In certain embodiments, R^{2a} is Br. In certain embodiments, R^{2a} is I.

In certain embodiments, R^{2a} is CH₃. In certain embodiments, R^{2a} is CF₃. In certain embodiments, R^{2a} is SO₃H. In certain embodiments, R² is N(CH₃)₂. In certain embodiments, R^{2a} is N(CH₂CH₃)₂. In certain embodiments, R^{2a} is —(CH₂)₂C(═O)OH. In certain embodiments, R^{2a} is B(OH)₂. In certain embodiments, R^{2a} is

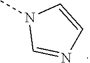

In certain embodiments, R^{2a} is

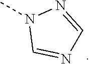

In certain embodiments, R^{2a} is

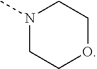

In certain embodiments, R^{2b} is H. In certain embodiments, R^{2b} is F. In certain embodiments, R^{2b} is Cl. In certain embodiments, R^{2b} is Br. In certain embodiments, R^{2b} is I. In certain embodiments, R^{2b} is CH₃. In certain embodiments, R^{2b} is CF₃. In certain embodiments, R^{2b} is SO₃H. In certain embodiments, R^{2b} is N(CH₃)₂. In certain embodiments, R^{2b} is N(CH₂CH₃)₂. In certain embodiments, R^{2b} is —(CH₂)₂C(═O)OH. In certain embodiments, R^{2b} is B(OH)₂. In certain embodiments, R^{2b} is

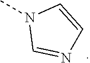

In certain embodiments, R^{2b} is

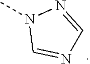

In certain embodiments, R^{2b} is

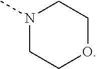

In certain embodiments, R^{2e} is H. In certain embodiments, R^{2e} is F. In certain embodiments, R^{2e} is Cl. In certain embodiments, R^{2e} is Br. In certain embodiments, R^{2e} is I. In certain embodiments, R^{2e} is CH₃. In certain embodiments, R^{2e} is CF₃. In certain embodiments, R^{2e} is SO₃H. In certain embodiments, R^{2e} is N(CH₃)₂. In certain embodiments. R^{2e} is N(CH₂CH₃)₂. In certain embodiments, R^{2e} is —(CH₂)₂C(═O)OH. In certain embodiments, R^{2e} is B(OH)₂. In certain embodiments, R^{2e} is

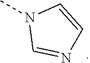

In certain embodiments, R^{2e} is

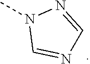

In certain embodiments, R^{2e} is

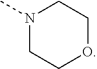

In certain embodiments, R^{2d} is H. In certain embodiments, R^{2d} is F. In certain embodiments, R^{2d} is Cl. In certain embodiments, R^{2d} is Br. In certain embodiments, R^{2d} is I. In certain embodiments, R^{2d} is CH₃. In certain embodiments, R^{2d} is CF₃. In certain embodiments, R^{2d} is SO₃H. In certain embodiments, R^{2d} is N(CH₃)₂. In certain embodiments, R^{2d} is N(CH₂CH₃)₂. In certain embodiments, R^{2d} is —(CH₂)₂C(═O)OH. In certain embodiments R^{2d} is B(OH)₂. In certain embodiments, R^{2d} is

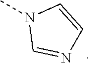

In certain embodiments, R^{2d} is

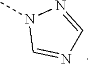

In certain embodiments, R^{2d} is

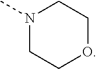

In certain embodiments, R^{3a} is H. In certain embodiments, R^{3a} is F. In certain embodiments, R^{3a} is Cl. In certain embodiments, R^{3a} is Br. In certain embodiments, R^{3a} is I. In certain embodiments, R^{3a} is CH₃. In certain embodiments, R^{3a} is CF₃. In certain embodiments, R^{3a} is SO₃H. In certain embodiments, R^{3a} is N(CH₃)₂. In certain embodiments, R^{3a} is N(CH₂CH₃)₂. In certain embodiments, R^{3a} is —(CH₂)₂C(═O)OH. In certain embodiments, R^{3a} is B(OH)₂. In certain embodiments, R^{3a} is

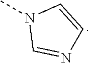

In certain embodiments, R^{3a} is

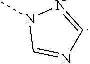

In certain embodiments, R^{3a} is

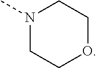

In certain embodiments, R^{3b} is H. In certain embodiments, R^{3b} is F. In certain embodiments, R^{3b} is Cl. In certain embodiments, R^{3b} is Br. In certain embodiments, R^{3b} is I. In certain embodiments, R^{3b} is CH₃. In certain embodiments, R^{3b} is CF₃. In certain embodiments, R^{3b} is SO₃H. In certain embodiments, R^{3b} is N(CH₃)₂. In certain embodiments, R^{3b} is N(CH₂CH₃)₂. In certain embodiments, R^{3b} is —(CH₂)₂C(═O) OH. In certain embodiments, R^{3b} is B(OH)₂. In certain embodiments, R^{3b} is

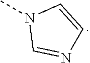

In certain embodiments, R^{3b} is

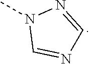

In certain embodiments, R^{3b} is

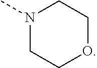

In certain embodiments, R^{3c} is H. In certain embodiments, R^{3c} is F. In certain embodiments, R^{3c} is Cl. In certain embodiments, R^{3c} is Br. In certain embodiments, R^{3c} is I. In certain embodiments, R^{3c} is CH₃. In certain embodiments, R^{3c} is CF₃. In certain embodiments, R^{3c} is SO₃H. In certain embodiments, R^{3c} is N(CH₃)₂. In certain embodiments, R^{3c} is N(CH₂CH₃)₂. In certain embodiments, R^{3c} is —(CH₂)₂C(═O) OH. In certain embodiments, R^{3c} is B(OH)₂. In certain embodiments, R^{3c} is

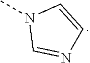

In certain embodiments, R^{3c} is

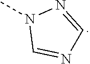

In certain embodiments, R^{3c} is

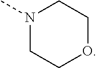

In certain embodiments, R^{3d} is H. In certain embodiments, R^{3d} is F. In certain embodiments, R^{3d} is Cl. In certain embodiments, R^{3d} is Br. In certain embodiments, R^{3d} is I. In certain embodiments, R^{3d} is CH₃. In certain embodiments, R^{3d} is CF₃. In certain embodiments, R^{3d} is SO₃H. In certain embodiments, R^{3d} is N(CH₃)₂. In certain embodiments, R^{3d} is N(CH₂CH₃)₂. In certain embodiments, R^{3d} is —(CH₂)₂C(═O)OH. In certain embodiments, R^{3d} is B(OH)₂. In certain embodiments, R^{3d} is

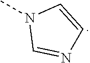

In certain embodiments, R^{3d} is

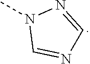

In certain embodiments, R^{3d} is

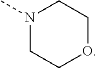

In certain embodiments, R^{2a} is H, R^{2b} is Br, R^{2c} is H, and R^{2d} is H. In certain embodiments, R^{2a} is H, R^{2b} is H, R^{2c} is Br, and R^{2d} is H. In certain embodiments, R^{2a} is H. R^{2b} is N(CH₃)₂, R^{2c} is H, and R^{2d} is H. In certain embodiments, R^{2a} is H, R^{2b} is N(CH₂CH₃)₂, R^{2c} is H, and R^{2d} is H.

In certain embodiments, R^{3a} is H, R^{3b} is NO₂, R^{3c} is H, and R^{3d} is H. In certain embodiments, R^{3a} is H, R^{3b} is CH₃, R^{3c} is H, and R^{3d} is H. In certain embodiments, R^{3a} is H, R^{3b} is

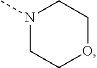

R^{3c} is H, and R^{3d} is H. In certain embodiments, R^{3a} is H, R^{3b} is H, R^{3c} is

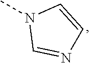

and R^{3d} is H. In certain embodiments, R^{3a} is H, R^{3b} is H, R^{3c} is SO₃H, and R^{3d} is H. In certain embodiments, R^{3a} is H, R^{3b} is H, R^{3c} is

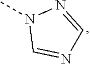

and R^{3d} is H. In certain embodiments, R^{3a} is H, R^{3b} is N(CH₃)₂, R^{3c} is H, and R^{3d} is H. In certain embodiments, R^{3a} is H, R^{3b} is N(CH₂CH₃)₂, R^{3c} is H, and R^{3d} is H.

In certain embodiments, the compound of formula (I) is:

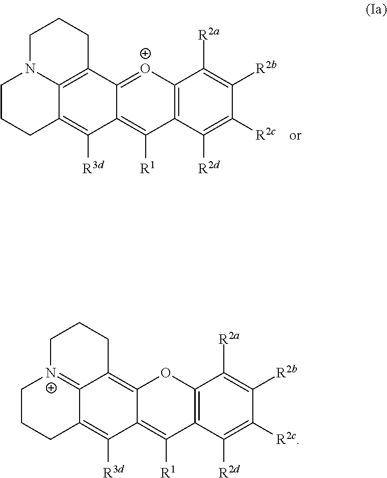

In certain embodiments, R^{3d} is H.

In certain embodiments, R^{2a} is H, R^{2b} is Br, R^{2c} is H, and R^{2d} is H. In certain embodiments, R^{2a} is H, R^{2b} is F, R^{2c} is H, and R^{2d} is H. In certain embodiments, R^{2a} is H, R^{2b} is Cl, R^{2c} is H, and R^{2d} is H. In certain embodiments, R^{2a} is H, R^{2b} is I, R^{2c} is H, and R^{2d} is H.

In certain embodiments, R^{2a} is Br, R^{2b} is H, R^{2c} is H, and R^{2d} is H. In certain embodiments, R^{2a} is H, R^{2b} is H, R^{2c} is Br, and R^{2d} is H. In certain embodiments, R^{2a} is Br, R^{2b} is H, R^{2c} is Br, and R^{2d} is H. In certain embodiments, R^{2a} is H, R^{2b} is F, R^{2c} is F, and R^{2d} is H. In certain embodiments, R^{2a} is H, R^{2b} is F, R^{2c} is Cl, and R² is H. In certain embodiments, R^{2a} is H, R^{2b} is F, R^{2c} is Br, and R^{2d} is H. In certain embodiments, R^{2a} is H, R^{2b} is Cl, R^{2c} is F, and R^{2d} is H. In certain embodiments, R^{2a} is H, R^{2b} is Cl, R^{2c} is Cl, and R^{2d} is H. In certain embodiments, R^{2a} is H, R^{2b} is Cl, R^{2c} is Br, and R^{2d} is H. In certain embodiments, R^{2a} is H, R^{2b} is Br, R^{2c} is F, and R^{2d} is H. In certain embodiments, R^{2a} is H, R^{2b} is Br, R^{2c} is Cl, and R^{2d} is H. In certain embodiments, R^{2a} is H, R^{2b} is I, R^{2c} is Cl, and R^{2d} is H. In certain embodiments, R^{2a} is H, R^{2b} is CF₃, R^{2c} is Br, and R^{2d} is H. In certain embodiments, R^{2a} is F, R^{2b} is F, R^{2c} is H, and R^{2d} is H. In certain embodiments, R^{2a} is F, R^{2b} is Cl, R^{2c} is H, and R^{2d} is H. In certain embodiments, R^{2a} is F, R^{2b} is Br, R^{2c} is H, and R^{2d} is H. In certain embodiments, R^{2a} is Cl, R^{2b} is F, R^{2c} is H, and R^{2d} is H. In certain embodiments, R^{2a} is Cl, R^{2b} is Cl, R^{2c} is H, and R^{2d} is H. In certain embodiments, R^{2a} is H, R^{2b} is NO₂, R^{2c} is H, and R^{2d} is H. In certain embodiments, R^{2a} is H, R^{2b} is N(CH₃)₂, R^{2c} is H, and R^{2d} is H. In certain embodiments, R^{2a} is H, R^{2b} is —(CH₂)₂C(═O)OH, R^{2c} is H, and R^{2d} is H. In certain embodiments, R^{2a} is H, R^{2b} is CH₃, R^{2c} is H, and R^{2d} is H. In certain embodiments, R^{2a} is H, R^{2b} is B(OH)₂, R^{2c} is H, and R^{2d} is H.

In certain embodiments, the compound of formula (I) is:

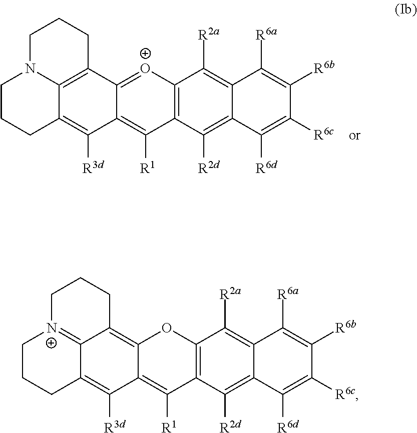

wherein R^{6a}, R^{6b}, R^{6c}, and R^{6d}, if present, are each independently selected from the group consisting of H, C₁-C₆ alkyl, C₁-C₆ haloalkyl, halogen, CN, and NO₂.

In certain embodiments, R^{3d} is H.

In certain embodiments, R^{2a} is H, R^{6a} is H, R^{6b} is H, R^{6c} is Br, R^{6d} is H, and R^{2d} is H.

In certain embodiments, R^{2a} is Br, R^{6a} is H, R^{6b} is H, R^{6c} is H, R^{6d} is H, and R^{2d} is H.

In certain embodiments, at least one of R^{4a}, R^{4b}, R^{4c}, R^{4d}, and R^{4e} is H. In certain embodiments, at least two of R^{4a}, R^{4b}, R^{4c}, R^{4d}, and R^{4e} are H. In certain embodiments, at least three of R^{4a}, R^{4b}, R^{4c}, R^{4d}, and R^{4e} are H. In certain embodiments, four of R^{4a}, R^{4b}, R^{4c}, R^{4d}, and R^{4e} are H.

In certain embodiments, the compound of formula (I) is:

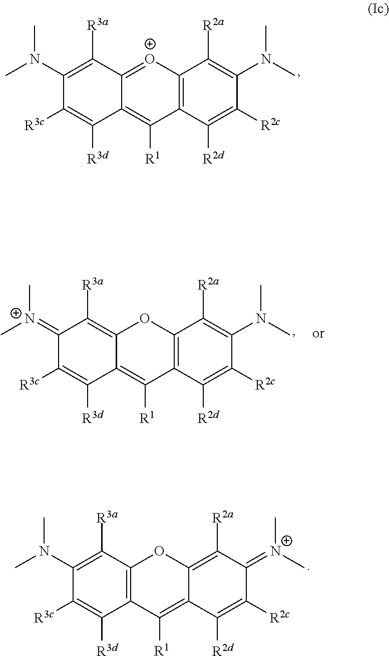

In certain embodiments, R^{3a}, is H, R^{3c} is H, and R^{3d} is H.

In certain embodiments, R^{2a} is H, R^{2c} is H, and R^{2d} is H.

In certain embodiments, at least one of R^{4a}, R^{4b}, R^{4c}, R^{4d}, and R^{4e} is H. In certain embodiments, at least two of R^{4a}, R^{4b}, R^{4c}, R^{4d}, and R^{4e} are H. In certain embodiments, at least three of R^{4a}, R^{4b}, R^{4c}, R^{4d}, and R^{4e} are H. In certain embodiments, four of R^{4a}, R^{4b}, R^{4c}, R^{4d}, and R^{4e} are H.

In certain embodiments, the compound of formula (I) is:

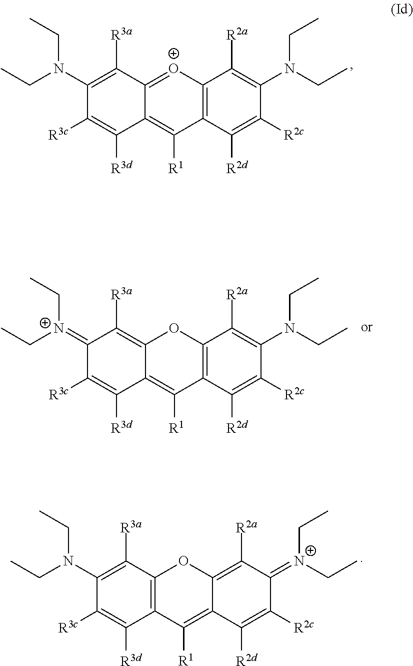

In certain embodiments, R^{3a}, is H, R^{3c} is H, and R^{3d} is H.

In certain embodiments, R^{2a} is H, R^{2c} is H, and R^{2d} is H.

In certain embodiments, at least one of R^{4a}, R^{4b}, R^{4c}, R^{4d}, and R^{4e} is H. In certain embodiments, at least two of R^{4a}, R^{4b}, R^{4c}, R^{4d}, and R^{4e} are H. In certain embodiments, at least three of R^{4a}, R^{4b}, R^{4c}, R^{4d}, and R^{4e} are H. In certain embodiments, four of R^{4a}, R^{4b}, R^{4c}, R^{4d}, and R^{4e} are H.

In certain embodiments, R¹ is

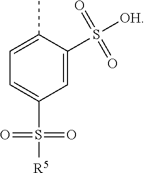

In certain embodiments, R¹ is

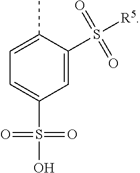

In certain embodiments, L comprises n instances of Z, wherein:
each occurrence of Z is independently selected from the group consisting of —O—, —N(R⁷)—, —S—, —S(═O)—, —S(═O)₂—, —C(═O)—, optionally substituted C₁-C₁₀ alkylenyl, optionally substituted C₁-C₁₀ heteroalkylenyl, optionally substituted C₃-C₈ cycloalkylenyl, optionally substituted C₂-C₈ heterocycloalkylenyl, optionally substituted C₁-C₁₀ alkenylenyl, optionally substituted C₃-C₈ cycloalkenylenyl, optionally substituted C₂-C₈ heterocycloalkenylenyl, optionally substituted C₆-C₁₀ arylenyl, and optionally substituted C₂-C₁₀ heteroarylenyl;
R⁷ is selected from the group consisting of H and C₁-C₆ alkyl; and
n is an integer ranging from 0 to 500.

In certain embodiments, Z is —NH—. In certain embodiments, Z is —S—. In certain embodiments, Z is —O—. In certain embodiments, Z is —(CH₂)₀₋₁₀—. In certain embodiments, Z is —(CH₂)₂O—. In certain embodiments, Z is —O(CH₂)₂—. In certain embodiments. Z is —C(═O)—.

In certain embodiments, Z is

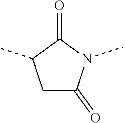

In certain embodiments, Z is

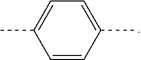

In certain embodiments, Z is

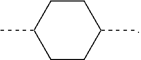

In certain embodiments, L is a bond. In certain embodiments, L is —NH—. In certain embodiments, L is

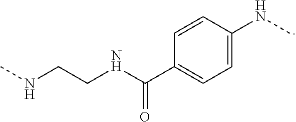

In certain embodiments, L is

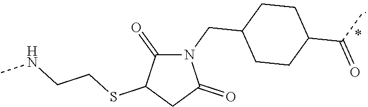

In certain embodiments, A is H. In certain embodiments, A is an imaging agent. In certain embodiments, A is a therapeutic agent.

In certain embodiments, A is a chromophore label. In certain embodiments. A is a fluorophore label. In certain embodiments, A is a fluorescent label. In certain embodiments, A is a bioluminescent label. In certain embodiments, A is a chemiluminescent label. In certain embodiments, A is isotopically labeled. In certain embodiments, a is a polymeric macromolecule.

In certain embodiments, the therapeutic agent is a small molecule. In certain embodiments, the therapeutic agent is a polypeptide. In certain embodiments, the therapeutic agent is a protein. In certain embodiments, the therapeutic agent is an aptamer. In certain embodiments, the compound is a bioconjugate. In certain embodiments, the compound is an immunoconjugate.

In certain embodiments, the small molecule is a compound useful for the treatment of cancer.

In certain embodiments, the therapeutic agent is selected from the group consisting of colchicine, deferoxamine, paclitaxel (taxol), tofacitinib, methotrexate, hydrocortisone, prednisone, triiodothyronine, cyclophosphamide, amphotericin B, vancomycin, doxorubicin, mitoxantrone, imatinib, darunavir, and fosamprenavir.

In certain embodiments, the therapeutic agent comprises at least one modification and/or derivatization. In certain embodiments, the modification and/or derivatization is a modification and/or derivatization which is necessary to for conjugation and/or covalent modification with the linker. In certain embodiments, the modification comprises removal of a carbonyl group from a heteroatom (e.g., deacetylation) or addition of a heteroatom to an aromatic ring (e.g., nucleophilic aromatic substitution or electrophilic aromatic substitution).

In certain embodiments, the therapeutic modification and/or derivatization comprises bond

[symbol]

In certain embodiments A is a polymeric macromolecule. In certain embodiments, the polymeric macromolecule is a compound of formula (III):

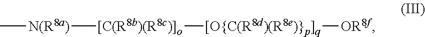

wherein:
R^{8a} and R^{8f} are each independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
each occurrence of R^{8b}, R^{8c}, R^{8d}, and R^{8e} is independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
o is an integer selected from 1, 2, 3, 4, and 5;
p is an integer selected from 1, 2, 3, 4, and 5;
q is an integer ranging from 1 to 500.

In certain embodiments, R^{8a} is H. In certain embodiments, R^{8b} is H. In certain embodiments, R^{8c} is H. In certain embodiments, R^{8d} is H. In certain embodiments, R^{8e} is H. In certain embodiments, R^{8f} is H. In certain embodiments, R^{8f} is Me. In certain embodiments, o is 2. In certain embodiments, p is 2. In certain embodiments, q is an integer ranging from 10 to 500.

In certain embodiments, A is

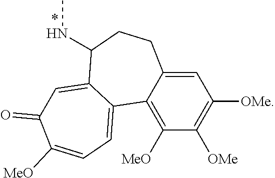

In certain embodiments, A is

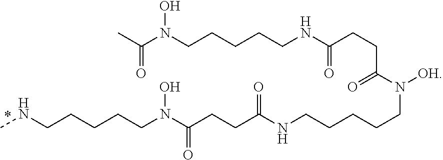

In certain embodiments, A is

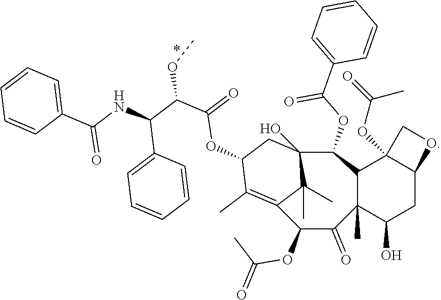

In certain embodiments. A is

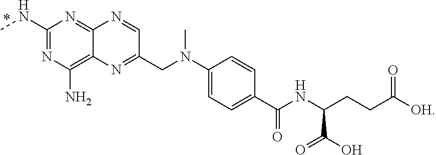

In certain embodiments, A is

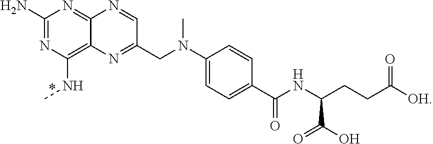

In certain embodiments, A is and

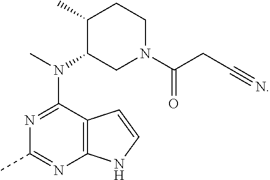

In certain embodiments. A is

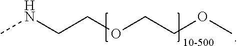

In certain embodiments, R⁵ is

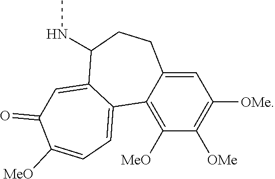

In certain embodiments, R⁵ is

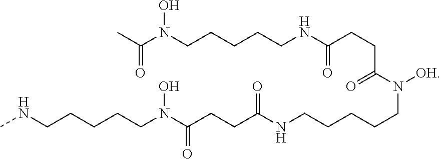

In certain embodiments, R⁵ is

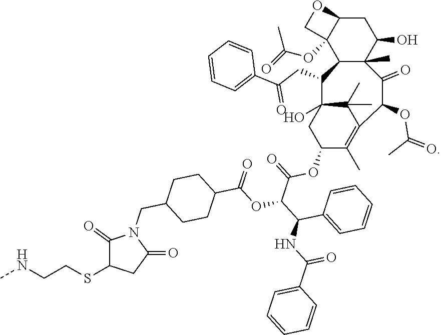

In certain embodiments, R⁵ is

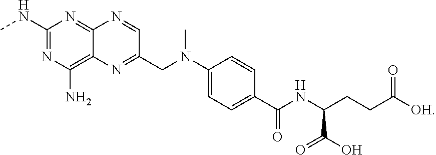

In certain embodiments, R⁵ is

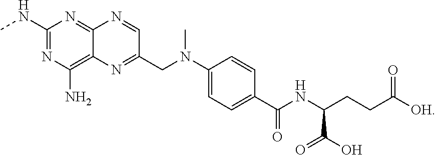

In certain embodiments R⁵ is

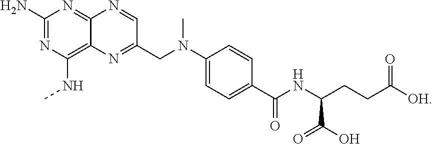

In certain embodiments, R⁵ is

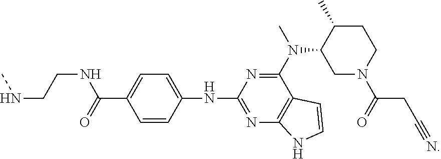

In certain embodiments, R⁵ is

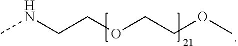

In certain embodiments, R⁵ is

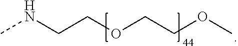

In certain embodiments, R⁵ is

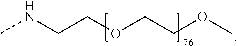

In certain embodiments, R⁵ is

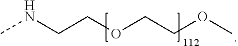

In certain embodiments, R⁵ is

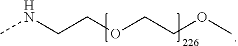

In certain embodiments, R⁵ is

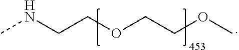

In certain embodiments, each occurrence of optionally substituted alkyl, optionally substituted haloalkyl, optionally substituted cycloalkyl, optionally substituted heterocycloalkyl, optionally substituted heterocycloalkenyl, optionally substituted aryl, optionally substituted heteroaryl, optionally substituted alkylenyl, optionally substituted cycloalkylenyl, optionally substituted heterocycloalkylenyl, optionally substituted alkenylenyl, optionally substituted cycloalkenylenyl, optionally substituted heterocycloalkenylenyl, optionally substituted arylenyl, and optionally substituted heteroarylenyl is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂ OR^{I}, N(R^{I})(R^{II}), C₁-C₆ haloalkoxy, C₃-C₈ halocycloalkoxy, aryl, heteroaryl, (C₁-C₆ alkylenyl)C(═O)N(R^{I})(R^{II}), (C₁-C₆ alkylenyl)C(═O)OR^{I}, O(C₁-C₃ alkylenyl)C(═O)OR^{II}, O(C₁-C₃ alkylenyl)C(═O)N(R^{I})(R^{II}), C(═O)R, C(═O)OR^{I}, OC(═O)R^{I}, OC(═O)OR^{I}, SR^{I}, S(═O)R^{I}, S(═O)₂R^{I}, S(═O)₂N(R^{I})(R^{II}), S(═O)₂NR^{I}C(═O)NHR^{II}, N(R^{I})S(═O)₂R^{II}, N(R^{I})C(═O)R^{II}, and C(═O)NR^{I}R^{II}, wherein R^{I} and R^{II} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

In certain embodiments, the isotopologue thereof comprises at least one radioactive tracer selected from the group consisting of ¹¹C, ¹³N, ¹⁵O, ¹⁸F, ¹²⁴I, ¹³¹I, and ¹³⁵I.

In certain embodiments, the compound is selected from the group consisting of:

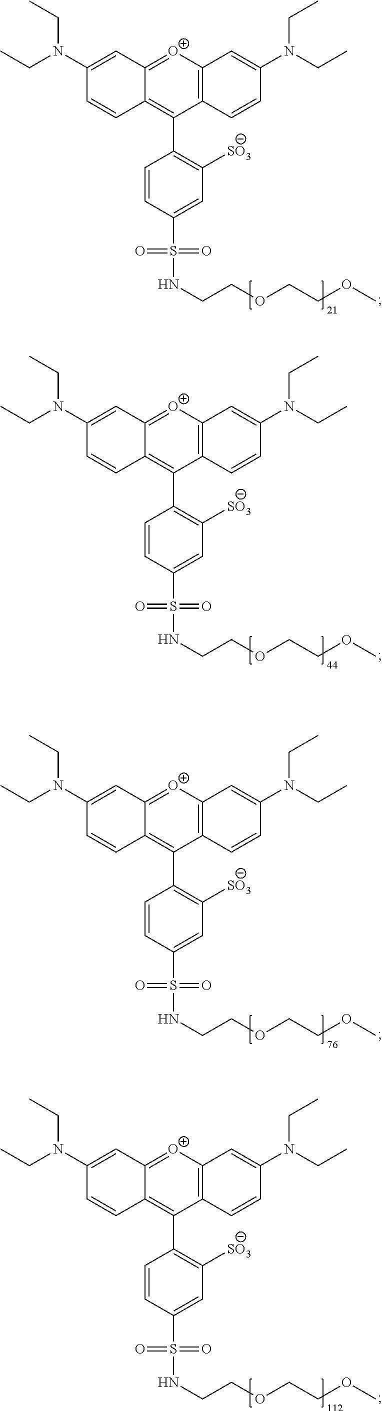

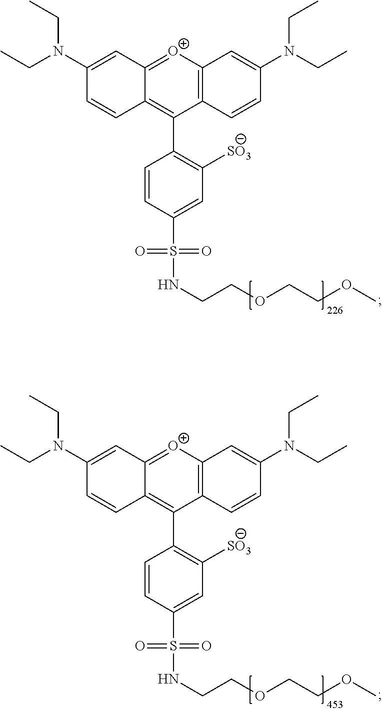

2-(3,6-bis(dimethylamino)xanthylium-9-yl)-5-(N-(1,2,3,10-tetramethoxy-9-oxo-5,6,7,9-tetrahydrobenzo[a]heptalen-7-yl)sulfamoyl)benzenesulfonate;
2-(3,6-bis(diethylamino)xanthylium-9-yl)-5-(N-(1,2,3,10-tetramethoxy-9-oxo-5,6,7,9-tetrahydrobenzo[a]heptalen-7-yl)sulfamoyl)benzenesulfonate;
2-(3,6-bis(dimethylamino)xanthylium-9-yl)-5-(N-(3,14,25-trihydroxy-2,10,13,21,24-pentaoxo-3,9,14,20,25-pentaazatriacontan-30-yl)sulfamoyl)benzenesulfonate;
5-(N-(2-((1-((4-((((1R,2S)-1-benzamido-3-(((2aS,4R,4aR,6S,9R,11R,12R,12aS,12bR)-6,12b-diacetoxy-12-(benzoyloxy)-4,11-dihydroxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-9-yl)oxy)-3-oxo-1-phenylpropan-2-yl)oxy)carbonyl)cyclohexyl)methyl)-2,5-dioxopyrrolidin-3-yl)thio)ethyl)sulfamoyl)-2-(3,6-bis(dimethylamino)xanthylium-9-yl)benzenesulfonate;
(S)-5-(N-(4-amino-6-(((4-((1,3-dicarboxypropyl)carbamoyl)phenyl)(methyl)amino)methyl)pteridin-2-yl)sulfamoyl)-2-(12-bromo-1,2,3,5,6,7-hexahydrochromeno[2,3-f]pyrido[3,2,1-ij]quinolin-4-ium-9-yl)benzenesulfonate;
(R)-5-(N-(2-amino-6-(((4-((1,3-dicarboxypropyl)carbamoyl)phenyl)(methyl)amino)methyl)pteridin-4-yl)sulfamoyl)-2-(12-bromo-1,2,3,5,6,7-hexahydrochromeno[2,3-f]pyrido[3,2,1-ij]quinolin-4-ium-9-yl)benzenesulfonate; and
5-(N-(2-(4-((4-(((3R,4R)-1-(2-cyanoacetyl)-4-methylpiperidin-3-yl)(methyl)amino)-7H-pyrrolo[2,3-d]pyrimidin-2-yl)amino)benzamido)ethyl)sulfamoyl)-2-(6-(diethylamino)-3-(diethyliminio)-3H-xanthen-9-yl)benzenesulfonate.

In another aspect, the present disclosure provides a of formula (II), or a salt, solvate, tautomer, geometric isomer, or isotopologue thereof, and any combinations thereof:

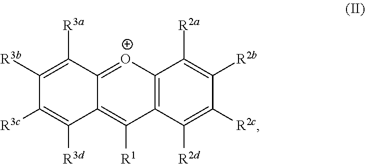

wherein:
R¹ is

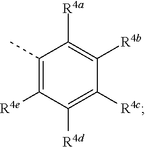

R^{2a}, R^{2b}, R^{2c}, and R^{2d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B},
   wherein two vicinal substituents selected from the group consisting of R^{2a}, R^{2b}, R^{2c}, and R^{2d} can combine with the atoms to which they are bound to form an optionally substituted C₆-C₁₀ aryl or optionally substituted C₂-C₁₀ heteroaryl, and
   wherein no more than three of R^{2a}, R^{2b}, R^{2c}, and R^{2d} are H;
R^{3a}, R^{3b}, R^{3c}, R^{3d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D},
   wherein two vicinal substituents selected from the group consisting of R^{3a}, R^{3b}, R^{3c}, and R^{3d} can combine with the atoms to which they are bound to form an optionally substituted C₆-C₁₀ aryl or optionally substituted C₂-C₁₀ heteroaryl, and
   wherein no more than three of R^{3a}, R^{3b}, R^{3c}, and R^{3d} are H;
R^{4a}, R^{4b}, R^{4c}, R^{4d}, and R^{4e} are each independently selected from the group consisting of H, optionally substituted C₁-C₆ alkyl, CN, NO₂, S(═O)₂OH, and S(═O)₂X,
   wherein at least one of R^{4a}, R^{4b}, R^{4c}, R^{4d}, and R^{4e} is S(═O)₂OH, and/or
   wherein at least one of R^{4a}, R^{4b}, R^{4c}, R^{4d}, and R^{4e} is S(═O)₂X;
X is a halogen:
R^{A} and R^{B}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{A} and R^{B} can independently with one of R^{2a}, R^{2b}, R^{2c}, and R^{2d} to form an optionally substituted C₅-C₈ heterocycloalkyl or C₅-C₈ heterocycloalkenyl; and
R^{C} and R^{D}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{C} and R^{D} can independently with one of R^{3a}, R^{3b}, R^{3e}, and R^{3d} to form an optionally substituted C₅-C₈ heterocycloalkyl or C₅-C₈ heterocycloalkenyl.

In certain embodiments, at least two of R^{4a}, R^{4b}, R^{4c}, R^{4d}, and R^{4e} is S(═O)₂OH, or a salt thereof.

In certain embodiments, R^{2a} is H. In certain embodiments, R^{2a} is F. In certain embodiments, R^{2a} is Cl. In certain embodiments, R^{2a} is Br. In certain embodiments, R^{2a} is I.

In certain embodiments, R^{2a} is CH₃. In certain embodiments, R^{2a} is CF₃. In certain embodiments, R^{2a} is SO₃H. In certain embodiments, R² is N(CH₃)₂. In certain embodiments, R^{2a} is N(CH₂CH₃)₂. In certain embodiments, R^{2a} is —(CH₂)₂C(═O)OH. In certain embodiments, R^{2a} is B(OH)₂. In certain embodiments, R^{2a} is

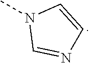

In certain embodiments, R^{2a} is

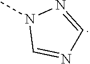

In certain embodiments, R^{2a} is

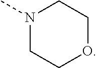

In certain embodiments, R^{2b} is H. In certain embodiments, R^{2b} is F. In certain embodiments, R^{2b} is Cl. In certain embodiments, R^{2b} is Br. In certain embodiments, R^{2b} is I. In certain embodiments, R^{2b} is CH₃. In certain embodiments, R^{2b} is CF₃. In certain embodiments, R^{2b} is SO₃H. In certain embodiments, R^{2b} is N(CH₃)₂. In certain embodiments, R^{2b} is N(CH₂CH₃)₂. In certain embodiments, R^{2b} is —(CH₂)₂C(═O)OH. In certain embodiments, R^{2b} is B(OH)₂. In certain embodiments, R^{2b} is

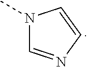

In certain embodiments, R^{2b} is

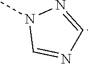

In certain embodiments, R^{2b} is

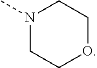

In certain embodiments, R^{2c} is H. In certain embodiments, R^{2c} is F. In certain embodiments, R^{2c} is Cl. In certain embodiments, R^{2c} is Br. In certain embodiments, R^{2e} is I. In certain embodiments, R^{2c} is CH₃. In certain embodiments, R^{2c} is CF₃. In certain embodiments, R^{2e} is SO₃H. In certain embodiments, R^{2e} is N(CH₃)₂. In certain embodiments, R^{2c} is N(CH₂CH₃)₂. In certain embodiments, R^{2c} is —(CH₂)₂C(═O)OH. In certain embodiments, R^{2c} is B(OH)₂. In certain embodiments, R^{2e} is

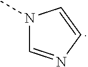

In certain embodiments, R^{2c} is

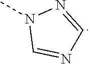

In certain embodiments, R^{2e} is

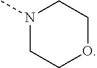

In certain embodiments, R^{2d} is H. In certain embodiments, R^{2d} is F. In certain embodiments, R^{2d} is Cl. In certain embodiments, R^{2d} is Br. In certain embodiments, R^{2d} is I. In certain embodiments, R^{2d} is CH₃. In certain embodiments, R^{2d} is CF₃. In certain embodiments, R^{2d} is SO₃H. In certain embodiments, R^{2d} is N(CH₃)₂. In certain embodiments, R^{2d} is N(CH₂CH₃)₂. In certain embodiments, R^{2d} is —(CH₂)₂C(═O)OH. In certain embodiments R^{2d} is B(OH)₂. In certain embodiments, R^{2d} is

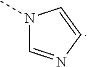

In certain embodiments, R^{2d} is

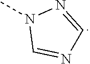

In certain embodiments, R^{2d} is

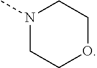

In certain embodiments, R^{3a} is H. In certain embodiments, R^{3a} is F. In certain embodiments, R^{3a} is Cl. In certain embodiments, R^{3a} is Br. In certain embodiments, R^{3a} is I. In certain embodiments, R^{3a} is CH₃. In certain embodiments, R^{3a} is CF₃. In certain embodiments, R^{3a} is SO₃H. In certain embodiments, R^{3a} is N(CH₃)₂. In certain embodiments, R^{3a} is N(CH₂CH₃)₂. In certain embodiments, R^{3a} is —(CH₂)₂C(═O)OH. In certain embodiments, R³ is B(OH)₂. In certain embodiments, R^{3a} is

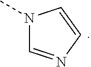

In certain embodiments, R^{3a} is

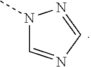

In certain embodiments, R^{3a} is

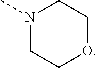

In certain embodiments, R^{3b} is H. In certain embodiments, R^{3b} is F. In certain embodiments, R^{3b} is Cl. In certain embodiments, R^{3b} is Br. In certain embodiments, R^{3b} is I. In certain embodiments, R^{3b} is CH₃. In certain embodiments, R^{3b} is CF₃. In certain embodiments, R^{3b} is SO₃H. In certain embodiments, R^{3b} is N(CH₃)₂. In certain embodiments, R^{3b} is N(CH₂CH₃)₂. In certain embodiments, R^{3b} is —(CH₂)₂C(═O)OH. In certain embodiments, R^{3b} is B(OH)₂. In certain embodiments R^{3b} is

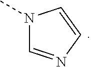

In certain embodiments, R^{3b} is

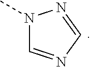

In certain embodiments, R^{3b} is

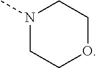

In certain embodiments, R^{3e} is H. In certain embodiments, R^{3e} is F. In certain embodiments, R^{3e} is Cl. In certain embodiments, R^{3e} is Br. In certain embodiments, R^{3e} is I. In certain embodiments, R^{3e} is CH₃. In certain embodiments, R^{3e} is CF₃. In certain embodiments, R^{3e} is SO₃H. In certain embodiments, R^{3e} is N(CH₃)₂. In certain embodiments, R^{3e} is N(CH₂CH₃)₂. In certain embodiments, R^{3e} is —(CH₂)₂C(═O)OH. In certain embodiments, R^{3e} is B(OH)₂. In certain embodiments, R^{3e} is

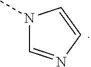

In certain embodiments, R^{3e} is

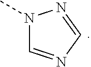

In certain embodiments, R^{3e} is

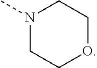

In certain embodiments, R^{3d} is H. In certain embodiments, R^{3d} is F. In certain embodiments, R^{3d} is Cl. In certain embodiments, R^{3d} is Br. In certain embodiments, R^{3d} is I. In certain embodiments, R^{3d} is CH₃. In certain embodiments, R^{3d} is CF₃. In certain embodiments, R^{3d} is SO₃H. In certain embodiments, R^{3d} is N(CH₃)₂. In certain embodiments, R^{3d} is N(CH₂CH₃)₂. In certain embodiments, R^{3d} is —(CH₂)₂C(═O)OH. In certain embodiments R^{3d} is B(OH)₂. In certain embodiments, R^{3d} is

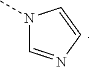

In certain embodiments, R^{3d} is

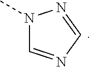

In certain embodiments, R^{3d} is

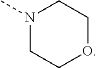

In certain embodiments, R^{2a} is H, R^{2b} is Br, R^{2c} is H, and R^{2d} is H. In certain embodiments, R^{2a} is H, R^{2b} is H, R^{2c} is Br, and R^{2d} is H. In certain embodiments, R^{2a} is H, R^{2b} is N(CH₃)₂, R^{2c} is H, and R^{2d} is H. In certain embodiments, R^{2a} is H, R^{2b} is N(CH₂CH₃)₂, R^{2c} is H, and R^{2d} is H.

In certain embodiments, R^{3a} is H, R^{3b} is NO₂, R^{3c} is H, and R^{3d} is H. In certain embodiments, R^{3a} is H, R^{3b} is CH₃, R^{3c} is H, and R^{3d} is H. In certain embodiments, R^{3a} is H. R^{3b} is

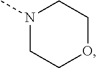

R^{3c} is H, and R^{3d} is H. In certain embodiments, R^{3a} is H, R^{3b} is H, R^{3e} is

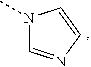

and R^{3d} is H. In certain embodiments, R^{3a} is H, R^{3b} is H, R^{3e} is SO₃H, and R^{3d} is H. In certain embodiments, R^{3a} is H, R^{3b} is H, R^{3e} is

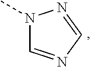

and R^{3d} is H. In certain embodiments, R^{3a} is H, R^{3b} is N(CH₃)₂, R^{3e} is H, and R^{3d} is H. In certain embodiments, R^{3a} is H, R^{3b} is N(CH₂CH₃)₂, R^{3e} is H, and R^{3d} is H.

In certain embodiments, the compound of formula (II) is:

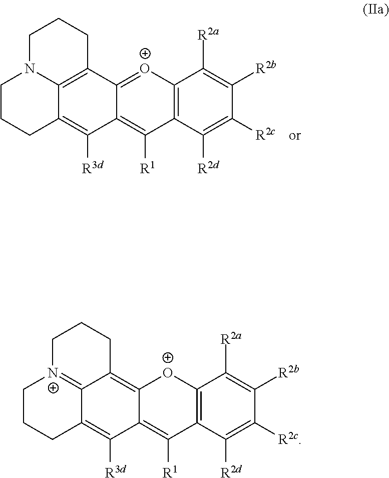

In certain embodiments, R^{3d} is H.

In certain embodiments, R^{2a} is H, R^{2b} is Br, R^{2c} is H, and R^{2d} is H. In certain embodiments, R^{2a} is H, R^{2b} is F, R^{2c} is H, and R^{2d} is H. In certain embodiments, R^{2a} is H, R^{2b} is Cl, R^{2c} is H, and R^{2d} is H. In certain embodiments, R^{2a} is H, R^{2b} is I, R^{2c} is H, and R^{2d} is H. In certain embodiments, R^{2a} is Br, R^{2b} is H, R^{2c} is H, and R^{2d} is H. In certain embodiments, R^{2a} is H, R^{2b} is H, R^{2c} is Br, and R^{2d} is H. In certain embodiments, R^{2a} is Br, R^{2b} is H, R^{2c} is Br, and R^{2d} is H. In certain embodiments, R^{2a} is H, R^{2b} is F, R^{2c} is F, and R^{2d} is H. In certain embodiments, R^{2a} is H, R^{2b} is F, R^{2c} is Cl, and R^{2d} is H. In certain embodiments, R^{2a} is H, R^{2b} is F, R^{2c} is Br, and R^{2d} is H. In certain embodiments, R^{2a} is H, R^{2b} is Cl, R^{2c} is F, and R^{2d} is H. In certain embodiments, R^{2a} is H, R^{2b} is Cl, R^{2C} is Cl, and R^{2d} is H. In certain embodiments, R^{2a} is H, R^{2b} is Cl, R^{2c} is Br, and R^{2d} is H. In certain embodiments, R^{2a} is H, R^{2b} is Br, R^{2e} is F, and R^{2d} is H. In certain embodiments, R^{2a} is H, R^{2b} is Br, R^{2c} is Cl, and R^{2d} is H. In certain embodiments, R^{2a} is H, R^{2b} is I, R^{2c} is Cl, and R^{2d} is H. In certain embodiments, R^{2a} is H, R^{2b} is CF₃, R^{2c} is Br, and R^{2d} is H. In certain embodiments, R^{2a} is F, R^{2b} is F, R^{2c} is H, and R^{2d} is H. In certain embodiments, R^{2a} is F, R^{2b} is Cl, R^{2c} is H, and R^{2d} is H. In certain embodiments, R^{2a} is F, R^{2b} is Br, R^{2c} is H, and R^{2d} is H. In certain embodiments, R^{2a} is Cl, R^{2b} is F, R^{2c} is H, and R^{2d} is H. In certain embodiments, R^{2a} is Cl, R^{2b} is Cl, R^{2c} is H, and R^{2d} is H. In certain embodiments, R^{2a} is H, R^{2b} is NO₂, R^{2c} is H, and R^{2d} is H. In certain embodiments, R^{2a} is H, R^{2b} is N(CH₃)₂, R^{2c} is H, and R^{2d} is H. In certain embodiments, R^{2a} is H, R^{2b} is —(CH₂)₂C(═O)OH, R^{2c} is H, and R^{2a} is H. In certain embodiments, R^{2a} is H, R^{2b} is CH₃, R^{2c} is H, and R^{2d} is H. In certain embodiments, R^{2a} is H, R^{2b} is B(OH)₂, R^{2c} is H, and R^{2d} is H.

In certain embodiments, the compound of formula (II) is:

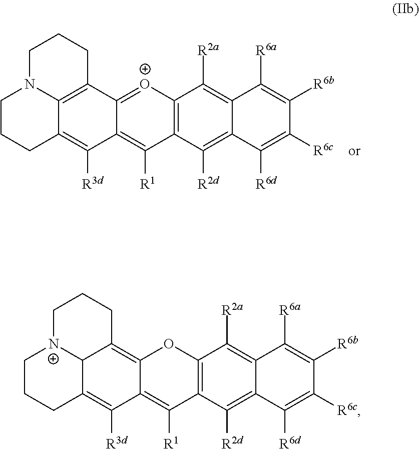

wherein R^{6a}, R^{6b}, R^{6c}, and R^{6d}, if present, are each independently selected from the group consisting of H C₁-C₆ alkyl, C₁-C₆ haloalkyl, halogen, CN, and NO₂.

In certain embodiments, R^{3d} is H.

In certain embodiments, R^{2a} is H, R^{6a} is H, R^{6b} is H, R^{6c} is Br, R^{6d} is H, and R^{2d} is H.

In certain embodiments, R^{2a} is Br, R^{6a} is H, R^{6b} is H, R^{6c} is H, R^{6d} is H, and R^{2d} is H.

In certain embodiments, at least one of R^{4a}, R^{4b}, R^{4c}, R^{4d}, and R^{4e} is H. In certain embodiments, at least two of R^{4a}, R^{4b}, R^{4c}, R^{4d}, and R^{4e} are H. In certain embodiments, at least three of R⁴, R^{4b}, R^{4c}, R^{4d}, and R^{4e} are H. In certain embodiments, four of R^{4a}, R^{4b}, R^{4c}, R^{4d}, and R^{4e} are H.

In certain embodiments, the compound of formula (II) is:

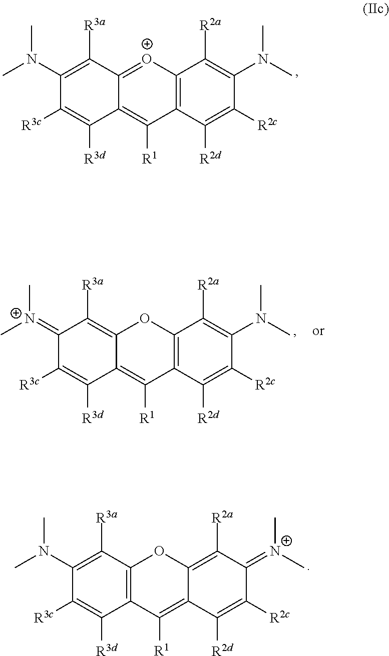

In certain embodiments, R^{3a}, is H, R^{3c} is H, and R^{3d} is H.

In certain embodiments, R^{2a} is H, R^{2c} is H, and R^{2d} is H.

In certain embodiments, at least one of R^{4a}, R^{4b}, R^{4c}, R^{4d}, and R^{4e} is H. In certain embodiments, at least two of R^{4a}, R^{4b}, R^{4c}, R^{4d}, and R^{4e} are H. In certain embodiments, at least three of R^{4a}, R^{4b}, R^{4c}, R^{4d}, and R^{4e} are H. In certain embodiments, four of R^{4a}, R^{4b}, R^{4c}, R^{4d}, and R^{4e} are H.

In certain embodiments, the compound of formula (I) is:

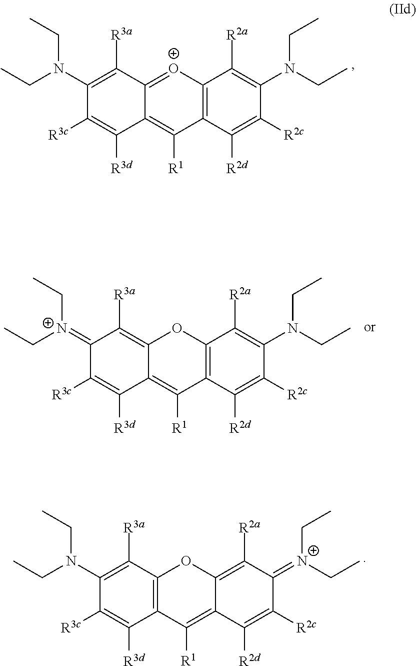

In certain embodiments, R^{3a}, is H, R^{3c} is H, and R^{3d} is H.

In certain embodiments, R^{2a} is H, R^{2e} is H, and R^{2d} is H.

In certain embodiments, at least one of R^{4a}, R^{4b}, R^{4c}, R^{4d}, and R^{4e} is H. In certain embodiments, at least two of R^{4a}, R^{4b}, R^{4c}, R^{4d}, and R^{4e} are H. In certain embodiments, at least three of R^{4a}, R^{4b}, R^{4c}, R^{4d}, and R^{4e} are H. In certain embodiments, four of R^{4a}, R^{4b}, R^{4c}, R^{4d}, and R^{4e} are H.

In certain embodiments, R¹ is

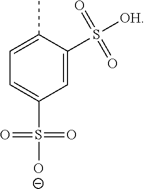

In certain embodiments, R¹ is

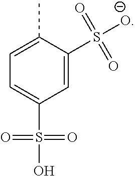

In certain embodiments, each occurrence of optionally substituted alkyl, optionally substituted haloalkyl, optionally substituted cycloalkyl, optionally substituted heterocycloalkyl, optionally substituted heterocycloalkenyl, optionally substituted aryl, and optionally substituted heteroaryl is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂ OR^{I}, N(R^{I})(R^{II}), C₁-C₆ haloalkoxy, C₃-C₈ halocycloalkoxy, aryl, heteroaryl. (C₁-C₆ alkylenyl)C(═O)N(R^{I})(R^{II}), (C₁-C₆ alkylenyl)C(═O)OR^{I}, O(C₁-C₃ alkylenyl)C(═O)OR^{II}, O(C₁-C₃ alkylenyl)C(═O)N(R^{I})(R^{II}), C(═O)R^{I}, C(═O)OR^{I}, OC(═O)R^{I}, OC(═O)OR^{I}, SR^{I}, S(═O)R^{I}, S(═O)₂R^{I}, S(═O)₂N(R^{I})(R^{II}), S(═O)₂NRC(═O)NHR^{II}, N(R^{I})S(═O)₂R^{II}, N(R^{I})C(═O)R^{II}, and C(═O)NR^{I}R^{II}, wherein R^{I} and R^{II} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

In certain embodiments, the isotopologue thereof comprises at least one radioactive tracer selected from the group consisting of ¹¹C, ¹³N, ¹⁵O, ¹⁸F, ¹²⁴I, ¹³¹I, and ¹³⁵I.

In certain embodiments, the compound of formula (II) is selected from the group consisting of
2-(12-bromo-1,2,3,5,6,7-hexahydrochromeno[2,3-f]pyrido[3,2,1-ij]quinolin-4-ium-9-yl)-5-sulfobenzenesulfonate;
2-(12-fluoro-1,2,3,5,6,7-hexahydrochromeno[2,3-f]pyrido[3,2,1-ij]quinolin-4-ium-9-yl)-5-sulfobenzenesulfonate;
2-(12-chloro-1,2,3,5,6,7-hexahydrochromeno[2,3-f]pyrido[3,2,1-ij]quinolin-4-ium-9-yl)-5-sulfobenzenesulfonate;
2-(12-iodo-1,2,3,5,6,7-hexahydrochromeno[2,3-f]pyrido[3,2,1-ij]quinolin-4-ium-9-yl)-5-sulfobenzenesulfonate;
2-(13-bromo-1,2,3,5,6,7-hexahydrochromeno[2,3-f]pyrido[3,2,1-ij]quinolin-4-ium-9-yl)-5-sulfobenzenesulfonate;
2-(11-bromo-1,2,3,5,6,7-hexahydrochromeno[2,3-f]pyrido[3,2,1-ij]quinolin-4-ium-9-yl)-5-sulfobenzenesulfonate;
2-(11,13-dibromo-1,2,3,5,6,7-hexahydrochromeno[2,3-f]pyrido[3,2,1-ij]quinolin-4-ium-9-yl)-5-sulfobenzenesulfonate;
2-(11,12-difluoro-1,2,3,5,6,7-hexahydrochromeno[2,3-f]pyrido[3,2,1-ij]quinolin-4-ium-9-yl)-5-sulfobenzenesulfonate;
2-(11-chloro-12-fluoro-1,2,3,5,6,7-hexahydrochromeno[2,3-f]pyrido[3,2,1-ij]quinolin-4-ium-9-yl)-5-sulfobenzenesulfonate;
2-(11-bromo-12-fluoro-1,2,3,5,6,7-hexahydrochromeno[2,3-f]pyrido[3,2,1-ij]quinolin-4-ium-9-yl)-5-sulfobenzenesulfonate;
2-(12-chloro-11-fluoro-1,2,3,5,6,7-hexahydrochromeno[2,3-f]pyrido[3,2,1-ij]quinolin-4-ium-9-yl)-5-sulfobenzenesulfonate;
2-(11,12-dichloro-1,2,3,5,6,7-hexahydrochromeno[2,3-f]pyrido[3,2,1-ij]quinolin-4-ium-9-yl)-5-sulfobenzenesulfonate;
2-(11-bromo-12-chloro-1,2,3,5,6,7-hexahydrochromeno[2,3-f]pyrido[3,2,1-ij]quinolin-4-ium-9-yl)-5-sulfobenzenesulfonate;
2-(12-bromo-11-fluoro-1,2,3,5,6,7-hexahydrochromeno[2,3-f]pyrido[3,2,1-ij]quinolin-4-ium-9-yl)-5-sulfobenzenesulfonate;
2-(12-bromo-11-chloro-1,2,3,5,6,7-hexahydrochromeno[2,3-f]pyrido[3,2,1-ij]quinolin-4-ium-9-yl)-5-sulfobenzenesulfonate;
2-(11-chloro-12-iodo-1,2,3,5,6,7-hexahydrochromeno[2,3-f]pyrido[3,2,1-ij]quinolin-4-ium-9-yl)-5-sulfobenzenesulfonate;
2-(12,13-difluoro-1,2,3,5,6,7-hexahydrochromeno[2,3-f]pyrido[3,2,1-ij]quinolin-4-ium-9-yl)-5-sulfobenzenesulfonate;
2-(12-chloro-13-fluoro-1,2,3,5,6,7-hexahydrochromeno[2,3-f]pyrido[3,2,1-ij]quinolin-4-ium-9-yl)-5-sulfobenzenesulfonate;
2-(12-bromo-13-fluoro-1,2,3,5,6,7-hexahydrochromeno[2,3-f]pyrido[3,2,1-ij]quinolin-4-ium-9-yl)-5-sulfobenzenesulfonate;
2-(13-chloro-12-fluoro-1,2,3,5,6,7-hexahydrochromeno[2,3-f]pyrido[3,2,1-ij]quinolin-4-ium-9-yl)-5-sulfobenzenesulfonate;
2-(12,13-dichloro-1,2,3,5,6,7-hexahydrochromeno[2,3-f]pyrido[3,2,1-ij]quinolin-4-ium-9-yl)-5-sulfobenzenesulfonate;
2-(3-bromo-6-nitroxanthylium-9-yl)-5-sulfobenzenesulfonate;
2-(3-bromo-6-methylxanthylium-9-yl)-5-sulfobenzenesulfonate;
2-(3-bromo-6-morpholinoxanthylium-9-yl)-5-sulfobenzenesulfonate;
2-(6-bromo-2-(1H-imidazol-1-yl)xanthylium-9-yl)-5-sulfobenzenesulfonate;
2-(6-bromo-2-sulfoxanthylium-9-yl)-5-sulfobenzenesulfonate;
2-(6-bromo-2-(1H-1,2,4-triazol--yl)xanthylium-9-yl)-5-sulfobenzenesulfonate;
2-(12-nitro-1,2,3,5,6,7-hexahydrochromeno[2,3-f]pyrido[3,2,1-ij]quinolin-4-ium-9-yl)-5-sulfobenzenesulfonate;
2-(12-(dimethylamino)-1,2,3,5,6,7-hexahydrochromeno[2,3-f]pyrido[3,2,1-ij]quinolin-4-ium-9-yl)-5-sulfobenzenesulfonate;
2-(12-(2-carboxyethyl)-1,2,3,5,6,7-hexahydrochromeno[2,3-f]pyrido[3,2,1-ij]quinolin-4-ium-9-yl)-5-sulfobenzenesulfonate;
2-(2-bromo-6-(dimethylamino)xanthylium-9-yl)-5-sulfobenzenesulfonate;
2-(2-bromo-6-(diethylamino)xanthylium-9-yl)-5-sulfobenzenesulfonate;
2-(12-methyl-2,3,6,7-tetrahydro-1H,5H-chromeno[2,3-f]pyrido[3,2,1-ij]quinolin-14-ium-9-yl)-5-sulfobenzenesulfonate;
2-(12-borono-2,3,6,7-tetrahydro-1H,5H-chromeno[2,3-f]pyrido[3,2,1-ij]quinolin-14-ium-9-yl)-5-sulfobenzenesulfonate;
2-(3-bromo-6-(dimethylamino)xanthylium-9-yl)-5-sulfobenzenesulfonate;
2-(3,6-bis(dimethylamino)xanthylium-9-yl)-5-sulfobenzenesulfonate;
2-(12-bromo-1,2,3,5,6,7-hexahydrobenzo[6,7]chromeno[2,3-f]pyrido[3,2,1-ij]quinolin-4-ium-9-yl)-5-sulfobenzenesulfonate;
2-(15-bromo-1,2,3,5,6,7-hexahydrobenzo[6,7]chromeno[2,3-f]pyrido[3,2,1-ij]quinolin-4-ium-9-yl)-5-sulfobenzenesulfonate; and
2-(3,6-bis(diethylamino)xanthylium-9-yl)-5-sulfobenzenesulfonate.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992): Carey & Sundberg, Advanced Organic Chemistry 4th Ed., Vols. A and B (Plenum 2000.2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from allyl, benzyl (Bn), benzyloxycarbonyl (Cbz), allyloxycarbonyl (Alloc), methyl, ethyl, t-butyl, t-butyldimethylsilyl (TBDMS), 2-(trimethylsilyl)ethoxycarbonyl (Teoc), t-butyloxycarbonyl (Boc), para-methoxybenzyl (PMB), triphenylmethyl (trityl), acetyl, and fluorenylmethoxycarbonyl (FMOC).

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag. New York, NY, 1994, which are incorporated herein by reference for such disclosure.

A compound of formula (I) and/or (II) can be prepared, for example, according to the synthetic methods outlined in Schemes 1-13.

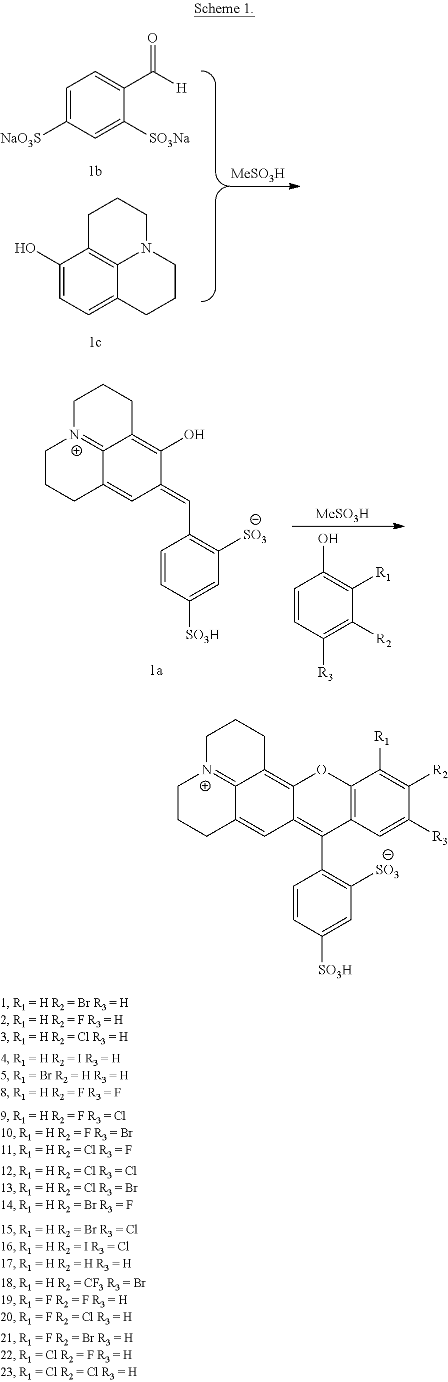

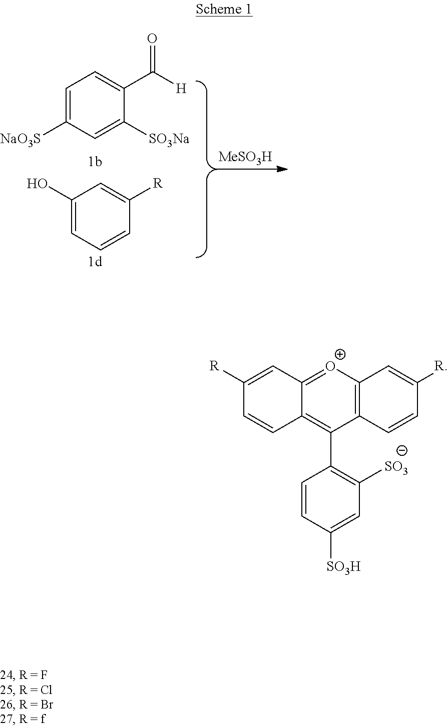

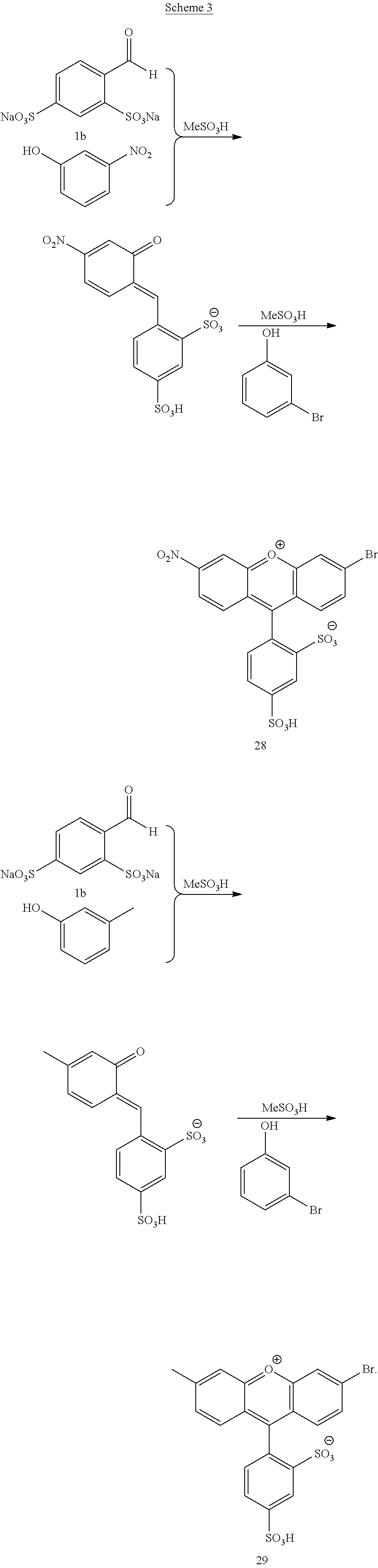

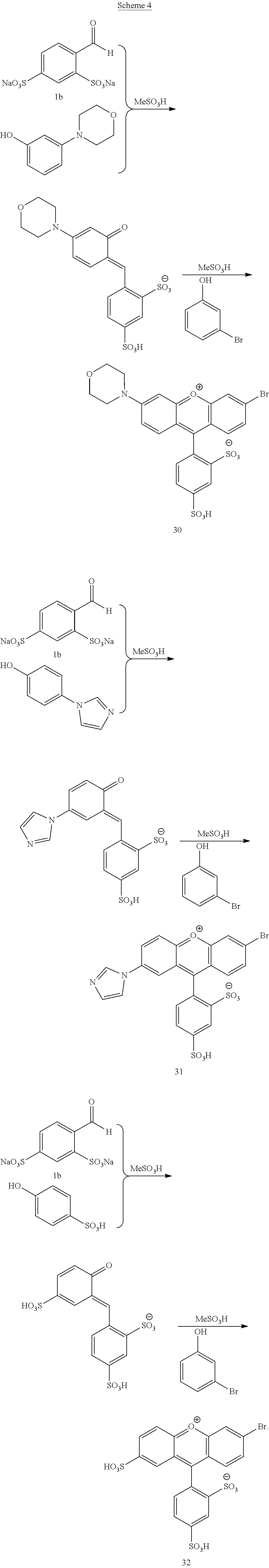

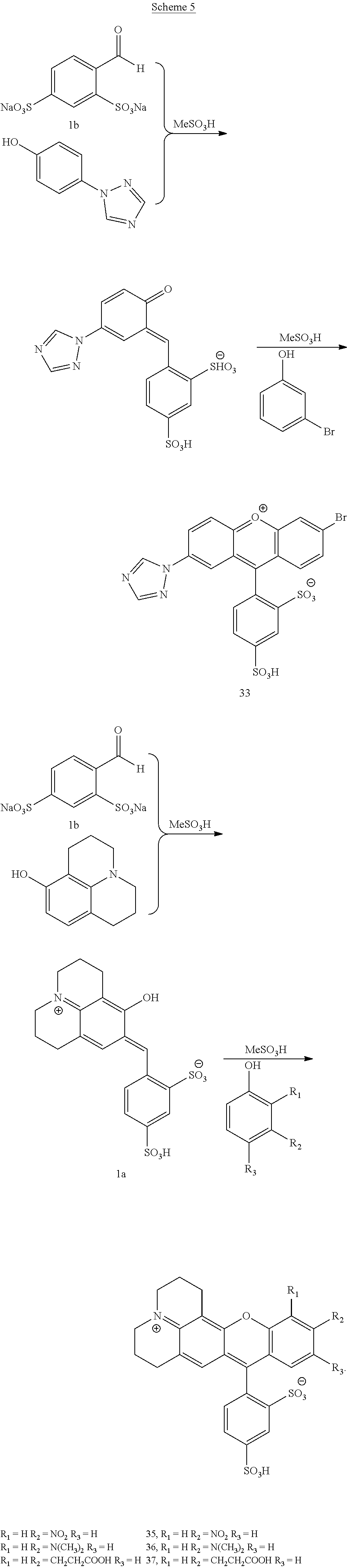

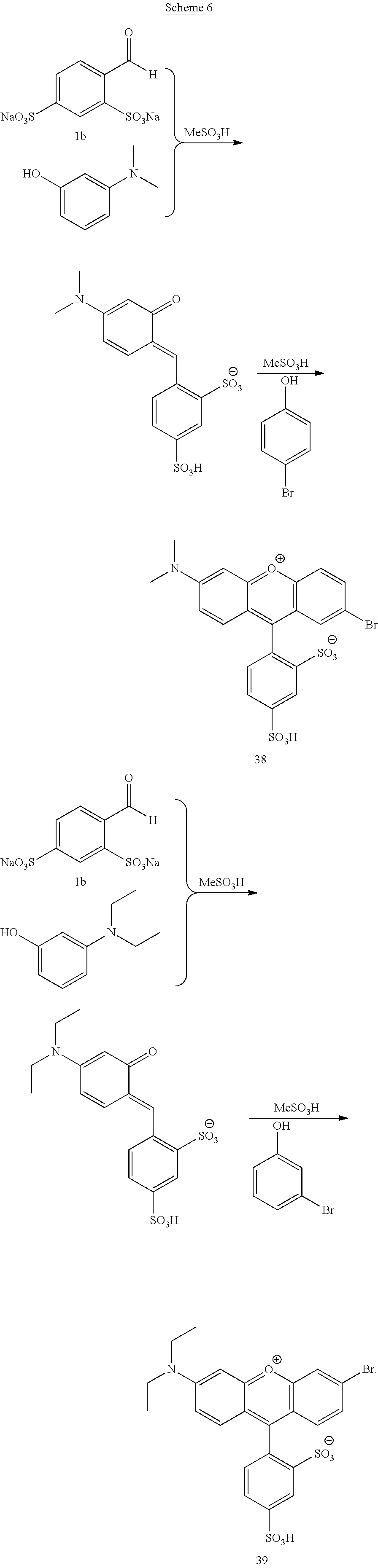

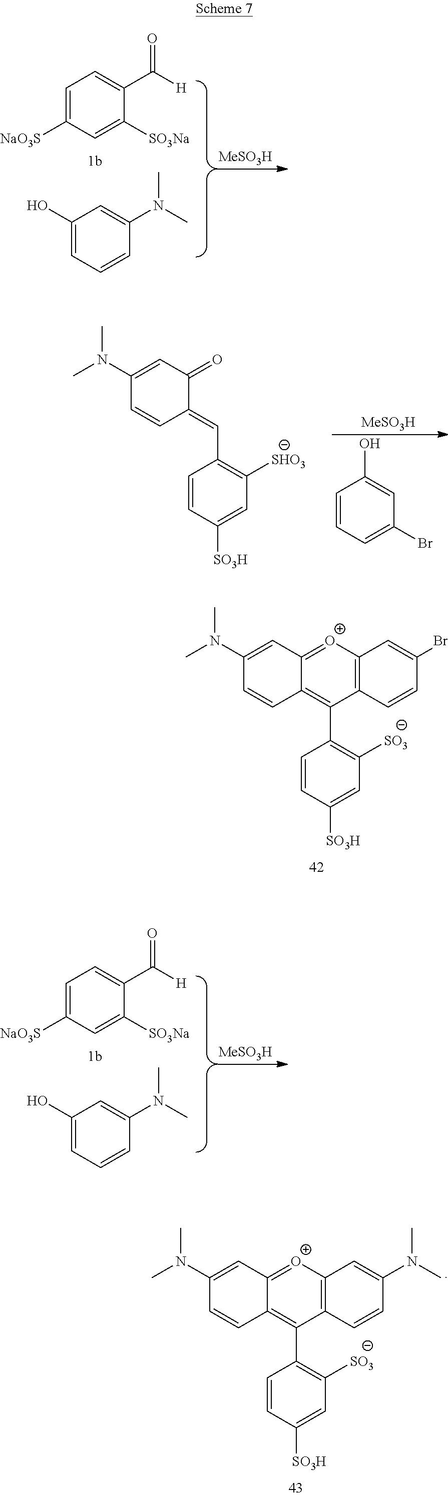

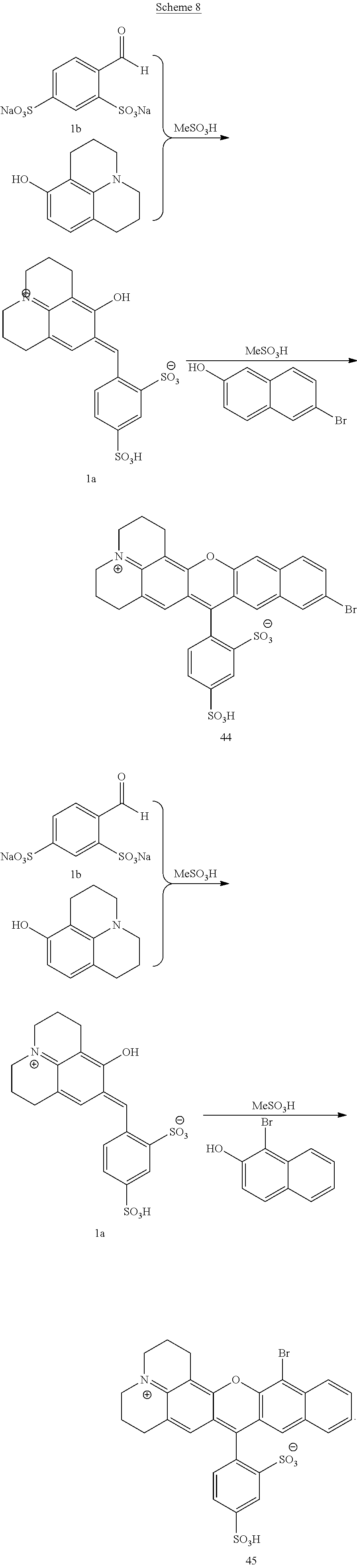

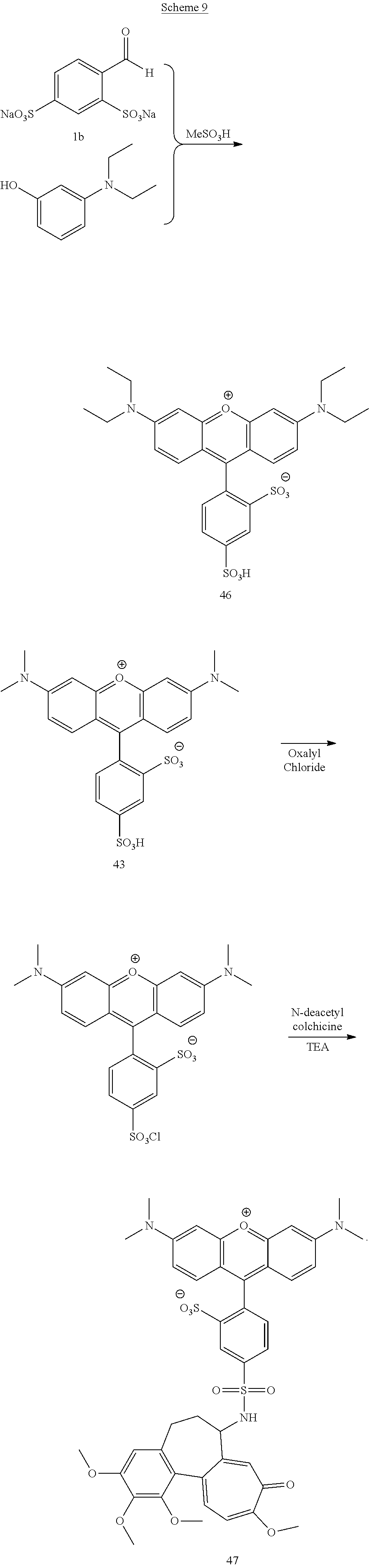

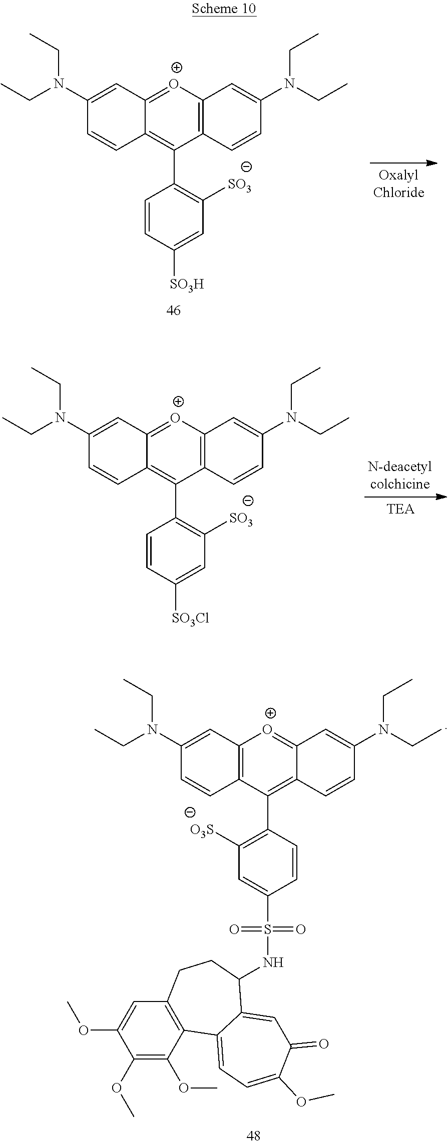

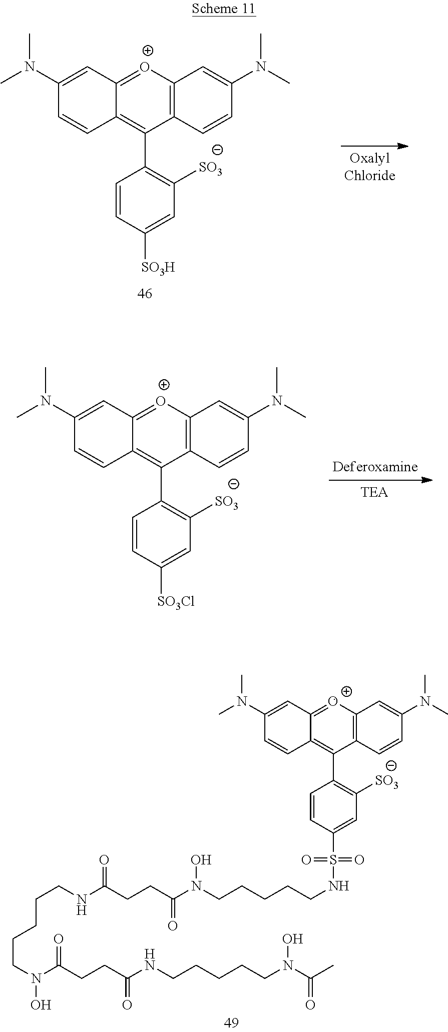

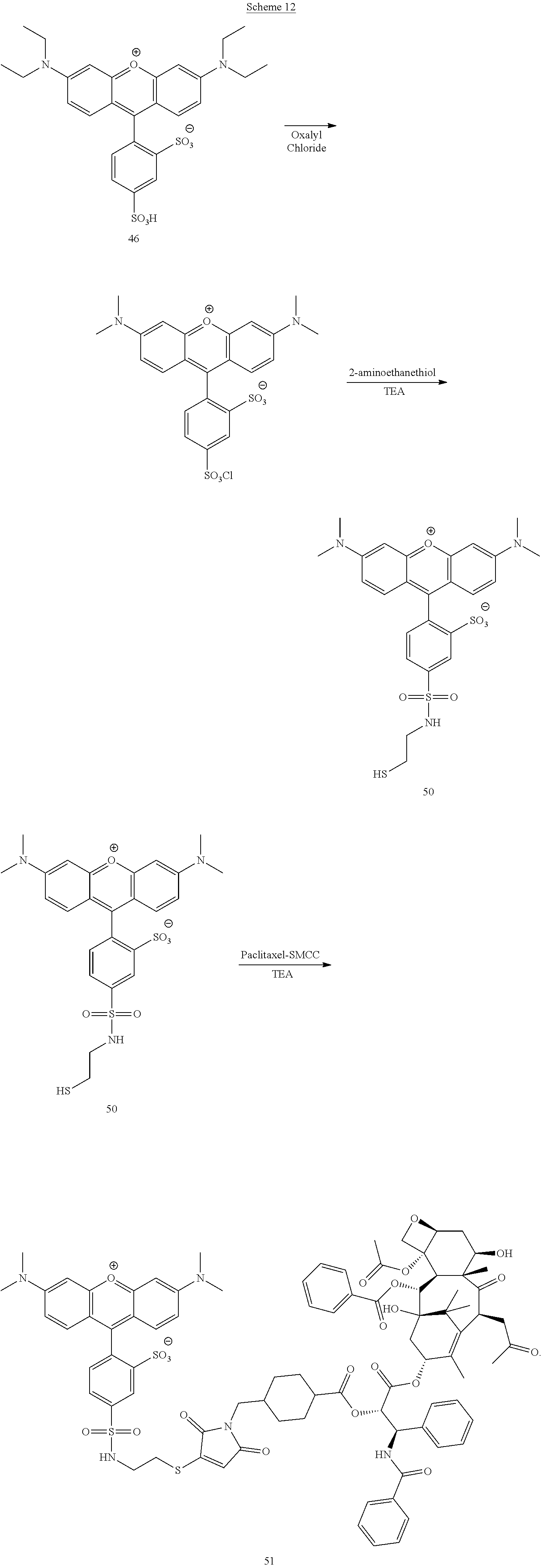

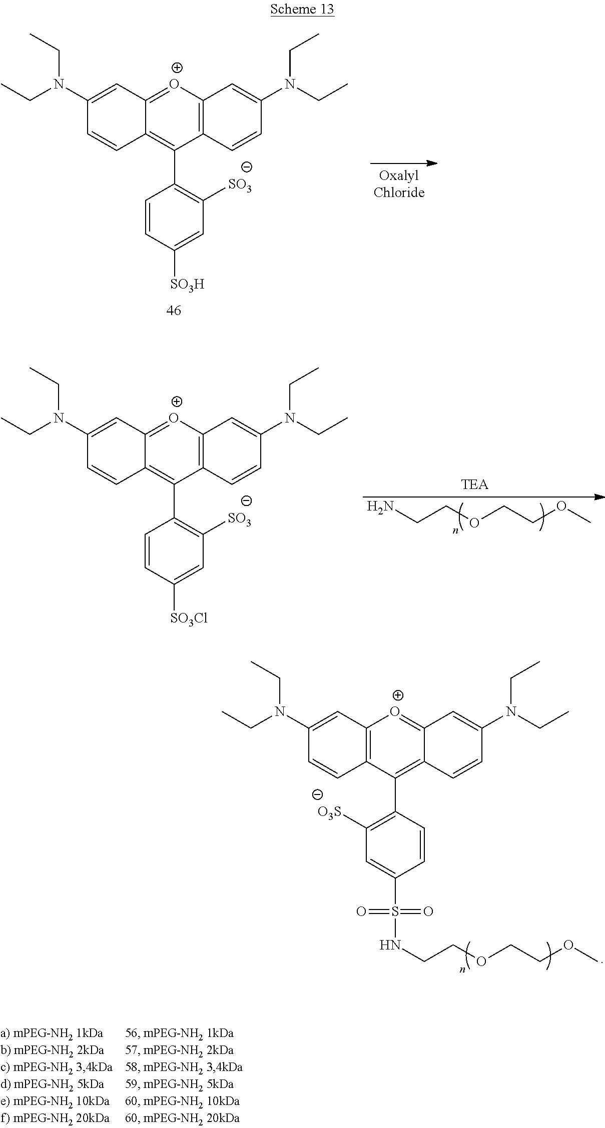

### Expressing Slco1a4 or SLCO1A2 in Cells

Slco1a4 or SLCO1A2 can be expressed in cells that do not naturally express these proteins as an exogenous protein. Methods of expressing an exogenous protein in a cell are numerous and known in the art. The exogenous protein can be introduced into the cells in the form of a DNA, an mRNA and/or a protein. The expression of the exogenous proteins can be either stable or transient, as well.

For example, the expression of exogenous Slco1a4 or SLCO1A2 can be introduced by genome engineering. A DNA sequence including a coding sequence of the protein(s), as well as a constitutively active promoter or a cell-type specific promoter that drives the expression of the protein(s), can be introduced by a DNA recombination technology. Such DNA sequences can be inserted by an integrase-based insertion, a CRISPR-based homologous recombination, and the like. Alternatively, if the genomic DNA of the cell already include a coding sequence for Slco1a4 or SLCO1A2, only the constitutively active promoter or the cell-type specific promoter needs to be inserted upstream of the coding sequence. Genome editing technology is described in, for example. Yin et al. (Nat Rev Drug Discov, 2017 June: 16(6):387-399).

Alternatively, a nucleic acid encoding Slco1a4 or SLCO1A2 can be introduced into the cell and the proteins expressed without being incorporated into the genome. For example, mRNA or DNA molecules encoding Slco1a4 or SLCO1A2, which are not configured to be integrated into the genome, can be introduced into the cells to induce transient production of Slco1a4 or SLCO1A2. The transient production of Slco1a4 or SLCO1A2 can be done by introducing an mRNA encoding the proteins, a plasmid including the coding sequence of Slco1a4 or SLCO1A2, or the like.

Furthermore. Slco1a4 or SLCO1A2 can be introduced into the cell as protein(s).

Methods of delivering nucleic acids or proteins into cells are well known in the art. The Slco1a4 or SLCO1A2 can be delivered into cells by, for example, an injection, a liposome-based nanoparticle, a micelle, a metal nanoparticle, a viral vector, an electroporation, and the like.

Kit for Delivering Compounds into Cells

As described elsewhere herein, the present study discovered that two proteins (Slco1a4 in mice and SLCO1A2 in humans) or nucleic acids encoding the same, when combined with the compounds herein or drug conjugates thereof, allows for the specific delivery of the compounds/drug conjugates into cells in which the two proteins are introduced.

Accordingly, in some aspects, the present study is directed to a kit for delivering a compound into a cell. In some embodiments, the kit is a kit for performing a gene therapy method.

In some embodiments, the kit includes the compounds herein or the drug conjugates thereof, as well as the Slco1a4 protein, the SLCO1A2 protein, and/or a nucleic acid encoding the same.

In some embodiments, the compounds herein, the drug conjugates thereof, the Slco1a4 protein, the SLCO1A2 protein, and nucleic acid encoding the same are the same as or similar to those described elsewhere herein, such as in the “Method of Delivering Compound into Cell” section.

In some embodiments, the kit further includes a component for delivering the Slco1a4 protein, the SLCO1A2 protein, or the nucleic acid encoding the same into a cell. Non-limiting examples of such components include a lipid component for forming a liposome (see e.g., Large et al., Adv Drug Deliv Rev, 2021 September; 176:113851), a metal nanoparticle for delivery (see e.g., Chandrakala et al., Emergent Mater, 2022:5(6):1593-1615), a micelle (see e.g., Majumder, et al., Ther Deliv, 2020 October; 11(10):613-635), a viral vector, an electroporation cuvette, a microinjector, and the like.

### Vectors

Vectors can increase the stability of the nucleic acids, make the delivery easier, or allow the expression of the nucleic acids or protein products thereof in the cells.

Therefore, in some embodiments, the nucleic acid for expressing Slco1a4 or SLCO1A2 in cells is incorporated into a vector.

In some embodiments, the instant specification relates to a vector, including the nucleic acid sequence of the instant specification or the construct of the instant specification. The choice of the vector will depend on the host cell in which it is to be subsequently introduced. In certain embodiments, the vector of the instant specification is an expression vector. Suitable host cells include a wide variety of prokaryotic and eukaryotic host cells. In certain embodiments, the expression vector is selected from the group consisting of a viral vector, a bacterial vector, and a mammalian cell vector. Prokaryote- and/or eukaryote-vector based systems can be employed for use with the instant specification to produce polynucleotide, or their cognate polypeptides. Many such systems are commercially and widely available.

In some embodiments, the vector is a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

In some embodiments, the viral vector is a suitable adeno-associated virus (AAV), such as the AAV1-AAV8 family of adeno-associated viruses. In some embodiments, the viral vector is a viral vector that can infect a human. The desired nucleic acid sequence, such as the nucleic acids that encoding the proteins herein, can be inserted between the inverted terminal repeats (ITRs) in the AAV. In various embodiments, the viral vector is an AAV2 or an AAV8. The promoter can be a thyroxine binding globulin (TBG) promoter. In various embodiments, the promoter is a human promoter sequence that enables the desired nucleic acid expression in the liver. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4. AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. In various embodiments, the instant specification contemplates an AAV8 viral vector (recombinant or non-recombinant) containing a desired nucleic acid expression sequence and at least one promoter sequence that, when administered to a subject, causes elevated systemic expression of the desired nucleic acid. In some embodiments, the viral vector is a recombinant or non-recombinant AAV2 or AAV5 containing any of the desired nucleic acid expression sequences described herein.

In some embodiments, the vector in which the nucleic acid sequence is introduced is a plasmid that is or is not integrated in the genome of a host cell when it is introduced in the cell. Illustrative, non-limiting examples of vectors in which the nucleotide sequence of the instant specification or the gene construct of the instant specification can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of the instant specification, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

### Administration/Dosage/Formulations

In another aspect, the present disclosure provides a pharmaceutical composition comprising at least one compound of the present disclosure and a pharmaceutically acceptable carrier. In certain embodiments, the pharmaceutical composition further comprises at least one additional therapeutically effective agent.

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated: each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1.500 mg, about 30 mg to about 1000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5.000 mg, or less than about 3,000 mg, or less than about 2.000 mg, or less than about 1000 mg, or less than about 500 mg, or less than about 200 mg, or less than about 50 mg. Similarly, in some embodiments, a dose of a second compound as described herein is less than about 1000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, restricted release, delayed release and pulsatile release formulations.

In certain embodiments, the term “restricted release” as used herein in the context of pharmaceutical compositions and/or formulations, may refer to surgically placed diffusion restricted biomaterials which restrict release of a compound contained in the composition and/or formulation to a specific target area of the body, as described in literature, including PubMed ID Nos. 33982891, 32613185, and 35805978. In certain embodiments, restricted release adhesive gels may be utilized to wounds to prevent excessive bone formation where muscle tissue is intended to be preserved.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors. The skilled artisan is similarly able to determine appropriate dosages for antibody-drug conjugates, based on the half-life and daily maximum exposure achievable with the compounds of the disclosure.

A suitable dose of a compound described herein can be in the range of from about 0.01 mg to about 5,000 mg per day, such as from about 0.1 mg to about 1000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously: alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

Various embodiments of the present application can be better understood by reference to the following Examples which are offered by way of illustration. The scope of the present application is not limited to the Examples given herein.

### Materials and Methods

### Generation of Transporter Plasmids for Subsequent Cell Transfections and in Utero Electroporation

Given that most candidate transporter plasmids are not commercially available, standard molecular biology techniques will be used to generate cDNA from mouse brain tissue by synthesizing RNA primers from publicly available gene sequences, isolating RNA and synthesizing cDNA through a standard reverse transcriptase kit. The synthesized cDNA will be inserted into a mammalian vector (pcDNA3.1) with FLAG tag fusion and GFP co-expression for mammalian cell overexpression and in utero electroporation. AAV2 viral vectors expressing certain transporters and co-expressing GFP will be used in selected in vivo experiments when desired cells cannot be in utero electroporated.

### CRISPR/Cas9 Knock Out of Candidate Transporter Genes

Standard techniques will be utilized to design and produce single guide RNAs using the online IDT tool (integrated DNA technologies). Briefly, two sgRNAs targeting different exons will be designed for each target gene and cloned into pX458 vector (Addgene #48138) for plasmid in utero electroporation and AAV-sgRNA-Cre vector (Addgene #60229) for viral infection. To clone the sgRNAs into vectors, two complementary oligos for each sgRNA were designed, adding two overhanging sequences. For stable adult expression of plasmids following in utero electroporation, it is necessary to design constructs with the PiggyBac transposon.

### Cell Culture, Transfection, Dye Uptake and Immunohistochemistry

HEK293 cells are cultured and seeded at ~60% confluency in 24-well plates and are transiently transfected with 250 ng of a candidate transporter and 1 μL transfection reagent (JetPRIME). After 2 days, cells are washed and novel fluorescent probes at different concentrations are added to the transport medium and incubated at 37° C., for at least 30 minutes. To terminate the transport, cells are washed with PBS and fixed with 4% paraformaldehyde. The colocalization of probe uptake and cells expressing the transporters is investigated by immunostaining the transporter which has a fused FLAG tag, using anti-FLAG 488. Before confocal imaging, nuclei are labeled with Hoechst 33342 and cells are mounted and imaged on a Leica SP8 confocal microscope.

### In Utero Plasmid Electroporation (IUE)

IUE is performed at E14 or E15 as previously done. Briefly, timed pregnant mice are anaesthetized with ketamine/xylazine. The abdominal region is shaved, sterilized and a 3 cm midline incision is made in the skin and abdominal muscle. The uterine horns are exposed, and the lateral ventricle of each embryo is pressure injected (Picospritzer II, General Valve) with plasmid DNA (~0.5 pl volume per embryo) at a concentration of 1 μg/ml followed by electroporation with tweezertrodes (50 V, 4-50 ms pulses with I s pulse interval. BTX Harvard Apparatus). The embryos are placed back in the mother womb, and the muscle and skin are sutured. Electroporated pups are aged to postnatal day 30. Imaging is performed through a craniotomy over the transfected hemisphere following dye labeling.

### Adeno-Associated Virus (AAV) Production and Injection

AAV viruses are routinely produced following procedures described previously using a two-plasmid helper free system. Briefly, transfer plasmid with the target gene and helper plasmid are co-transfected into HEK293 cells using JetPRIME reagents. Cells are collected 4-5 days after transfection. Viruses are extracted from the cell lysate and purified by iodixanol gradient ultracentrifugation and tittered by transfection assay. To express candidate transporters in vivo in astrocytes and neurons, AAV vectors with a titer ~10E7 are delivered via injection into the subarachnoid space as previously described in the literature, resulting in widespread labeling of cortical layer II/III and V neurons or superficial astrocytes.

The Adeno-associated virus (AAV) for expression of SLCO1A2 was custom-made (VectorBuilders). Briefly, The AAV8 virus carrying the pAAV-CAG>hSLCO1A2:P2A:EGFP construct was produced by cloning the hSLCO1A2 gene variant downstream of the CAG promoter in the pAAV8 expression vector. A P2A sequence was inserted to link the hSLCO1A2 gene with the EGFP coding sequence. The constructed vector was transfected into HEK293 cells, and the virus produced was harvested and ultra-purified for a final titer >10¹³ GC/mL. AAV virus were injected into the mouse subarachnoid space using a previously described method. Briefly, mice were anesthetized with ketamine-xylazine. A skull window, approximately 1 mm in diameter, was created using a high-speed drill at coordinates 6 mm anteroposterior and 3 mm mediolateral from bregma. Subsequently, a 30-gauge needle was carefully used to lift a small piece of thinned skull, exposing the underlying dura. The stock solutions of AAV were diluted in fresh sterile phosphate-buffered saline (PBS) solution (1:20, v/v) at room temperature, then kept on ice and adjusted to a titer of 10¹² GC/mL. The AAV solutions were loaded into a Tygon tube, connected to a polypropylene tip with an outer diameter of 70 μm, which was further attached to a programmable syringe pump with a Hamilton syringe. The tip was gently inserted into the subarachnoid space and secured with cyanoacrylate glue. A volume of 10 μL of the AAV was injected at a controlled speed of 0.2 μL per minute. Following the injection, the tip was carefully removed, and the incision on the mouse's scalp was sutured. The mice were placed on a heating pad to aid in their recovery. Three weeks after the subarachnoid injection, the mice were imaged to assess stable fluorescent expression.

### In Vivo Two Photon Imaging of Fluorophore Uptake

Briefly, animals are anaesthetized via intraperitoneal injections of ketamine/xylazine or via inhaled isoflurane. A region of the skull (3×3 mm) is gently removed with a high-speed drill and the underlying dura is removed. A small glass coverslip is placed over the skull to allow long term optical access for in vivo imaging. Labeling with fluorescent dyes is performed either via intravenous injections or topically application to the cortical surface for 20 min prior to glass cover placement. After recovery, in vivo images are acquired using a two-photon microscope (Bruker Technologies) equipped with a multimodal (fixed wavelength 1040 nm and tunable wavelength) InSight X3 two-photon laser (Spectra Physics) and 20× water immersion objective (Zeiss 1.0N.A.). The two-photon laser is appropriately tuned to excite particular fluorescent dyes and proteins as needed. In selected cases, following in vivo imaging, mice will be sacrificed and perfused for follow up immunohistochemistry and high-resolution confocal imaging (Leica SP8) to further confirm colocalization of dyes and cells expressing particular transporters.

One-month-old wild-type mice were anesthetized using a ketamine/xylazine solution at doses of 100 mg/kg and 10 mg/kg, respectively. The fur around the skull area was shaved, and buprenex (0.1 mg per kg) and carprofen (5 mg per kg) were administered subcutaneously. Mice were kept on a heating pad at 37° C., and anesthesia was periodically monitored. The skin was treated with povidone-iodine solution, followed by cleaning with ethanol, and eye ointment was applied. A small skin section was excised to expose the skull. Thinning of the skull was performed in a circular area, followed by delicately lifting of the remaining skull without harm to the underlying brain. Within the circular region, the dura was gently removed using fine forceps, and a 4 mm cover glass was softly pressed onto the brain surface and affixed to the skull. Fluorescent dyes were applied either through intravenous injections or topically to the cortical surface for 20 minutes before placing the glass cover. A customized head bar was attached either by adhesive (for acute imaging) or permanently implanted (using dental cement, for chronic imaging) onto the skull. Mice were imaged under a two-photon microscope (Prairie Technologies) with a mode-locked MaiTai tunable laser (Spectra Physics). Imaging was performed with a 20× water immersion objective (Zeiss, 1.0NA). Images were captured at depths up to 300 μm from the pial surface using excitation wavelengths ranging from 750 to 950 nm as previously described. Mice undergoing chronic imaging received a recovery period on a heating pad post-surgery, and buprenex (0.1 mg per kg) and carprofen (5 mg per kg) were administered for 3 days.

### Experimental Mice

The following transgenic mice currently maintained in an animal facility: Tie2GFP (endothelial labeling), Aldhlll-Cre (Astrocyte labeling), PDGFRPCre and NG2Cre (pericytes and oligodendrocyte precursor cells). RCE:loxp (floxed GFP reporter) (JAX #32037) will be crossed with Cre lines. For gene knockout of transporters, whenever not possible by in utero or viral mediated CRISPR/Cas9 delivery (due to difficulty transfecting endothelial cells or pericytes), the generation of global transporter knockout mice will be outsourced, unless they become available through investigator collaboration or a commercial source. Male and female mice 1 to 3 months of aged were used for certain experiments. In certain experiments, the following mouse lines were used: wild-type C57BL/6, TIE2-GFP (JAX #003658) and Oatp1a/1b Cluster knockout mice (Taconic Biosciences #10707).

### Chemical Synthesis

All chemical reactions were carried out under normal conditions without exclusion of air or moisture, unless otherwise stated. All commercially available reagents and solvents were obtained from common suppliers [Ambeed, TCI Chemicals, Thermo Scientific Chemicals, Acros organics, MilliporeSigma, Nanocs, BroadPharm] and used without further purification unless otherwise reported. Dichloromethane (CH₂Cl₂), chloroform (CHCl₃), N,N-dimethylformamide (DMF), tetrahydrofuran (THF), acetonitrile (MeCN), and ethanol (CH₃CH₂OH) were dried over alumina and stored in molecular sieves. Triethylamine (Et₃N) and N,N-Diisopropylethylamine (i-Pr₂NEt) were distilled over calcium hydride (CaH₂) under a nitrogen atmosphere prior to use. Deionized water was used for reactions and extraction mediums. HPLC grade solvents were used for all other chromatography. To diversify and functionalize the chemicals, some starting materials were modified into different salts. Typically, the reactants (10 mM) and catalysts (10 μM) were dissolved in dimethyl sulfoxide (DMSO, 0.5 mL) in 1-dram clear glass vials and vortexed for 1 minute. To increase throughput, the reactions (5 vials per round) were heated under microwave irradiation (800 W) for 3 minutes, resulting in a yield of 20-95%. During the reaction, most of the volatile substances evaporated, leaving DMSO as the common solvent. Without further purification, chemical batches for in vivo screening were prepared by mixing 10 compounds together in equal proportions. Each batch was diluted in PBS (1:15 v/v), vortexed for 2 minutes, centrifuged, and the supernatant was collected for in-vivo screening. Certain abbreviations used herein include: BEH, ethylene bridged hybrid: Boc, tert-butoxycarbonyl; calcd, calculated; ESI, electrospray ionization; HRMS, high-resolution mass spectrometry; LCMS, liquid chromatography mass spectrometry; MS, mass spectrometry: NaOH, sodium hydroxide; NHS, N-hydroxysuccinimide; NMR, nuclear magnetic resonance; PC, photocleavable; PEG, polyethylene glycol; SQD2, single quadrupole detector 2; TEA, triethylamine: TFA, trifluoroacetic acid; TLC, thin layer chromatography: UPLC, ultra-high-performance liquid chromatography; and UV, ultraviolet.

### Thin Layer Chromatography (TLC) and Column Chromatography

Analytical thin-layer chromatography (TLC) was performed using SILICYCLE® Inc. glass-backed silica gel hard layer with 20×20 cm size (F254, 250 μm thickness) and developed plates were visualized using a UV lamp and/or stained with Iodine (12), potassium permanganate (KMnO₄), p-anisaldehyde (CH₃OC₆H₄CHO), phosphomolybdic acid (12MoO₃·H₃PO₄·xH₂O) or ninhydrin. Normal phase flash column chromatography was conducted using either silica gel 60 A (32-63 microns) or an automated Biotage® Isolera™ One flash purification system equipped with a 10 g SNAP Ultra (HP Sphere, 25 μm silica) cartridge. Whichever column chromatography was performed, the desired fractions (confirmed by TLC or UV) were collected and concentrated under reduced pressure to obtain the product.

### Nuclear Magnetic Resonance (NMR)

All NMR data were acquired at ambient temperature, unless otherwise indicated. NMR solvents, chloroform-D (CDCl₃) and methanol-d₄ (CD₃OD) were purchased from Cambridge Isotopes Laboratories, Inc, and used as received. CD₃OD ampules were used immediately upon opening. NMR spectra were processed with MestReNova software (v. 10.0.2) using the baseline and phasing correction features. Multiplicities and coupling constants were calculated using the multiplet analysis feature with automated and/or manual intervention as necessary, 1H NMR spectra were obtained on Agilent 400 MHz, 500 MHz, or 600 MHz spectrometers. Proton chemical shifts (δ) are reported in ppm and referenced to residual solvent peaks for CDCl3 (δ 7.26 ppm) and CD₃OD (δ 4.87 ppm). Proton data are reported as chemical shift, multiplicity (noted as singlet (s), doublet (d), triplet (t), quartet (q), pentet (p), heptet (hept), multiplet (m), broad singlet (bs), doublet of doublets (dd), doublet of doublet of doublets (ddd), doublet of doublet of triplets (ddt), doublet of triplets (dt), doublet of triplet of triplets (dtt), etc.) coupling constants [Hz], and integration, 13C NMR spectra were obtained on Agilent 400(101) MHz, 500 (126) MHz, or 600 (150) MHz spectrometers with full proton decoupling. Carbon chemical shifts (δ) are reported in ppm and referenced to residual solvent peaks for CDCl3 (δ 77.16 ppm) and CD₃₀D (δ 49.00 ppm) with multiplicity and coupling constants [Hz] indicated when present.

### Mass Spectrometry

High-resolution mass spectrometry (HRMS) was conducted using a Waters Xevo Q-TOF high-resolution Mass Spectrometry using ESI.

### Fluorescence Spectrometer

Fluorescence spectra were recorded at room temperature on an Agilent Cary Eclipse fluorescence spectrophotometer. The data was analyzed with WinFLR software.

### Combinatorial Fluorophore Batch Screening

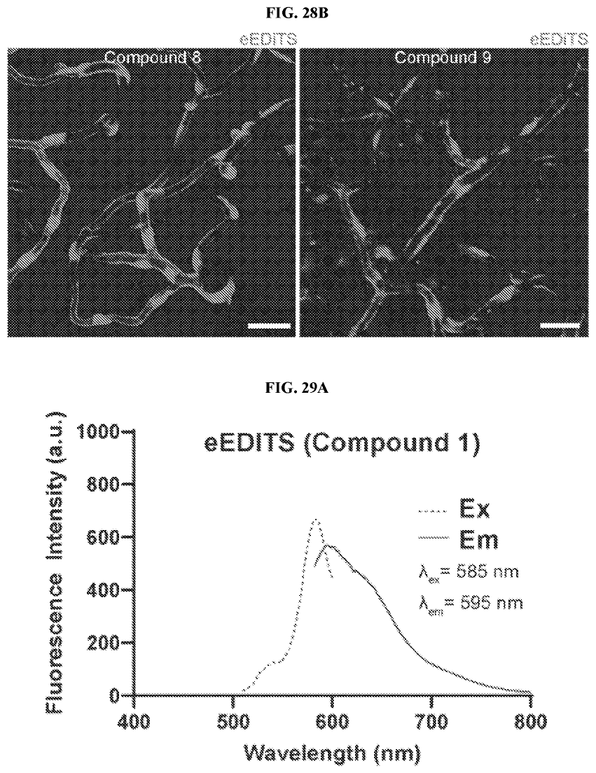

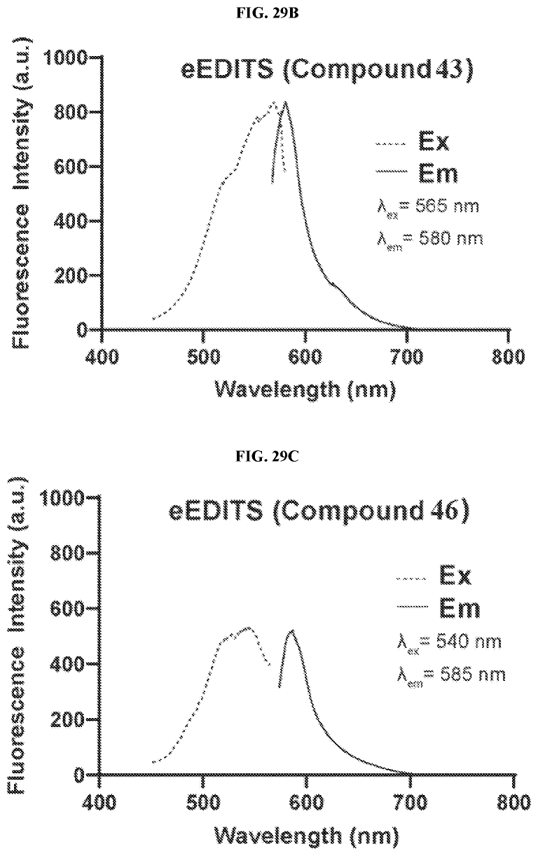

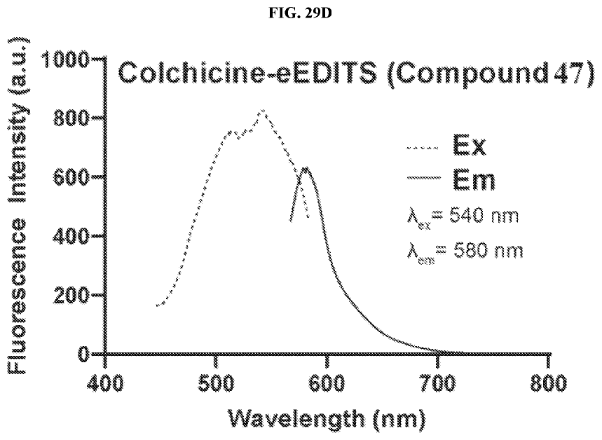

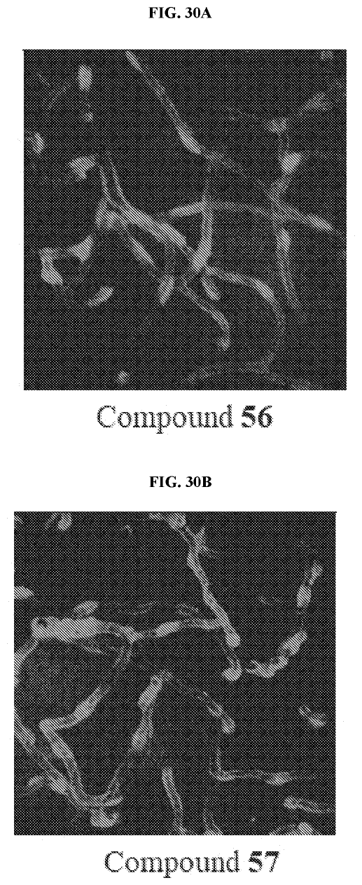

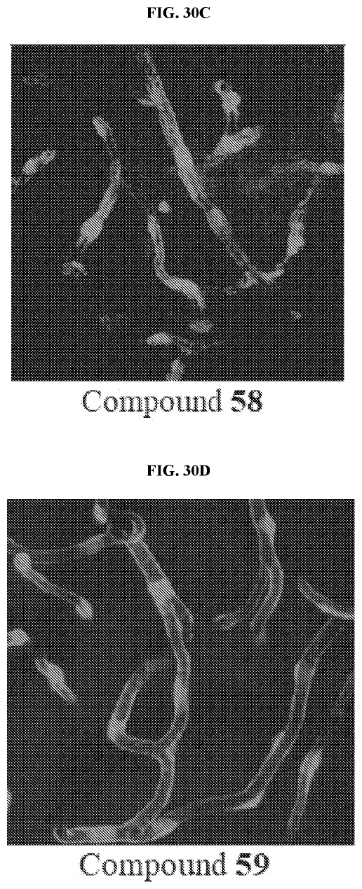

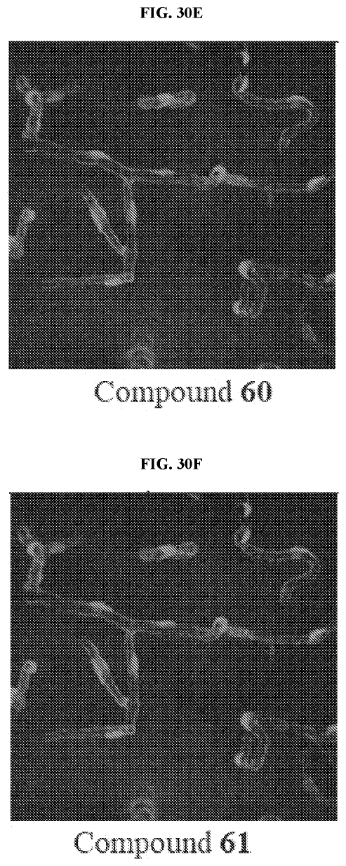

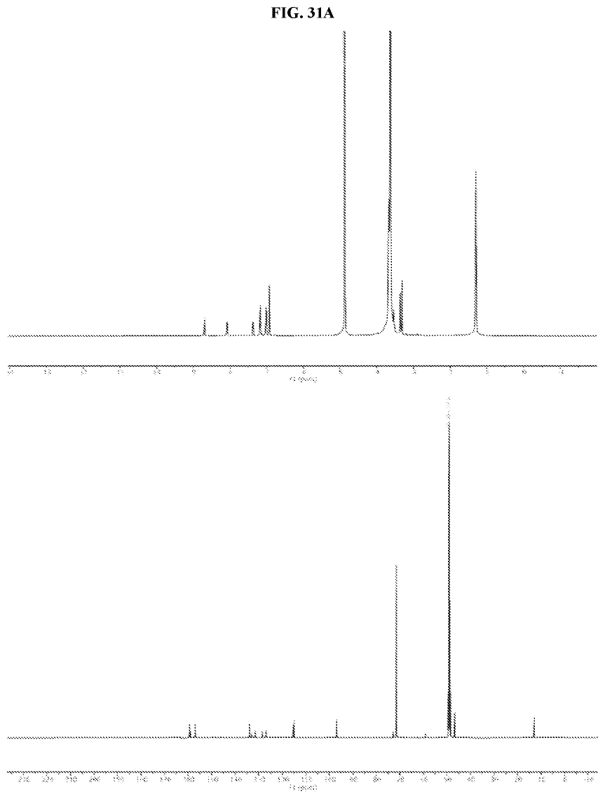

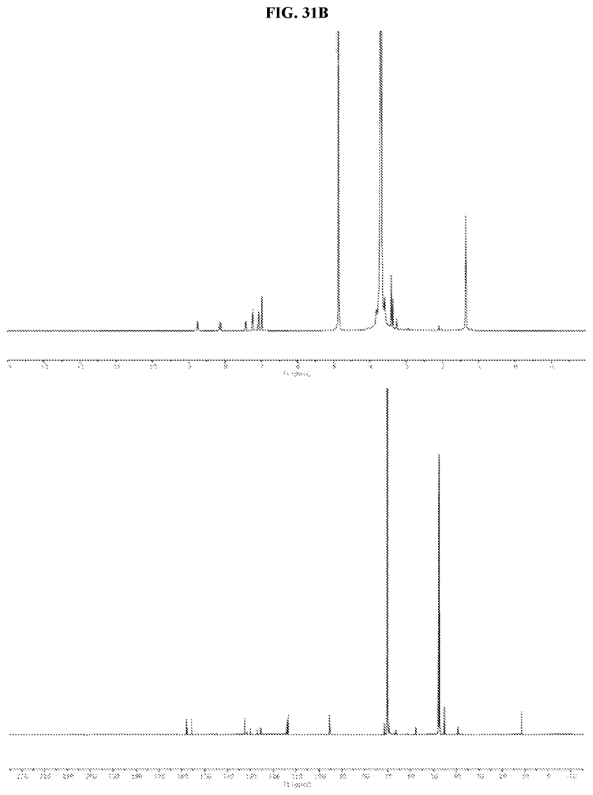

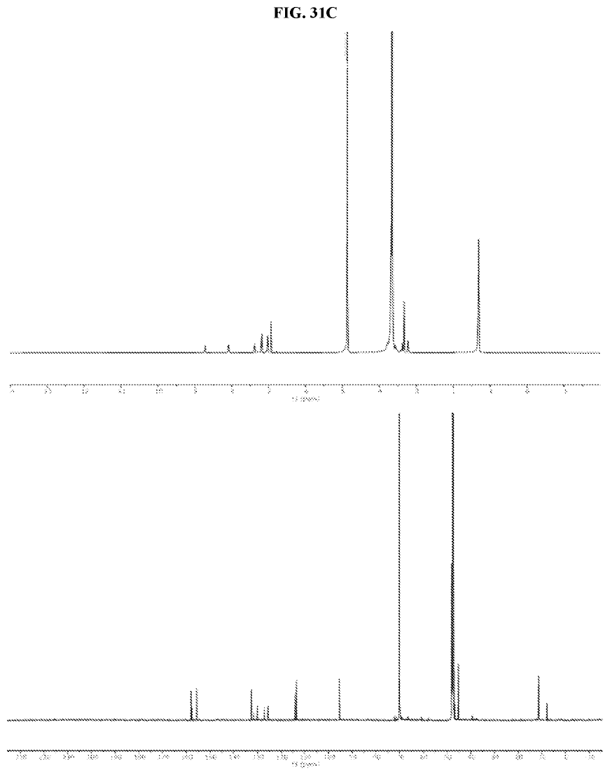

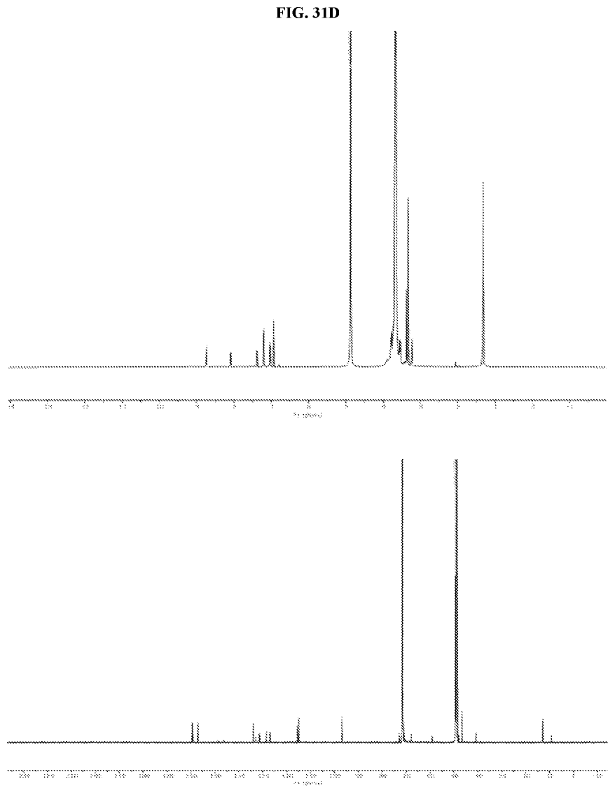

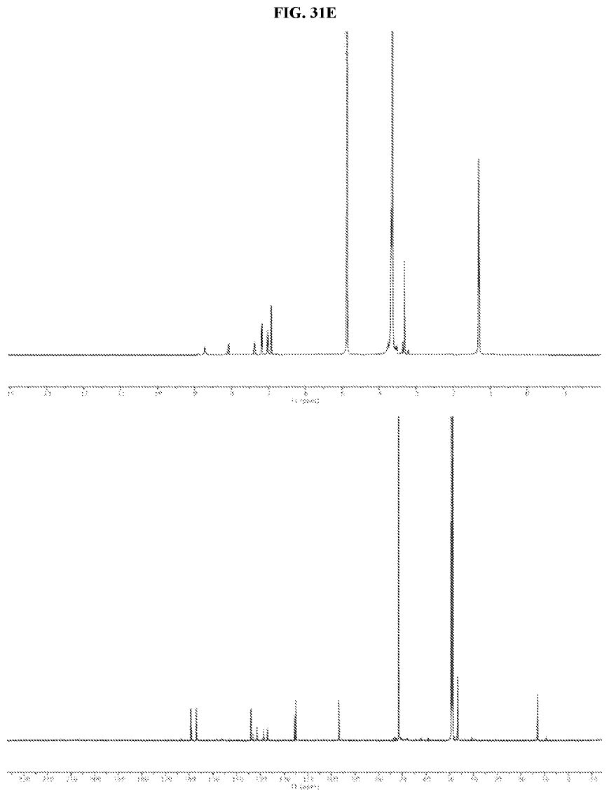

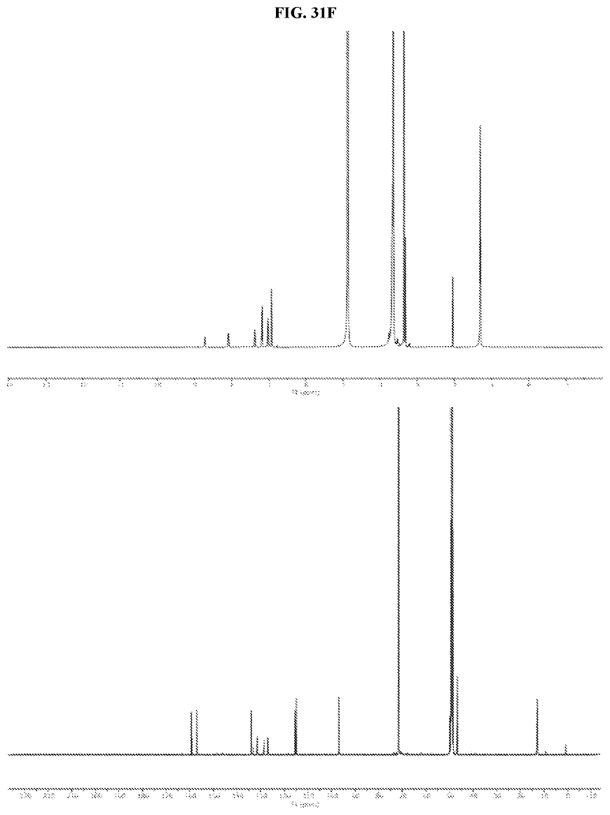

Batches of 10 compounds were applied through the craniotomy to the cortical surface before placing a cover glass window. No additional washings were performed, and the cover glass was placed, allowing up to 3 hours of imaging of the cortical surface with a two-photon microscope. Following imaging, the data were analyzed to identify potential patterns of cell-specific labeling. The majority of batches did not exhibit specific cell patterns but showed diffuse cellular or interstitial space labeling. Batches demonstrating any specific cellular labeling patterns were selected for further refinement. Positive batches underwent three additional rounds of imaging, with each batch being sequentially split, until a single compound displaying cell-type-specific labeling was identified. As these compounds were not initially purified, the labeling may have resulted from fluorescent byproducts of synthesis. Consequently, large-scale synthesis, purification and characterization (FIGS. 29A-29D) of the identified compounds were conducted. The purified compounds were subsequently retested using in vivo imaging to confirm cell type specificity.

### In Vitro and In Vivo Compound Uptake Experiments

For in vitro experiments, HEK293 cells were cultured and seeded in 24-well plates, reaching approximately 60% confluency. For transient transfection, 250 ng of a candidate transporter plasmid and 1 μL of transfection reagent (JetPRIME) were added to the cells. After a 2-day incubation period, the cells were washed, and novel fluorescent probes were introduced into the transport medium at various concentrations. The cells were then incubated at 37° C., for a minimum of 30 minutes to allow for uptake. To stop the transport process, the cells were washed with PBS and fixed using 4% paraformaldehyde. Immunostaining of the transporter, which was fused with a FLAG tag, was performed using an anti-FLAG 488 antibody. Before confocal imaging, the cell nuclei were labeled with Hoechst 33342. Finally, the cells were mounted and imaged using a Leica SP8 confocal microscope.

For in vivo assessment of compound biodistribution, eEDiTS were injected through the tail vein (0.1 mM, 70 μL) in Tie2-GFP endothelial reporter mice. After, 2.5 hrs, mice were perfused, and various organs (spleen, heart, skeletal muscle, liver, kidney) were extracted for sectioning and confocal imaging. Special attention was placed to the uptake of eEDiTS by endothelial cells as well as the parenchyma of each organ and analyzed semi-quantitatively.

### Assessment of Pharmacological Effects In Vitro

NIH3T3 were utilized to evaluate the pharmacological activity of the Colchicine-eEDiTS conjugate given their natural property of allowing uptake of eEDiTS. In brief, these cells were treated with varying concentrations of eEDiTS, Colchicine-eEDiTS, or Colchicine for a duration of 12 hours. Confocal microscopy of the different treatment groups allowed us to visualize Hoechst dye-labeled nuclei to quantify the proportion of cells with multinucleated phenotype which is known to occur with Colchicine treatments. In addition, immunofluorescence immunolabeling with anti-tubulin antibody was used to confirm the drug effects on patterns of microtubule polymerization.

### Cell Free Tubulin Polymerization Assay

A one-step fluorescence-based tubulin polymerization assay was used (Cytoskeleton, Inc. #BK011P) as previously described to compare the effects of eEDiTS, Colchicine-eEDiTS, Colchicine, and Vehicle. Briefly, the assay is conducted at a controlled temperature of 39° C., for optimal tubulin polymerization in a pre-warmed 96-well plate with continuous orbital shaking. Changes in fluorescence intensity (λₑₓ=350 nm and λₑₘ=435 nm) were measured using a plate reader (Infinite 200Pro instrument, Tecan i-control application) up to 30 minutes after compound administration.

### Assessment of In Vivo Compound Toxicity

Colchicine-eEDiTS, eEDiTS or Colchicine were assessed for toxicity using both systemic intraperitoneal administration and intradermal injection at varying concentrations. To evaluate systemic toxicity, mouse weight was monitored daily, focusing on P20 mice in their active growth stage. For local toxicity assessment, the mouse fur was shaved to stimulate follicular stem cell division and subsequent hair regrowth. Compounds were injected intradermally on the right lower back quadrant and the left quadrant was used as a control. Hair regrowth was monitored for 12 days with serial photography followed by quantification using NIH Image J Plot Profile Plug-In, yielding pixel intensity profiles. Changes in pixel intensity where compared between injected (right quadrant) versus noninjected (left quadrant) by using the following formula:

(Noninjected intensity−Injected intensity)/Noninjected intensity×100%. (Eq. 1)

### Immunofluorescence Staining and Confocal Microscopy

Brains were postfixed overnight at 4° C., in 4% paraformaldehyde and sectioned with a vibratome (Leica VT1000) at 50 μm thickness prior to immunofluorescence staining. For quantification of Slco1a4 immunofluorescence a tangential sectioning approach was also used to preserve pial vasculature and compare patterns of expression in larger pial and smaller intraparenchymal vessels. To block nonspecific binding, the tissue sections were treated with a solution of 1×PBS containing 5% normal donkey serum and 0.1% Triton X-100 at room temperature. Both primary and secondary antibodies were diluted in a solution of 1×PBS containing 5% normal donkey serum and 0.1% Triton X-100. The tissue sections were incubated with the primary antibodies overnight at 4° C., followed by incubation with the secondary antibodies for 2 hours at room temperature. A Leica SP8 confocal microscope was used for high-resolution confocal microscopy imaging. The applicable laser excitation wavelengths and acousto-optical beam splitter settings were employed for optimal fluorophore excitation, emission separation, and detection. The microscope was equipped with either a 20× water immersion objective (1.0 NA, Leica) or a 63× oil immersion objective (1.40 NA, Leica).

### Example 1: Chemical Synthesis

Compound 1a. 4-Formylbenzene-1,3-disulfonic acid disodium salt hydrate (50 mg, 0.16 mmol) and 8-hydroxyjulolidine (30 mg, 0.16 mmol) were added to methanesulfonic acid (250 μL) in a 20-mL scintillation vial. The mixture was stirred at 160° C., for 20 min using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (66 mg, 95%). ¹H NMR (400 MHz, DMSO-D₆, δ): 9.10 (s, 1H), 8.17 (t, J=1.5 Hz, 1H), 7.70 (dt, J=7.8, 1.6 Hz, 1H), 7.35 (d. J=7.8 Hz, 1H), 7.11 (s, 1H), 3.71 (d, J=5.5 Hz, 4H), 2.67 (m, 4H), 1.93 (m, 4H). ¹C NMR (126 MHz, DMSO-D₆, δ): 160.4, 158.0, 149.4, 149.0, 148.0, 131.5, 131.3, 130.8, 126.0, 125.7, 125.4, 124.7, 107.2, 52.7, 51.8, 26.1, 20.4, 19.90, 19.1. ESI-MS (m/z): [M−H]⁻ cacld for C₁₉H₁₈NO₇S₂, 436.0603. found, 436.0604.

Compound 1. Compound 1a (65 mg, 0.15 mmol) and 3-bromophenol (26 mg, 0.15 mmol) were added to methanesulfonic acid (250 μL) in a 20-mL scintillation vial. The mixture was stirred at 160° C., for 18 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (44 mg, 50%). ¹H NMR (500 MHz, Methanol-D₄, δ): 8.72 (s, 1H), 8.10 (d, J=7.9 Hz, 1H), 8.03 (d, J=1.9 Hz, 1H), 7.55 (d, J=8.7 Hz, 1H), 7.40 (d, J=7.9 Hz, 1H), 7.16 (d, J=8.7 Hz, 1H), 6.92 (s, 1H), 3.72 (dt, J=20.5, 6.0 Hz, 4H), 3.10 (d, J=6.9 Hz, 2H), 2.77 (d, J=16.3 Hz, 2H), 2.15 (d, J=6.7 Hz, 2H), 2.03 (q, J=14.7 Hz, 3H). ¹³C NMR (126 MHz, Methanol-D₄, δ): 157.4, 155.1, 154.5, 154.3, 146.4, 132.3, 132.0, 131.6, 130.2, 130.2, 130.0, 129.9, 128.7, 127.0, 122.3, 121.4, 121.1, 107.3, 53.4, 53.0, 28.2, 25.7, 21.3, 20.4, 20.3. ESI-MS (m/z): [M−H]⁻ cacld for C₂₅H₁₉BrNO₇S₂, 587.9792. found, 587.9788.

Compound 1e. Anhydrous dichloromethane (5 mL) was added to compound 1 (10 mg, 0.02 mmol) in a flame dried round bottom flask. The flask was then placed on an ice bath before adding oxalyl chloride (23 μL, 0.18 mmol) and dimethylformamide (0.5 mL). The reaction was stirred overnight under room temperature and the solvent was removed to obtain a dark violet compound (11 mg, 99%). ¹H NMR (600 MHz, Methanol-D₄, δ): δ 8.72 (s, 1H), 8.07 (d, J=7.9 Hz, 1H), 7.33 (d, J=7.8 Hz, 1H), 7.12 (d, J=9.4 Hz, 1H), 6.96 (dd, J=9.4, 2.3 Hz, 1H), 6.90 (d. J=2.3 Hz, 1H), 6.77 (s, 1H), 3.56 (dt. J=26.2, 5.8 Hz, 4H), 3.26 (s, 3H), 3.08 (t, J=6.4 Hz, 2H), 2.71 (dtt, J=22.3, 14.9, 6.5 Hz, 2H), 2.10 (p, J=6.6 Hz, 2H), 1.95 (td, J=12.2, 11.5, 6.7 Hz, 2H). ¹³C NMR (151 MHz, Methanol-D₄, δ): 158.4, 157.9, 157.5, 154.3, 153.7, 146.2, 133.5, 132.9, 131.6, 129.3, 128.4, 127.0, 125.7, 115.9, 114.7, 114.1, 106.1, 97.0, 52.2, 51.7, 49.0, 40.7, 28.4, 21.7, 20.8. ESI-MS (m/z): [M−H]⁻ cacld for C₂₅H₁₈BrClNO₆S₂, 605.9526. found, 605.9522.

Compound 2. Compound 1a (65 mg, 0.15 mmol) and 3-fluorophenol (19 mg, 0.15 mmol) were added to methanesulfonic acid (250 μL) in a 20-mL scintillation vial. The mixture was stirred at 160° C., for 24 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (33 mg, 42%). ¹H NMR (500 MHz, Methanol-D₄, δ): 8.68 (d, J=1.7 Hz, 1H), 8.06 (dd, J=7.8, 1.8 Hz, 1H), 7.56 (dd, J=9.2, 2.5 Hz, 1H), 7.36 (d. J=7.9 Hz, 1H), 7.29 (dd, J=9.0, 6.0 Hz, 1H), 7.17 (td, J=8.7, 2.5 Hz, 1H), 6.88 (d, J=1.8 Hz, 1H), 3.68 (dt, J=21.0, 5.8 Hz, 4H), 3.07 (m, 2H), 2.73 (m, 2H), 2.11 (t, J=6.1 Hz, 2H), 1.98 (m, 2H). ¹³C NMR (126 MHz, Methanol-D₄, δ): 168.6, 166.9, 157.1, 155.8, 154.9, 148.5, 146.4, 133.6, 132.5, 131.6, 130.3, 129.8, 128.7, 127.0, 120.6, 120.2, 115.2, 115.1, 107.1, 105.1, 53.3, 52.8, 28.2, 21.3, 20.4. ESI-MS (m/z): [M−H]⁻ cacld for C₂₅H₁₉FNO₇S₂, 528.0592. found, 528.0580 Compound 3. Compound 1a (65 mg, 0.15 mmol) and 3-chlorophenol (17 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (38 mg, 46%). ¹H NMR (500 MHz, DMSO-D₆, δ): 8.11 (d, J=1.9 Hz, 1H), 7.55 (dd. J=7.9, 2.0 Hz, TH), 7.04 (d, J=8.0 Hz, 1H), 6.88 (s, 1H), 6.81 (d, J=2.3 Hz, 1H), 6.78 (d, J=8.6 Hz, 1H), 6.65 (dd, J=8.6, 2.3 Hz, 1H), 6.35 (s, 1H), 3.43 (m, 4H), 2.75 (m, 2H), 2.64 (dd, J=15.7, 8.6 Hz, 2H), 2.01 (t, J=6.9 Hz, 4H). ¹³C NMR (126 MHz, DMSO-D₆, δ): 156.5, 151.7, 146.0, 144.8, 137.3, 134.0, 131.6, 130.6, 128.9, 126.3, 125.9, 124.2, 116.7, 113.5, 51.9, 51.5, 48.6, 42.2, 39.4, 39.2, 39.0, 24.6, 20.6, 19.8, 19.1. ESI-MS (m/z): [M−H]⁻ cacld for C₂₅H₁₉ClNO₇S₂, 544.0297. found, 544.0244.

Compound 4. Compound 1a (65 mg, 0.15 mmol) and 3-Iodophenol (33 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (40 mg, 42%), ¹H NMR (500 MHz, Methanol-D₄, δ): 8.71 (dd, J=3.9, 1.8 Hz, 1H), 8.22 (dd. J=3.9, 1.7 Hz, TH), 8.10 (m, 1H), 7.74 (ddd, J=8.5, 3.9, 1.7 Hz, TH), 7.39 (dd, J=8.0, 3.5 Hz, TH), 6.96 (dd, J=8.5, 3.6 Hz, 1H), 6.90 (d, J=3.5 Hz, 1H), 4.60 (m, 2H), 3.71 (m, 6H), 3.10 (m, 2H), 2.15 (dt, J=15.5, 5.0 Hz, 2H), 2.01 (m, 2H). ¹³C NMR (126 MHz, Methanol-D₄, δ): 157.4, 155.4, 154.4, 153.8, 148.5, 146.4, 135.9, 132.4, 131.6, 131.6, 130.2, 130.2, 128.7, 127.2, 122.7, 121.5, 107.2, 102.6, 53.4, 52.9, 49.0, 28.2, 21.3, 20.4, 20.3. ESI-MS (m/z): [M−H]⁻ cacld for C₂₅H₁₉INO₇S₂, 635.9563. found, 635.9611.

Compound 5. Compound 1a (65 mg, 0.15 mmol) and 2-bromophenol (26 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (37 mg, 42%). ¹H NMR (500 MHz, Methanol-D₄, δ): 8.72 (s, 1H), 8.09 (d, J=7.9 Hz, 1H), 8.05 (d, J=8.0 Hz, 1H), 7.40 (d, J=7.8 Hz, 1H), 7.30 (t, J=7.9 Hz, 1H), 7.24 (dd, J=8.0, 1.4 Hz, 1H), 6.93 (s, 1H), 3.75 (dt, J=25.5, 6.1 Hz, 5H), 3.18 (t, J=6.5 Hz, 2H), 2.78 (m, 2H), 2.18 (d, J=12.2 Hz, 3H), 2.03 (m, 2H). ¹³C NMR (151 MHz, Methanol-D₄, δ): 157.6, 155.2, 154.6, 150.6, 138.9, 132.5, 131.6, 131.1, 130.5, 130.5, 130.2, 128.7, 127.0, 124.6, 121.8, 111.5, 107.6, 53.5, 53.0, 49.0, 28.1, 21.3, 20.6, 20.3 ESI-MS (m/z): [M−H]⁻ cacld for C₂₅H₁₉BrNO₇S₂, 587.9792. found, 587.9712.

Compound 6. Compound 1a (65 mg, 0.15 mmol) and 4-bromophenol (26 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (31 mg, 35%). ¹H NMR (500 MHz, Methanol-D₄, δ): 8.72 (d, J=3.7 Hz, 1H), 8.11 (d, J=7.9 Hz, 1H), 7.90 (dt, J=9.6, 2.9 Hz, 1H), 7.69 (dd, J=9.3, 3.2 Hz, 1H), 7.40 (dd, J=8.1, 3.1 Hz, 1H), 7.28 (s, OH), 6.91 (s, 1H), 3.72 (m, 4H), 3.09 (d, J=6.4 Hz, 2H), 2.77 (d, J=16.5 Hz, 2H), 2.13 (s, 3H), 2.00 (s, 3H). ¹³C NMR (151 MHz, Methanol-D₄, δ): 157.5, 154.6, 154.1, 153.3, 146.5, 138.5, 132.5, 132.2, 131.6, 130.6, 130.2, 128.8, 127.0, 124.7, 121.9, 120.0, 118.9, 107.2, 53.5, 53.0, 49.0, 28.2, 21.3, 20.3, 20.3. ESI-MS (m/z): [M−H]⁻ cacld for C₂₅H₁₉BrNO₇S₂, 587.9792. found, 587.9749.

Compound 7. Compound 1a (65 mg, 0.15 mmol) and 2,4-dibromophenol (38 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (20 mg, 20%). ¹H NMR (500 MHz, Methanol-D₄, δ): 8.70 (d, J=1.9 Hz, 1H), 8.18 (d, J=2.3 Hz, 1H), 8.09 (dd, J=7.8, 1.9 Hz, 1H), 7.40 (d, J=7.9 Hz, 1H), 7.21 (d, J=2.4 Hz, 1H), 6.89 (s, 1H), 3.75 (dt, J=25.3, 5.8 Hz, 4H), 3.14 (t, J=6.3 Hz, 2H), 2.77 (m, 2H), 2.15 (dd, J=10.2, 4.5 Hz, 2H), 2.06-1.91 (m, 2H). ¹³C NMR (151 MHz, Methanol-D₄, δ): 157.8, 154.2, 149.7, 140.4, 132.1, 131.9, 131.6, 131.2, 130.2, 129.2, 128.8, 127.0, 125.6, 122.5, 118.5, 112.7, 108.0, 53.7, 53.2, 28.1, 22.6, 21.7, 21.2, 20.5, 20.2. ESI-MS (m/z): [M−H]⁻ cacld for C₂₅H₁₈Br₂NO₇S₂, 665.8897. found, 665.8835.

Compound 8. Compound 1a (65 mg, 0.15 mmol) and 3,4-difluorophenol (20 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (16 mg, 20%). ¹H NMR (500 MHz, Methanol-D₄, δ): 8.68 (d, J=1.8 Hz, 1H), 8.06 (dd, J=7.9, 1.7 Hz, 1H), 7.79 (dd, J=10.4, 6.3 Hz, 1H), 7.38 (d, J=7.8 Hz, 1H), 7.03 (dd, J=10.4, 8.4 Hz, 1H), 6.87 (d, J=4.0 Hz, 1H), 3.71 (dt, J=21.0, 5.9 Hz, 4H), 3.07 (t, J=6.4 Hz, 2H), 2.74 (q, J=7.0 Hz, 2H), 2.11 (p. J=6.3 Hz, 2H), 1.98 (d. J=6.1 Hz, 2H). ¹³C NMR (151 MHz. Methanol-D₄, δ): 157.2, 154.7, 154.2, 151.2, 146.4, 132.2, 131.6, 130.5, 129.9, 128.8, 127.0, 121.4, 117.7, 117.5, 107.5, 107.3, 53.4, 53.0, 49.1, 48.9, 28.2, 21.7, 21.2, 20.3, 20.3. ESI-MS (m/z): [M−H]⁻ cacld for C₂₅H₁₈F₂NO₇S₂, 546.0498. found, 546.0577.

Compound 9. Compound 1a (65 mg, 0.15 mmol) and 4-chloro-3-fluorophenol (22 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (38 mg, 45%). ¹H NMR (500 MHz, Methanol-D₄, δ): 6 8.74 (s, 1H), 8.13 (d, J=7.9 Hz, 1H), 7.77 (d, J=9.2 Hz, 1H), 7.42 (d, J=7.8 Hz, 1H), 7.27 (d, J=7.8 Hz, 1H), 6.92 (s, 1H), 3.75 (dt, J=20.4, 5.8 Hz, 4H), 3.11 (t, J=6.4 Hz, 2H), 2.78 (tq, J=14.5, 7.2, 4.7 Hz, 2H), 2.15 (p, J=6.2 Hz, 2H), 2.03 (dp, J=13.4, 6.4 Hz, 2H). ¹³C NMR (151 MHz, Methanol-D₄, δ): 163.3, 161.3, 157.4, 154.6, 146.5, 132.0, 132.0, 131.6, 130.4, 130.2, 128.8, 127.0, 121.4, 121.0, 120.0, 119.8, 107.6, 106.7, 106.5, 53.5, 53.0, 28.1, 21.2, 20.3, 20.3. ESI-MS (m/z): [M−H]⁻ cacld for C₂₅H₁₈ClFNO₇S₂, 562.0203. found, 562.0279.

Compound 10. Compound 1a (65 mg, 0.15 mmol) and 4-bromo-3-fluorophenol (29 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (26 mg, 28%). ¹H NMR (500 MHz, Methanol-D₄, δ): 8.69 (s, 1H), 8.08 (dd, J=8.0, 1.6 Hz, 1H), 7.70 (d, J=8.7 Hz, 1H), 7.37 (dd, J=12.4, 7.6 Hz, 2H), 6.87 (s, 1H), 3.72 (dt, J=20.6, 5.9 Hz, 4H), 3.06 (t. J=6.4 Hz, 2H), 2.74 (dt, J=11.6, 7.1 Hz, 2H), 2.12 (t, J=6.1 Hz, 2H), 1.98 (q, J=6.0 Hz, 2H). ¹³C NMR (151 MHz, Methanol-D₄, δ): 157.1, 155.7, 154.9, 133.7, 133.6, 132.5, 131.6, 130.2, 129.8, 128.7, 127.0, 120.2, 115.2, 115.1, 107.2, 105.1, 104.9, 53.3, 52.8, 49.5, 49.3, 49.0, 28.2, 21.3, 20.4, 20.3. ESI-MS (m/z): [M−H]⁻ cacld for C₂₅H₁₈BrFNO₇S₂, 605.9698. found, 605.9677.

Compound 11. Compound 1a (65 mg, 0.15 mmol) and 3-chloro-4-fluorophenol (22 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (25 mg, 30%). ¹H NMR (500 MHz, Methanol-D₄, δ): 8.67 (s, 1H), 8.07 (m, 1H), 7.97 (dd, J=6.2, 1.6 Hz, 1H), 7.38 (dd, J=7.9, 1.5 Hz, 1H), 6.96 (d, J=9.1 Hz, 1H), 6.86 (s, 1H), 3.72 (dt, J=19.8, 5.9 Hz, 4H), 3.06 (t, J=6.6 Hz, 2H), 2.74 (q, J=7.6 Hz, 2H), 2.12 (m, 4H). ¹³C NMR (151 MHz, Methanol-D₄, δ): 157.5, 156.9, 154.9, 154.5, 153.7, 150.3, 146.5, 132.0, 131.6, 130.8, 129.9, 128.8, 127.0, 123.1, 122.0, 120.2, 116.3, 116.1, 111.4, 107.4, 53.5, 53.1, 28.2, 21.2, 20.3. ESI-MS (m/z): [M−H]⁻ cacld for C₂₅H₁₈ClFNO₇S₂, 562.0203. found, 562.0276.

Compound 12. Compound 1a (65 mg, 0.15 mmol) and 3,4-dichlorophenol (25 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (36 mg, 42%). ¹H NMR (500 MHz, Methanol-D₄, δ): 8.58 (s, 1H), 7.84 (d, J=8.2 Hz, 1H), 7.25 (d, J=8.1 Hz, 1H), 6.93 (d, J=9.2 Hz, 2H), 6.79 (s, 1H), 6.64 (s, 1H), 3.49 (d, J=20.0 Hz, 4H), 2.86 (dtd, J=39.0, 21.6, 19.3, 12.0 Hz, 4H), 2.18 (d, J=20.5 Hz, 4H). ¹³C NMR (151 MHz, Methanol-D₄, δ): 156.0, 153.2, 133.4, 132.4, 131.7, 131.6, 131.5, 130.6, 128.3, 126.3, 122.5, 122.0, 120.2, 117.7, 54.3, 54.0, 49.6, 49.3, 48.8, 48.6, 41.7, 25.8, 21.7, 21.3, 20.6. ESI-MS (m/z): [M−H]⁻ cacld for C₂₅H₁₈C₁₂NO₇S₂, 577.9907. found, 577.9981.

Compound 13. Compound 1a (65 mg, 0.15 mmol) and 4-bromo-3-chlorophenol (31 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (37 mg, 40%). ¹H NMR (500 MHz, Methanol-D₄, δ): 8.73 (s, 1H), 8.10 (dd, J=7.8, 1.5 Hz, 1H), 7.87 (d, J=2.0 Hz, 1H), 7.41 (td, J=6.4, 3.0 Hz, 2H), 7.25 (d, J=8.7 Hz, 1H), 6.92 (d, J=1.7 Hz, 1H), 3.74 (m, 5H), 3.11jj (m, 2H), 2.77 (tq, J=15.5, 8.1, 5.6 Hz, 2H), 2.15 (m, 2H), 2.02 (h, J=6.4 Hz, 2H). ¹³C NMR (151 MHz, Methanol-D₄, δ): 155.9, 153.8, 153.2, 153.1, 145.0, 140.3, 131.0, 130.8, 130.1, 128.8, 128.7, 127.3, 125.7, 125.6, 120.6, 119.9, 116.6, 105.8, 52.0, 51.5, 26.7, 19.8, 18.9, 18.9. ESI-MS (m/z): [M−H]⁻ cacld for C₂₅H₁₈BrClNO₇S₂, 621.9402. found, 621.9476.

Compound 14. Compound 1a (65 mg, 0.15 mmol) and 3-bromo-4-fluorophenol (29 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (41 mg, 45%), ¹H NMR (500 MHz, Methanol-D₄, δ): 8.68 (s, 1H), 8.09 (m, 2H), 7.38 (dd, J=7.9, 1.6 Hz, 1H), 6.92 (dd, J=8.6, 1.6 Hz, 1H), 6.86 (d, J=1.7 Hz, 1H), 3.71 (dt, J=19.6, 5.9 Hz, 4H), 3.06 (t, J=6.4 Hz, 2H), 2.74 (t, J=6.0 Hz, 2H), 2.18-2.05 (m, 2H), 1.99 (dd, J=9.7, 3.3 Hz, 2H). ¹³C NMR (151 MHz, Methanol-D₄, δ): 157.8, 157.5, 156.2, 154.5, 153.9, 150.3, 132.0, 130.8, 130.0, 128.8, 127.0, 123.7, 123.7, 123.2, 122.1, 117.6, 115.9, 115.7, 107.4, 53.6, 53.1, 28.2, 21.3, 20.3, 18.4. ESI-MS (m/z): [M−H]⁻ cacld for C₂₅H₁₈BrFNO₇S₂, 605.9698. found, 605.9641.

Compound 15. Compound 1a (65 mg, 0.15 mmol) and 3-bromo-4-chlorophenol (31 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (45 mg, 48%). ¹H NMR (500 MHz, Methanol-D₄, δ): 8.70 (s, 1H), 8.16 (s, 1H), 8.09 (m, 1H), 7.39 (d, J=7.8 Hz, 1H), 7.21 (s, 1H), 6.87 (s, 1H), 3.72 (dt, J=19.9, 6.0 Hz, 4H), 3.06 (m, 2H), 2.75 (tq, J=16.8, 10.2, 8.2 Hz, 2H), 2.12 (t, J=6.2 Hz, 2H), 1.99 (s, 2H). ¹³C NMR (151 MHz, Methanol-D₄, δ): 157.7, 154.3, 153.4, 152.2, 132.6, 132.1, 131.9, 131.6, 130.7, 130.6, 130.3, 129.5, 128.9, 127.0, 123.6, 123.4, 122.2, 111.4, 107.6, 53.6, 53.1, 28.1, 21.2, 20.3, 20.3. ESI-MS (m/z): [M−H]⁻ cacld for C₂₅H₂₀BrClNO₇S₂, 621.9402. found, 621.9478.

Compound 16. Compound 1a (65 mg, 0.15 mmol) and 4-chloro-3-iodophenol (38 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (42 mg, 42%). ¹H NMR (500 MHz, Methanol-D₄, δ): 8.67 (m, 1H), 8.32 (m, 1H), 8.07 (dt, J=8.1, 2.2 Hz, 1H), 7.37 (m, 1H), 7.12 (m, 1H), 6.83 (m, 1H), 3.69 (m, 4H), 3.02 (dt, J=8.4, 4.5 Hz, 2H), 2.72 (m, 2H), 2.09 (m, 2H), 1.96 (m, 2H). ¹³C NMR (151 MHz, Methanol-D₄, δ): 157.7, 154.2, 153.5, 151.6, 136.2, 131.9, 131.7, 130.7, 130.2, 129.9, 128.8, 128.8, 127.0, 125.8, 125.6, 124.3, 122.2, 107.6, 106.3, 53.6, 53.1, 28.2, 21.2, 20.3, 20.3. ESI-MS (m/z): [M−H]⁻ cacld for C₂₅H₁₈ClINO₇S₂, 669.9263. found, 669.9337.

Compound 17. Compound 1a (65 mg, 0.15 mmol) and phenol (14 mg, 0.15 mmol) were added to methanesulfonic acid (250 L) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (29 mg, 38%). ¹H NMR (600 MHz, Methanol-D₄, δ): 8.73 (s, 1H), 8.55 (s, 1H), 7.44 (t, J=8.8 Hz, 2H), 7.17 (t, J=7.9 Hz, 1H), 7.12 (s, 1H), 7.08 (m, 2H), 6.91 (d. J=6.4 Hz, 2H), 3.72 (d. J=25.9 Hz, 4H), 3.10 (d, 0.1=10.2 Hz, 2H), 2.75 (m, 2H), 2.13 (d, J=7.1 Hz, 3H), 2.00 (m, 2H). ³C NMR (151 MHz, Methanol-D₄, δ): 157.1, 156.4, 155.0, 154.5, 136.3, 131.8, 131.6, 131.0, 130.2, 129.8, 128.6, 127.0, 126.5, 123.1, 121.2, 117.9, 116.2, 106.8, 53.3, 52.8, 30.7, 28.2, 24.2, 21.3, 20.4. ESI-MS (m/z): [M−H]⁻ cacld for C₂₅H₂₀NO₇S₂, 510.0687. found, 510.0611.

Compound 18. Compound 1a (65 mg, 0.15 mmol) and 4-bromo-3-(trifluoromethyl)phenol (36 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (36 mg, 35%). ¹H NMR (500 MHz, Methanol-D₄, δ): 8.75 (s, 1H), 8.11 (d, J=8.0 Hz, 1H), 7.84 (t, J=7.8 Hz, 1H), 7.78 (d, J=8.3 Hz, 1H), 7.41 (q, J=7.5 Hz, 1H), 7.30 (d, J=8.1 Hz, 1H), 6.95 (s, 1H), 3.72 (d. J=21.0 Hz, 4H), 3.16 (d, J=25.5 Hz, 2H), 2.79 (d, J=21.3 Hz, 4H), 2.28-1.91 (m, 4H). ¹³C NMR (151 MHz, Methanol-D₄, δ): 167.6, 157.8, 154.9, 152.5, 135.1, 131.6, 130.3, 128.7, 127.0, 127.0, 126.0, 124.2, 122.6, 120.2, 119.0, 116.6, 107.3, 69.1, 53.7, 49.0, 40.2, 31.6, 28.1, 24.0, 20.3, 14.4, 11.4. ESI-MS (m/z): [M−H]⁻ cacld for C₂₆H₁₈BrF₃NO₇S₂, 655.9666. found, 655.9633.

Compound 19. Compound 1a (65 mg, 0.15 mmol) and 2,3-difluorophenol (20 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (32 mg, 36%). ¹H NMR (500 MHz, Methanol-D₄, δ): 8.71 (t, J=2.0 Hz, 1H), 8.09 (dt, J=7.8, 2.0 Hz, 1H), 7.40 (dd, J=7.9, 2.3 Hz, 1H), 7.32 (q. J=9.1 Hz, 1H), 7.08 (t, J=6.8 Hz, 1H), 6.92 (d, J=2.1 Hz, 1H), 3.75 (d, J=21.3 Hz, 4H), 3.11 (d, J=6.9 Hz, 2H), 2.78 (m, 2H), 2.31-1.85 (m, 4H), 13C NMR (151 MHz, Methanol-D₄, δ): 157.6, 155.5, 154.6, 154.1, 146.5, 144.0, 132.1, 131.6, 130.5, 130.3, 128.7, 127.0, 126.8, 121.2, 115.1, 114.9, 111.4, 108.0, 53.6, 53.1, 49.0, 28.1, 21.2, 20.3, 20.3. ESI-MS (m/z): [M−H]⁻ cacld for C₂₅H₁₈F₂NO₇S₂, 546.0498. found, 546.0444.

Compound 20. Compound 1a (65 mg, 0.15 mmol) and 3-chloro-2-fluorophenol (22 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (36 mg, 42%). ¹H NMR (500 MHz, Methanol-D₄, δ): 8.42 (d, J=3.3 Hz, 2H), 7.67 (m, 1H), 7.16 (dd, J=8.2, 3.4 Hz, 1H), 6.90 (d. J=3.3 Hz, 1H), 6.69 (td, J=8.6, 3.4 Hz, 2H), 6.51 (dd, J=9.1, 3.1 Hz, 1H), 6.43 (d, J=3.3 Hz, 1H), 3.66-3.28 (m, 4H), 2.94-2.43 (m, 4H), 2.15-1.91 (m, 4H). ¹³C NMR (151 MHz, Methanol-D₄, δ): 151.6, 149.1, 147.4, 144.7, 144.6, 143.7, 143.6, 139.3, 132.3, 131.2, 130.2, 129.8, 127.1, 124.7, 124.2, 121.6, 121.0, 118.9, 114.5, 53.0, 42.9, 24.3, 20.2, 19.7, 19.0. ESI-MS (m/z): [M−H]⁻ cacld for C₂₅H₁₈ClFNO₇S₂, 562.0203. found, 562.0244.

Compound 21. Compound 1a (65 mg, 0.15 mmol) and 3-bromo-2-fluorophenol (29 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (41 mg, 45%). ¹H NMR (600 MHz, Methanol-D₄, δ): 8.42 (d, J=3.9 Hz, 1H), 7.66 (t, J=5.8 Hz, 1H), 7.16 (dd, J=8.0, 3.8 Hz, 1H), 6.88 (d, J=4.0 Hz, 1H), 6.73 (td, J=8.6, 4.0 Hz, 1H), 6.53 (dd, J=8.9, 3.8 Hz, 1H), 6.48 (s, 1H), 3.39 (d, J=27.6 Hz, 4H), 2.89-2.45 (m, 4H), 2.23-1.84 (m, 4H). ¹³C NMR (151 MHz, Methanol-D₄, δ): 153.1, 151.5, 149.6, 145.9, 145.8, 145.0, 140.9, 133.8, 133.1, 132.6, 131.3, 128.7, 128.4, 126.1, 122.4, 120.3, 116.7, 113.7, 54.0, 49.0, 46.6, 25.8, 21.7, 21.1, 20.4. ESI-MS (m/z): [M−H]⁻ cacld for C₂₅H₁₈BrFNO₇S₂, 605.9698. found, 605.9141.

Compound 22. Compound 1a (65 mg, 0.15 mmol) 2-chloro-3-fluorophenol (22 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (40 mg, 47%). ¹H NMR (600 MHz, Methanol-D₄, δ):8.66 (d, J=2.0 Hz, 1H), 8.05 (dd, J=7.9, 1.8 Hz, 1H), 7.37 (d, J=7.9 Hz, 1H), 7.28 (t, J=8.8 Hz, 1H), 7.20 (dd, J=9.2, 5.7 Hz, 1H), 6.88 (s, 1H), 3.72 (dt, J=29.6, 5.9 Hz, 4H), 3.12 (t, J=6.5 Hz, 2H), 2.73 (ddt, J=22.8, 16.0, 8.6 Hz, 2H), 2.13 (p, J=7.1, 6.6 Hz, 2H), 1.98 (dt, J=13.5, 7.3 Hz, 2H). ¹³C NMR (126 MHz, Methanol-D₄, δ): 163.9, 162.8, 161.8, 154.6, 154.3, 151.2, 148.7, 146.5, 132.2, 131.6, 130.9, 130.9, 130.4, 130.3, 128.7, 127.0, 121.1, 114.7, 107.9, 53.1, 49.0, 28.1, 21.2, 20.4, 20.3. ESI-MS (m/z): [M−H]⁻ cacld for C₂₅H₁₈ClFNO₇S₂, 562.0203. found, 562.0277.

Compound 23. Compound 1a (65 mg, 0.15 mmol) 2,3-dichlorophenol (24 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (43 mg, 49%). ¹H NMR (600 MHz, Methanol-D₄, δ): 8.66 (m, 1H), 8.06 (dd, J=7.9, 1.8 Hz, 1H), 7.50 (dd, J=8.9, 2.0 Hz, 1H), 7.12 (dt, J=8.9, 2.2 Hz, 1H), 6.88 (s, 1H), 3.73 (dt. J=29.5, 5.9 Hz, 4H), 3.12 (t, J=6.6 Hz, 2H), 2.89-2.62 (m, 2H), 2.13 (p, J=6.9, 6.5 Hz, 2H), 1.98 (q, J=7.5, 7.0 Hz, 2H). ¹³C NMR (151 MHz, Methanol-D₄, δ): 157.7, 154.2, 150.5, 148.6, 146.4, 139.6, 132.1, 131.6, 131.6, 130.8, 130.2, 129.6, 128.7, 127.4, 127.0, 123.1, 122.0, 121.7, 111.4, 53.6, 53.1, 28.1, 21.2, 20.5, 20.3. ESI-MS (m/z): [M−H]⁻ cacld for C₂₅H₁₈C₁₂NO₇S₂, 577.9907. found, 577.9987.

Compound 24, 4-Formylbenzene-1,3-disulfonic acid disodium salt hydrate (50 mg, 0.16 mmol) and 3-fluorophenol (36 mg, 0.32 mmol) were added to methanesulfonic acid (250 μL) in a 20-mL scintillation vial. The mixture was stirred at 160° C., for 24 h using the sealed tube and cooled it to room temperature before adding ethyl acetate (15 mL) to it. The precipitate formed was collected by suction filtration to give a brown powder (18 mg, 25%). ¹H NMR (500 MHz, Methanol-D₄, δ): 8.56 (s, 1H), 7.96 (m, 1H), 7.39 (d, J=9.4 Hz, 2H), 7.31 (d, J=7.8 Hz, 1H), 7.23 (d, J=2.4 Hz, 1H), 7.12 (d, J=2.4 Hz, 2H), 7.00 (dd. J=9.2, 2.4 Hz, 2H). ¹³C NMR (151 MHz, Methanol-D₄, δ): 177.4, 172.5, 168.2, 163.0, 160.9, 148.9, 146.1, 135.6, 131.9, 131.1, 128.5, 128.4, 126.9, 126.5, 123.6, 120.7, 118.7, 103.0, 102.9, 49.0. ESI-MS (m/z): [M−H]⁻ cacld for C₁₉H₉F₂O₇S₂, 450.9763. found, 450.9777.

Compound 25. 4-Formylbenzene-1,3-disulfonic acid disodium salt hydrate (50 mg, 0.16 mmol) and 3-chlorophenol (41 mg, 0.32 mmol) were added to methanesulfonic acid (250 μL) in a 20-mL scintillation vial. The mixture was stirred at 160° C., for 24 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a brown powder (22 mg, 28%). ¹H NMR (500 MHz, Methanol-D₄, δ): 8.68 (d, J=7.5 Hz, 1H), 8.51 (s, 1H), 8.33 (s, 1H), 8.14 (ddd, J=15.7, 7.9, 1.6 Hz, 1H), 7.89 (s, 1H), 7.76 (m, 2H), 7.45 (m, 2H), ¹³C NMR (151 MHz, Methanol-D₄, δ): 176.4, 163.5, 159.0, 157.2, 152.5, 135.8, 134.8, 134.2, 131.2, 130.8, 129.9, 128.6, 126.7, 124.6, 123.3, 120.2, 49.0, 30.3, 23.8, 18.4. ESI-MS (m/z): [M−H]⁻ cacld for C₁₉H₉Cl₂O₇S₂, 482.9172. found, 482.9111.

Compound 26. 4-Formylbenzene-1,3-disulfonic acid disodium salt hydrate (50 mg, 0.16 mmol) and 3-bromophenol (55 mg, 0.32 mmol) were added to methanesulfonic acid (250 μL) in a 20-mL scintillation vial. The mixture was stirred at 160° C., for 24 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a reddish powder (25 mg, 27%). ¹H NMR (600 MHz, Methanol-D₄, δ): 8.71 (s, 1H), 8.42 (s, 1H), 8.12 (d. J=7.8 Hz, 1H), 7.85 (d. J=9.0 Hz, 1H), 7.72 (d. J=9.4 Hz, 1H), 7.57 (d, J=9.0 Hz, 1H), 7.47 (d, J=7.8 Hz, 1H), 7.39 (s, 1H), 7.31 (d, J=9.4 Hz, 1H). ¹³C NMR (151 MHz, Methanol-D₄, δ): 177.2, 170.4, 163.1, 156.4, 148.5, 137.1, 136.5, 133.7, 132.2, 131.2, 131.0, 128.6, 127.0, 123.3, 123.0, 122.6, 122.5, 121.8, 103.2, 49.0. ESI-MS (m/z): [M−H]⁻ cacld for C₁₉H₉Br₂O₇S₂, 570.8162. found, 570.8166.

Compound 27. 4-Formylbenzene-1,3-disulfonic acid disodium salt hydrate (50 mg. 0.16 mmol) and 3-iodophenol (70 mg, 0.32 mmol) were added to methanesulfonic acid (250 μL) in a 20-mL scintillation vial. The mixture was stirred at 160° C., for 24 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a brown powder (27 mg, 25%). ¹H NMR (500 MHz, Methanol-D₄, δ): 8.71 (s, 1H), 8.63 (d, J=1.5 Hz, 1H), 8.13 (d, J=7.9 Hz, 1H), 8.05 (d, J=8.8 Hz, 1H), 7.72 (d, J=9.4 Hz, 1H), 7.47 (d, J=7.8 Hz, 1H), 7.38 (m, 1H), 7.36 (s, 1H), 7.32 (d, J=9.5 Hz, 1H). ¹³C NMR (151 MHz, Methanol-D₄, δ):177.3, 170.2, 163.1, 156.4, 137.2, 136.4, 133.7, 132.2, 131.8, 131.2, 130.9, 128.6, 126.9, 123.3, 123.0, 122.5, 121.8, 103.2, 49.0. ESI-MS (m/z): [M−H]⁻ cacld for C₁₉H₉I₂O₇S₂. 666.7885. found, 666.7955.

Compound 28. 4-Formylbenzene-1,3-disulfonic acid disodium salt hydrate (50 mg, 0.16 mmol) and 3-nitrophenol (22 mg, 0.16 mmol) were added to methanesulfonic acid (250 μL) in a 20-mL scintillation vial. The mixture was stirred at 160° C., for 20 min using the sealed tube and cooled it to room temperature before adding ethyl acetate (15 mL) to it. The precipitate formed was collected by suction filtration and combined with 3-bromophenol (28 mg, 0.16 mmol) and methanesulfonic acid (250 μL) in a 20-mL scintillation vial. The reaction content was again stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a red powder (18 mg, 21%). ¹H NMR (500 MHz, Methanol-D₄, δ): 8.74 (s, 1H), 8.45 (s, 1H), 8.15 (d, J=7.9 Hz, 1H), 7.88 (d, J=8.9 Hz, 1H), 7.76 (d, J=9.4 Hz, 1H), 7.60 (d, J=8.9 Hz, 1H), 7.48 (d, J=7.8 Hz, 1H), 7.42 (d. J=2.2 Hz, 1H), 7.34 (d. J=9.4 Hz, 1H). ¹³C NMR (151 MHz. Methanol-D₄, δ): 177.3, 170.2, 163.1, 156.4, 145.5, 137.2, 136.4, 133.7, 132.2, 131.2, 130.9, 128.6, 126.9, 123.3, 123.0, 122.5, 121.8, 121.8, 103.2, 49.0. ESI-MS (m/z): [M−H]⁻ cacld for C₁₉H₉BrNO₉S₂, 537.8908. found, 537.8988.

Compound 29. 4-Formylbenzene-1,3-disulfonic acid disodium salt hydrate (50 mg, 0.16 mmol) and 3-methylphenol (17 mg, 0.16 mmol) were added to methanesulfonic acid (250 μL) in a 20-mL scintillation vial. The mixture was stirred at 160° C., for 20 min using the sealed tube and cooled it to room temperature before adding ethyl acetate (15 mL) to it. The precipitate formed was collected by suction filtration and combined with 3-bromophenol (28 mg, 0.16 mmol) and methanesulfonic acid (250 μL) in a 20-mL scintillation vial. The reaction content was again stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a red powder (16 mg, 20%). ¹H NMR (500 MHz, Methanol-D₄, δ): 8.62 (dq, J=5.3, 3.6, 2.8 Hz, 1H), 8.06 (m, 2H), 7.91 (m, 1H), 7.66 (m, 4H), 7.40 (m, 2H), 2.71 (d, J=9.5 Hz, 3H). ¹³C NMR (126 MHz, Methanol-D₄, δ): 175.2, 162.1, 159.9, 159.2, 158.0, 149.7, 146.4, 140.7, 134.1, 133.4, 132.7, 131.8, 130.8, 128.4, 126.9, 124.5, 123.9, 123.4, 119.4, 23.5. ESI-MS (m/z): [M−H]⁻ cacld for C₂₀H₁₂BrO₇S₂, 506.9213. found, 506.9110.

Compound 30, 4-Formylbenzene-1,3-disulfonic acid disodium salt hydrate (50 mg, 0.16 mmol) and 3-morpholinophenol (29 mg, 0.16 mmol) were added to methanesulfonic acid (250 μL) in a 20-mL scintillation vial. The mixture was stirred at 160° C., for 20 min using the sealed tube and cooled it to room temperature before adding ethyl acetate (15 mL) to it. The precipitate formed was collected by suction filtration and combined with 3-bromophenol (28 mg, 0.16 mmol) and methanesulfonic acid (250 μL) in a 20-mL scintillation vial. The reaction content was again stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a brown powder (19 mg, 20%). ¹H NMR (500 MHz, Methanol-D₄, δ): 8.73 (d, J=1.5 Hz, 1H), 8.12 (dd. J=7.8, 1.6 Hz, 1H), 8.06 (d. J=1.5 Hz, 1H), 7.65 (m, 1H), 7.46 (m, 2H), 7.38 (d, J=9.3 Hz, 1H), 7.32 (m, 2H), 4.03 (m, 5H), 3.83 (m, 3H). ¹³C NMR (151 MHz, Methanol-D₄, δ): 161.3, 161.2, 160.3, 155.0, 148.9, 146.4, 135.8, 132.8, 132.2, 131.7, 131.3, 130.6, 128.7, 126.9, 122.6, 121.5, 121.5, 119.2, 111.4, 98.0, 67.5, 65.6, 56.2. ESI-MS (m/z): [M−H]⁻ cacld for C₂₃H₁₇BrNO₈S₂, 577.9584. found, 577.9658.

Compound 31. 4-Formylbenzene-1,3-disulfonic acid disodium salt hydrate (50 mg, 0.16 mmol) and 4-(Imidazol-1-yl)phenol (26 mg, 0.16 mmol) were added to methanesulfonic acid (250 L) and methanesulfonic acid (250 L) in a 20-mL scintillation vial. The mixture was stirred at 160° C., for 20 min using the sealed tube and cooled it to room temperature before adding ethyl acetate (15 mL) to it. The precipitate formed was collected by suction filtration and combined with 3-bromophenol (28 mg, 0.16 mmol) in a 20-mL scintillation vial. The reaction content was again stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a red powder (20 mg, 22%). ¹H NMR (600 MHz, Methanol-D₄, δ): 9.12 (s, 1H), 8.54 (s, 1H), 7.74 (m, 2H), 7.57 (d. J=1.9 Hz, 1H), 7.29 (m, 2H), 7.15 (d, J=7.8 Hz, 1H), 7.03 (s, 1H), 6.98 (d, J=2.4 Hz, 1H), 6.88 (d, J=8.5 Hz, 1H), 6.71 (m, 1H), 6.59 (dd. J=8.5, 2.5 Hz, 1H). ¹³C NMR (151 MHz, Methanol-D₄, δ): 158.5, 158.1, 135.5, 133.9, 132.9, 132.1, 128.3, 127.9, 127.0, 126.7, 125.1, 124.5, 123.5, 123.3, 122.1, 121.3, 121.3, 117.8, 114.9, 111.4, 49.6, 30.8, 30.3. ESI-MS (m/z): [M−H]⁻ cacld for C₂₂H₁₂BrN₂O₇S₂, 558.9275. found, 558.9249.

Compound 32. 4-Formylbenzene-1,3-disulfonic acid disodium salt hydrate (50 mg, 0.16 mmol) and 4-hydroxybenzenesulfonic acid (28 mg, 0.16 mmol) were added to methanesulfonic acid (250 μL) in a 20-mL scintillation vial. The mixture was stirred at 160° C., for 20 min using the sealed tube and cooled it to room temperature before adding ethyl acetate (15 mL) to it. The precipitate formed was collected by suction filtration and combined with 3-bromophenol (28 mg, 0.16 mmol) and methanesulfonic acid (250 μL) in a 20-mL scintillation vial. The reaction content was again stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a red powder (18 mg, 20%). ¹H NMR (600 MHz, Methanol-D₄, δ): 8.67 (s, 1H), 8.40 (s, 1H), 8.10 (t, J=10.4 Hz, 1H), 7.82 (d, J=9.0 Hz, 1H), 7.70 (d, J=9.4 Hz, 1H), 7.54 (d, J=8.8 Hz, 1H), 7.43 (d, J=8.0 Hz, 1H), 7.37 (s, 1H), 7.29 (d, J=9.4 Hz, 1H). ¹³C NMR (151 MHz, Methanol-D₄, δ): 177.1, 170.4, 163.1, 156.4, 149.4, 146.3, 137.2, 136.5, 133.7, 132.2, 131.2, 130.9, 128.6, 126.9, 123.3, 123.0, 122.5, 121.8, 111.4, 103.2. ESI-MS (mi/z): [M−H]⁻ cacld for C₁₉H₁₀BrO₁₀S₃, 572.8625. found, 572.8611.

Compound 33. 4-Formylbenzene-1,3-disulfonic acid disodium salt hydrate (50 mg, 0.16 mmol) and 4′-(1H-1,2,4-triazol-1-yl)phenol (26 mg, 0.16 mmol) were added to methanesulfonic acid (250 μL) in a 20-mL scintillation vial. The mixture was stirred at 160° C., for 20 min using the sealed tube and cooled it to room temperature before adding ethyl acetate (15 mL) to it. The precipitate formed was collected by suction filtration and combined with 3-bromophenol (28 mg, 0.16 mmol) and methanesulfonic acid (250 μL) in a 20-mL scintillation vial. The reaction content was again stirred at 160° C., for 20 h using the sealed tube and cooled it to room temperature before adding ethyl acetate (15 mL) to it. The precipitate formed was collected by suction filtration and residue was purified by a flash column chromatograph over silica gel with 5:1 dichloromethane/methanol as the eluent to give a brown powder (18 mg, 20%). ¹H NMR (600 MHz. Methanol-D₄, δ): 10.32 (s, 1H), 9.01 (s, 1H), 8.68 (m, 1H), 8.40 (m, 1H), 8.11 (m, 1H), 7.83 (dd, J=8.9, 1.8 Hz, 1H), 7.70 (d, J=9.4 Hz, 1H), 7.56 (dd, J=18.9, 8.6 Hz, 1H), 7.46 (t, J=6.9 Hz, 1H), 7.38 (m, 1H), 7.29 (dd, J=9.4, 2.2 Hz, 1H), 6.86 (dd, J=17.9, 9.1 Hz, 1H). ¹³C NMR (151 MHz, Methanol-D₄, δ): 175.8, 168.8, 161.6, 155.0, 144.9, 135.6, 135.0, 132.2, 130.7, 129.8, 129.5, 127.1, 125.5, 122.3, 122.0, 121.5, 121.1, 120.3, 116.0, 101.8, 43.7, 29.3, 13.6. ESI-MS (m/z): [M−H]⁻ cacld for C₂₁H₁₁BrN₃O₇S₂, 559.9227. found, 559.9205.

Compound 35. Compound 1a (65 mg, 0.15 mmol) and 3-nitrophenol (21 mg, 0.15 mmol) were added to methanesulfonic acid (250 μL) in a 20-mL scintillation vial. The mixture was stirred at 160° C., for 16 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (37 mg, 45%). ¹H NMR (500 MHz, Methanol-D₄, δ): 8.69 (s, 1H), 8.07 (d, J=7.7 Hz, 1H), 7.38 (d, J=7.8 Hz, 1H), 7.14 (s, 1H), 6.94 (s, 1H), 6.52 (s, 1H), 3.72 (d, J=24.7 Hz, 4H), 3.13 (d, J=6.2 Hz, 2H), 2.77 (m, 2H), 2.13 (p, J=6.6, 5.8 Hz, 2H), 2.02 (m, 2H). ¹³C NMR (151 MHz, Methanol-D₄, δ): 157.2, 155.1, 154.6, 148.7, 148.4, 146.4, 142.6, 142.0, 132.6, 131.6, 130.7, 129.8, 128.7, 127.1, 124.5, 121.9, 114.4, 107.3, 57.3, 53.4, 52.9, 30.8, 28.2, 21.3, 20.4. ESI-MS (m/z): [M−H]⁻ cacld for C₂₅H₁₉N₂O₉S₂, 555.0537. found, 555.0522.

Compound 36. Compound 1a (65 mg, 0.15 mmol) and 3-(dimethylamino)phenol (21 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (33 mg, 40%). ¹H NMR (600 MHz, Methanol-D₄, δ): 8.72 (s, 1H), 8.07 (m, 1H), 7.32 (d, J=7.8 Hz, 1H), 7.11 (d, J=9.4 Hz, 1H), 6.95 (dd, J=9.5, 2.4 Hz, 1H), 6.90 (d, J=2.3 Hz, 1H), 6.76 (s, 1H), 3.57 (t, J=5.9 Hz, 2H), 3.53 (t, J=6.0 Hz, 2H), 3.07 (m, 2H), 2.71 (ddt, J=32.4, 16.2, 8.3 Hz, 2H), 2.09 (p. J=6.6 Hz, 2H), 1.94 (dd. J=13.3, 7.2 Hz, 2H). ¹³C NMR (151 MHz, Methanol-D₄, δ): 158.4, 157.9, 157.4, 154.3, 153.7, 132.9, 131.6, 129.3, 128.5, 127.0, 125.7, 115.9, 114.7, 114.1, 106.1, 97.0, 52.2, 51.7, 40.7, 28.4, 24.2, 21.7, 20.8, 20.8. ESI-MS (m/z): [M−H]⁻ cacld for C₂₇H₂₅N₂O₇S₂, 553.1102. found, 553.1106.

Compound 37. Compound 1a (65 mg, 0.15 mmol) and 3-(3-hydroxyphenyl)propionic acid (25 mg, 0.15 mmol) were added to methanesulfonic acid (250 μL) in a 20-mL scintillation vial. The mixture was stirred at 160° C., for 17 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (40 mg, 46%). ¹H NMR (600 MHz, Methanol-D₄, δ): 8.70 (s, 1H), 8.07 (d, J=8.0 Hz, 1H), 7.98 (d, J=8.4 Hz, 1H), 7.39 (d. J=7.6 Hz, 1H), 7.05 (m, 2H), 6.87 (s, 1H), 4.02 (m, 2H), 3.54 (dt, J=31.3, 5.7 Hz, 4H), 3.18 (q, J=5.3 Hz, 2H), 2.76 (qd, J=16.6, 15.7, 8.5 Hz, 2H), 2.13 (p, J=7.0, 6.5 Hz, 2H), 1.98 (m, 2H), 0.89 (dd, J=20.9, 7.7 Hz, 2H). ¹³C NMR (151 MHz, Methanol-D₄, δ): 174.4, 156.9, 156.7, 155.0, 154.7, 151.5, 131.6, 131.0, 130.1, 129.5, 128.6, 127.3, 127.0, 126.5, 121.5, 120.7, 117.5, 117.0, 106.7, 53.2, 52.7, 49.0, 35.4, 32.1, 28.2, 21.4, 20.5, 20.4. ESI-MS (m/z): [M−H]⁻ cacld for C₂₈H₂₄NO₉S₂, 582.0893. found, 582.0890.

Compound 38. 4-Formylbenzene-1,3-disulfonic acid disodium salt hydrate (50 mg, 0.16 mmol) and 3-(dimethylamino)phenol (22 mg, 0.16 mmol) were added to methanesulfonic acid (250 μL) in a 20-mL scintillation vial. The mixture was stirred at 160° C., for 25 min using the sealed tube and cooled it to room temperature before adding ethyl acetate (15 mL) to it. The precipitate formed was collected by suction filtration and residue was purified by a flash column chromatograph over silica gel with 10:1 dichloromethane/methanol as the eluent to give a reddish powder (60 mg, 94%). The reddish powder (60 mg, 0.15 mmol) and 4-bromophenol (26 mg, 0.15 mmol) were added to methanesulfonic acid (250 μL) in a 20-mL scintillation vial. The mixture was stirred at 160° C., for 24 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (36 mg, 45%). ¹H NMR (600 MHz, Methanol-D₄, δ): 8.71 (s, 1H), 8.11 (d, J=7.8 Hz, 1H), 7.98 (m, 1H), 7.70 (dd, J=8.9, 2.5 Hz, 1H), 7.43 (d, J=8.1 Hz, 1H), 7.35 (m, 3H), 7.13 (m, 1H), 3.46 (d, J=52.7 Hz, 6H). ¹³C NMR (151 MHz, Methanol-D₄, δ): 162.2, 160.7, 158.6, 153.8, 139.9, 135.2, 133.0, 131.7, 131.4, 128.8, 127.0, 124.8, 121.9, 120.3, 120.0, 119.5, 97.8, 46.1, 42.4, 42.0, 30.7. ESI-MS (m/z): [M−H]⁻ cacld for C₂₁H₁₅BrNO₇S₂, 535.9482. found, 535.9488.

Compound 39. 4-Formylbenzene-1,3-disulfonic acid disodium salt hydrate (50 mg, 0.16 mmol) and 3-Diethylaminophenol (26 mg, 0.16 mmol) were added to methanesulfonic acid (250 μL) in a 20-mL scintillation vial. The mixture was stirred at 160° C., for 25 min using the sealed tube and cooled it to room temperature before adding ethyl acetate (15 mL) to it. The precipitate formed was collected by suction filtration and residue was purified by a flash column chromatograph over silica gel with 10:1 dichloromethane/methanol as the eluent to give a reddish powder (65 mg, 95%). The reddish powder (64 mg, 0.15 mmol) and 3-bromophenol (26 mg, 0.15 mmol) were added to methanesulfonic acid (250 μL) in a 20-mL scintillation vial. The mixture was stirred at 160° C., for 22 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (40 mg, 47%). ¹H NMR (600 MHz, Methanol-D₄, δ): 8.56 (s, 1H), 8.09 (s, 1H), 7.70 (d, J=8.3 Hz, 2H), 7.39 (s, 1H), 7.35 (d, J=5.7 Hz, 1H), 7.14 (dd, J=23.7, 8.5 Hz, 2H), 7.05 (d, J=7.8 Hz, 1H), 6.79 (s, 1H), 3.66 (s, 4H), 1.12 (q, J=10.8, 9.1 Hz, 6H). ¹³C NMR (151 MHz, Methanol-D₄, δ): 163.3, 152.7, 151.8, 148.2, 137.9, 134.4, 133.4, 132.9, 129.6, 128.6, 128.4, 125.9, 124.6, 122.1, 120.4, 118.4, 118.1, 111.8, 97.7, 54.9, 38.6, 30.7, 10.7. ESI-MS (m/z): [M−H]⁻ cacld for C₂₃H₁₉BrNO₇S₂, 563.9788. found, 563.9789.

Compound 40. Compound 1a (65 mg, 0.15 mmol) and m-cresol (16 mg, 0.15 mmol) were added to methanesulfonic acid (250 L) in a 20-mL scintillation vial. The mixture was stirred at 160° C., for 24 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (32 mg, 40%). ¹H NMR (500 MHz, Methanol-D₄, δ): 8.73 (s, 1H), 8.10 (dd, J=8.2, 3.6 Hz, 1H), 7.63 (d. J=3.6 Hz, 1H), 7.38 (dd, J=8.0, 3.6 Hz, 1H), 7.27 (dd, J=8.7, 3.5 Hz, 1H), 7.18 (dd. J=8.4, 3.5 Hz, 1H), 6.93 (d, J=3.4 Hz, 1H), 3.70 (dt, J=21.8, 5.7 Hz, 4H), 3.13 (q, J=6.4, 5.3 Hz, 2H), 2.77 (m, 2H), 2.57 (d, J=3.6 Hz, 3H), 2.16 (m, 2H), 2.00 (m, 2H). ¹³C NMR (126 MHz, Methanol-D₄, δ): 156.9, 156.8, 155.0, 154.8, 149.2, 148.0, 132.9, 131.6, 130.8, 130.1, 130.0, 129.3, 128.6, 128.2, 127.0, 121.0, 120.4, 117.8, 106.6, 86.3, 53.1, 52.6, 28.3, 22.2, 21.4, 20.4. ESI-MS (m/z): [M−H]⁻ cacld for C₂₆H₂₂NO₇S₂, 524.0840. found, 524.0844.

Compound 41. Compound 1a (65 mg, 0.15 mmol) and 3-hydroxyphenylboronic acid (21 mg, 0.15 mmol) were added to methanesulfonic acid (250 μL) in a 20-mL scintillation vial. The mixture was stirred at 160° C., for 22 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (40 mg, 48%). ¹H NMR (600 MHz, Methanol-D₄, δ): 8.71 (s, 1H), 8.08 (m, 1H), 7.82 (m, 1H), 7.76 (d, J=8.4 Hz, 1H), 7.38 (m, 1H), 7.27 (m, 1H), 6.93 (m, 1H), 3.70 (dt, J=26.5, 6.0 Hz, 4H), 3.11 (m, 2H), 2.77 (m, 2H), 2.14 (q, J=6.6, 6.1 Hz, 4H). ¹′C NMR (151 MHz, Methanol-D₄, δ): 155.7, 155.0, 153.5, 153.1, 134.8, 131.4, 130.2, 129.6, 128.7, 128.4, 127.2, 125.6, 125.1, 121.7, 119.8, 116.7, 116.5, 105.4, 51.8, 51.4, 44.9, 26.8, 19.9, 19.0, 18.9. ESI-MS (m/z): [M−H]⁻ cacld for C₂₅H₂₁BNO₉S₂, 554.0755. found, 554.0759.

Compound 42. 4-Formylbenzene-1,3-disulfonic acid disodium salt hydrate (50 mg, 0.16 mmol) and 3-(dimethylamino)phenol (22 mg, 0.16 mmol) were added to methanesulfonic acid (250 μL) in a 20-mL scintillation vial. The mixture was stirred at 160° C., for 25 min using the sealed tube and cooled it to room temperature before adding ethyl acetate (15 mL) to it. The precipitate formed was collected by suction filtration and residue was purified by a flash column chromatograph over silica gel with 10:1 dichloromethane/methanol as the eluent to give a reddish powder (60 mg, 94%). The reddish powder (60 mg, 0.15 mmol) and 3-bromophenol (26 mg, 0.15 mmol) were added to methanesulfonic acid (250 μL) in a 20-mL scintillation vial. The mixture was stirred at 160° C., for 22 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (39 mg, 49%). ¹H NMR (600 MHz, Methanol-D₄, δ): 8.69 (d, J=1.7 Hz, 1H), 8.09 (dd, J=7.9, 1.7 Hz, 1H), 8.01 (d, J=1.8 Hz, 1H), 7.60 (dt, J=8.7, 1.4 Hz, 1H), 7.42 (d. J=7.8 Hz, 1H), 7.34 (m, 2H), 7.24 (m, 1H), 7.12 (d, J=1.9 Hz, 1H), 3.47 (d, J=9.6 Hz, 6H). ¹³C NMR (151 MHz, Methanol-D₄, δ): 160.7, 159.1, 158.3, 153.4, 147.4, 145.0, 133.9, 131.2, 130.4, 130.3, 129.9, 129.0, 127.2, 125.5, 121.0, 119.9, 118.2, 116.7, 96.4, 40.9, 16.9. ESI-MS (m/z): [M−H]⁻ cacld for C₂₁H₁₅BrNO₇S₂, 535.9482. found, 535.9483.

Compound 43, 4-Formylbenzene-1,3-disulfonic acid disodium salt hydrate (50 mg, 0.16 mmol) and 3-(dimethylamino)phenol (44 mg, 0.32 mmol) were added to methanesulfonic acid (350 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (36 mg, 45%). ¹H NMR (600 MHz, DMSO-D₆, δ): 8.17 (s, 1H), 7.63 (d, J=7.9 Hz, 1H), 7.05 (d, J=7.8 Hz, 1H), 6.95 (s, 4H), 6.81 (s, 2H), 3.07 (s, 12H). ¹³C NMR (151 MHz, DMSO-D₆, δ): 206.9, 160.7, 157.3, 157.2, 149.6, 147.1, 133.0, 129.4, 129.2, 125.9, 125.6, 118.5, 114.3, 114.1, 110.0, 96.0, 49.0, 40.9, 31.1. ESI-MS (m/z): [M−H]⁻ cacld for C₂₃H₂₁N₂O₇S₂, 501.0862. found, 501.0866.

Compound 44. Compound 1a (65 mg, 0.15 mmol) and 6-Bromo-2-naphthol (33 mg, 0.15 mmol) were added to methanesulfonic acid (250 μL) in a 20-mL scintillation vial. The mixture was stirred at 160° C., for 20 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (42 mg, 44%). ¹H NMR (600 MHz, Methanol-D₄, δ): 8.81 (s, 1H), 8.31 (dd, J=9.2, 3.2 Hz, 1H), 8.20 (q, J=2.2 Hz, 1H), 8.12 (dd, J=8.0, 2.8 Hz, 1H), 7.92 (dd, J=9.0, 3.3 Hz, 1H), 7.43 (dq, J=9.4, 2.2 Hz, 1H), 7.33 (dd, J=7.9, 3.3 Hz, 1H), 7.18 (dd, J=9.3, 3.0 Hz, 1H), 6.90 (m, 1H), 3.69 (d, J=29.2 Hz, 4H), 3.16 (q, J=6.4, 5.2 Hz, 2H), 2.90-2.68 (m, 2H), 2.22-1.94 (m, 4H). ¹³C NMR (151 MHz, Methanol-D₄, δ): 156.7, 156.0, 155.9, 153.7, 148.8, 145.8, 138.6, 136.4, 134.5, 132.8, 132.6, 130.4, 130.2, 129.9, 129.7, 129.7, 129.5, 127.6, 121.9, 121.6, 119.7, 117.8, 105.7, 52.9, 52.5, 28.5, 21.3, 20.4, 20.3. ESI-MS (m/z): [M−H]⁻ cacld for C₂₉H₂₁BrNO₇S₂, 638.0021. found, 638.0027.

Compound 45. Compound 1a (65 mg, 0.15 mmol) and 1-Bromo-2-naphthol (33 mg, 0.15 mmol) were added to methanesulfonic acid (250 μL) in a 20-mL scintillation vial. The mixture was stirred at 160° C., for 22 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (38 mg, 40%). ¹H NMR (600 MHz, Methanol-D₄, δ): 8.82 (s, 1H), 8.32 (dd, J=9.4, 3.8 Hz, 1H), 8.21 (q, J=2.3 Hz, 1H), 8.13 (d, J=8.6 Hz, 1H), 7.93 (dd, J=9.3, 4.1 Hz, 1H), 7.44 (dt, J=9.4, 2.6 Hz, 1H), 7.34 (dd, J=8.1, 3.7 Hz, 1H), 7.19 (dd, J=9.5, 3.8 Hz, 1H), 6.90 (d, J=3.6 Hz, 1H), 3.76-3.63 (m, 4H), 3.16 (q, J=6.4, 5.7 Hz, 2H), 2.88-2.68 (m, 2H), 2.22-1.94 (m, 4H). ¹³C NMR (151 MHz, Methanol-D₄, δ): 156.7, 156.0, 155.9, 153.7, 138.6, 136.4, 134.5, 132.8, 132.6, 130.9, 130.4, 130.2, 129.9, 129.7, 129.5, 127.6, 127.2, 121.9, 121.6, 119.7, 117.8, 113.5, 105.7, 52.9, 52.5, 28.5, 21.3, 20.4, 20.3. ESI-MS (m/z): [M−H]⁻ cacld for C₂₉H₂₁BrNO₇S₂, 638.0021. found, 638.0029.

Compound 46. 4-Formylbenzene-1,3-disulfonic acid disodium salt hydrate (50 mg, 0.16 mmol) and 3-diethylaminophenol (53 mg, 0.32 mmol) were added to methanesulfonic acid (350 L) in a 20-mL scintillation vial. The mixture was stirred at 160° C., for 20 h using the sealed tube and cooled it to room temperature before adding ethyl acetate (15 mL) to it. The precipitate formed was collected by suction filtration and the residue was purified by preparative reverse-phase column chromatography (XBridge Prep OBD C8 5 μm 19 mm×100 mm column, 5-100% acetonitrile/water) to give a reddish powder (38 mg, 43%). ¹H NMR (600 MHz, Methanol-d₄, δ): 8.72 (s, 1H), 8.08 (m, 1H), 7.37 (d, J=7.3 Hz, 1H), 7.18 (d, J=9.3 Hz, 2H), 7.01 (d, J=9.0 Hz, 2H), 6.92 (d, J=2.3 Hz, 2H), 3.67 (q, J=7.3 Hz, 8H), 1.30 (t, 12H). ¹³C NMR (151 MHz, Methanol-D₄, δ): 159.4, 159.1, 157.1, 133.9, 133.1, 131.5, 128.5, 127.0, 115.5, 114.9, 96.8, 46.8, 12.8. ESI-MS (m/z): [M−H]⁻ cacld for C₂₇H₂₉N₂O₇S₂, 557.6640. found, 557.6608.

Compound 47. A mixer of anhydrous dichloromethane (2.0 mL) and anhydrous dimethylformamide (1.0 mL) was added to compound 43 (10 mg, 0.02 mmol) in a flame dried round bottom flask. The flask was then placed on an ice bath before dropwise addition of oxalyl chloride (16 μL, 0.18 mmol). The reaction was stirred overnight under room temperature and the solvent was removed under reduced pressure to obtain a dark violet compound (10 mg, 99%). It was then dissolved in dimethylformamide (3.0 mL) with triethylamine (28 μL, 0.2 mmol) and N-deacetyl colchicine (7.1 mg, 0.02 mmol) was added to it. The mixture was stirred at 40° C., overnight (>8 hours) and monitored with TLC. Upon completion of the reaction, the compound was concentrated under reduced pressure and purified by preparative reverse-phase column chromatography (XBridge Prep OBD C8 5 μm 19 mm×100 mm column, 5-100% acetonitrile/water) to give a violet powder (13 mg, 75%). ¹H NMR (400 MHz, Methanol-d₄, δ): 8.67 (s, 1H), 8.04 (d, J=7.7 Hz, 1H), 7.41 (d, J=10.8 Hz, 1H), 7.32 (d, J=7.9 Hz, 1H), 7.28-7.04 (m, 8H), 6.76 (s, 1H), 4.01 (s, 3H), 3.87 (s, 3H), 3.84 (s, 3H), 3.65 (s, 3H), 3.33 (d. J=13.1 Hz, 12H), 2.70 (m, 1H), 2.41 (m, 3H), 2.00 (d, J=10.8 Hz, 1H). ¹³C NMR (151 MHz. Methanol-D₄, δ): 179.7, 165.8, 163.3, 160.3, 160.0, 159.7, 158.0, 155.8, 154.2, 152.1, 147.9, 146.3, 142.8, 139.1, 137.6, 135.2, 134.3, 132.5, 131.4, 131.0, 128.6, 127.0, 125.7, 118.2, 117.6, 117.3, 117.0, 115.6, 109.0, 108.9, 108.1, 97.4, 61.7, 61.5, 57.4, 56.7, 54.6, 43.9, 41.4, 37.0, 30.0, 14.4, 12.7. ESI-MS (m/z): [M−H]⁻ cacld for C₄₃H₄₂N₃O₁₁S₂, 840.9470. found, 840.9388.

Compound 48. A mixer of anhydrous dichloromethane (2.0 mL) and anhydrous dimethylformamide (1.0 mL) was added to compound 46 (11 mg, 0.02 mmol) in a flame dried round bottom flask. The flask was then placed on an ice bath before dropwise addition of oxalyl chloride (16 μL, 0.18 mmol). The reaction was stirred overnight under room temperature and the solvent was removed under reduced pressure to obtain a dark violet compound (11 mg, 99%). It was then dissolved in dimethylformamide (3.0 mL) with triethylamine (28 μL, 0.2 mmol) and N-deacetyl colchicine (7.1 mg, 0.02 mmol) was added to it. The mixture was stirred at 40° C., overnight (>8 hours) and monitored with TLC. Upon completion of the reaction, the compound was concentrated under reduced pressure and purified by preparative reverse-phase column chromatography (XBridge Prep OBD C8 5 μm 19 mm×100 mm column, 5-100% acetonitrile/water) to give a violet powder (13 mg, 71%). ¹H NMR (600 MHz, Methanol-d₄, δ): 8.69 (s, 1H), 8.07 (m, 1H), 7.45 (d, J=10.8 Hz, 1H), 7.36 (d, J=7.7 Hz, 1H), 7.30-7.21 (m, 2H), 7.16 (t, J=9.9 Hz, 2H), 7.01 (m, 2H), 6.92 (d, J=2.3 Hz, 2H), 6.80 (s, 1H), 4.03 (s, 3H), 3.87 (dd, J=14.5, 1.7 Hz, 6H), 3.67 (d, J=6.9 Hz, 11H), 2.75 (dd, J=13.0, 5.6 Hz, 1H), 2.45 (dtt, J=32.0, 13.0, 6.5 Hz, 2H), 2.06 (d, J=6.5 Hz, 1H), 1.30 (t, J=7.0 Hz, 12H). ¹³C NMR (151 MHz, Methanol-D₄, δ): 180.1, 165.7, 163.3, 159.4, 159.0, 157.1, 155.7, 152.1, 148.3, 147.7, 146.1, 142.8, 138.7, 137.2, 135.2, 133.9, 133.0, 131.5, 131.0, 128.5, 127.0, 125.8, 115.5, 115.1, 114.9, 108.9, 96.8, 61.7, 61.5, 57.3, 56.7, 54.5, 46.8, 37.0, 30.0, 12.8. ESI-MS (m/z): [M−H]⁻ cacld for C₄₇H₅₀N₃O₁₁S₂, 897.0445. found, 897.0456.

Compound 49. A mixer of anhydrous dichloromethane (2.0 mL) and anhydrous dimethylformamide (1.0 mL) was added to compound 43 (10 mg, 0.02 mmol) in a flame dried round bottom flask. The flask was then placed on an ice bath before dropwise addition of oxalyl chloride (16 μL, 0.18 mmol). The reaction was stirred overnight under room temperature and the solvent was removed under reduced pressure to obtain a dark violet compound (10 mg, 99%). It was then dissolved in dimethylformamide (3.0 mL) with triethylamine (28 μL, 0.2 mmol) and deferoxamine (11.2 mg, 0.02 mmol) was added to it. The mixture was stirred at 50° C., overnight (>8 hours) and monitored with TLC. Upon completion of the reaction, the compound was concentrated under reduced pressure and purified by preparative reverse-phase column chromatography (XBridge Prep OBD C8 5 μm 19 mm×100 mm column, 5-100% acetonitrile/water) to give a violet powder (17 mg, 80%). ¹H NMR (600 MHz, Methanol-d₄, δ): 8.72 (s, 1H), 8.11-8.06 (m, 1H), 7.37 (d, J=7.6 Hz, 1H), 7.20 (d, J=9.3 Hz, 2H), 7.05-7.01 (m, 2H), 6.91 (d, J=2.2 Hz, 2H), 3.60 (s, 16H), 3.30 (s, 10H), 3.17 (q. J=5.7, 4.5 Hz, 10H), 2.79-2.75 (m, 9H), 2.46 (d, J=7.4 Hz, 10H), 2.10 (s, 6H), 1.63 (q, J=7.3 Hz, 8H), 1.53 (q, J=7.1, 6.6 Hz, 11H), 1.37-1.30 (m, 13H). ¹³C NMR (151 MHz, Methanol-D₄, δ): 174.9, 174.5, 173.5, 159.8, 159.2, 159.0, 148.3, 146.2, 133.7, 133.0, 131.5, 128.5, 127.0, 115.6, 115.0, 97.1, 40.9, 40.8, 40.4, 40.3, 31.5, 30.0, 28.9, 27.3, 24.9, 24.9, 20.3. ESI-MS (m/z): [M−H]⁻ cacld for C₄₈H₆₈N₈O₁₄S₂, 1046.2322. found, 1046.2147.

Compound 50. A mixer of anhydrous dichloromethane (2.0 mL) and anhydrous dimethylformamide (1.0 mL) was added to compound 43 (10 mg, 0.02 mmol) in a flame dried round bottom flask. The flask was then placed on an ice bath before dropwise addition of oxalyl chloride (16 μL, 0.18 mmol). The reaction was stirred overnight under room temperature and the solvent was removed under reduced pressure to obtain a dark violet compound (10 mg, 99%). It was then dissolved in dimethylformamide (3.0 mL) with triethylamine (28 μL, 0.2 mmol) and 2-aminoethanethiol (1.6 mg, 0.02 mmol) was added to it. The mixture was stirred at 50° C., overnight (>8 hours) and monitored with TLC. Upon completion of the reaction, the compound was concentrated under reduced pressure and purified by preparative reverse-phase column chromatography (XBridge Prep OBD C8 5 μm 19 mm×100 mm column, 5-100% acetonitrile/water) to give a violet powder (10 mg, 92%). ¹H NMR (600 MHz, Methanol-d₄, δ): 8.69 (s, 1H), 8.06 (d, J=7.8 Hz, 1H), 7.36 (d, J=7.7 Hz, 1H), 7.23 (d, J=9.5 Hz, 1H), 7.14 (q, J=9.2 Hz, 3H), 7.09-6.98 (m, 1H), 3.34 (m, 4H), 3.29 (s, J=2.5 Hz, 9H). ¹³C NMR (151 MHz, Methanol-D₄, δ): 163.2, 160.1, 159.9, 159.7, 159.5, 158.2, 154.2, 133.9, 132.2, 131.5, 130.9, 128.5, 127.0, 118.2, 117.4, 116.9, 116.6, 116.4, 109.4, 97.4, 97.0, 44.3, 43.8, 41.2, 18.8. ESI-MS (m/z): [M−H]⁻ cacld for C₂₅H₂₇N₃O₆S₃, 562.5122. found, 562.1122.

Compound 51. The compound 50 (6 mg, 0.01 mmol) was dissolved in dimethylformamide (1.0 mL) with triethylamine (28 μL, 0.2 mmol) and Paclitaxel-SMCC (11 mg, 0.01 mmol) was added to it. The mixture was stirred at 35° C., overnight (>8 hours) and monitored with TLC. Upon completion of the reaction, the content was concentrated under reduced pressure and purified by automated normal phase chromatography (Biotage®, SNAP Ultra 10 g; dichloromethane/methanol 50/50) to obtain a red powder (14 mg, 85%). ¹³C NMR (151 MHz, DMSO-D₆, δ): 202.4, 178.6, 175.8, 174.3, 169.7, 169.1, 168.8, 167.0, 166.4, 165.2, 155.7, 149.2, 146.6, 139.3, 137.2, 134.2, 133.5, 133.4, 133.0, 132.6, 131.5, 131.5, 129.9, 129.5, 129.1, 128.7, 128.4, 127.7, 127.4, 125.5, 125.2, 114.0, 113.7, 95.9, 83.6, 80.3, 76.7, 76.6, 75.3, 74.7, 74.4, 70.8, 70.4, 69.8, 67.4, 57.4, 55.3, 53.9, 52.3, 46.1, 43.4, 42.9, 42.0, 41.7, 39.9, 39.8, 39.7, 39.5, 39.4, 39.2, 39.1, 38.1, 36.5, 35.7, 35.2, 34.4, 31.3, 29.8, 29.0, 28.8, 28.7, 28.4, 27.9, 27.6, 26.3, 23.2, 22.7, 22.4, 22.1, 21.4, 20.7, 13.9, 10.8, 9.8. ESI-MS (m/z): [M−H]⁻ cacld for C₈₄H₉₀N₅O₂₃S₃, 1633.1120. found, 1633.2212.

Compounds 52a/52b. Compound 1e (10 mg, 0.02 mmol) was dissolved in anhydrous dichloromethane (5 mL) followed by addition of N,N-diisopropylamine (20 μL, 0.16 mmol) in a flame dried round bottom flask. Methotrexate (9.0 mg, 0.02 mmol) was dissolved in 1 mL of dimethyl sulfoxide and added to the round bottom flask. The mixture was stirred at 40° C., overnight (>8 hours). The reaction content was concentrated in a rotary evaporator and residue was purified by a flash column chromatograph over silica gel with 5:1 dichloromethane/methanol as the eluent to give a reddish powder (8.2 mg, 40%). ESI-MS (m/z): [M−H]⁻ cacld for C₄₅H₃₉BrN₉O₁₁S₂, 1024.1399. found, 1017.2284.

Compound 55. A mixture of anhydrous dichloromethane (2.0 mL) and anhydrous dimethylformamide (1.0 mL) was added to compound 43 (10 mg, 0.02 mmol) in a flame dried round bottom flask. The flask was then placed on an ice bath before dropwise addition of oxalyl chloride (16 μL, 0.18 mmol). The reaction was stirred overnight under room temperature and the solvent was removed under reduced pressure to obtain a dark violet compound 54 (10 mg, 99%). Next, the compound 54 dissolved in dimethylformamide (3.0 mL) with triethylamine (28 μL, 0.2 mmol) and N-Boc-ethylenediamine (3.2 mg, 0.02 mmol) was added to it. The mixture was stirred at 50° C., overnight (>8 hours) and monitored with TLC. Upon completion of the reaction, the compound was concentrated under reduced pressure and purified by preparative reverse-phase column chromatography (XBridge Prep OBD C8 5 μm 19 mm×100 mm column, 5-100% acetonitrile/water) to give a violet powder (11.5 mg, 90%). ¹H NMR (600 MHz, Methanol-d₄, δ): 8.72 (d, J=1.8 Hz, 1H), 8.09 (dt, J=7.9, 1.6 Hz, 1H), 7.86 (s, 1H), 7.37 (d, J=7.8 Hz, 1H), 7.19 (d, J=9.5 Hz, 2H), 7.02 (m, 2H), 6.87 (t, J=3.0 Hz, 2H), 3.46 (t, J=5.6 Hz, 2H), 3.37 (m, 2H), 3.27 (d, J=1.7 Hz, 12H), 1.45 (s, 9H). ¹³C NMR (151 MHz, Methanol-D₄, δ): 159.8, 159.7, 159.2, 159.1, 158.9, 158.8, 158.8, 158.7, 158.5, 156.8, 148.3, 146.2, 133.6, 133.0, 132.9, 131.5, 128.5, 126.9, 115.6, 115.0, 97.2, 97.2, 80.7, 80.5, 43.1, 41.3, 40.9, 39.0, 28.7, 28.7. ESI-MS (m/z): [M+H]⁺ cacld for C₃₀H₃₇N₄O₈S₂, 645.1978. found, 645.1987.

Compound 56. A mixture of anhydrous dichloromethane (2.0 mL) and anhydrous dimethylformamide (1.0 mL) was added to compound 46 (10 mg, 0.02 mmol) in a flame dried round bottom flask. The flask was then placed on an ice bath before dropwise addition of oxalyl chloride (16 μL, 0.18 mmol). The reaction was stirred overnight under room temperature and the solvent was removed under reduced pressure to obtain a dark violet compound (10 mg, 99%). It was then dissolved in dimethylformamide (3.0 mL) with triethylamine (28 μL, 0.2 mmol) and methoxy PEG amine [1 kDa](20 mg, 0.02 mmol) was added to it. The mixture was stirred at 40° C., overnight (>8 hours) and monitored with TLC. Upon completion of the reaction, the compound was concentrated under reduced pressure and purified by preparative reverse-phase column chromatography (XBridge Prep OBD C8 5 μm 19 mm×100 mm column, 5-100% acetonitrile/water) to give a violet powder (25 mg, 81%).

Compounds 57-61 were prepared according to the method described for compound 56 using a methoxy PEG amine having a molecular weight of about 2 kDa, about 3.4 kDa, about 5 kDa, about 10 kDa, and about 20 kDa, respectively.

TABLE 1
Exemplary Compounds

| Cmpd | Structure | Nomenclature | Target |
|---|---|---|---|
| 1 | 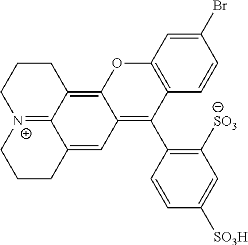 | 2-(12-bromo-1,2,3,5,6,7- hexahydrochromeno[2,3- f]pyrido[3,2,1-ij]quinolin- 4-ium-9-yl)-5-sulfoben- zenesulfonate | Endothelial |
| 2 | 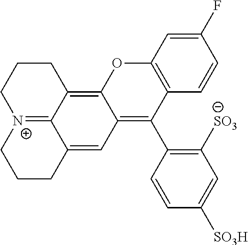 | 2-(12-fluoro-1,2,3,5,6,7- hexahydrochromeno[2,3- f]pyrido[3,2,1-ij]quinolin- 4-ium-9-yl)-5-sulfoben- zenesulfonate | Endothelial |
| 3 | 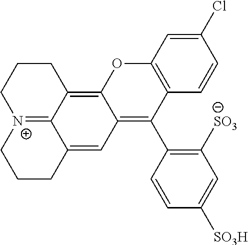 | 2-(12-chloro-1,2,3,5,6,7- hexahydrochromeno[2,3- f]pyrido[3,2,1-ij]quinolin- 4-ium-9-yl)-5-sulfoben- zenesulfonate | Endothelial |
| 4 | 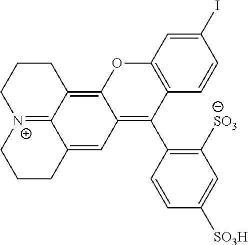 | 2-(12-iodo-1,2,3,5,6,7- hexahydrochromeno[2,3- flpyrido[3,2,1-ij]quinolin- 4-ium-9-yl)-5-sulfoben- zenesulfonate | Endothelial |
| 5 | 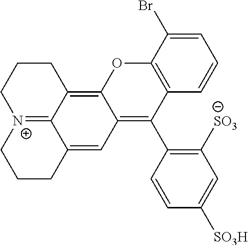 | 2-(13-bromo-1,2,3,5,6,7- hexahydrochromeno[2,3- f]pyrido[3,2,1-ij]quinolin- 4-ium-9-yl)-5-sulfoben- zenesulfonate | Endothelial |
| 6 | 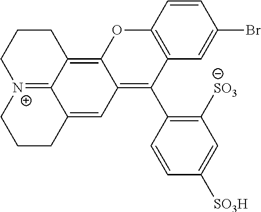 | 2-(11-bromo-1,2,3,5,6,7- hexahydrochromeno[2,3- f]pyrido[3,2,1-ij]quinolin- 4-ium-9-yl)-5-sulfoben- zenesulfonate | Endothelial |
| 7 | 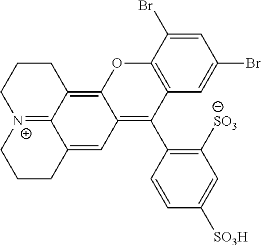 | 2-(11,13-dibromo- 1,2,3,5,6,7-hexahydro- chromeno[2,3-f]pyrido [3,2,1-ij]quinolin-4-ium- 9-yl)-5-sulfobenzene- sulfonate | Endothelial |
| 8 | 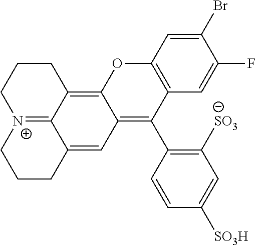 | 2-(11,12-difluoro- 1,2,3,5,6,7-hexahydro- chromeno[2,3-f]pyrido [3,2,1-ij]quinolin-4-ium- 9-yl)-5-sulfobenzene- sulfonate | Endothelial |
| 9 | 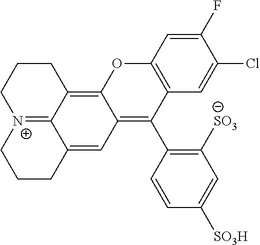 | 2-(11-chloro-12-fluoro- 1,2,3,5,6,7-hexahydro- chromeno[2,3-f]pyrido [3,2,1-ij]quinolin-4-ium- 9-yl)-5-sulfobenzene- sulfonate | Endothelial |
| 10 | 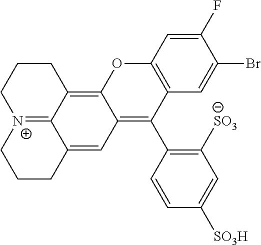 | 2-(11-bromo-12-fluoro- 1,2,3,5,6,7-hexahydro- chromeno[2,3-f]pyrido [3,2,1-ij]quinolin-4-ium- 9-yl)-5-sulfobenzene- sulfonate | Endothelial |
| 11 | 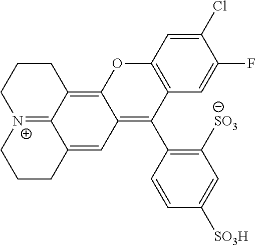 | 2-(12-chloro-11-fluoro- 1,2,3,5,6,7-hexahydro- chromeno[2,3-f]pyrido [3,2,1-ij]quinolin-4-ium- 9-yl)-5-sulfobenzene- sulfonate | Endothelial |
| 12 | 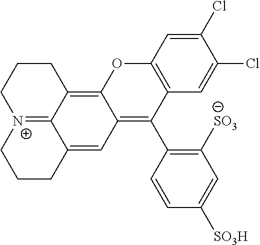 | 2-(11,12-dichloro- 1,2,3,5,6,7-hexahydro- chromeno[2,3-f]pyrido [3,2,1-ij]quinolin-4-ium- 9-yl)-5-sulfobenzene- sulfonate | Endothelial |
| 13 | 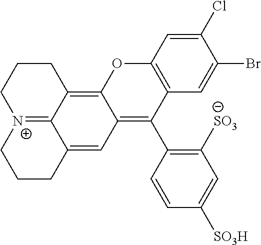 | 2-(11-bromo-12-chloro- 1,2,3,5,6,7-hexahydro- chromeno[2,3-f]pyrido [3,2,1-ij]quinolin-4-ium- 9-yl)-5-sulfobenzene- sulfonate | Endothelial |
| 14 | 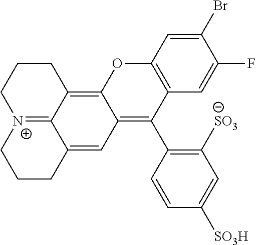 | 2-(12-bromo-11-fluoro- 1,2,3,5,6,7-hexahydro- chromeno[2,3-f]pyrido [3,2,1-ij]quinolin-4-ium- 9-yl)-5-sulfobenzene- sulfonate | Endothelial |
| 15 | 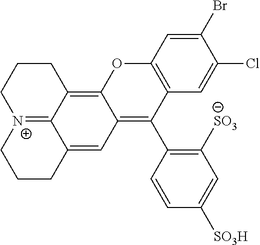 | 2-(12-bromo-11-chloro- 1,2,3,5,6,7-hexahydro- chromeno[2,3-f]pyrido [3,2,1-ij]quinolin-4-ium- 9-yl)-5-sulfobenzene- sulfonate | Endothelial |
| 16 | 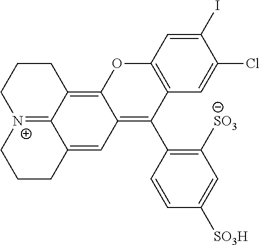 | 2-(11-chloro-12-iodo- 1,2,3,5,6,7-hexahydro- chromeno[2,3-f]pyrido [3,2,1-ij]quinolin-4-ium- 9-yl)-5-sulfobenzene- sulfonate | Endothelial |
| 17 | 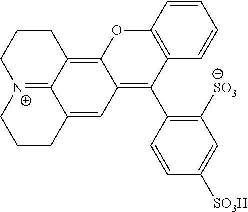 | 2-(1,2,3,5,6,7-hexahydro- chromeno[2,3-f]pyrido [3,2,1-ij]quinolin-4-ium- 9-yl)-5-sulfobenzene- sulfonate | — |
| 18 | 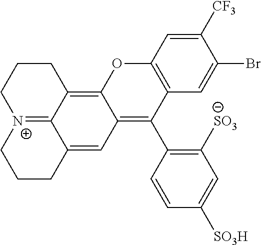 | 2-(11-bromo-12-(tri- fluoromethyl)-1,2,3,5,6,7- hexahydrochromeno[2,3- f]pyrido[3,2,1-ij]quinolin- 4-ium-9-yl)-5-sulfoben- zenesulfonate | — |
| 19 | 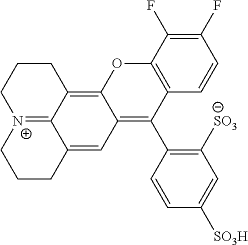 | 2-(12,13-difluoro- 1,2,3,5,6,7-hexahydro- chromeno[2,3-f]pyrido [3,2,1-ij]quinolin-4-ium- 9-yl)-5-sulfobenzene- sulfonate | Endothelial |
| 20 | 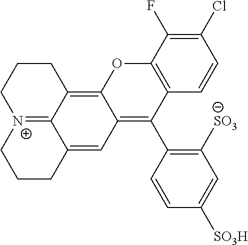 | 2-(12-chloro-13-fluoro- 1,2,3,5,6,7-hexahydro- chromeno[2,3-f]pyrido [3,2,1-ij]quinolin-4-ium- 9-yl)-5-sulfobenzene- sulfonate | Endothelial |
| 21 | 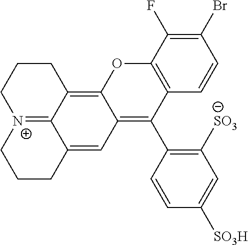 | 2-(12-bromo-13-fluoro- 1,2,3,5,6,7-hexahydro- chromeno[2,3-f]pyrido [3,2,1-ij]quinolin-4-ium- 9-yl)-5-sulfobenzene- sulfonate | Endothelial |
| 22 | 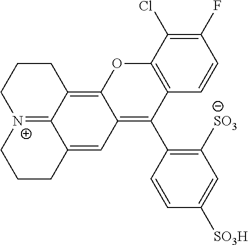 | 2-(13-chloro-12-fluoro- 1,2,3,5,6,7-hexahydro- chromeno[2,3-f]pyrido [3,2,1-ij]quinolin-4-ium- 9-yl)-5-sulfobenzene- sulfonate | Endothelial |
| 23 | 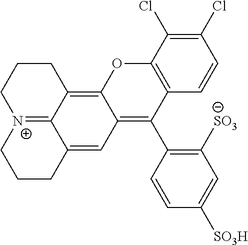 | 2-(12,13-dichloro- 1,2,3,5,6,7-hexahydro- chromeno[2,3-f]pyrido [3,2,1-ij]quinolin-4-ium- 9-yl)-5-sulfobenzene- sulfonate | Endothelial |
| 24 | 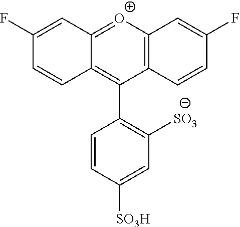 | 2-(3,6-difluoroxanthyl- ium-9-yl)-5-sulfoben- zenesulfonate | — |
| 25 | 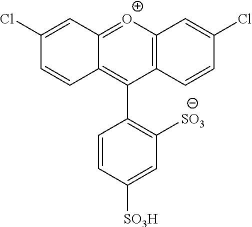 | 2-(3,6-dichloroxanthyl- ium-9-yl)-5-sulfoben- zenesulfonate | — |
| 26 | 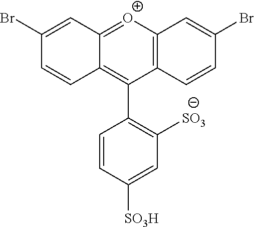 | 2-(3,6-dibromoxanthyl- ium-9-yl)-5-sulfoben- zenesulfonate | — |
| 27 | 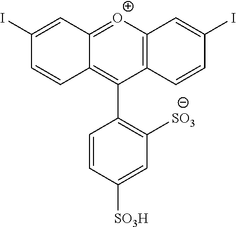 | 2-(3,6-diiodoxanthyl- ium-9-yl)-5-sulfoben- zenesulfonate | — |
| 28 | 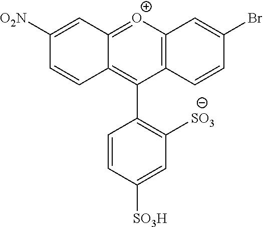 | 2-(3-bromo-6-nitroxanth- ylium-9-yl)-5-sulfoben- zenesulfonate | Endothelial |
| 29 | 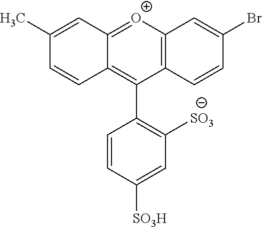 | 2-(3-bromo-6-ethylxanth- ylium-9-yl)-5-sulfoben- zenesulfonate | Endothelial |
| 30 | 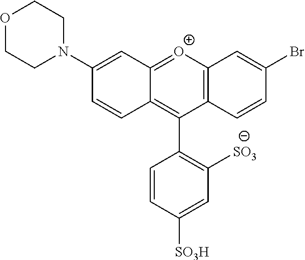 | 2-(3-bromo-6-morpho- linoxanthylium-9-yl)-5- sulfobenzenesulfonate | Endothelial |
| 31 | 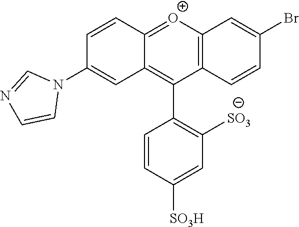 | 2-(6-bromo-2-(1H-imid- azol-1-yl)xanthylium-9- yl)-5-sulfobenzene- sulfonate | Endothelial |
| 32 | 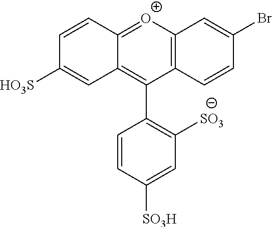 | 2-(6-bromo-2-sulfo- xanthylium-9-yl)-5- sulfobenzenesulfonate | Endothelial |
| 33 | 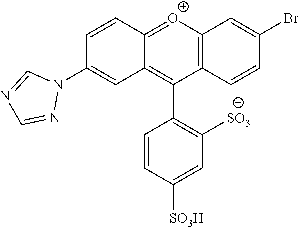 | 2-(6-bromo-2-(1H-1,2,4- triazol-1-yl)xanthylium- 9-yl)-5-sulfobenzene- sulfonate | Endothelial |
| 35 | 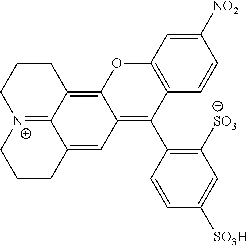 | 2-(12-nitro-1,2,3,5,6,7- hexahydrochromeno[2,3- f]pyrido[3,2,1-ij]quinolin- 4-ium-9-yl)-5-sulfoben- zenesulfonate | Astrocyte |
| 36 | 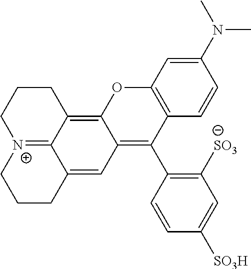 | 2-(12-(dimethylamino)- 1,2,3,5,6,7-hexahydro- chromeno[2,3-f]pyrido [3,2,1-ij]quinolin-4-ium- 9-yl)-5-sulfobenzene- sulfonate | Astrocyte |
| 37 | 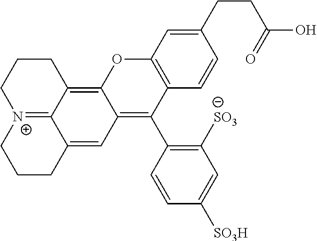 | 2-(12-(2-carboxyethyl)- 1,2,3,5,6,7-hexahydro- chromeno[2,3-f]pyrido [3,2,1-ij]quinolin-4-ium- 9-yl)-5-sulfobenzene- sulfonate | Astrocyte |
| 38 | 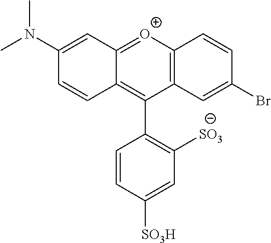 | 2-(2-bromo-6-(dimethyl- amino)xanthylium-9-yl)- 5-sulfobenzenesulfonate | Astrocyte |
| 39 | 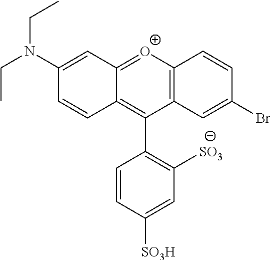 | 2-(2-bromo-6-(diethyl- amino)xanthylium-9-yl)- 5-sulfobenzenesulfonate | Endothelial |
| 40 | 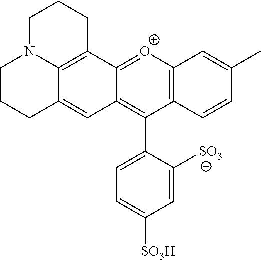 | 2-(12-methyl-2,3,6,7- tetrahydro-1H,5H- chromeno[2,3-f]pyrido [3,2,1-ij]quinolin-14- ium-9-yl)-5-sulfoben- zenesulfonate | Endothelial |
| 41 | 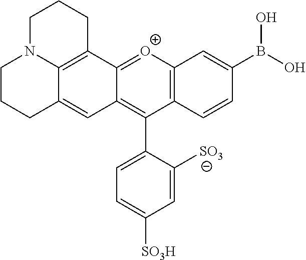 | 2-(12-borono-2,3,6,7- tetrahydro-1H,5H- chromeno[2,3-f]pyrido [3,2,1-ij]quinolin-14- ium-9-yl)-5-sulfoben- zenesulfonate | Endothelial |
| 42 | 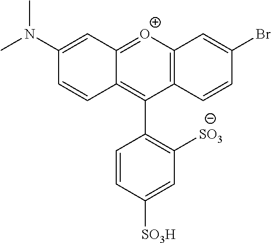 | 2-(3-bromo-6-(dimethyl- amino)xanthylium-9-yl)- 5-sulfobenzenesulfonate | Endothelial |
| 43 | 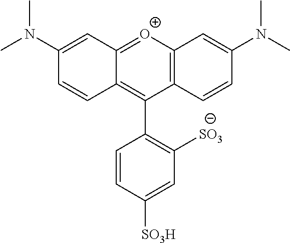 | 2-(3,6-bis(dimethyl- amino)xanthylium-9-yl)- 5-sulfobenzenesulfonate | Endothelial |
| 44 | 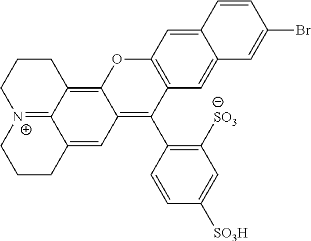 | 2-(12-bromo-1,2,3,5,6,7- hexahydrobenzo[6,7] chromeno[2,3-f]pyrido [3,2,1-ij]quinolin-4-ium- 9-yl)-5-sulfobenzene- sulfonate | Endothelial |
| 45 | 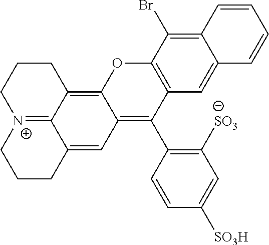 | 2-(15-bromo-1,2,3,5,6,7- hexahydrobenzo[6,7] chromeno[2,3-f]pyrido [3,2,1-ij]quinolin-4-ium- 9-yl)-5-sulfobenzene- sulfonate | Endothelial |
| 46 | 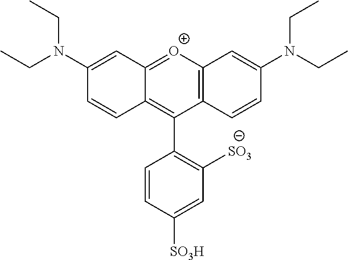 | 2-(3,6-bis(diethylamino)- xanthylium-9-yl)-5- sulfobenzenesulfonate | Endothelial |

TABLE 2A
Exemplary Compounds

| Cmpd | Structure | Nomenclature |
|---|---|---|
| 47 | 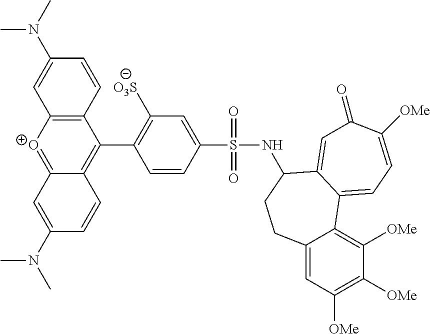 | 2-(3,6-bis(dimethyl- amino)xanthylium-9- yl)-5-(N-(1,2,3,10- tetramethoxy-9-oxo- 5,6,7,9-tetrahydro- benzo[a]heptalen-7- yl)sulfamoyl)benzene- sulfonate |
| 48 | 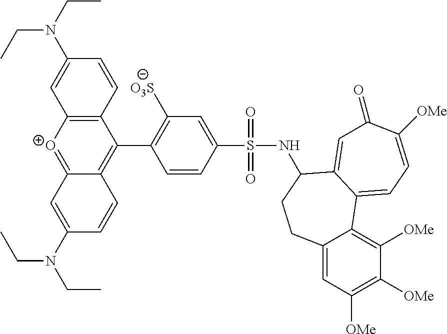 | 2-(3,6-bis(diethyl- amino)xanthylium-9- yl)-5-(N-(1,2,3,10- tetramethoxy-9-oxo- 5,6,7,9-tetrahydro- benzo[a]heptalen-7- yl)sulfamoyl)benzene- sulfonate |
| 49 | 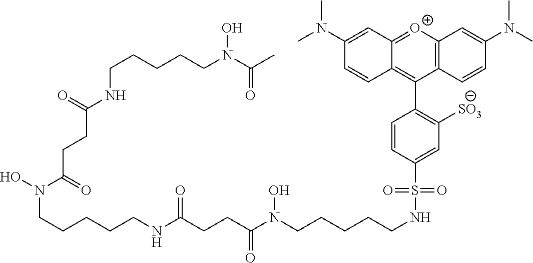 | 2-(3,6-bis(dimethyl- amino)xanthylium- 9-yl)-5-(N-(3,14,25- trihydroxy-2,10,13, 21,24-pentaoxo-3,9, 14,20,25-pentaaza- triacontan-30-yl)- sulfamoyl)benzene- sulfonate |
| 50 | 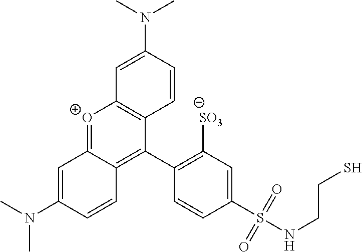 | 2-(3,6-bis(dimethyl- amino)xanthylium-9- yl)-5-(N-(2-mercapto- ethyl)sulfamoyl)ben- zenesulfonate |

| | |
|---|---|
| 51 | 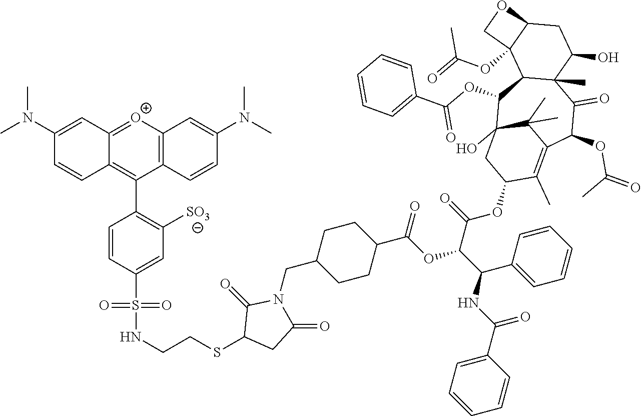 |
| | 5-(N-(2-((1-((4-((((1R,2S)-1-benzamido-3-(((2aS,4R,4aR,6S,9R,11R,12R,12aS,12bR)-6,12b-diacetoxy- |
| | 12-(benzoyloxy)-4,11-dihydroxy-4a,8,13,13-tetramethyl-5-oxo-2a,3,4,4a,5,6,9,10,11,12,12a,12b- |
| | dodecahydro-1H-7,11-methanocyclodeca[3,4]benzo[1,2-b]oxet-9-yl)oxy)-3-oxo-1-phenylpropan-2-yl) |
| | oxy)carbonyl)cyclohexyl)methyl)-2,5-dioxopyrrolidin-3-yl)thio)ethyl)sulfamoyl)-2-(3,6-bis(dimeth- |
| | ylamino)xanthylium-9-yl)benzenesulfonate |
| 52a | 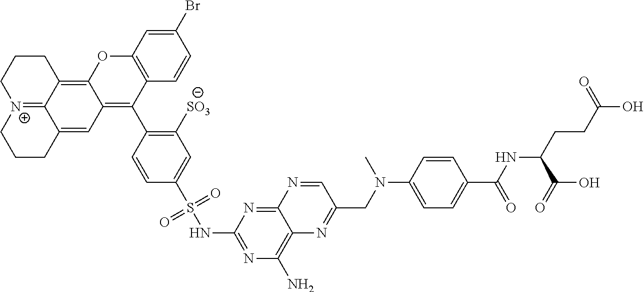 |
| | (S)-5-(N-(4-amino-6-(((4-((1,3-dicarboxypropyl)carbamoyl)phenyl)(methyl)amino)methyl)pteridin- |
| | 2-yl)sulfamoyl)-2-(12-bromo-1,2,3,5,6,7-hexahydrochromeno[2,3-f]pyrido[3,2,1-ij]quinolin-4-ium- |
| | 9-yl)benzenesulfonate |
| 52b | 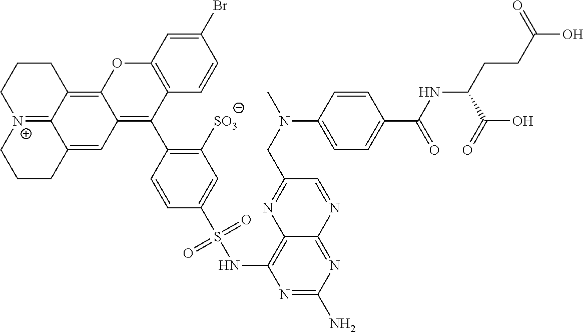 |
| | (R)-5-(N-(2-amino-6-(((4-((1,3-dicarboxypropyl)carbamoyl)phenyl)(methyl)amino)methyl)- |
| | pteridin-4-yl)sulfamoyl)-2-(12-bromo-1,2,3,5,6,7-hexahydrochromeno[2,3-f]pyrido[3,2,1-ij] |
| | quinolin-4-ium-9-yl)benzenesulfonate |
| 53 | 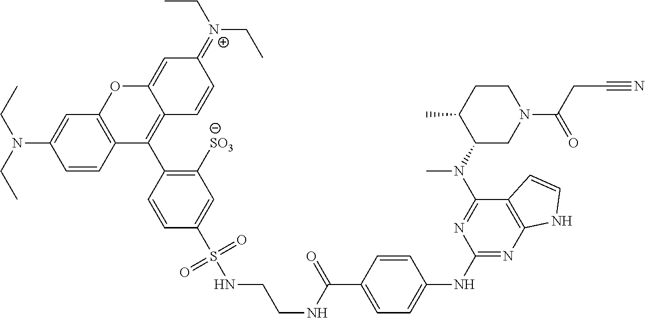 |
| | 5-(N-(2-(4-((4-(((3R,4R)-1-(2-cyanoacetyl)-4-methylpiperidin-3-yl)(methyl)amino)-7H-pyrrolo[2,3-d] |
| | pyrimidin-2-yl)amino)benzamido)ethyl)sulfamoyl)-2-(6-(diethylamino)-3-(diethyliminio)-3H-xanthen- |
| | 9-yl)benzenesulfonate |
| 54 | 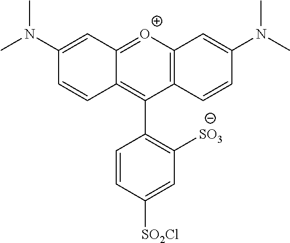 |
| | 2-(3,6-bis(dimethylamino)xanthylium-9-yl)-5- |
| | (chlorosulfonyl)benzenesulfonate |
| 55 | 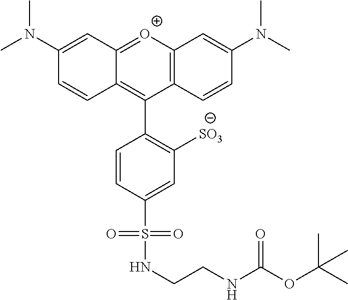 |
| | 2-(3,6-bis(dimethylamino)xanthylium-9-yl)-5-(N-(2-((tert- |
| | butoxycarbonyl)amino)ethyl)sulfamoyl)benzenesulfonate |

Compounds in Table 2A exhibit Endothelial specificity.

TABLE 2B
Exemplary compounds

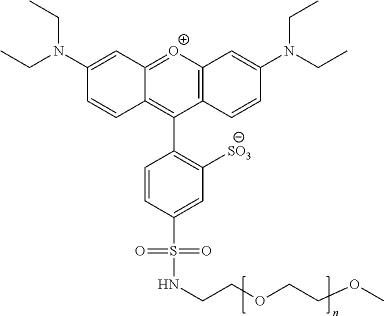

| Cmpd | Alternative Name | 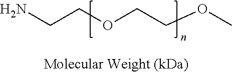 | n |
|---|---|---|---|
| 56 | mPEG-NH₂ 1 kDa | 1 kDa | n = 21 |
| 57 | mPEG-NH₂ 2 kDa | 2 kDa | n = 44 |
| 58 | mPEG-NH₂ 3.4 kDa | 3.4 kDa | n = 76 |
| 59 | mPEG-NH₂ 5 kDa | 5 kDa | n = 112 |
| 60 | mPEG-NH₂ 10 kDa | 10 kDa | n = 226 |
| 61 | mPEG-NH₂ 20 kDa | 20 kDa | n = 453 |

Compounds in Table 2B exhibit Endothelial specificity.

Compounds 56-61 were prepared from the corresponding PEG-amine derivatives, which are characterized by their respective molecular weights in kDa. Accordingly, one of ordinary skill in the art appreciates that the ‘n’ values provided herein are approximate. In certain embodiments, the value provided for “n” may vary by 1, 2, 3, 4, 5, 6, 7, 8, 9, or 10.

In certain embodiments, compound 56 comprises n=21±1. In certain embodiments, compound 56 comprises n=21±2. In certain embodiments, compound 56 comprises n=21±3. In certain embodiments, compound 56 comprises n=21±4. In certain embodiments, compound 56 comprises n=21±5. In certain embodiments, compound 56 comprises n=21±6. In certain embodiments, compound 56 comprises n=21±7. In certain embodiments, compound 56 comprises n=21±8. In certain embodiments, compound 56 comprises n=21±9. In certain embodiments, compound 56 comprises n=21±10.

In certain embodiments, compound 57 comprises n=44±1. In certain embodiments, compound 57 comprises n=44±2. In certain embodiments, compound 57 comprises n=44±3. In certain embodiments, compound 57 comprises n=44±4. In certain embodiments, compound 57 comprises n=44±5. In certain embodiments, compound 57 comprises n=44±6. In certain embodiments, compound 57 comprises n=44±7. In certain embodiments, compound 57 comprises n=44±8. In certain embodiments, compound 57 comprises n=44±9. In certain embodiments, compound 57 comprises n=44±10.

In certain embodiments, compound 58 comprises n=76±1. In certain embodiments, compound 58 comprises n=76±2. In certain embodiments, compound 58 comprises n=76±3. In certain embodiments, compound 58 comprises n=76±4. In certain embodiments, compound 58 comprises n=76±5. In certain embodiments, compound 58 comprises n=76±6. In certain embodiments, compound 58 comprises n=76±7. In certain embodiments, compound 58 comprises n=76±8. In certain embodiments, compound 58 comprises n=76±9. In certain embodiments, compound 58 comprises n=76±10.

In certain embodiments, compound 59 comprises n=112±1. In certain embodiments, compound 59 comprises n=112±2. In certain embodiments, compound 59 comprises n=112±3. In certain embodiments, compound 59 comprises n=112±4. In certain embodiments, compound 59 comprises n=112±5. In certain embodiments, compound 59 comprises n=112=6. In certain embodiments, compound 59 comprises n=112±7. In certain embodiments, compound 59 comprises n=112±8. In certain embodiments, compound 59 comprises n=112±9. In certain embodiments, compound 59 comprises n=112±10.

In certain embodiments, compound 60 comprises n=226±1. In certain embodiments, compound 60 comprises n=226±2. In certain embodiments, compound 60 comprises n=226±3. In certain embodiments, compound 60 comprises n=226±4. In certain embodiments, compound 60 comprises n=226±5. In certain embodiments, compound 60 comprises n=226±6. In certain embodiments, compound 60 comprises n=226±7. In certain embodiments, compound 60 comprises n=226±8. In certain embodiments, compound 60 comprises n=226±9. In certain embodiments, compound 60 comprises n=226±10.

In certain embodiments, compound 61 comprises n=453±1. In certain embodiments, compound 61 comprises n=453±2. In certain embodiments, compound 61 comprises n=453±3. In certain embodiments, compound 61 comprises n=453±4. In certain embodiments, compound 61 comprises n=453±5. In certain embodiments, compound 61 comprises n=453±6. In certain embodiments, compound 61 comprises n=453±7. In certain embodiments, compound 61 comprises n=453±8. In certain embodiments, compound 61 comprises n=453±9. In certain embodiments, compound 61 comprises n=453±10.

Certain exemplary embodiments of compounds 56-61 are defined in Table 2C.

TABLE 2C
Exemplary compounds

| Cmpd | Structure |
|---|---|
| 56 | 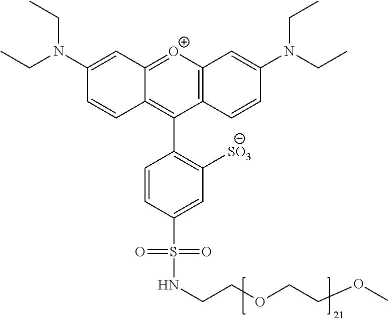 |
| 57 | 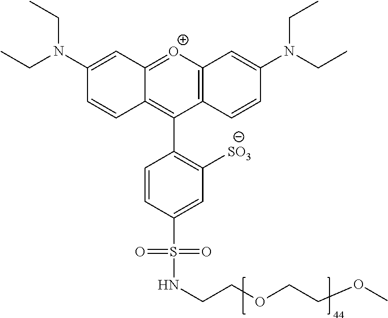 |
| 58 | 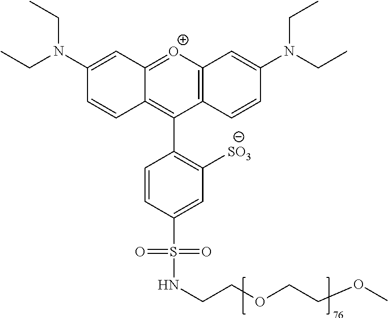 |
| 59 | 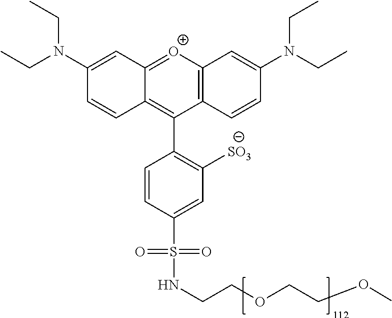 |
| 60 | 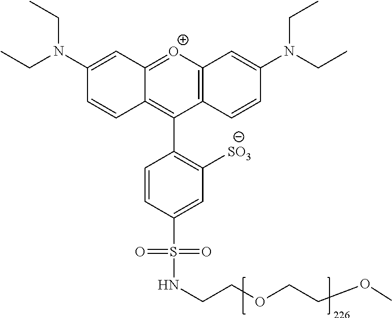 |
| 61 | 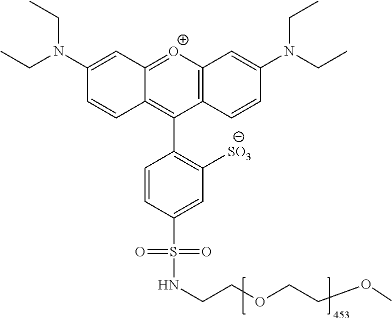 |

Compounds in Table 2C exhibit Endothelial specificity.

### Example 2: Generation of a Combinatorial Fluorophore Library

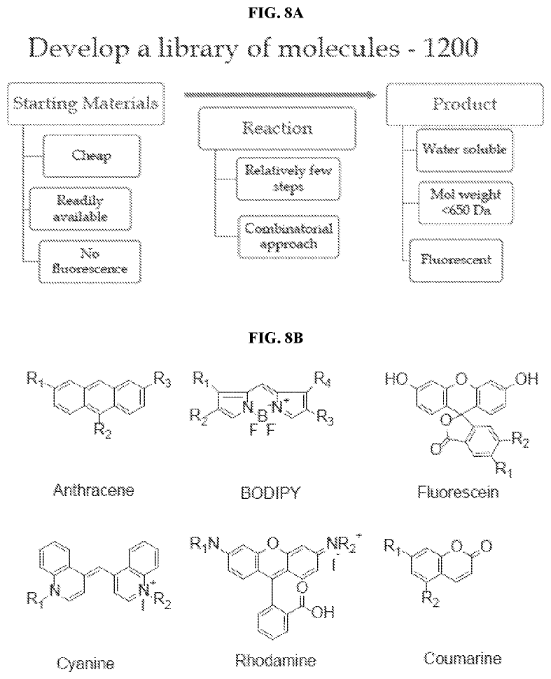

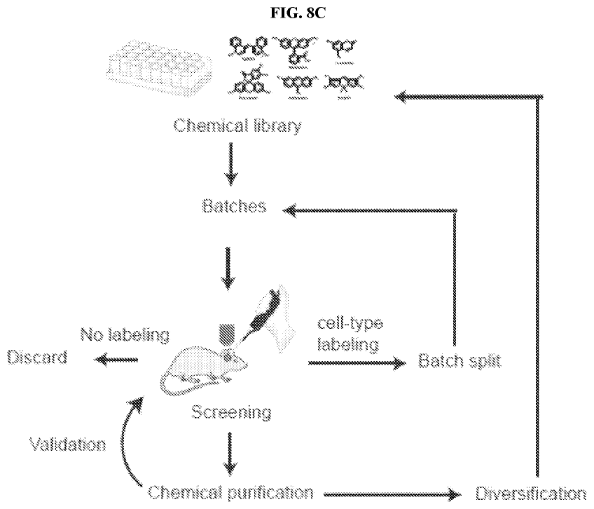

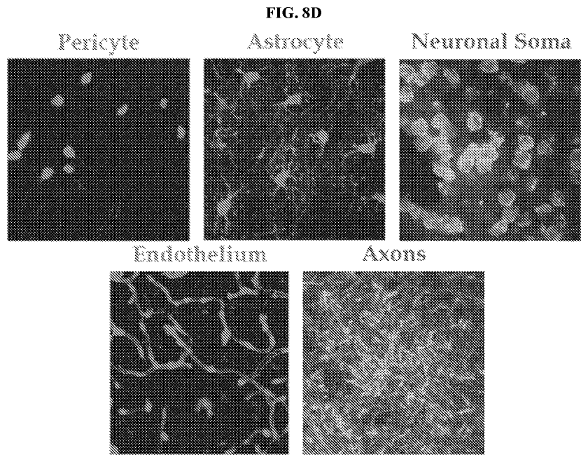

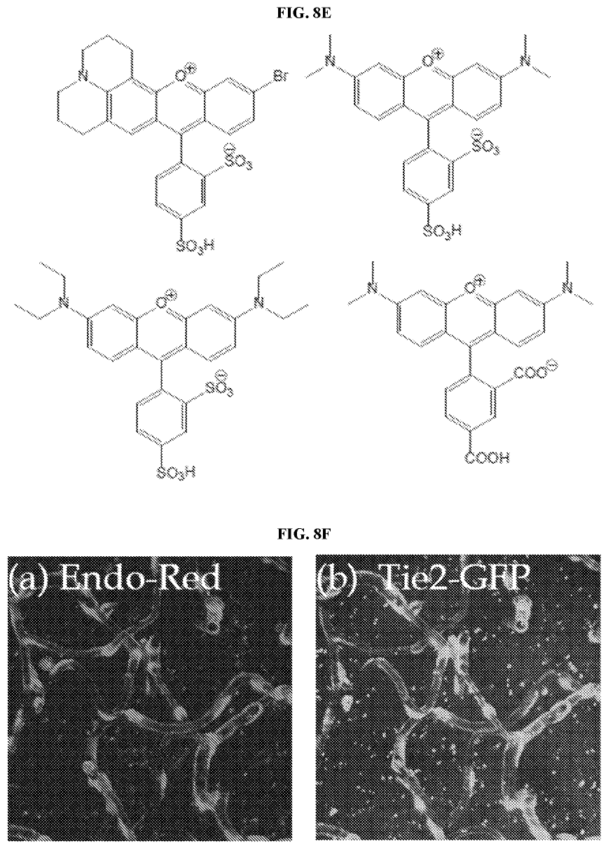

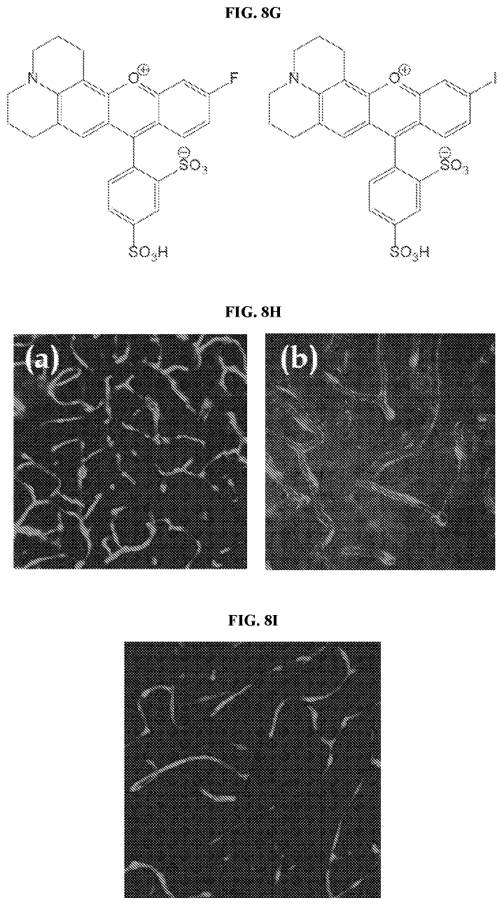

It has been hypothesized that novel small molecules (SMs) that can selectively enter certain brain cell types by screening a library of small fluorescent compounds through optical imaging in the live mouse brain. To achieve this goal, a chemical library of ~1200 fluorescent SMs were generated through a combinatorial chemistry approach. Compounds comprising widely used fluorophore backbones were synthesized (e.g., cyanine, rhodamine, coumarin, fluorescein, anthracene, and BODIPY) with a diversity of functional groups, resulting in compounds ranging from 300 to 600 Da and variable degrees of hydrophilicity (FIGS. 8A-8B). To enhance synthesis efficiency and ease of chemical modification, short synthetic routes and microwave-based reactions were utilized. Furthermore, this strategy did not require cumbersome compound purification as starting materials that were non-fluorescent were used, thus allowing imaging-based screening, without the confounding presence of fluorescent reactants.

### Example 3: Library Screening Through Optical Imaging in the Live Mouse Brain

It was reasoned that the best strategy to identify fluorophores with specific affinity to certain cell types and transport mechanisms would be by direct screening in the intact in vivo brain microenvironment. A straightforward screening method was designed by direct topical brain administration through a cranial window preparation. This strategy allowed the use of much smaller amounts of compounds than would have otherwise been required with intravenous administration. Furthermore, it eliminated issues of variable blood brain barrier (BBB) permeability, which would have severely limited the effectiveness of the screen. To improve screening efficiency, a pooling strategy was devised whereby ten unpurified compounds were dissolved at micromolar concentrations in artificial cerebrospinal fluid (ACSF) with dimethyl sulfoxide (DMSO) (3% v/v). Batches of compounds were then applied topically to the mouse brain for 20 minutes and thoroughly washed to reduce nonspecific interstitial space fluorescence. This was followed by high-resolution intravital two-photon imaging to visualize the various patterns of intracellular fluorophore uptake (FIG. 8C).

Imaging of these compounds revealed a variety of labeling patterns, with the vast majority of batches showing diffuse labeling of the interstitial space and a few cases with clear cellular specificity. Batches of compounds that showed cell-specific labeling were selected for further iterative screening by splitting them into compound subgroups until individual fluorophores with the unique labeling selectivity were identified. However, given that these compounds were impure, cellular labeling could have been partly due to fluorescent reaction byproducts. Therefore, a scale-up synthesis was performed for positive hits, followed by chromatographic purification. The purified compounds were then topically reapplied to the brain and imaged in vivo to validate their selective labeling properties. After this extensive intravital screen of ~1200 molecules only ten compounds were identified that showed selective uptake to either neurons, astrocytes, pericytes or endothelium, highlighting the requirement for exquisitely precise molecular interactions between transport mechanisms and small molecules (FIG. 8D).

### Example 4: Selectivity and Specificity of Exemplary Compounds with Intravenous Administration

For all mouse experiments, cranial windows were used. Animals were anaesthetized via intraperitoneal injections of 100 mg kg⁻¹ ketamine and 10 mg kg⁻¹ xylazine or via inhaled isoflurane. A region of the skull (3×3 mm) was gently removed with a high-speed drill and the underlying dura was removed. A small size 0 glass coverslip was placed over the skull to allow long term optical access for in vivo imaging.

For intravenous injections, the cranial window preparation was placed first and followed by retroorbital sinus IV injection of 50 microliters of compound 1 at 20 mg/ml concertation.

In vivo images were acquired using a laser scanning Leica SP-5 confocal or a two-photon microscope (Prairie Technologies) equipped with a mode-locked MaiTai two-photon laser (Spectra Physics) and x 20 water immersion objective (Zeiss 1.0 numerical aperture). Excitation wavelengths for 2 photon microscopy were 800 to 900 nm and for confocal microscopy 480 to 516 nm. All experiments were done in CD-1 or C57B6 mice ages 2-4 months.

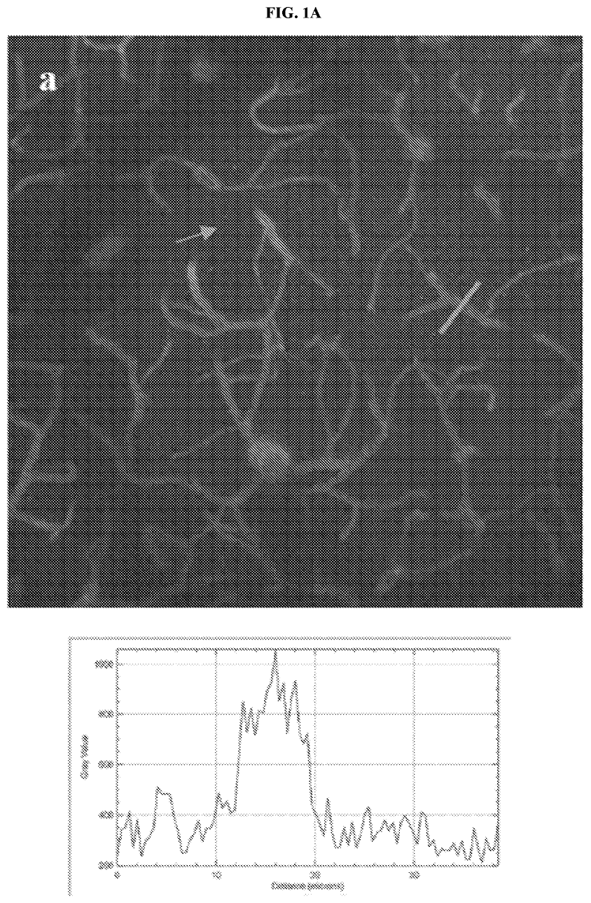

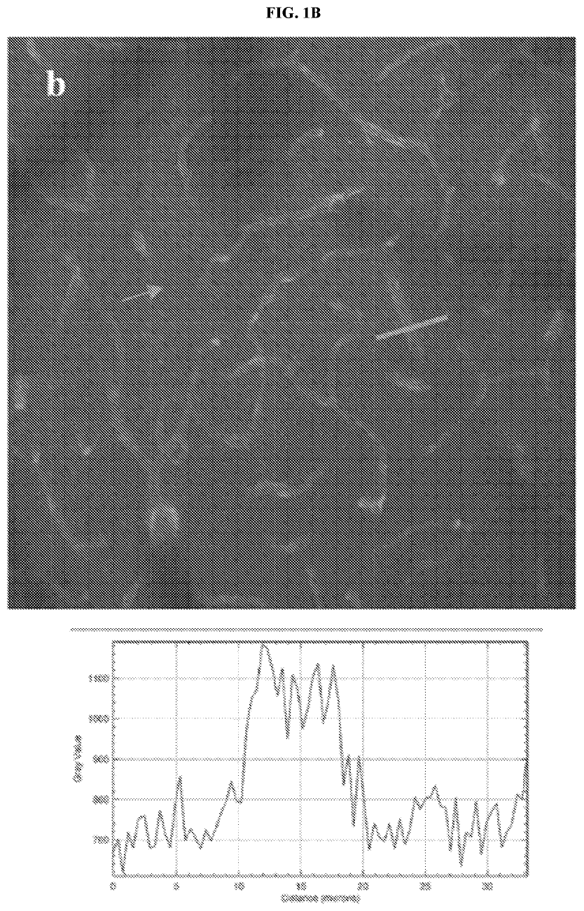

In vivo two photon imaging of the mouse brain cortex following intravenous administration of the exemplary compound 1 leads to robust brain endothelial labeling and concurrent extravasation of the compound into the interstitial brain parenchyma. In FIGS. 1A-1B blue arrows point to the interstitial space where there is a marked increase in fluorescence comparing 10 min (FIG. 1A) and 130 min (FIG. 1B) post injection images. Below each image there is a fluorescence intensity plot along a line profile (blue line) across capillary vessels and adjacent interstitial space. These data show a marked increase in the interstitial space fluorescence. As evidenced by FIGS. 1A-1B, the compounds according to the invention readily cross the blood brain barrier despite their hydrophilic nature.

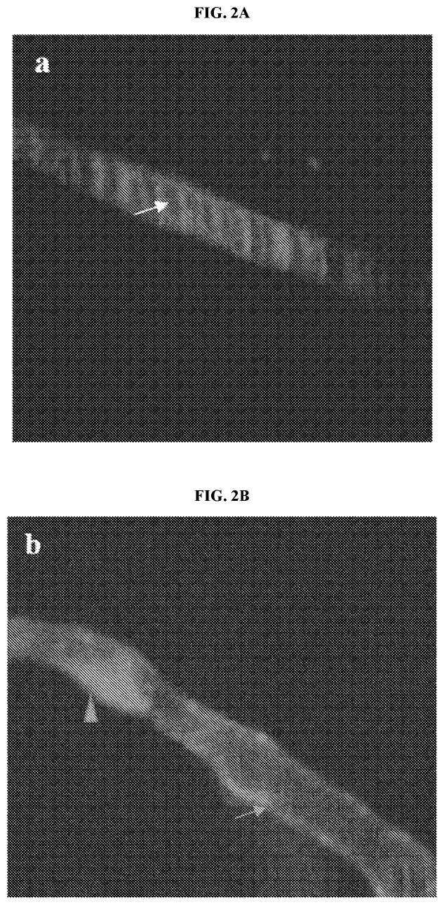

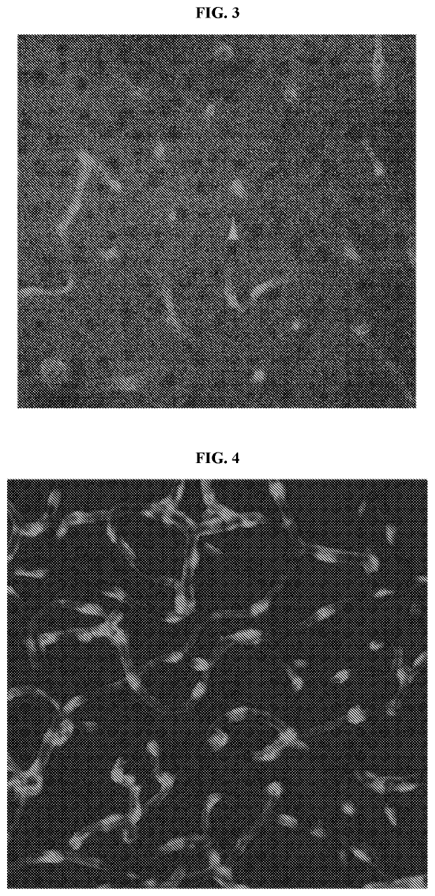

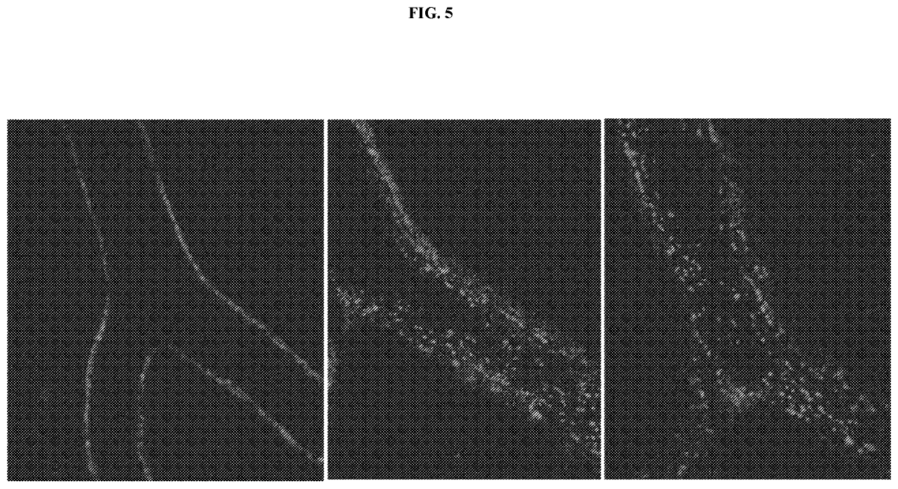

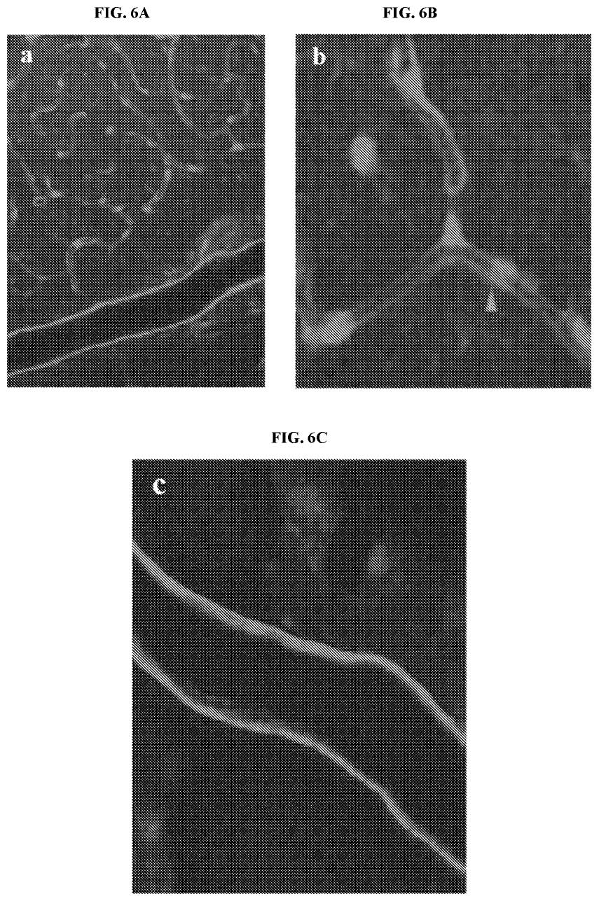

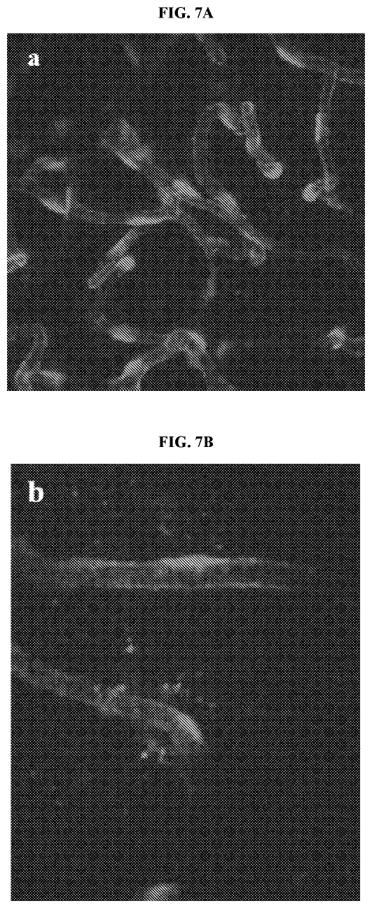

FIGS. 2A-2B depicts in vivo two photon imaging at 10 min (FIG. 2A), and 130 min (FIG. 2B). The white arrow in FIG. 2A points to the compound of the invention within the brain capillary lumen at 10 minutes IV post injection. At 130 mins (FIG. 2B), there is a marked uptake of the compound into the capillary endothelial wall (blue arrow). A brightly labeled endothelial cell body is also seen (blue arrowhead). During this interval there is also a relative decline in the intravascular fluorescence as evidenced by the loss of the intermittent bright and dark stripes seen in FIG. 2A (white arrow). As demonstrated by FIGS. 2A-2B, the exemplary compounds rapidly transfer from the capillary lumen to the endothelial wall.

As shown in FIG. 3, 2-hours post intravenous injection of compound 1, a significant portion of the injected compound 1 is located in the brain interstitial space. The lack of cell body labeling (blue arrowhead pointing to black circular cell bodies on the red background) is observed. This indicates that the dye does not enter other cells except for endothelial cells and crosses the BBB and distributes in the interstitial space. As evidenced by FIG. 3, the exemplary compound is excluded from non-endothelial cells.

Using a lower concentration of intravenous compound 1 at 1.1 mg in 120 μL of PBS, at around 2 hours post injection, a bright punctate labeling becomes apparent in the endothelium (FIG. 5). Without wishing to be bound by theory, one of the possible mechanisms of uptake and transport of the compound into and out of the endothelium is through vesicular transport. This may include transcytosis mechanisms that are well known to occur at the endothelium of the brain. These vesicles could be critical for releasing the cargo into the brain interstitial space. As evidenced by FIG. 5, the compound may be transported via vesicular transcytosis. However, other alternatives for transport may be possible, such as through solute carriers and membrane transporters that do not involve vesicular transport.

### Example 5: Selectivity and Specificity of Exemplary Compounds with Topical Administration

For all mouse experiments, cranial windows were used. Animals were anaesthetized via intraperitoneal injections of 100 mg kg⁻¹ ketamine and 10 mg kg⁻¹ xylazine or via inhaled isoflurane. A region of the skull (3×3 mm) was gently removed with a high-speed drill and the underlying dura was removed. A small size 0 glass coverslip was placed over the skull to allow long term optical access for in vivo imaging.

For topical administration, the various compounds of the invention were applied to the cortex prior to glass coverslip placement with a micropipette at compound concentrations of 10 mg/ml and left for 15 minutes followed by washing with PBS×3 for 5 minutes each washing.

Highly specific endothelial labeling by the compounds of the invention is demonstrated by FIG. 4, which depicts the results of a direct topical application of compound 1 to the brain surface through a craniotomy in a live mouse. Highly specific labeling of endothelial cells within capillaries occurs 20 minutes after application of the compound. Although all brain cells are exposed to the compound, only endothelial cells are labeled proving remarkable specificity of the compound. Like in the intravenous administration experiment (EXAMPLE 1), topical application also leads to strong labeling at the cell body but also at endothelial processes covering the totality of the microvascular extension.

### Example 6: Mechanisms of Intracellular Uptake Specificity

Proof-of-concept efforts to identify the transport mechanisms were focused on a specific core hit that demonstrated exquisite endothelial uptake selectivity (Endo-Red). It was found that Endo-Red was able to enter the cytoplasm of endothelial cells within tens of minutes following topical brain application and did not label any other cell type despite the use of high compound concentrations. Endo-Red was able to enter all endothelial cells, including in arterioles, capillaries, and venules within the brain parenchyma. Interestingly, Endo Red was completely excluded from endothelial cells of immediately adjacent vessels outside of the brain in the pial surface, suggesting a very specific pattern of transporter expression in endothelium of the blood brain barrier. A similar endothelial labeling pattern was observed following intraocular injection into the eye, whereby endothelial cells in retinal blood vessels including arterioles, venules and capillaries were rapidly labeled.

Potential mechanisms of cell uptake of Endo-Red were then investigated. Given its relatively high molecular weight (585 Da) and hydrophilicity, it was hypothesized that the uptake was mediated through membrane transporters rather than passive diffusion. Furthermore, it was hypothesized that the cell uptake selectivity was due to differential transporter enrichment in endothelium of the brain and retina. To better understand the mechanisms, various publicly available single-cell transcriptome RNA seq databases were mined to select potential transporter candidates with expression patterns restricted to brain and retinal endothelial cells. Eight organic solute carrier (SLCO) transporter candidates with brain endothelium expression. The properties of these transporters were then tested by overexpressing them in human embryonic kidney (HEK) 293 cells (a cell line with low endogenous transporter expression) followed by in vitro Endo-Red uptake experiments. Using this strategy, it was discovered that only SLCO1A4 expression led to increased Endo-Red uptake and that this was proportional to transporter expression levels. To demonstrate the role of the endogenous Slco1a4 transporter more definitively in Endo-Red uptake in vivo, a mouse with deletion of the Slco1a/1b gene cluster, including the Slco1a4 gene (the only gene from this cluster expressed in the brain) was tested. Application of Endo-red to the cortical surface in this mouse, followed by in vivo two photon brain imaging, showed a complete loss of the endothelial labeling.

TABLE 3
solute carriers enriched in endothelial cells

| Transporters | Endothelial Cells (G1) | Other Cells (G2) | G1/G2 |
|---|---|---|---|
| Slc52a3 | 117.34 | 0.05 | 2400.03 |
| Slco1a4 | 2367.71 | 9.46 | 250.34 |
| Slc19a3 | 198.42 | 1.71 | 116.34 |
| Slc38a5 | 266.04 | 2.77 | 96.01 |
| Slc16a4 | 198.99 | 2.29 | 86.85 |
| Slco1c1 | 1246.03 | 18.01 | 69.17 |
| Slc7a5 | 771.96 | 14.18 | 54.44 |

To determine the potential for future human translation of these compounds, it was further examined whether the cellular uptake properties would be preserved in cells expressing SLCO1A2, which is the human orthologue of Slco1a4. It was found that like with Slco1a4, overexpression of SLCO1A2 in HEK293 cells, demonstrated that this transporter was sufficient to render cells permeable to Endo-Red. Thus, without wishing to be bound by any theory, these data support the role of Slco1a4 as the sole mechanism of intracellular transport of Endo-Red in mice and suggests the possibility of using SLCO1A2 to pharmacologically target cells expressing this transporter in humans.

### Example 7: Pharmacological Access to Neural Cells Through Gene Therapy-Mediated Transporter Expression

The hypothesis that this technology could be applied to render brain cells that normally do not express the transporter, susceptible to selective bifunctional SM uptake. To achieve this, in utero-electroporation was used to ectopically express Slco1a4 or SLCO1A2 in cortical neurons in live mice. The uptake properties of Endo-Red topically administered to the brain were examined through intravital 2-photon imaging. Consistent with the hypothesis, a marked Endo-Red uptake was observed by neuronal cell bodies and distal dendrites after either Slco1a4 or SLCO1A2 neuronal overexpression. Associated with this, a relative decrease in endothelial uptake was also observed, which may be due to a greatly expanded volume of distribution within neurons.

Given the presence of the blood brain barrier (BBB) which is known to restrict the entry of most small molecules into the brain, it was tested whether Endo-red could cross the BBB to access neurons. To test this, Endo-Red was injected intravenously in mice that had been in utero electroporated to express Slco1a4 or SLCO1A2 in neurons. In vivo imaging following IV Endo-Red administration demonstrated robust uptake by cortical neuronal cell bodies and dendrites. Given the possibility that the craniotomy preparation could disrupt the BBB, it was further confirmed neuronal Endo-red uptake by histologically imaging in mice that did not undergo surgery.

To test whether these properties could be applied to other organs outside of the brain, a similar experiment was performed by electroporation of Slco1a4 or SCLO1A2 in the mouse retina. Like in the brain, it was demonstrated that ectopic transporter expression rendered diverse retinal populations permeable to Endo-Red, including retinal ganglion cells and photoreceptors. Taken together, this suggests the exciting possibility of using gene therapy to ectopically express membrane transporters to achieve chronic selective pharmacological access to specific cell populations of interest.

### Example 8: Development of Drug Conjugates that Retain Cell-Type Uptake Specificity

It was first sought to identify potential sites on Endo-red for drug conjugation. To achieve this, a strategy was developed to identify the essential chemical functional groups that were necessary and sufficient for endothelial cell-specific uptake. An iterative process consisting of functional group chemical diversification, followed by two photon imaging, which allowed a structure property relationship (SPR) analysis in the live brain. Through this iterative process, the consequences of about 50 Endo-Red modifications were systematically assessed on their intracellular uptake properties. It was found that a sulfonate group (—SO₃H) in Endo-red may be used as a potential site for reaction with amines, thus providing great flexibility for conjugation to linkers and pharmacological agents. To test the feasibility of the conjugation approach, the commonly used drugs colchicine, taxol and deferoxamine, were selected, which are not known to have any cell-type uptake specificity.

It was then assessed whether these new compounds acquired the cellular uptake specificity of Endo-Red. Using HEK293 cells transfected with Slco1a4/A2, it was shown that these bifunctional compounds were readily taken up by transporter-expressing cells. Furthermore, direct application of these compounds to the brain followed by two photon imaging confirmed their selective endothelial uptake in vivo. Likewise, ectopic expression of Slco1a4/A2 in neurons of brain and retina also rendered these cells permeable to the bifunctional pharmacological agents. The transport mechanism allows for delivery of molecules larger hydrophilic compounds up to 2 kDa, thus opening the range of compounds available for drug discovery. Taken together, these experiments demonstrate the feasibility of conjugation of Endo-Red compounds to a variety of drugs of interest while maintaining cell-uptake specificity.

### Example 9: Drug Conjugates Preserve Pharmacological Properties with Reduced Systemic Toxicity

Having shown preservation of cell type specificity after conjugation, it was then determined if the pharmacological properties of the bifunctional molecules can be preserved. For this proof of principle experiment, the drug Colchicine was chosen, given its relatively well-known mechanisms of action and robust effects that are easily quantified in cell-based assays. Furthermore, the chemistry of Colchicine has been extensively characterized, which facilitated the choice of functional groups for conjugation.

To test whether the pharmacological properties of colchicine were preserved after conjugation to Endo-Red, the conjugate-compound was applied using rapidly dividing NIH3T3 cells to assess Mitosis affected Multinucleated cells. These experiments demonstrated the preservation of pharmacological properties as well as transporter-mediated selectivity.

Colchicine is well known to cause bone marrow suppression and hair loss among other side effects in humans and is widely used as a model of neurotoxicity. Further, it is demonstrated herein that subcutaneous injections of an exemplary conjugate-compound completely preserved hair growth properties compared to Colchicine, despite the use of very high compound dosage (FIGS. 12A-12C).

### Example 9: Discovery of Compounds with Brain and Retina Endothelial Specificity

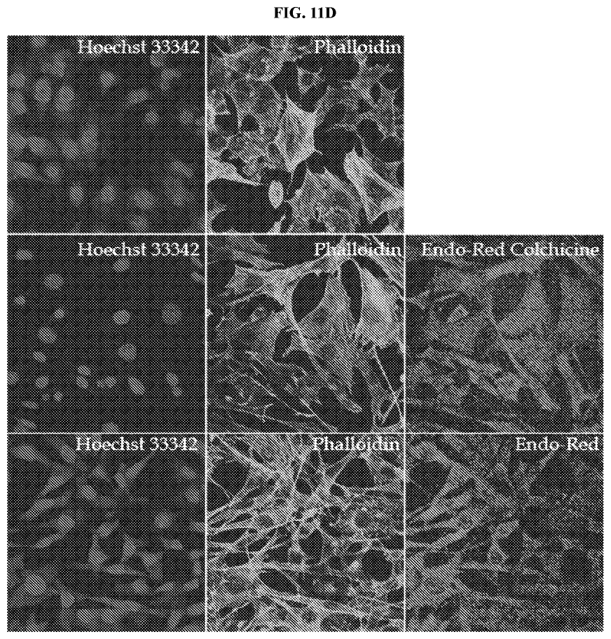

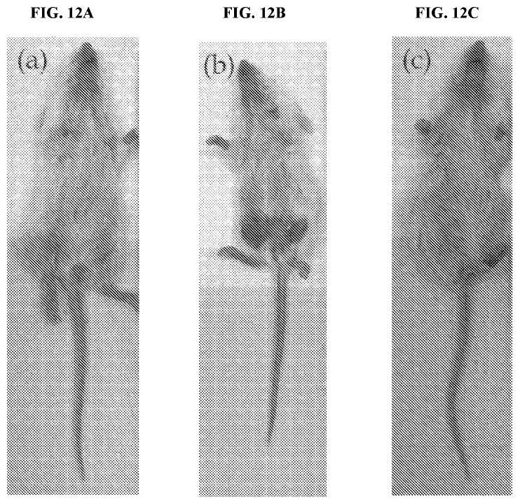

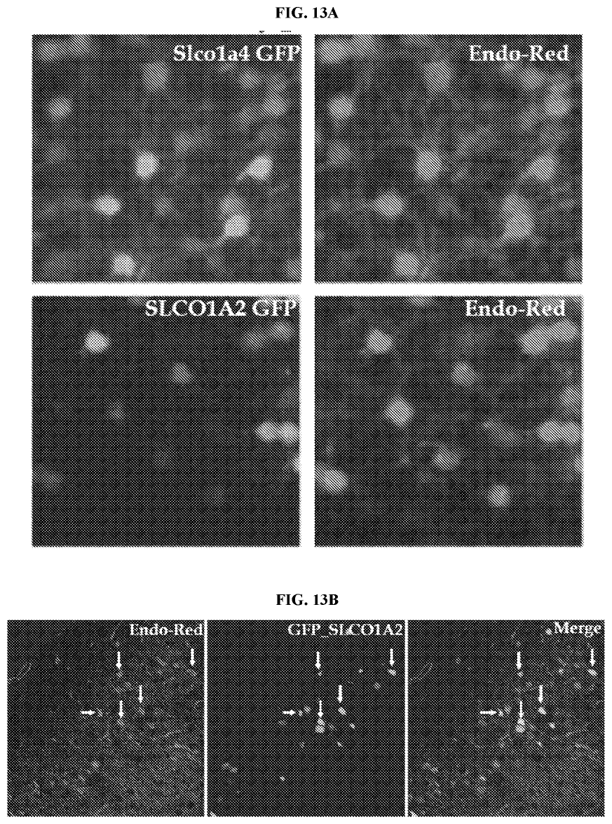

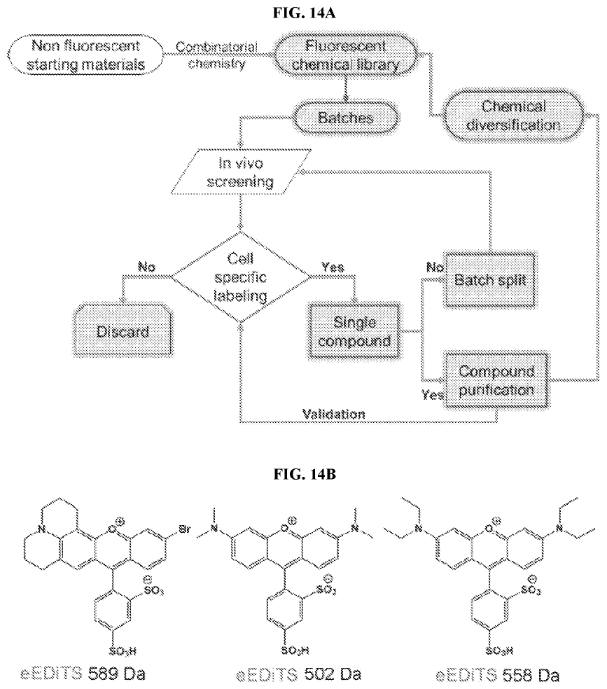

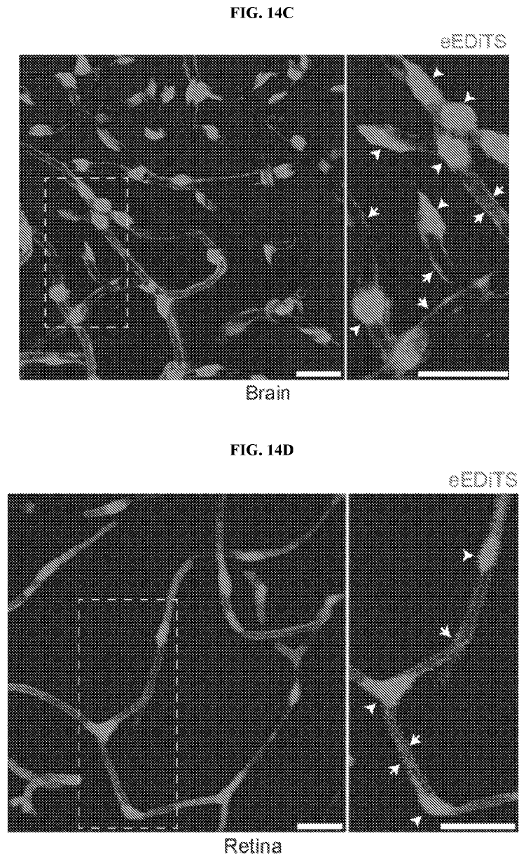

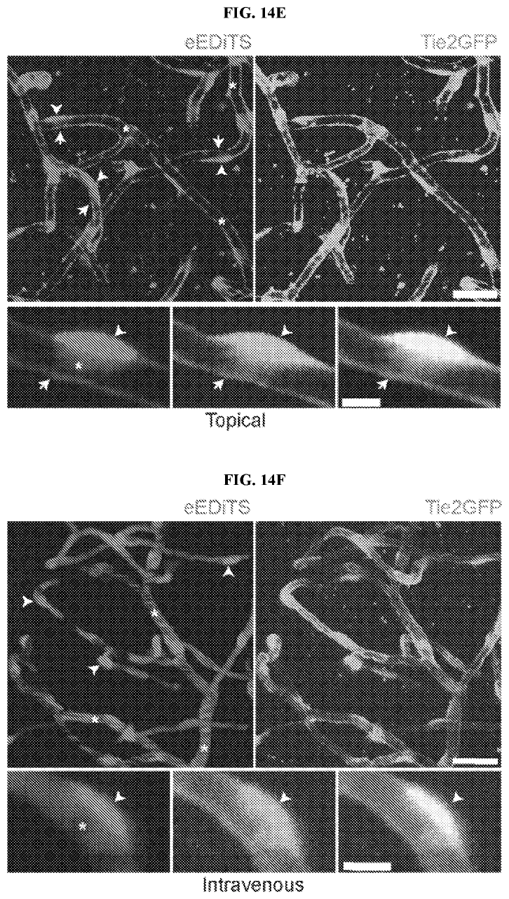

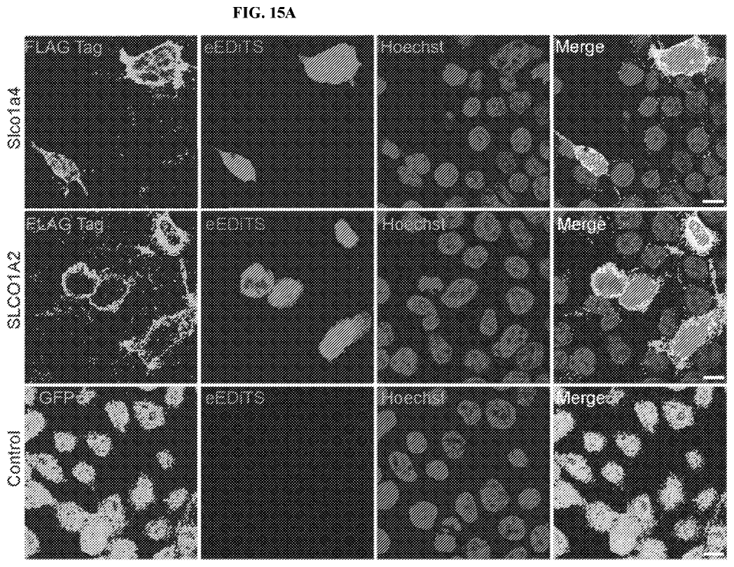

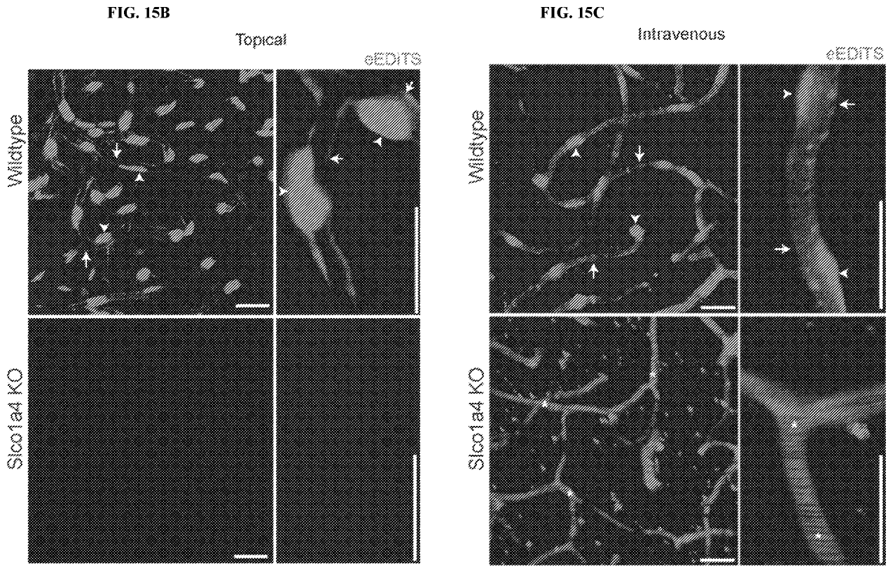

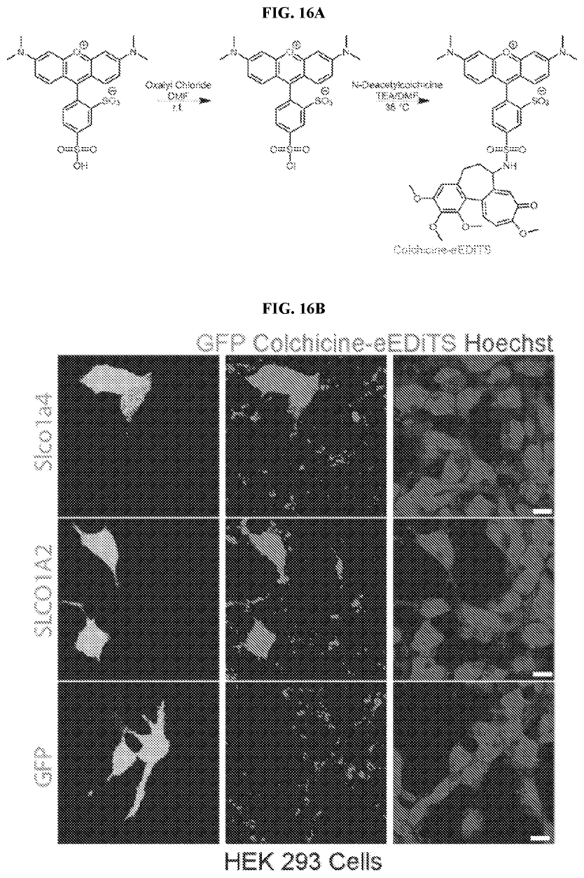

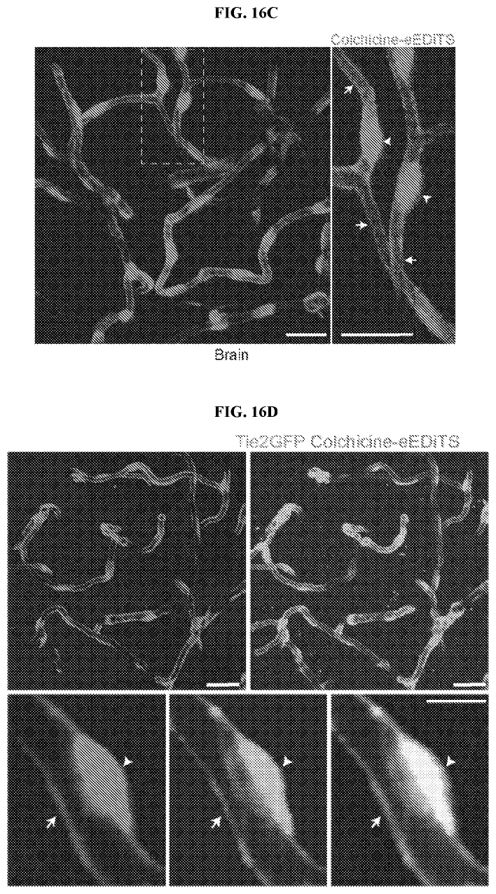

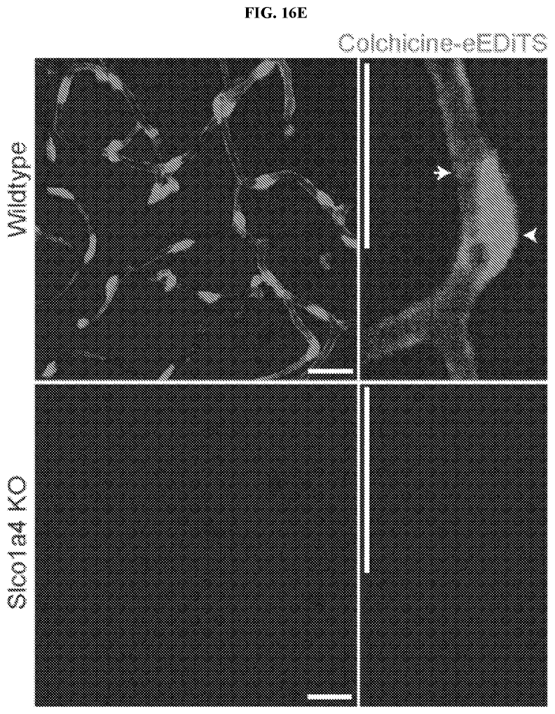

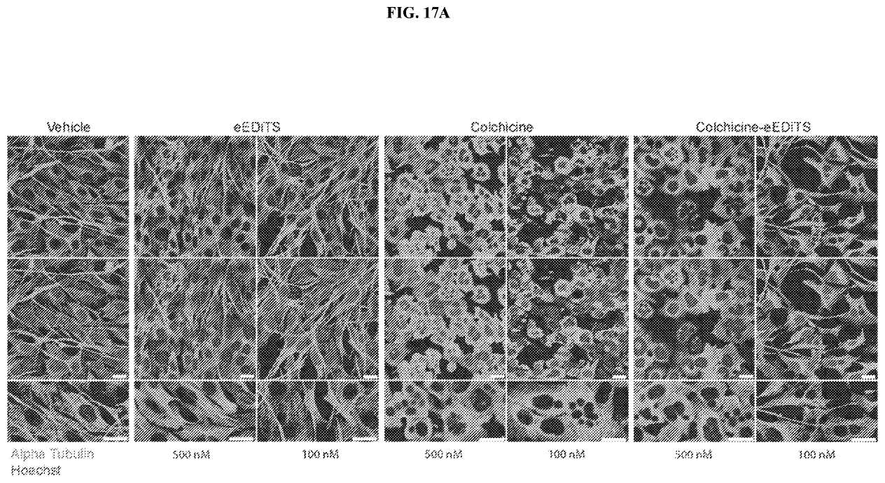

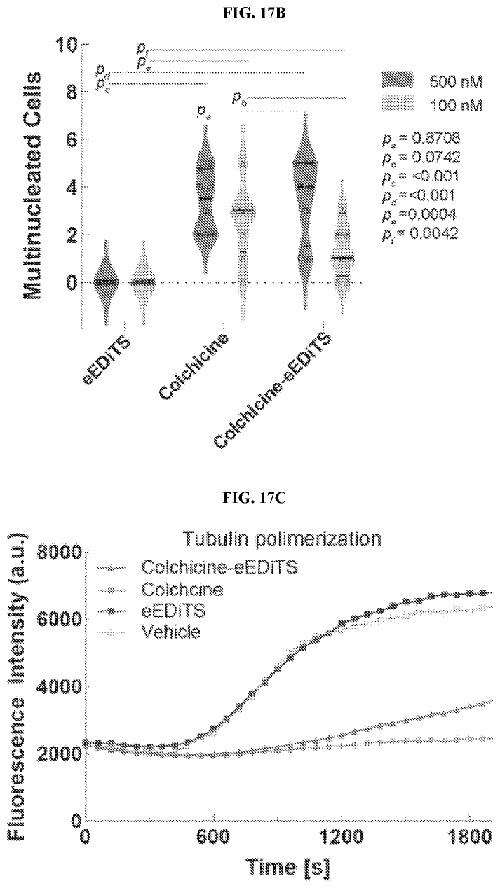

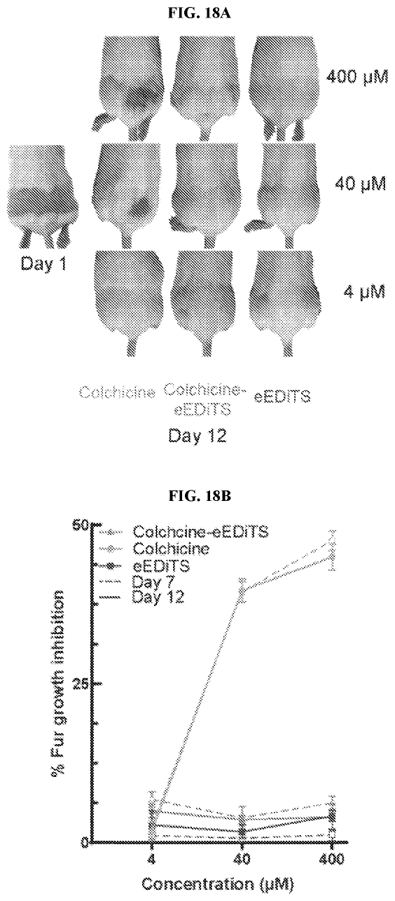

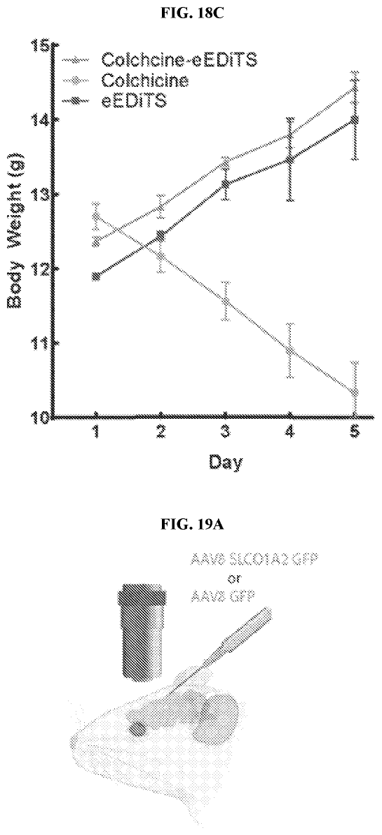

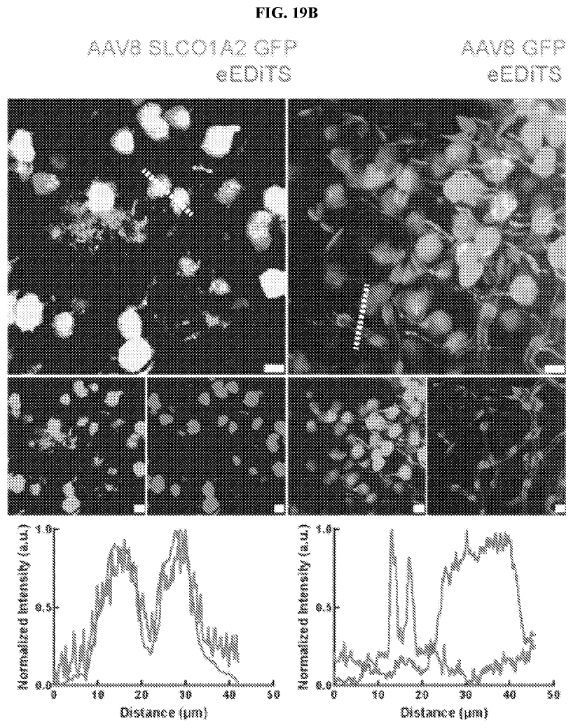

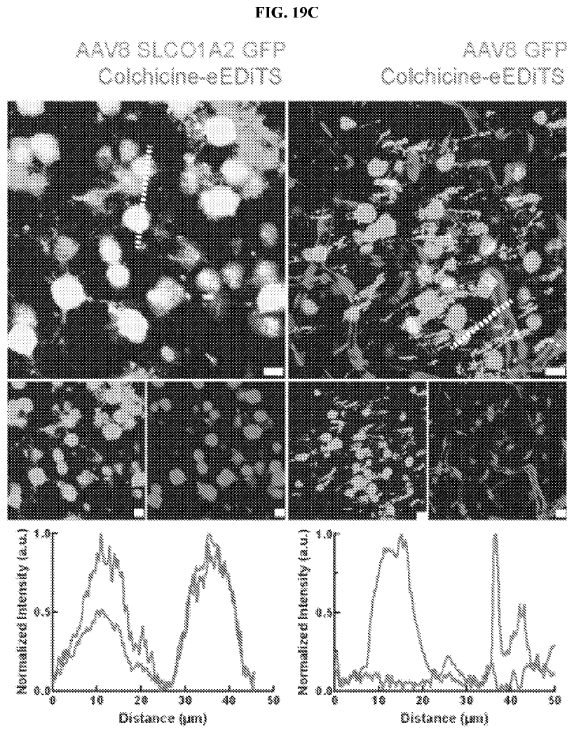

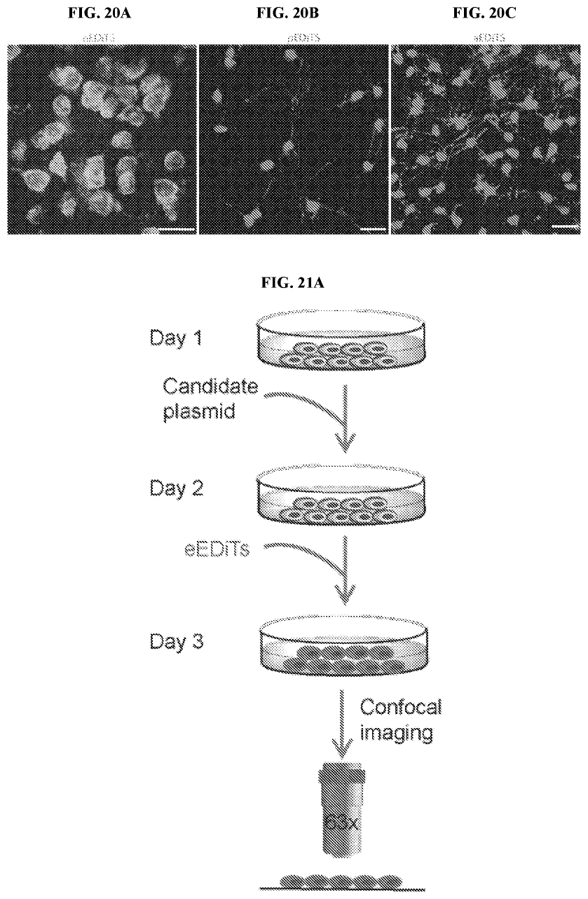

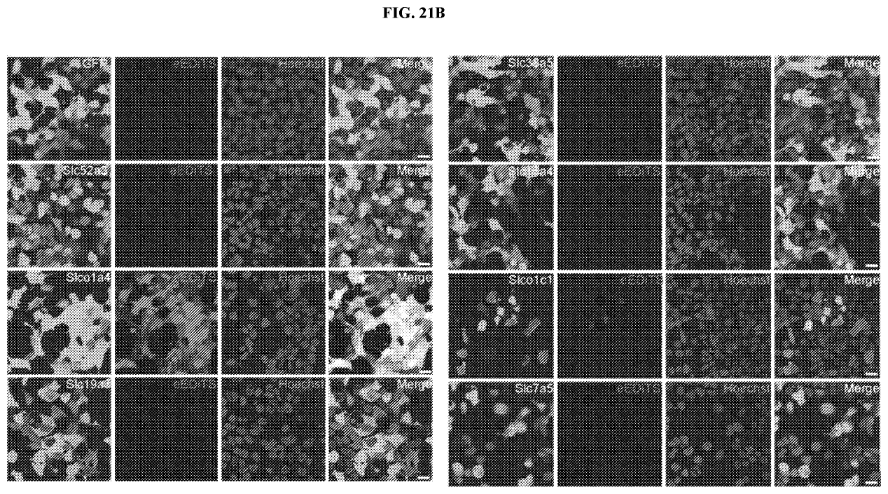

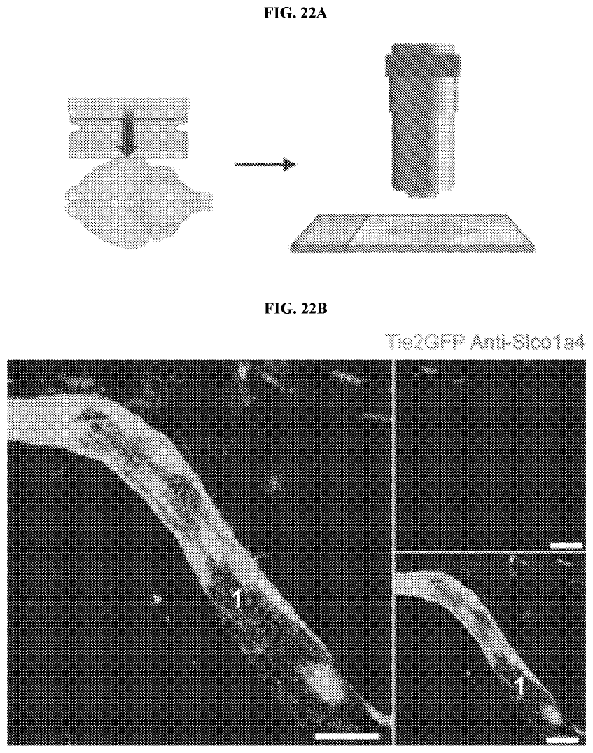

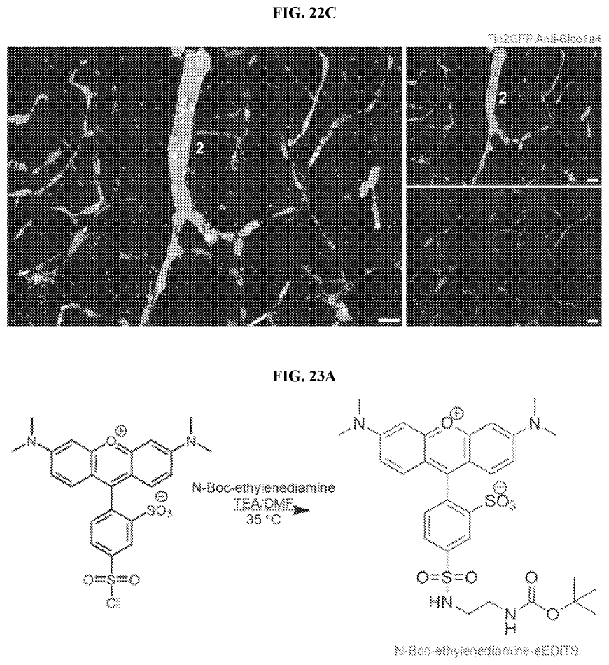

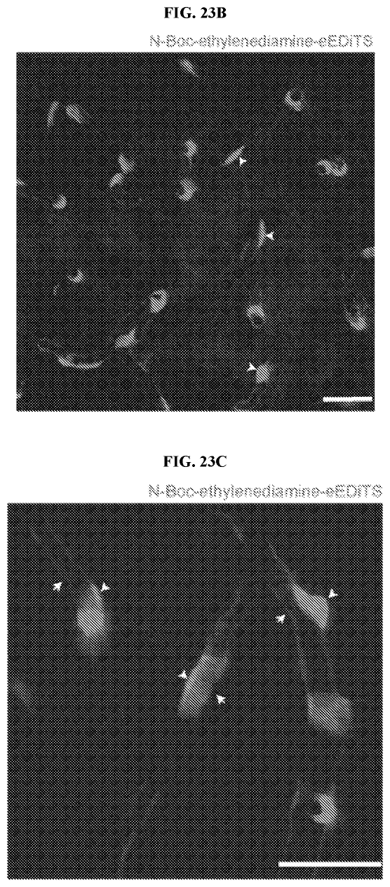

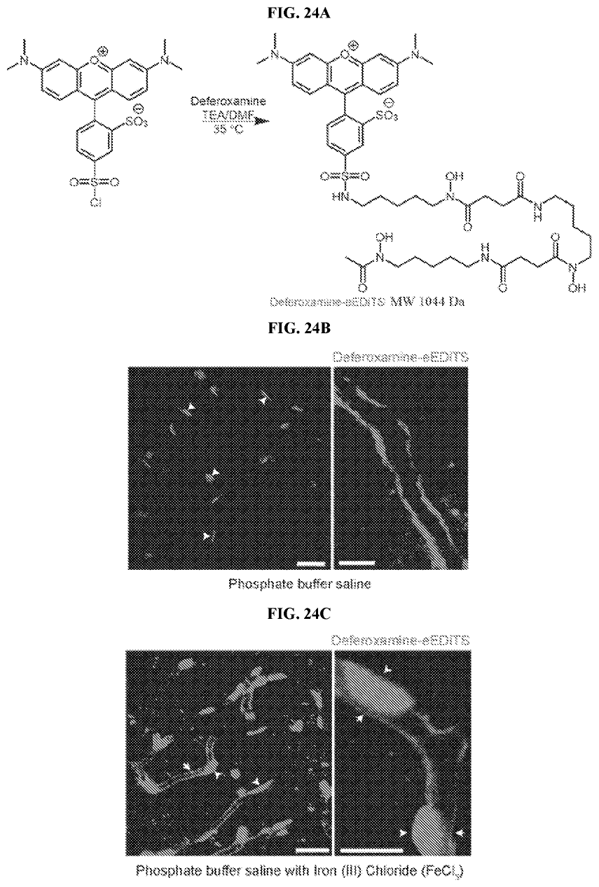

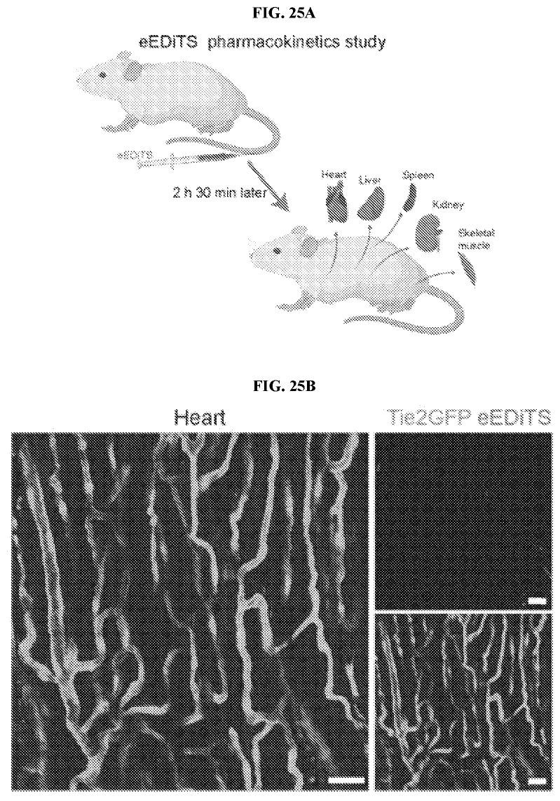

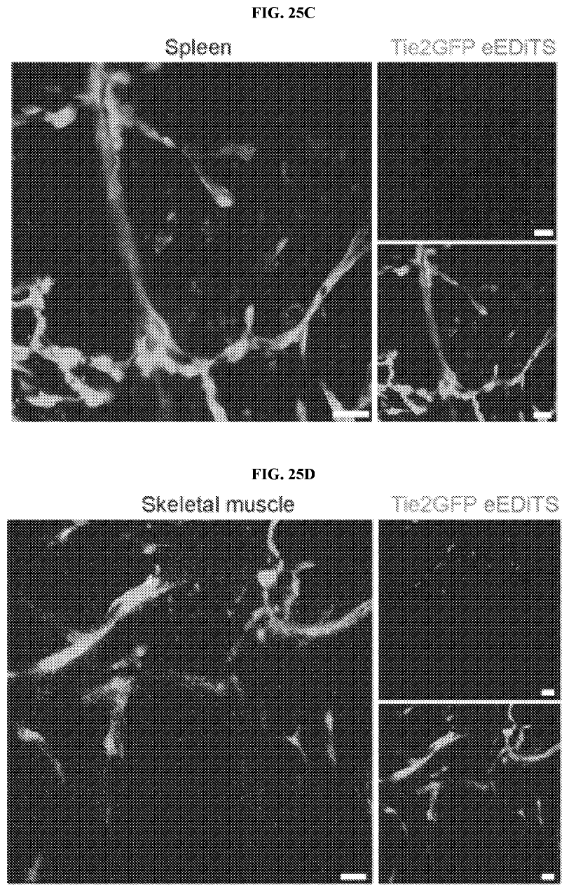

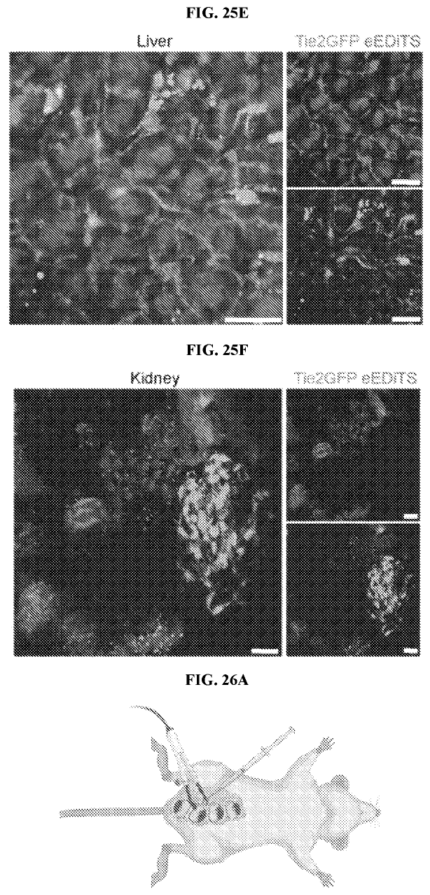

Following the initial screening, proof-of-concept investigations were focused on specific compounds which displayed exceptional selectivity and rapid uptake within endothelial cells in both the brain and retina (endothelial-specific compounds; eEDiTS) (FIGS. 14B-14D). This selectivity was demonstrated by the distinct morphology of endothelial cell bodies and processes (FIGS. 14E-14F) and the precise colocalization of labeling with endothelial cells using Tie2-GFP endothelial reporter transgenic mice (FIGS. 14E-14F).

Next, experiments were conducted using eEDiTS to explore the endothelial transport mechanism. Efficient uptake of eEDiTS was observed through both topical application to the abluminal side or intravascular administration to the luminal endothelium (FIGS. 14E-14F). Importantly, even at very high concentrations, eEDiTS did not label other cell types in the cortex, highlighting its specificity for endothelial cells (FIG. 14C). Moreover, eEDiTS selectively entered all types of endothelial cells, including those in arterioles, capillaries, and venules within the brain parenchyma (FIG. 14C). Administration of eEDiTS to mouse brain slices showed robust endothelial uptake throughout the brain including cortical, subcortical and white matter regions (Table 4). A similar pattern of endothelial labeling with eEDiTS was observed following intravitreally injection into the eye, resulting in rapid labeling of endothelial cells in retinal blood vessels, including arterioles, venules, and capillaries (FIG. 14D). Together these experiments demonstrate that eEDiTS compounds have exquisite affinity for brain and retina endothelial cells.

TABLE 3
eEDiTS labeling in different brain regions assessed
in a live sagittal brain slice preparation.

| Brain regions | Labeling score |
|---|---|
| Hippocampus | +++ |
| Cerebellum | ++ |
| Motor/Somatosensory cortex | +++ |
| White matter | + |
| Thalamus | + |
| Prefrontal cortex | ++ |
| Olfactory bulb | − |
| Hypothalamus | + |
| Visual cortex | ++ |

### Example 10: Cell Type-Specific Pharmacological Access Via Gene Therapy-Mediated Ectopic Transporter Expression

Next, the potential application of the technology described herein to enable selective eEDiTS uptake in cells that do not naturally express the membrane solute carrier was investigated. To achieve this, AAV-mediated gene therapy was performed in live mice (FIGS. 19A-19C) or in utero-electroporation (FIGS. 26A-26C) to ectopically express Slco1a4 or SLCO1A2 in neurons, which do not normally express these transporters. Next, the uptake of eEDiTS or Colchicine-eEDiTS conjugate was assessed in the transfected cells using intravital two-photon imaging. A marked uptake was observed after topical cortical administration of eEDITS or Colchicine-eEDiTS specifically in transfected neuronal cell bodies and dendrites (FIGS. 19B-19C and FIGS. 26B-26C).

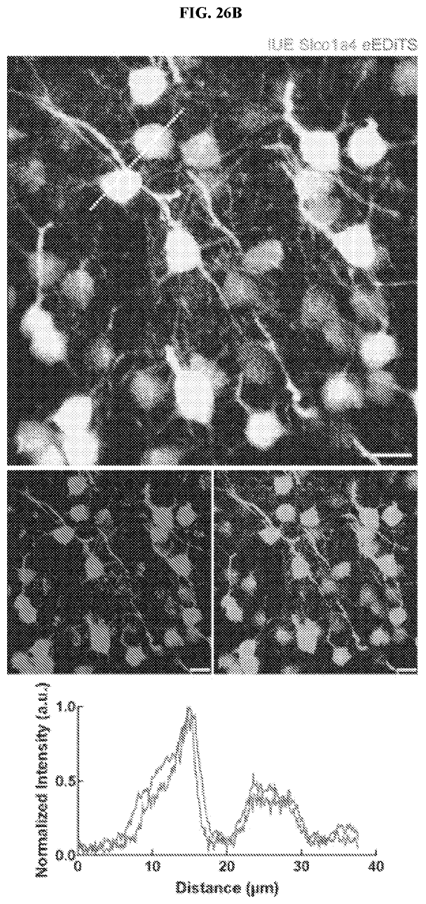

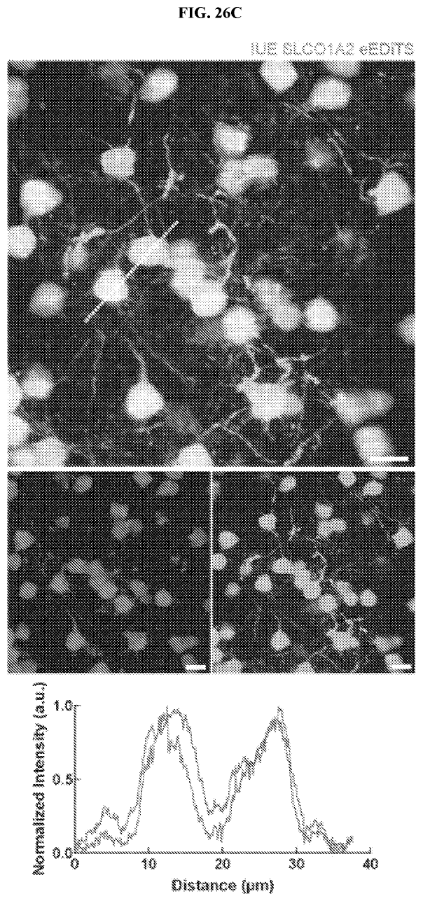

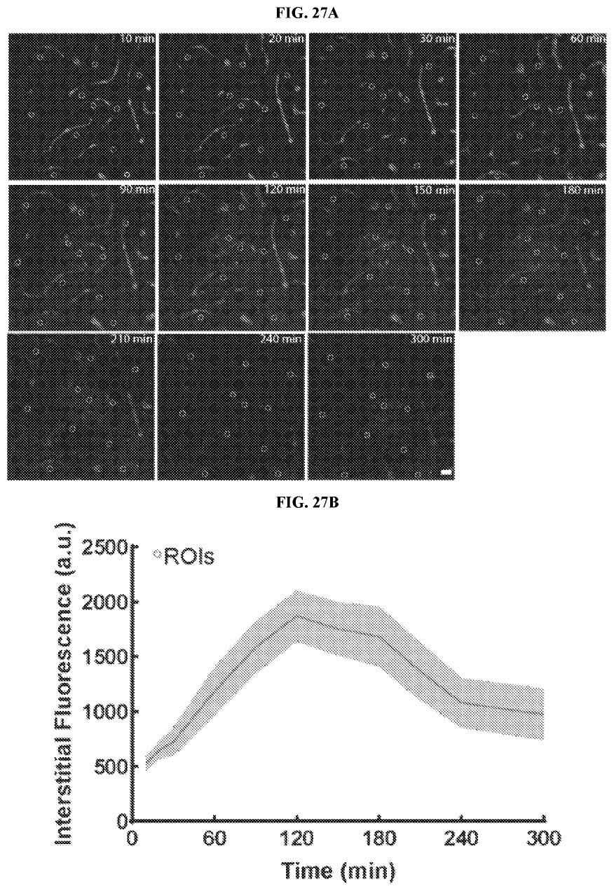

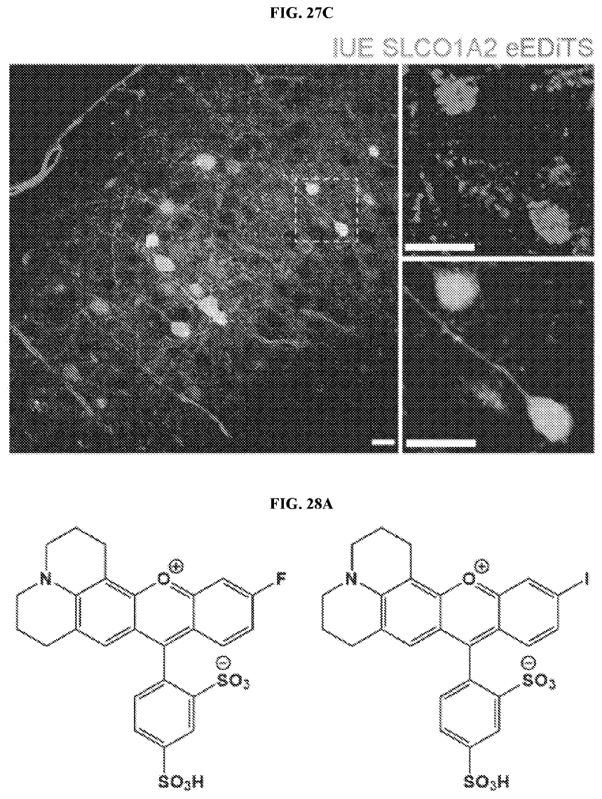

Interestingly, this neuronal uptake was accompanied by a marked reduction in the expected endothelial uptake, likely due to a massively expanded volume of distribution within neurons, redirecting eEDiTS away from the endothelium. Importantly, it is further demonstrated herein that systemic delivery allows for blood-brain barrier (BBB) crossing of eEDiTS (FIGS. 27A-27B) and its ultimate uptake by ectopically transfected neurons (FIG. 27C) and potentially other cell types. Overall, these findings suggest the potential feasibility of using gene therapy to ectopically express selected membrane transporters for chronic pharmacological access to any cell population of interest in the brain and throughout the body.

### Example 11: Selected Results

The clinical utility of otherwise powerful pharmacological agents can be severely limited due to undesired side effects in cell populations not directly involved in the disease process. Unfortunately, there are currently very few approaches to develop drugs with targeted pharmacological effects in selected cell types while sparing most other cells. The present disclosure relates in part to a broad platform to generate cell-type specific pharmacological agents. In one aspect, the discovery strategy described herein emerged from the unexpected observation that some fluorescent small molecules (SMs) had intriguing properties of selective uptake into certain brain cell types in vivo.

It was hypothesized that specific chemical functional groups within these SMs had affinity to membrane transporters which resulted in intracellular uptake selectivity. Thus, a strategy was devised to efficiently discover chemical functional groups that conferred cell-type and membrane transporter selectivity. For this, a combinatorial library of SMs with diverse fluorescent backbones and functional groups was generated and implemented large scale two photon microscopy-based SM screening in the live mouse brain. This intravital imaging approach to screen combinatorial fluorophore libraries provided unique advantages to discover SMs with cell-type selectivity in the native brain microenvironment, without preconceived knowledge about the transport mechanisms.

This approach led to the identification of SMs with various cell-type specificities including a family of endothelial-selective compounds (Endo-red) that enter cells through the solute carriers Slco1a4/A2. Through systematic diversification of chemical functional groups on Endo-Red, combined with in vivo brain imaging, the functional groups that were necessary and sufficient for endothelial and membrane transporter selectivity were identified. This knowledge allowed pursuit of further conjugation to pharmacological agents. Drug conjugation to Endo-Red conferred brain and retinal endothelial specificity to otherwise non-selective pharmacological agents, while retaining the pharmacological properties of the unconjugated drugs. Furthermore, gene-therapy-mediated ectopic expression of membrane transporters allowed conjugated drugs to be potentially targeted to any desired cell population.

For proof of principle conjugation experiments, agents such as colchicine and taxol, with extensive previous literature available about their critical functional groups and potential conjugation sites, were selected. This demonstrated that despite conjugation, the pharmacological properties of the bifunctional compounds could be preserved. However, conjugation to less known or novel compounds would require additional testing of their chemical functional groups to determine which ones are critical for their pharmacological properties and which ones are dispensable during conjugation.

It has been further shown that conjugation can include a cleavable linker which would allow the release of the native pharmacological compounds intracellularly.

Regarding the kinds of therapeutic agents that could be delivered, the proof of principle conjugation experiments demonstrated that cargo molecules ranging from 300 to 1700 Da preserved their cellular uptake specificity and were readily taken up by the target cells. In certain embodiments, large molecular weight therapeutic agents may be utilized, including but not limited to polynucleotide agents. The flexibility in cargo size that has been demonstrated could allow for conjugation of a variety of molecules to cleavable linkers for selective intracellular delivery of novel or FDA approved drugs in their native form.

Endo-Red conjugated drugs can cross the BBB despite their relatively large size and hydrophilicity. This is consistent with the presence of the Slco1a4 transporter on both luminal and abluminal endothelial membranes.

The remarkable uptake specificity of the molecules for various neural cell types including endothelium, pericytes, astrocytes and neurons suggest the possibility of future therapeutic applications for diverse brain and retina pathologies. However, an important factor that needs to be considered is that the transporter cell-type expression patterns can differ between species. For example, while SLCO1A2 is highly enriched in human brain and retinal endothelium, it is also expressed in oligodendrocytes and retinal pigmented epithelium (RPE).

Endothelial pathology is a signature of many retina and brain disorders. For example, age-related macular degeneration (AMD) is associated with extensive retinal neovascularization and vascular hyperpermeability as well as RPE degeneration. Endo-red conjugates with drugs targeting a variety of intracellular signaling pathways including receptor tyrosine kinases involved in VEGF or inflammatory pathways (JAK/STAT) could thus be developed. Current therapies for retinal disease mostly consist of intravitreal injectable antibodies that can treat the neovascularization aspects of the disease but not the neurodegenerative and RPE pathology.

This strategy can generate orally or topically bioavailable cell-type specific drugs for the treatment of common retinal diseases. Similar approaches can be taken to target endothelium and oligodendrocytes, the two cell types most affected in diseases such as vascular dementia and multiple sclerosis.

It is envisioned that, using these screening approaches, one can identify SMs with affinity to membrane transporters found in evolutionary removed species (i.e., invertebrates) and therefore very different from mammalian transporters. Gene therapy expression of such transporters could allow highly selective pharmacological targeting of cell populations with potentially no uptake by non-transfected mammalian cells.

Additional potential applications of these SMs could include the design of cell-type specific neuroimaging probes. For example, SMs that target astrocytes, neurons or other cell types can fluorinated or iodinated to generate radiotracers for positron emission tomography (PET) imaging. Certain solute carriers have been identified in GWAS studies as risk factors in a variety of diseases including neurodegeneration refs. For example, variants in SLCO1A2 were shown to increase risk of progressive supranuclear palsy (PSP) ref. Furthermore. SLCO1A2 levels have recently been shown to be a hallmark of Alzheimer's disease brain. Therefore, one can envision the generation of PET radiotracers to measure SLCO1 A2 or other transporters function in vivo as potential biomarkers of disease.

In certain embodiments, the results of labeling of endothelial cells in vivo with fluorinated (FIG. 7A) and iodinated (FIG. 7B) compounds 2 and compound 4 respectively (FIG. 8G). These results demonstrate that these compounds, which are readily converted into radiotracers, can be used for specific endothelial visualization using nuclear medicine imaging modalities.

In certain embodiments, results of labeling of retinal endothelial cells in vivo with compound 1 according to the invention. In vivo intraocular injection of compound 1 leads to rapid and highly specific labeling of retinal vasculature. This result demonstrates that the compounds of the invention can be utilized for imaging and delivery of molecules to the retina for a variety of applications including diagnostics and therapeutics.

In one set of experiments, HEK293 cells were transfected with a plasmid carrying a gene encoding the human solute carrier organic anion transporter family member 1A2 (SLCO1A2). In certain embodiments, only those cells expressing the carrier SLCO1A2 (as evidenced by anti-FLAG labeling-green) take up compound 1 (red). All other cells marked by Hoechst dye labeling (blue), do not take up the compound. This demonstrates exquisite cellular specificity of the compound.

In another set of experiments, HEK293 were transfected with a plasmid carrying a gene encoding the mouse solute carrier SLCO1A4. SLCO1A4 is the mouse orthologue of human SLCO1A2. In certain embodiments, only those cells expressing SLCO1A4 (as evidenced by anti-FLAG labeling-green) take up compound 1 (red). All other cells marked by Hoechst dye labeling (blue), do not take up compound 1. In addition, expression of a different solute carrier, solute carrier family 2 member 3 (SLC2A3), does not induce uptake. This demonstrates exquisite cellular and molecular specificity of the compound.

SLCO1A2 or SLCO1A4 was over-expressed in the live mouse brain by in utero electroporation. In certain embodiments, only those cells that express SLCO1A2 or SLCO1A4 (as evidenced by the GFP fluorescence (green)) take up compound 1 after topical cortical administration as described above. This demonstrates cellular and molecular specificity in vivo of the compound.

Compound 1 was also administered into a mouse in which SLCO1A4 has been deleted. This mouse lacks several organic anion transporting polypeptide (OATP) 1a/1b cluster of solute carriers including SLCO1A4. SLCO1A1, SLCO1A5, SLCO1A6, and SLCO1B2. In certain embodiments, there is a loss of endothelial uptake of compound 1 in the mouse. A complete disappearance of endothelial uptake was observed in the knockout mice (left panel) as compared to the control (right panel). Astrocyte uptake is seen in the knockout mice, likely by a compensatory mechanism.

In certain embodiments, compound 1 readily enters cancer cells in freshly excised breast surgical tissue. Membrane transport mechanism is expressed in breast cancer as seen by the membrane labeling with immunohistochemistry for SLCO1A2 (white). Injection of compound 1 into freshly excised cancerous breast tissue leads to rapid entry into cancer cells (red). Blue represents nuclear labeling with Hoechst dye.

Compounds of the invention may be used for various applications in the treatment or imaging of triple negative breast cancer.

Various compounds of the invention are evaluated for in vivo target engagement in patient-derived xenograft triple negative breast cancer (TNBC) mouse models. Compounds are also evaluated for imaging of breast cancer using in vivo IVIS Spectrum optical imaging. Compounds that exhibit in vivo cellular specificity are selected.

SPECT imaging with a radioactive fluorinated-compound is studied in xenograft TNBC mouse models. SPECT imaging is also conducted in a model of TNBC brain metastasis and in vivo cell killing with a radioactive compound is assessed.

Candidate compounds are iodinated and tested for in vivo tumor killing properties in mice. Candidate compounds are also conjugated with different chemotherapeutic agents and tested for receptor specificity and cell killing properties in vitro. Pharmacokinetics studies are performed in rodents with the iodinated compounds or compounds conjugated to a chemotherapeutic agent.

A library is developed in which selected chemotherapeutic agents are conjugated to compounds of the invention, and analogues thereof, with and without cleavable linkers, to afford conjugates of the present invention. Medicinal chemistry of the compound-drug conjugates is assessed and used for compound optimization. In vitro efficacy studies are performed to evaluate the cell killing and antiproliferation properties of the compound-drug conjugates. Lead compounds are selected from optimization in vivo in patient-derived cancer cell xenograft mouse models of glioblastoma and breast cancer with brain metastasis. A companion radioactive fluorinated version of select compound is developed for SPECT/PET imaging.

### Enumerated Embodiments

The following exemplary embodiments are provided, the numbering of which is not to be construed as designating levels of importance:

Embodiment 1 provides a method of delivering a compound into a cell, comprising:
expressing Slco1a4 or SLCO1A2 in the cell; and
contacting with the cell a compound of formula (I), or a salt, solvate, tautomer, geometric isomer, or isotopologue thereof, and any combinations thereof:

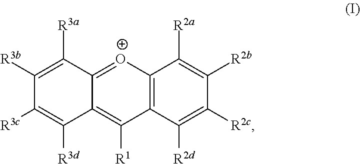

wherein:
R¹ is

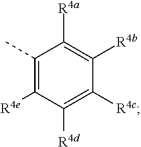

R^{2a}, R^{2b}, R^{2c}, and R^{2d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B},
   wherein two vicinal substituents selected from the group consisting of R^{2a}, R^{2b}, R^{2c}, and R^{2d} can combine with the atoms to which they are bound to form an optionally substituted C₆-C₁₀ aryl or optionally substituted C₂-C₁₀ heteroaryl, and
   wherein no more than three of R^{2a}, R^{2b}, R^{2c}, and R^{2d} are H;
R^{3a}, R^{3b}, R^{3c}, R^{3d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D},
   wherein two vicinal substituents selected from the group consisting of R^{3a}, R^{3b}, R^{3c}, and R^{3d} can combine with the atoms to which they are bound to form an optionally substituted C₆-C₁₀ aryl or optionally substituted C₂-C₁₀ heteroaryl, and
   wherein no more than three of R^{3a}, R^{3b}, R^{3c}, and R^{3d} are H;
R^{4a}, R^{4b}, R^{4c}, R^{4d}, and R^{4e} are each independently selected from the group consisting of H, optionally substituted C₁-C₆ alkyl, CN, NO₂, S(═O)₂OH, and S(═O)₂R⁵,
   wherein at least one of R^{4a}, R^{4b}, R^{4c}, R^{4d}, and R^{4e} is S(═O)₂OH, and
   wherein at least one of R^{4a}, R^{4b}, R^{4c}, R^{4d}, and R^{4e} is S(═O)₂R⁵;
R⁵ is at least one selected from the group consisting of H, OH, halogen, and

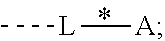

each occurrence of L is independently selected from the group consisting of a bond and a linker;
each occurrence of A is H, optionally substituted C₁-C₆ alkyl, an imaging agent, a polymeric macromolecule, or a therapeutic agent;
R^{A} and R^{B}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{A} and R^{B} can independently with one of R^{2a}, R^{2b}, R^{2c}, and R^{2d} to form an optionally substituted C₅-C₈ heterocycloalkyl or C₅-C₈ heterocycloalkenyl; and
R^{C} and R^{D}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{C} and R^{D} can independently with one of R^{3a}, R^{3b}, R^{3c}, and R^{3d} to form an optionally substituted C₅-C₈ heterocycloalkyl or C₅-C₈ heterocycloalkenyl.

Embodiment 2 provides the method of Embodiment 1, wherein R^{2a}, R^{2b}, R^{2c}, and R^{2d} are each independently selected from the group consisting of H, F, Cl, Br, I, CH₃, CF₃, SO₃H, N(CH₃)₂, N(CH₂CH₃)₂, —(CH₂)₂C(═O)OH, B(OH)₂,

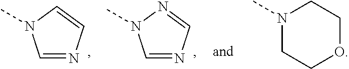

Embodiment 3 provides the method of Embodiment 1 or 2, wherein R^{3a}, R^{3b}, R^{3c}, and R^{3d} are each independently selected from the group consisting of H, F, Cl, Br, I, CH₃, CF₃, SO₃H, N(CH₃)₂, N(CH₂CH₃)₂, —(CH₂)₂C(═O)OH, B(OH)₂,

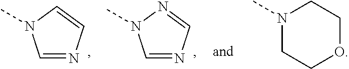

Embodiment 4 provides the method of any one of Embodiments 1-3, wherein one of the following applies:
(a) R^{2a} is H, R^{2b} is Br, R^{2c} is H, and R^{2d} is H;
(b) R^{2a} is H, R^{2b} is H, R^{2c} is Br, and R^{2d} is H;
(c) R^{2a} is H, R^{2b} is N(CH₃)₂, R^{2c} is H, and R^{2d} is H; and
(d) R^{2a} is H, R^{2b} is N(CH₂CH₃)₂, R^{2e} is H, and R^{2d} is H.

Embodiment 5 provides the method of any one of Embodiments 1-4, wherein one of the following applies:
(a) R^{3a} is H, R^{3b} is NO₂, R^{3e} is H, and R^{3d} is H;
(b) R^{3a} is H, R^{3b} is CH₃, R^{3e} is H, and R^{3d} is H;
(c) R^{3a} is H, R^{3b} is

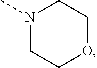

R^{3c} is H, and R^{3d} is H;
(d) R^{3a} is H, R^{3b} is H, R³c is

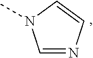

### and R^{3d} is H;

(e) R^{3a} is H, R^{3b} is H, R^{3c} is SO₃H, and R^{3d} is H;
(f) R^{3a} is H, R^{3b} is H, R^{3c} is

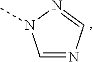

### and R^{3d} is H;

(g) R^{3a} is H, R^{3b} is N(CH₃)₂, R^{3e} is H, and R^{3d} is H; and
(h) R^{3a} is H, R^{3b} is N(CH₂CH₃)₂, R^{3e} is H, and R^{3d} is H.

Embodiment 6 provides the method of any one of Embodiments 1-5, wherein the compound of formula (I) is selected from the group consisting of:

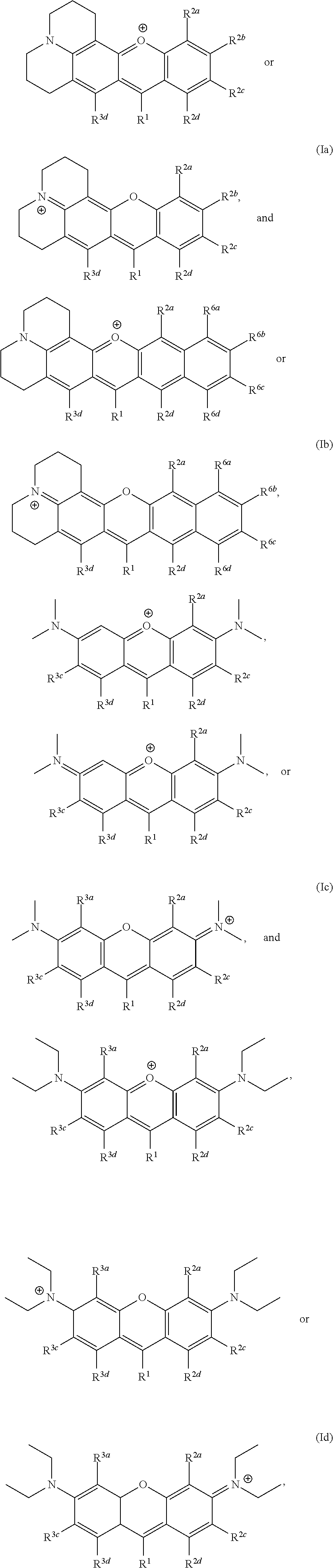

wherein R^{6a}, R^{6b}, R^{6c}, an R^{6d}, if present, are each independently selected from the group consisting of H, C₁-C₆ alkyl, C₁-C₆ haloalkyl, halogen, CN, and NO₂.

Embodiment 7 provides the method of any one of Embodiments 1-6, wherein R^{3d} is H.

Embodiment 8 provides the method of Embodiment 6 or 7, wherein one of the following applies:
(a) R^{2a} is H, R^{2b} is Br, R^{2c} is H, and R^{2d} is H:
(b) R^{2a} is H, R^{2b} is F, R^{2c} is H, and R^{2d} is H;
(c) R^{2a} is H, R^{2b} is Cl, R^{2c} is H, and R^{2d} is H;
(d) R^{2a} is H, R^{2b} is I, R^{2c} is H, and R^{2d} is H;
(e) R^{2a} is Br, R^{2b} is H, R^{2c} is H, and R^{2d} is H;
(f) R^{2a} is H, R^{2b} is H, R^{2c} is Br, and R^{2d} is H:
(g) R^{2a} is Br, R^{2b} is H, R^{2c} is Br, and R^{2d} is H;
(h) R^{2a} is H, R^{2b} is F, R^{2c} is F, and R^{2d} is H;
(i) R^{2a} is H, R^{2b} is F. R^{2c} is Cl, and R^{2d} is H:
(j) R^{2a} is H, R^{2b} is F, R^{2c} is Br, and R^{2d} is H;
(k) R^{2a} is H, R^{2b} is Cl, R^{2c} is F, and R^{2d} is H;
(l) R^{2a} is H, R^{2b} is Cl, R²e is Cl, and R^{2d} is H;
(m) R^{2a} is H, R^{2b} is Cl, R^{2c} is Br, and R^{2d} is H;
(n) R^{2a} is H, R^{2b} is Br, R^{2c} is F, and R^{2d} is H.
(o) R^{2a} is H, R^{2b} is Br, R^{2c} is Cl, and R^{2d} is H;
(p) R^{2a} is H, R^{2b} is I, R^{2c} is Cl, and R^{2d} is H;
(q) R^{2a} is H, R^{2b} is CF₃, R^{2c} is Br, and R^{2d} is H,
(r) R^{2a} is F, R^{2b} is F, R^{2c} is H, and R^{2d} is H;
(s) R^{2a} is F, R^{2b} is Cl, R^{2c} is H, and R^{2d} is H;
(t) R^{2a} is F, R^{2b} is Br, R^{2c} is H, and R^{2d} is H;
(u) R^{2a} is Cl, R^{2b} is F, R^{2c} is H, and R^{2d} is H;
(v) R^{2a} is Cl, R^{2b} is Cl, R^{2c} is H, and R^{2d} is H;
(w) R^{2a} is H, R^{2b} is NO₂, R^{2c} is H, and R^{2d} is H:
(x) R^{2a} is H, R^{2b} is N(CH₃)₂, R^{2c} is H, and R^{2d} is H;
(y) R^{2a} is H, R^{2b} is —(CH₂)₂C(═O)OH, R²e is H, and R^{2d} is H;
(z) R^{2a} is H, R^{2b} is CH₃, R^{2c} is H, and R^{2d} is H;
(aa) R^{2a} is H, R^{2b} is B(OH)₂, R^{2c} is H, and R^{2d} is H:
(ab) R^{2a} is H, R^{6a} is H, R^{6b} is H, R^{6e} is Br, Rid is H, and R^{2d} is H; and
(ac) R^{2a} is Br, R⁶a is H, R^{6b} is H, R^{6c} is H, R^{6d} is H, and R^{2d} is H.

Embodiment 9 provides the method of any one of Embodiments 1-8, wherein at least one of the following applies:
(a) at least one of R^{4a}, R^{4b}, R^{4c}, R^{4d}, and R^{4e} is H;
(b) at least two of R^{4a}, R^{4b}, R^{4c}, R^{4d}, and R^{4e} are H;
(c) at least three of R^{4a}, R^{4b}, R^{4c}, R^{4d}, and R^{4e} are H; and
(d) four of R^{4a}, R^{4b}, R^{4c}, R^{4d}, and R^{4e} are H.

Embodiment 10 provides the method of any one of Embodiments 1-9, wherein R¹ is selected from the group consisting of

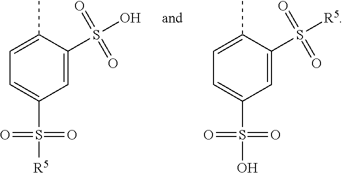

Embodiment 11 provides the method of any one of Embodiments 1-10, wherein L comprises n instances of Z, wherein: each occurrence of Z is independently selected from the group consisting of —O—, —N(R⁷)—, —S—, —S(═O)—, —S(═O)₂—, —C(═O)—, optionally substituted C₁-C₁₀ alkylenyl, optionally substituted C₁-C₁₀ heteroalkylenyl, optionally substituted C₃-C₈ cycloalkylenyl, optionally substituted C₂-C₈ heterocycloalkylenyl, optionally substituted C₁-C₁₀ alkenylenyl, optionally substituted C₃-C₈ cycloalkenylenyl, optionally substituted C₂-C₈ heterocycloalkenylenyl, optionally substituted C₆-C₁₀ arylenyl, and optionally substituted C₂-C₁₀ heteroarylenyl;
R⁷ is selected from the group consisting of H and C₁-C₆ alkyl; and
n is an integer ranging from 0 to 500.

Embodiment 12 provides the method of Embodiment 11, wherein each instance of Z is independently selected from the group consisting of —NH—, —S—, —O—, —(CH₂)₀₋₁₀—, —(CH₂)₂O—, —O(CH₂)₂—, —C(═O)—,

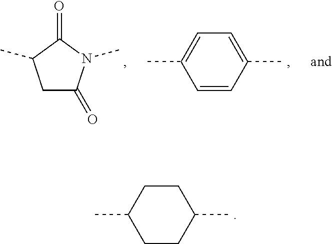

Embodiment 13 provides the method of any one of Embodiments 1-12, wherein L is selected from the group consisting of a bond, —NH—,

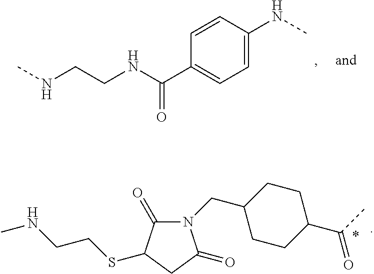

Embodiment 14 provides the method of any one of Embodiments 1-12, wherein the therapeutic agent is selected from the group consisting of a small molecule, polypeptide, protein, and aptamer.

Embodiment 15 provides the method of Embodiment 14, wherein the small molecule is a compound useful for the treatment of cancer.

Embodiment 16 provides the method of any one of Embodiments 1-15, wherein the therapeutic agent is selected from the group consisting of colchicine, deferoxamine, paclitaxel (taxol), tofacitinib, methotrexate, hydrocortisone, prednisone, triiodothyronine, cyclophosphamide, amphotericin B, vancomycin, doxorubicin, mitoxantrone, imatinib, darunavir, and fosamprenavir.

Embodiment 17 provides the method of any one of Embodiments 1-16, wherein the therapeutic agent comprises at least one modification and/or derivatization.

Embodiment 18 provides the method of any one of Embodiments 1-17, wherein the therapeutic modification and/or derivatization comprises bond

[symbol]

Embodiment 19 provides the method of any one of Embodiments 1-18, wherein A is selected from the group consisting of

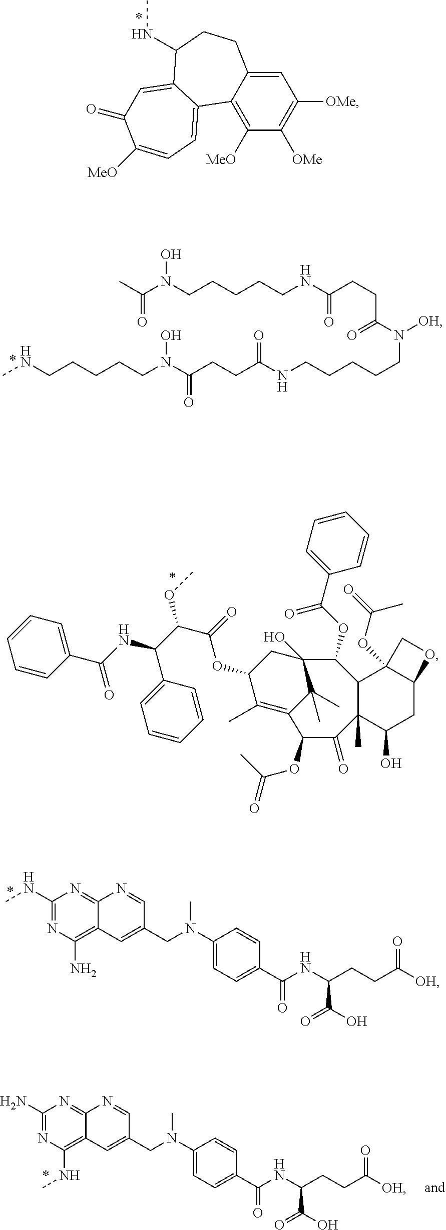

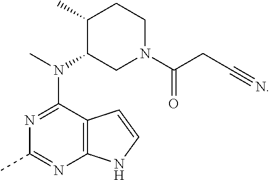

Embodiment 20 provides the method of any one of Embodiments 1-10, wherein A is a compound of formula (III):

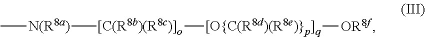

wherein:
R^{8a} and R^{8f} are each independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
each occurrence of R^{8b}, R^{8c}, R^{8d}, and R^{8e} is independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
is an integer selected from 1, 2, 3, 4, and 5;
p is an integer selected from 1, 2, 3, 4, and 5; and
q is an integer ranging from 1 to 500.

Embodiment 21 provides the method of Embodiment 20, wherein at least one of the following applies:
(a) R^{8a} is H;
(b) R^{8f} is selected from the group consisting of H and Me:
(c) each occurrence of R^{8a}, R^{8b}, R^{8c}, and R^{8e} is independently H;
(d) o is 2;
(e) p is 2; and
(f) q is 10 to 500.

Embodiment 22 provides the method of Embodiment 20 or 21, wherein A is

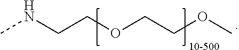

Embodiment 23 provides the method of any one of Embodiments 1-22, wherein R⁵ is selected from the group consisting of

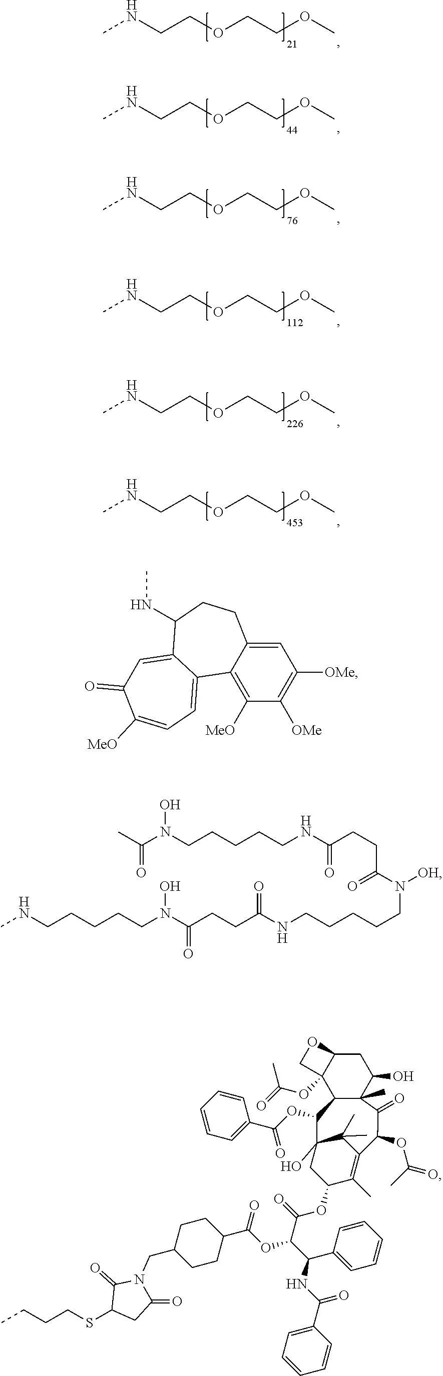

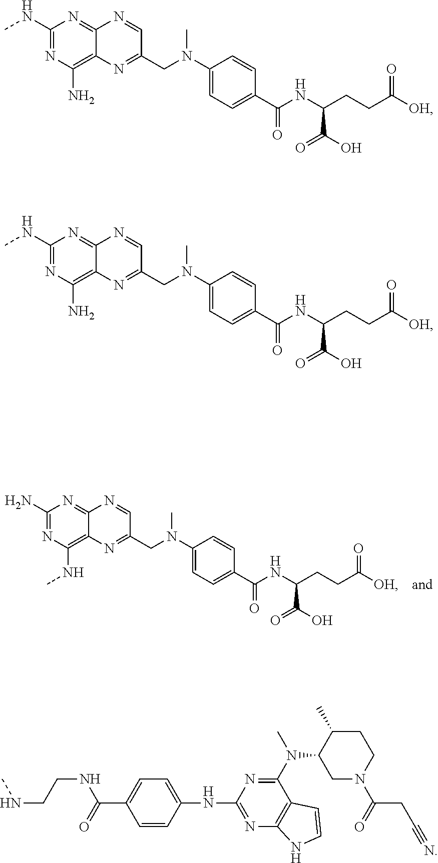

Embodiment 24 provides the method of any one of Embodiments 1-23, wherein each occurrence of optionally substituted alkyl, optionally substituted haloalkyl, optionally substituted cycloalkyl, optionally substituted heterocycloalkyl, optionally substituted heterocycloalkenyl, optionally substituted aryl, optionally substituted heteroaryl, optionally substituted alkylenyl, optionally substituted cycloalkylenyl, optionally substituted heterocycloalkylenyl, optionally substituted alkenylenyl, optionally substituted cycloalkenylenyl, optionally substituted heterocycloalkenylenyl, optionally substituted arylenyl, and optionally substituted heteroarylenyl is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂ ORI, N(R^{I})(R^{II}) C₁-C₆ haloalkoxy, C₃-C₈ halocycloalkoxy, aryl, heteroaryl, (C₁-C₆ alkylenyl)C(═O)N(R^{I})(R^{II}), (C₁-C₆ alkylenyl)C(═O)OR^{I}, O(C₁-C₃ alkylenyl)C(═O)OR^{II}, O(C₁-C₃ alkylenyl)C(═O)N(R^{I})(R^{II}), C(═O)R^{I}, C(═O)OR^{I}, OC(═O)R^{I}, OC(═O)OR^{I}, SR^{I}, S(═O)R^{I}, S(═O)₂R^{I}, S(═O)₂N(R^{I})(R^{II}), S(═O)₂NR^{I}C(═O)NHR^{II}, N(R^{I})S(═O)₂R^{II}, N(R^{I})C(═O)R^{II}, and C(O)NR^{I}R^{II}, wherein R^{I} and R^{II} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

Embodiment 25 provides the method of any one of Embodiments 1-24, wherein the isotopologue thereof comprises at least one radioactive tracer selected from the group consisting of ¹¹C, ³N, ¹⁵O, ¹⁸F, ¹²⁴I, ¹³¹I, and ¹³⁵I.

Embodiment 26 provides the method of any one of Embodiments 1-25, which is selected from the group consisting of.

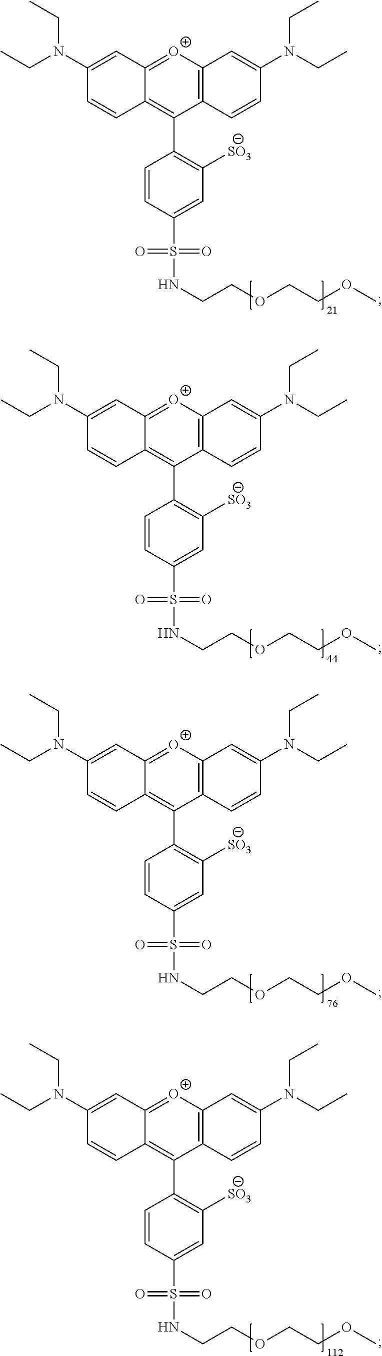

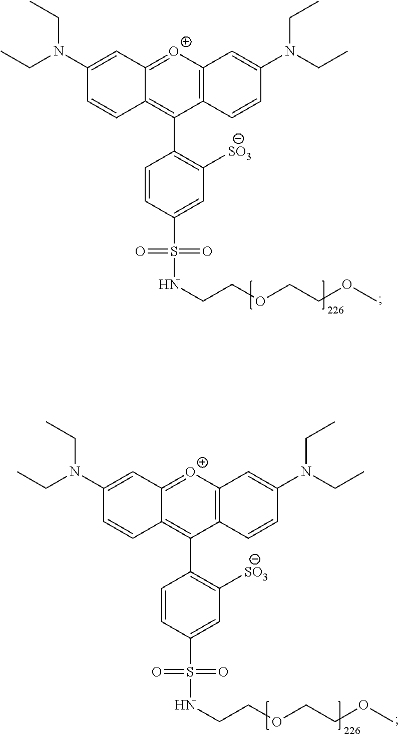

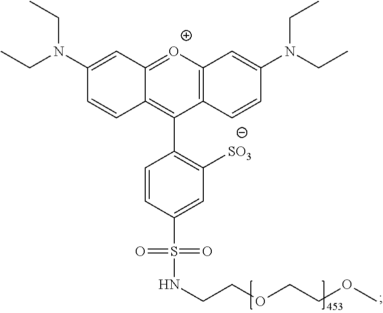

2-(3,6-bis(dimethylamino)xanthylium-9-yl)-5-(N-(1,2,3,10-tetramethoxy-9-oxo-5,6,7,9-tetrahydrobenzo[a]heptalen-7-yl)sulfamoyl)benzenesulfonate;
2-(3,6-bis(diethyl amino)xanthylium-9-yl)-5-(N-(1,2,3,10-tetramethoxy-9-oxo-5,6,7,9-tetrahydrobenzo[a]heptalen-7-yl)sulfamoyl)benzenesulfonate;
2-(3,6-bis(dimethylamino)xanthylium-9-yl)-5-(N-(3,14,25-trihydroxy-2,10,13,21,24-pentaoxo-3,9,1420,25-pentaazatriacontan-30-yl)sulfamoyl)benzenesulfonate;
5-(N-(2-((1-((4-((((1R,2S)-1-benzamido-3-(((2aS,4R,4aR,6S,9R,11R,12R,12aS,12bR)-6,12b-diacetoxy-12-(benzoyloxy)-4,11-dihydroxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-9-yl)oxy)-3-oxo-1-phenylpropan-2-yl)oxy)carbonyl)cyclohexyl)methyl)-2,5-dioxopyrrolidin-3-yl)thio)ethyl)sulfamoyl)-2-(3,6-bis(dimethylamino)xanthylium-9-yl)benzenesulfonate;
(S)-5-(N-(4-amino-6-(((4-((1,3-dicarboxypropyl)carbamoyl)phenyl)(methyl)amino)methyl)pteridin-2-yl)sulfamoyl)-2-(12-bromo-1,2,3,5,6,7-hexahydrochromeno[2,3-f]pyrido[3,2,1-ij]quinolin-4-ium-9-yl)benzenesulfonate;
(R)-5-(N-(2-amino-6-(((4-((1,3-dicarboxypropyl)carbamoyl)phenyl)(methyl)amino)methyl)pteridin-4-yl)sulfamoyl)-2-(12-bromo-1,2,3,5,6,7-hexahydrochromeno[2,3-f]pyrido[3,2,1-ij]quinolin-4-ium-9-yl)benzenesulfonate; and
5-(N-(2-(4-((4-(((3R,4R)-1-(2-cyanoacetyl)-4-methylpiperidin-3-yl)(methyl)amino)-7H-pyrrolo[2,3-d]pyrimidin-2-yl)amino)benzamido)ethyl)sulfamoyl)-2-(6-(diethylamino)-3-(diethyliminio)-3H-xanthen-9-yl)benzenesulfonate.

Embodiment 27 provides the method of any one of Embodiments 1-26, wherein expressing Slco1a4 or SLCO1A2 in the cell comprises:
delivering a Slco1a4 protein or a SLCO1A2 protein into the cell;
delivering an mRNA encoding Slco1a4 or a SLCO1A2 into the cell, wherein the mRNA comprises a constitutively active promoter or a promoter active in the cell;
delivering a DNA encoding Slco1a4 or a SLCO1A2 into the cell, wherein the DNA comprises a constitutively active promoter or a promoter active in the cell;
introducing a DNA encoding Slco1a4 or a SLCO1A2 into the genome of the cell, wherein the DNA comprises a constitutively active promoter or a promoter active in the cell that drives the expression of the Slco1a4 or SLCO1A2, or wherein the DNA is inserted downstream of a promoter active in the cell; or
introducing a constitutively active promoter or a promoter active in the cell to drive the expression of a Slco1a4 gene or a SLCO1A2 gene in the cell.

Embodiment 28 provides the method of any one of Embodiments 1-27, wherein the compound is co-formulated with a pharmaceutically acceptable carrier.

Embodiment 29 provides the method of any one of Embodiments 1-28, wherein the cell is in a subject, and wherein the method comprises administering to the subject an effective amount of the compound.

Embodiment 30 provides the method of Embodiment 29, wherein the compound is administered topically, intravenously, orally, intramuscularly, intrathecally, or intraperitoneally.

Embodiment 31 provides the method of any one of Embodiments 29-30, wherein the subject is a mammal.

Embodiment 32 provides the method of Embodiment 31, wherein the mammal is a human.

Embodiment 33 provides a kit for delivering a compound into a cell, comprising:
a Slco1a4 protein, a SLCO1A2 protein, or a nucleic acid encoding the same; and
a compound of formula (I), or a salt, solvate, tautomer, geometric isomer, or isotopologue thereof, and any combinations thereof:

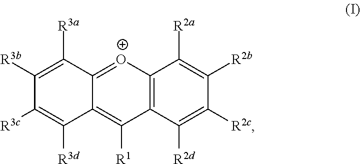

wherein:
R¹ is

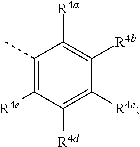

R^{2a}, R^{2b}, R^{2c}, and R^{2d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B},
   wherein two vicinal substituents selected from the group consisting of R^{2a}, R^{2b}, R^{2c}, and R^{2d} can combine with the atoms to which they are bound to form an optionally substituted C₆-C₁₀ aryl or optionally substituted C₂-C₁₀ heteroaryl, and
   wherein no more than three of R^{2a}, R^{2b}, R^{2c}, and R^{2d} are H;
R^{3a}, R^{3b}, R^{3c}, R^{3d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D},
   wherein two vicinal substituents selected from the group consisting of R^{3a}, R^{3b}, R^{3e}, and R^{3d} can combine with the atoms to which they are bound to form an optionally substituted C₆-C₁₀ aryl or optionally substituted C₂-C₁₀ heteroaryl, and
   wherein no more than three of R^{3a}, R^{3b}, R^{3c}, and R^{3d} are H;
R^{4a}, R^{4b}, R^{4c}, R^{4d}, and R^{4e} are each independently selected from the group consisting of H, optionally substituted C₁-C₆ alkyl, CN, NO₂, S(═O)₂OH, and S(═O)₂R⁵,
   wherein at least one of R^{4a}, R^{4b}, R^{4c}, R^{4d}, and R^{4e} is S(═O)₂OH, and
   wherein at least one of R^{4a}, R^{4b}, R^{4c}, R^{4d}, and R^{4e} is S(═O)₂R⁵;
R⁵ is at least one selected from the group consisting of H, OH, halogen, and

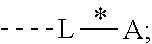

each occurrence of L is independently selected from the group consisting of a bond and a linker;
each occurrence of A is H, optionally substituted C₁-C₆ alkyl, an imaging agent, a polymeric macromolecule, or a therapeutic agent;
R^{A} and R^{B}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{A} and R^{B} can independently with one of R^{2a}, R^{2b}, R^{2c}, and R^{2a} to form an optionally substituted C₅-C₈ heterocycloalkyl or C₅-C₈ heterocycloalkenyl, and
R^{C} and R^{D}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
   wherein each of R^{C} and R^{D} can independently with one of R^{3a}, R^{3b}, R^{3c} and R^{3d} to form an optionally substituted C₅-C₅ heterocycloalkyl or C₅-C₈ heterocycloalkenyl.

Embodiment 34 provides the kit of Embodiment 33, wherein R^{2a}, R^{2b}, R^{2c}, and R^{2d} are each independently selected from the group consisting of H, F, Cl, Br, I, CH₃, CF₃, SO₃H, N(CH₃)₂, N(CH₂CH₃)₂, —(CH₂)₂C(═O)OH, B(OH)₂,

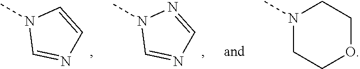

Embodiment 35 provides the kit of any one of Embodiments 33-34, wherein R^{3a}, R^{3b}, R^{3c}, and R^{3d} are each independently selected from the group consisting of H, F, Cl, Br, I, CH₃, CF₃, SO₃H, N(CH₃)₂, N(CH₂CH₃)₂, —(CH₂)₂C(═O)OH, B(OH)₂,

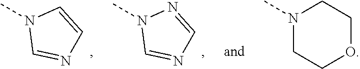

Embodiment 36 provides the kit of any one of Embodiments 33-35, wherein one of the following applies:
(a) R^{2a} is H, R^{2b} is Br, R^{2c} is H, and R^{2d} is H;
(b) R^{2a} is H, R^{2b} is H, R^{2c} is Br, and R^{2d} is H;
(c) R^{2a} is H, R^{2b} is N(CH₃)₂, R^{2c} is H, and R^{2d} is H; and
(d) R^{2a} is H, R^{2b} is N(CH₂CH₃)₂, R^{2c} is H, and R^{2d} is H.

Embodiment 37 provides the kit of any one of Embodiments 33-36, wherein one of the following applies:
(a) R^{3a} is H, R^{3b} is NO₂, R^{3c} is H, and R^{3d} is H;
(b) R^{3a} is H, R^{3b} is CH₃, R^{3c} is H, and R^{3d} is H;
(c) R^{3a} is H, R^{3b} is

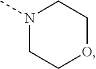

R^{3c} is H, and R^{3d} is H;
(d) R^{3a} is H, R^{3b} is H, R^{3c} is

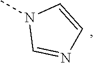

### and R^{3d} is H;

(e) R^{3a} is H, R^{3b} is H, R^{3c} is SO₃H, and R^{3d} is H;
(f) R^{3a} is H, R^{3b} is H, R^{3c} is

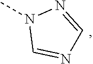

### and R^{3d} is H;

(g) R^{3a} is H, R^{3b} is N(CH₃)₂, R^{3c} is H, and R^{3d} is H; and
(h) R^{3a} is H, R^{3b} is N(CH₂CH₃)₂, R^{3c} is H, and R^{3d} is H.

Embodiment 38 provides the kit of any one of Embodiments 33-37, wherein the compound of formula (I) is selected from the group consisting of:

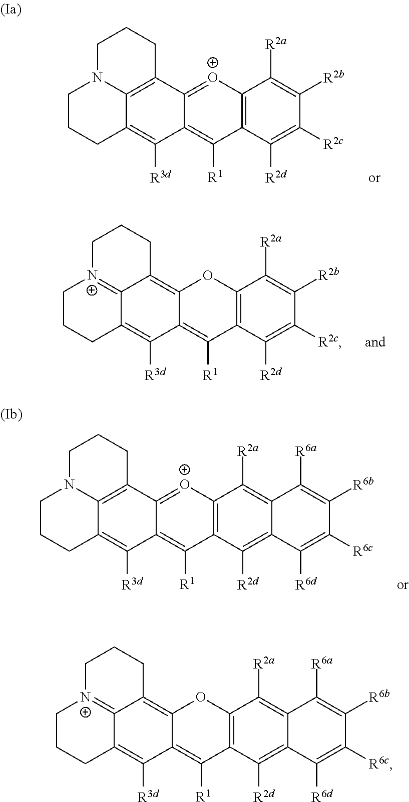

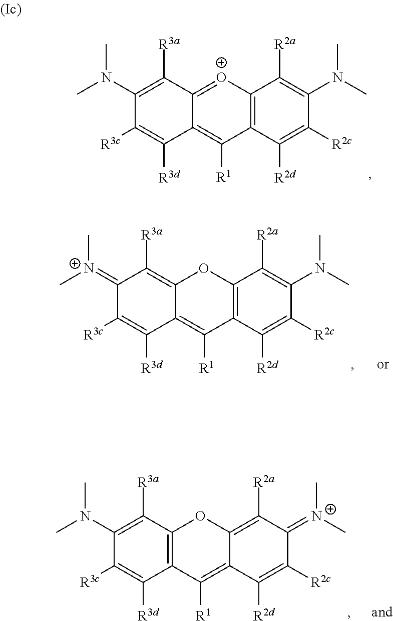

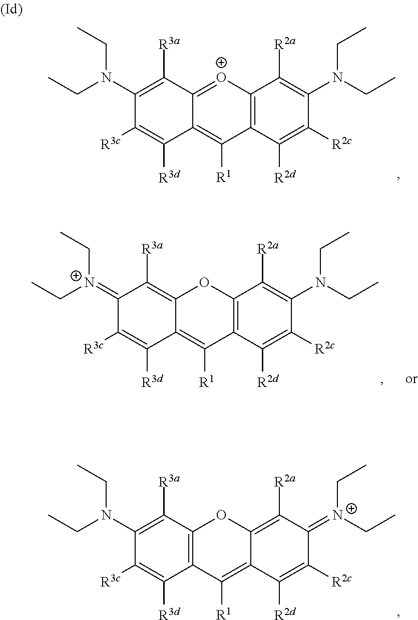

wherein R^{6a}, R^{6b}, R^{6c}, and R^{6d}, if present, are each independently selected from the group consisting of H. C₁-C₆ alkyl, C₁-C₆ haloalkyl, halogen, CN, and NO₂.

Embodiment 39 provides the kit of any one of Embodiments 33-36, wherein R^{3d} is H.

Embodiment 40 provides the kit of Embodiment 38 or 39, wherein one of the following applies:
(a) R^{2a} is H, R^{2b} is Br, R^{2c} is H, and R^{2d} is H;
(b) R^{2a} is H, R^{2b} is F, R^{2c} is H, and R^{2d} is H;
(c) R^{2a} is H, R^{2b} is Cl, R^{2c} is H, and R^{2d} is H;
(d) R^{2a} is H, R^{2b} is I, R^{2c} is H, and R^{2d} is H;
(e) R^{2a} is Br, R^{2b} is H, R^{2c} is H, and R^{2d} is H;
(f) R^{2a} is H, R^{2b} is H, R^{2c} is Br, and R^{2d} is H;
(g) R^{2a} is Br, R^{2b} is H, R^{2c} is Br, and R^{2d} is H;
(h) R^{2a} is H, R^{2b} is F, R^{2c} is F, and R^{2d} is H;
(i) R^{2a} is H, R^{2b} is F, R^{2c} is Cl, and R^{2d} is H:
(j) R^{2a} is H, R^{2b} is F, R^{2c} is Br, and R^{2d} is H:
(k) R^{2a} is H, R^{2b} is Cl, R^{2c} is F, and R^{2d} is H;
(l) R^{2a} is H, R^{2b} is Cl, R^{2c} is Cl, and R^{2d} is H;
(m) R^{2a} is H, R^{2b} is Cl, R^{2c} is Br, and R^{2d} is H;
(n) R^{2a} is H, R^{2b} is Br, R^{2c} is F, and R^{2d} is H;
(o) R^{2a} is H, R^{2b} is Br, R^{2c} is Cl, and R^{2d} is H;
(p) R^{2a} is H, R^{2b} is I, R^{2c} is Cl, and R^{2d} is H;
(q) R^{2a} is H, R^{2b} is CF₃, R^{2c} is Br, and R^{2d} is H;
(r) R^{2a} is F, R^{2b} is F, R^{2c} is H, and R^{2d} is H;
(s) R^{2a} is F, R^{2b} is Cl, R^{2c} is H, and R^{2d} is H;
(t) R^{2a} is F, R^{2b} is Br, R^{2c} is H, and R^{2d} is H;
(u) R^{2a} is Cl, R^{2b} is F, R^{2c} is H, and R^{2d} is H;
(v) R^{2a} is Cl, R^{2b} is Cl, R^{2c} is H, and R^{2d} is H;
(w) R^{2a} is H, R^{2b} is NO₂, R^{2c} is H, and R^{2d} is H;
(x) R^{2a} is H, R^{2b} is N(CH₃)₂, R^{2c} is H, and R^{2d} is H;
(y) R^{2a} is H, R^{2b} is —(CH₂)₂C(═O)OH, R^{2c} is H, and R^{2d} is H;
(z) R^{2a} is H, R^{2b} is CH₃, R^{2c} is H, and R^{2d} is H;
(aa) R^{2a} is H, R^{2b} is B(OH)₂, R^{2c} is H, and R^{2d} is H;
(ab) R^{2a} is H, R^{6a} is H, R^{6b} is H, R^{6c} is Br, R^{6d} is H, and R^{2d} is H; and
(ac) R^{2a} is Br, R^{6a} is H, R^{6b} is H, R^{6c} is H, R^{6d} is H, and R^{2d} is H.

Embodiment 41 provides the kit of any one of Embodiments 33-40, wherein at least one of the following applies:
(a) at least one of R^{4a}, R^{4b}, R^{4c}, R^{4d}, and R^{4e} is H;
(b) at least two of R^{4a}, R^{4b}, R^{4c}, R^{4d}, and R^{4e} are H;
(c) at least three of R^{4a}, R^{4b}, R^{4c}, R^{4d}, and R^{4e} are H; and
(d) four of R^{4a}, R^{4b}, R^{4c}, R^{4d}, and R^{4e} are H.

Embodiment 42 provides the kit of any one of Embodiments 33-41, wherein R¹ is selected from the group consisting of

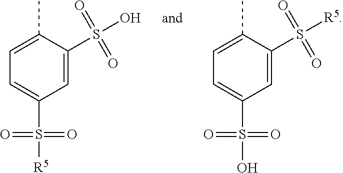

Embodiment 43 provides the kit of any one of Embodiments 33-42, wherein L comprises n instances of Z, wherein:
each occurrence of Z is independently selected from the group consisting of —O—, —N(R⁷)—, —S—, —S(═O)—, —S(═O)₂—, —C(═O)—, optionally substituted C₁-C₁₀ alkylenyl, optionally substituted C₁-C₁₀ heteroalkylenyl, optionally substituted C₃-C₈ cycloalkylenyl, optionally substituted C₂-C₈ heterocycloalkylenyl, optionally substituted C₁-C₁₀ alkenylenyl, optionally substituted C₃-C₈ cycloalkenylenyl, optionally substituted C₂-C₈ heterocycloalkenylenyl, optionally substituted C₆-C₁₀ arylenyl, and optionally substituted C₂-C₁₀ heteroarylenyl;
R⁷ is selected from the group consisting of H and C₁-C₆ alkyl; and
n is an integer ranging from 0 to 500.

Embodiment 44 provides the kit of Embodiment 43, wherein each instance of Z is independently selected from the group consisting of —NH—, —S—, —O—, —(CH₂)₀₋₁₀—, —(CH₂)₂O—, —O(CH₂)₂—, —C(═O)—,

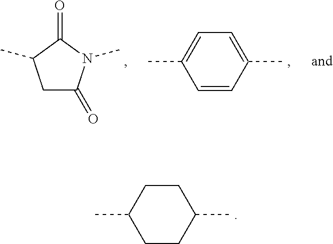

Embodiment 45 provides the kit of any one of Embodiments 33-43, wherein L is selected from the group consisting of a bond, —NH—,

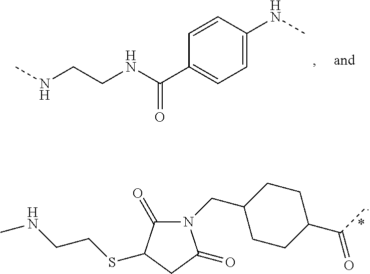

Embodiment 46 provides the kit of any one of Embodiments 33-45, wherein the therapeutic agent is selected from the group consisting of a small molecule, polypeptide, protein, and aptamer.

Embodiment 47 provides the kit of Embodiment 46, wherein the small molecule is a compound useful for the treatment of cancer.

Embodiment 48 provides the kit of any one of Embodiments 33-47, wherein the therapeutic agent is selected from the group consisting of colchicine, deferoxamine, paclitaxel (taxol), tofacitinib, methotrexate, hydrocortisone, prednisone, triiodothyronine, cyclophosphamide, amphotericin B, vancomycin, doxorubicin, mitoxantrone, imatinib, darunavir, and fosamprenavir.

Embodiment 49 provides the kit of any one of Embodiments 33-48, wherein the therapeutic agent comprises at least one modification and/or derivatization.

Embodiment 50 provides the kit of any one of Embodiments 33-49, wherein the therapeutic modification and/or derivatization comprises bond

[symbol]

Embodiment 51 provides the kit of any one of Embodiments 33-50, wherein A is selected from the group consisting of

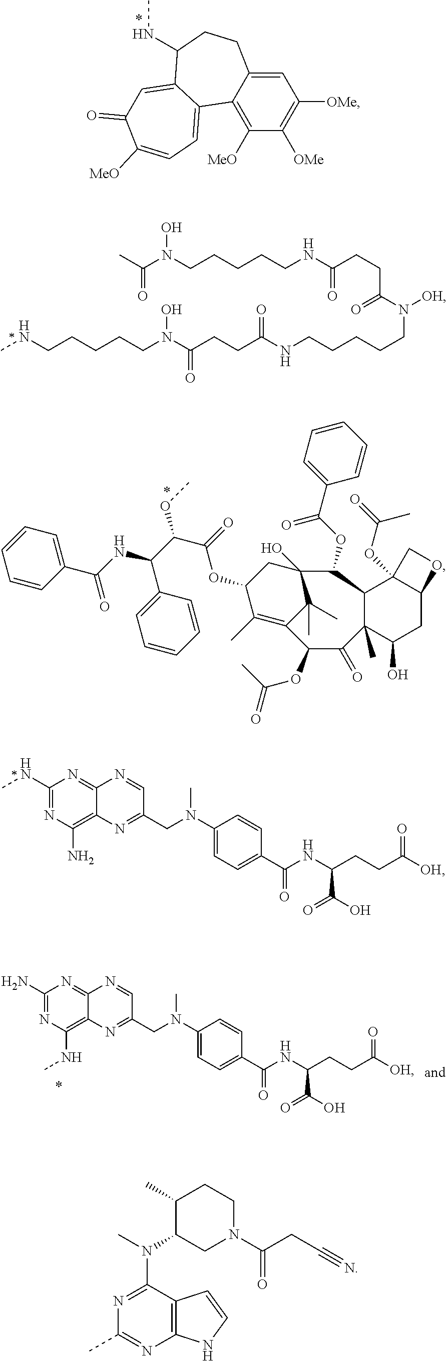

Embodiment 52 provides the kit of any one of Embodiments 33-42, wherein A is a compound of formula (LII):

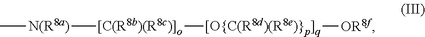

wherein:
R^{8a} and R^{8f} are each independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
each occurrence of R^{8b}, R^{8c}, R^{8d}, and R^{8e} is independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
is an integer selected from 1, 2, 3, 4, and 5;
p is an integer selected from 1, 2, 3, 4, and 5; and
q is an integer ranging from 1 to 500.

Embodiment 53 provides the kit of Embodiment 52, wherein at least one of the following applies:
(a) R^{8a} is H;
(b) R^{8f} is selected from the group consisting of H and Me;
(c) each occurrence of R^{8a}, R^{8b}, R^{8c}, and R^{8e} is independently H:
(d) o is 2;
(e) p is 2; and
(f) q is 10 to 500.

Embodiment 54 provides the kit of Embodiment 52 or 53, wherein A is

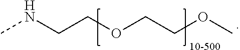

Embodiment 55 provides the kit of any one of Embodiments 33-54, wherein R⁵ is selected from the group consisting of

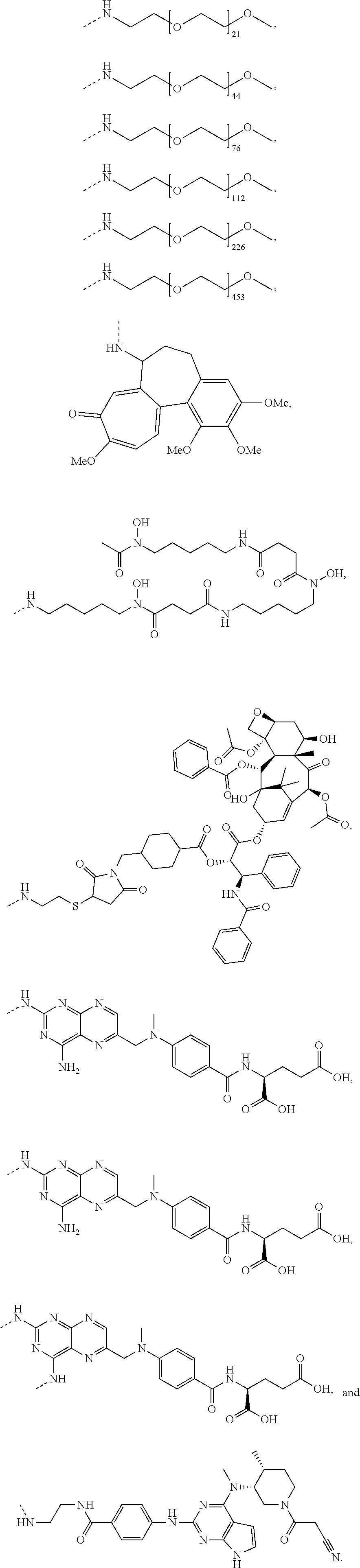

Embodiment 56 provides the kit of any one of Embodiments 33-55, wherein each occurrence of optionally substituted alkyl, optionally substituted haloalkyl, optionally substituted cycloalkyl, optionally substituted heterocycloalkyl, optionally substituted heterocycloalkenyl, optionally substituted aryl, optionally substituted heteroaryl, optionally substituted alkylenyl, optionally substituted cycloalkylenyl, optionally substituted heterocycloalkylenyl, optionally substituted alkenylenyl, optionally substituted cycloalkenylenyl, optionally substituted heterocycloalkenylenyl, optionally substituted arylenyl, and optionally substituted heteroarylenyl is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂ OR^{I}, N(R^{I})(R^{II}), C₁-C₆ haloalkoxy, C₃-C₈ halocycloalkoxy, aryl, heteroaryl, (C₁-C₆ alkylenyl)C(═O)N(R^{I})(R^{II}), (C₁-C₆ alkylenyl)C(═O)OR^{I}, O(C₁-C₃ alkylenyl)C(═O)OR^{II}, O(C₁-C₃ alkylenyl)C(═O)N(R^{I})(R^{II}), C(═O)R^{I}, C(═O)OR^{I}, OC(═O)R^{I}, OC(═O)OR^{I}, SR^{I}, S(═O)R^{I}, S(═O)₂R^{I}, S(═O)₂N(R^{I})(R^{II}), S(═O)₂NR^{I}C(═O)NHR^{II}, N(R^{I})S(═O)₂R^{II}, N(R^{I})C(═O)R^{II}, and C(═O)NR^{I}R^{II}, wherein R^{I} and R^{II} are each independently selected from the group consisting of H, —C(═O)(C₁-C₆ alkyl), C₁-C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

Embodiment 57 provides the kit of any one of Embodiments 33-56, wherein the isotopologue thereof comprises at least one radioactive tracer selected from the group consisting of ¹¹C, ³N, ¹⁵O, ¹⁸F, ¹²⁴I, ¹³¹I, and ¹³⁵I.

Embodiment 58 provides the kit of any one of Embodiments 33-57, which is selected from the group consisting of:

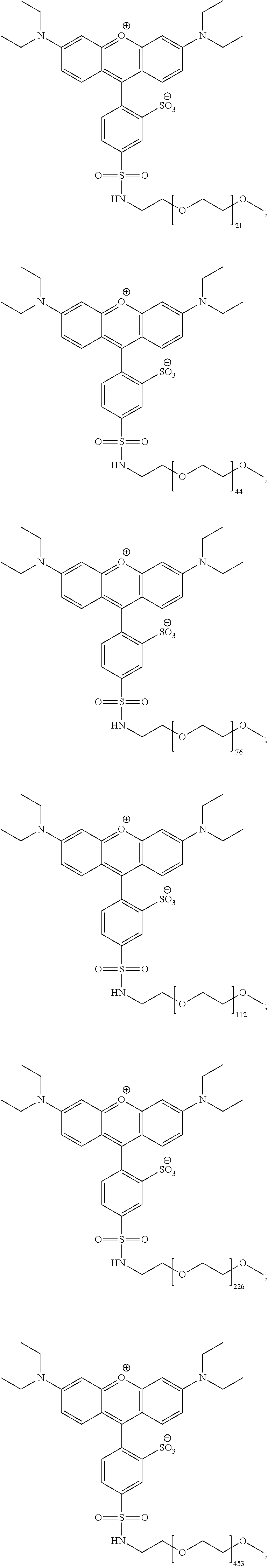

2-(3,6-bis(dimethylamino)xanthylium-9-yl)-5-(N-(1,2,3,10-tetramethoxy-9-oxo-5,6,7,9-tetrahydrobenzo[a]heptalen-7-yl)sulfamoyl)benzenesulfonate;
2-(3,6-bis(diethylamino)xanthylium-9-yl)-5-(N-(1,2,3,10-tetramethoxy-9-oxo-5,6,7,9-tetrahydrobenzo[a]heptalen-7-yl)sulfamoyl)benzenesulfonate;
2-(3,6-bis(dimethylamino)xanthylium-9-yl)-5-(N-(3,14,25-trihydroxy-2,10,13,21,24-pentaoxo-3,9,14,20,25-pentaazatriacontan-30-yl)sulfamoyl)benzenesulfonate;
5-(N-(2-((1-((4-((((1R,2S)-1-benzamido-3-(((2aS,4R,4aR,6S,9R,11R,12R,12aS,12bR)-6,12b-diacetoxy-12-(benzoyloxy)-4,11-dihydroxy-4a,8,13,13-tetramethyl-5-oxo-2a,3,4,4a,5,6,9,10,11,12,12a,12b-dodecahydro-1H-7,11-methanocyclodeca[3,4]benzo [1,2-b]oxet-9-yl)oxy)-3-oxo-1-phenylpropan-2-yl)oxy)carbonyl)cyclohexyl)methyl)-2,5-dioxopyrrolidin-3-yl)thio)ethyl)sulfamoyl)-2-(3,6-bis(dimethylamino)xanthylium-9-yl)benzenesulfonate;
(S)-5-(N-(4-amino-6-(((4-((1,3-dicarboxypropyl)carbamoyl)phenyl)(methyl)amino)methyl)pteridin-2-yl)sulfamoyl)-2-(12-bromo-1,2,3,5,6,7-hexahydrochromeno[2,3-f]pyrido[3,2,1-ij]quinolin-4-ium-9-yl)benzenesulfonate;
(R)-5-(N-(2-amino-6-(((4-((1,3-dicarboxypropyl)carbamoyl)phenyl)(methyl)amino)methyl)pteridin-4-yl)sulfamoyl)-2-(12-bromo-1,2,3,5,6,7-hexahydrochromeno[2,3-f]pyrido[3,2,1-ij]quinolin-4-ium-9-yl)benzenesulfonate; and
5-(N-(2-(4-((4-(((3R,4R)-1-(2-cyanoacetyl)-4-methylpiperidin-3-yl)(methyl)amino)-7H-pyrrolo[2,3-d]pyrimidin-2-yl)amino)benzamido)ethyl)sulfamoyl)-2-(6-(diethylamino)-3-(diethyliminio)-3H-xanthen-9-yl)benzenesulfonate.

Embodiment 59 provides the kit of any one of Embodiments 33-58, wherein at least one of the following applies:
the kit comprises the Slco1a4 protein or the SLCO1A2 protein;
the kit comprises an mRNA encoding Slco1a4 or a SLCO1A2, wherein the mRNA comprises a constitutively active promoter or a promoter active in the cell;
the kit comprises a DNA encoding Slco1a4 or a SLCO1A2, wherein the DNA comprises a constitutively active promoter or a promoter active in the cell;
the kit comprises a DNA encoding Slco1a4 or a SLCO1 A2 for being inserted into the genome of the cell, wherein the DNA comprises a constitutively active promoter or a promoter active in the cell that drives the expression of the Slco1a4 or SLCO1A2;
the kit comprises a DNA encoding Slco1a4 or a SLCO1A2 for being inserted into the genome of the cell, wherein the DNA is inserted downstream of a promoter active in the cell; or
the kit comprises a DNA encoding a constitutively active promoter or a promoter active in the cell, wherein the DNA is inserted upstream of a Slco1a4 gene or a SLCO1A2 gene to drive the expression of the Slco1a4 gene or the SLCO1A2 gene in the cell.

Embodiment 60 provides the kit of any one of Embodiments 33-59, wherein the compound is co-formulated with a pharmaceutically acceptable carrier.

Embodiment 61 provides the kit of any one of Embodiments 33-60, further comprising a component for delivering the Slco1a4 protein, the SLCO1A2 protein, or the nucleic acid encoding the same into the cell.

Embodiment 62 provides the kit of Embodiment 61, wherein the component Slco1a4 protein, the SLCO1A2 protein, or the nucleic acid encoding the same into the cell comprises a liposome, a metal nanoparticle, a micelle, a viral vector, an electroporation cuvette, or a microinjector.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A method of delivering a compound into a cell, comprising:
expressing Slco1a4 or SLCO1A2 in the cell; and
contacting with the cell a compound of formula (I), or a salt, solvate, tautomer, geometric isomer, or isotopologue thereof, and any combinations thereof: 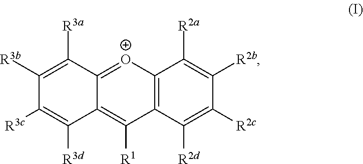
wherein:
R¹ is 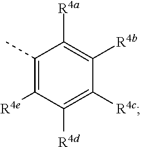
R^{2a}, R^{2b}, R^{2c}, and R^{2d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B},
wherein two vicinal substituents selected from the group consisting of R^{2a}, R^{2b}, R^{2c}, and R^{2d} can combine with the atoms to which they are bound to form an optionally substituted C₆-C₁₀ aryl or optionally substituted C₂-C₁₀ heteroaryl, and
wherein no more than three of R^{2a}, R^{2b}, R^{2c}, and R^{2d} are H;
R^{3a}, R^{3b}, R^{3c}, R^{3d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D},
wherein two vicinal substituents selected from the group consisting of R³a R^{3b}, R^{3c}, and R^{3d} can combine with the atoms to which they are bound to form an optionally substituted C₆-C₁₀ aryl or optionally substituted C₂-C₁₀ heteroaryl, and
wherein no more than three of R^{3a}, R^{3b}, R^{3c}, and R^{3d} are H;
R^{4a}, R^{4b}, R^{4c}, R^{4d}, and R^{4e} are each independently selected from the group consisting of H, optionally substituted C₁-C₆ alkyl, CN, NO₂, S(═O)₂OH, and S(═O)₂R⁵,
wherein at least one of R^{4a}, R^{4b}, R^{4c}, R^{4d}, and R^{4e} is S(═O)₂OH, and
wherein at least one of R^{4a}, R^{4b}, R^{4c}, R^{4d}, and R^{4e} is S(═O)₂R⁵;
R⁵ is at least one selected from the group consisting of H, OH, halogen, and 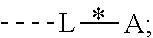
each occurrence of L is independently selected from the group consisting of a bond and a linker;
each occurrence of A is H, optionally substituted C₁-C₆ alkyl, an imaging agent, a polymeric macromolecule, or a therapeutic agent;
R^{A} and R^{B}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
wherein each of R^{A} and R^{B} can independently with one of R^{2a}, R^{2b}, R^{2c}, and R^{2d} to form an optionally substituted C₅-C₈ heterocycloalkyl or C₅-C₈ heterocycloalkenyl; and
R^{C} and R^{D}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
wherein each of R^{C} and R^{D} can independently with one of R^{3a}, R^{3b}, R^{3c}, and R^{3d} to form an optionally substituted C₅-C₈ heterocycloalkyl or C₅-C₈ heterocycloalkenyl.

2. The method of claim 1, wherein at least one of the following applies:
(a) R^{2a}, R^{2b}, R^{2c}, R^{2d}, R^{3a}, R^{3b}, R^{3c}, and R^{3d} are each independently selected from the group consisting of H, F, Cl, Br, I, CH₃, CF₃, SO₃H, N(CH₃)₂, N(CH₂CH₃)₂, —(CH₂)₂C(═O)OH, B(OH)₂, 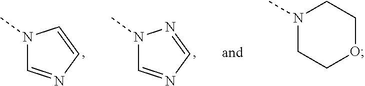
(b) R¹ is selected from the group consisting of 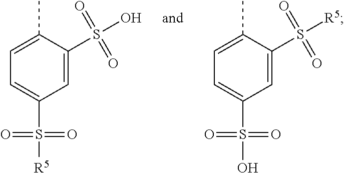
(c) R⁵ is selected from the group consisting of 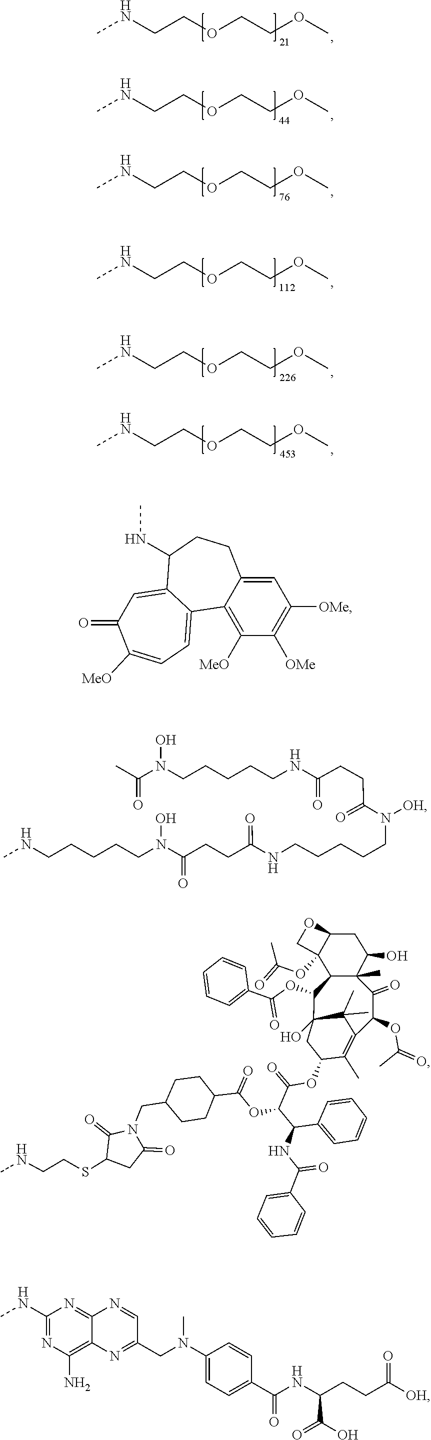 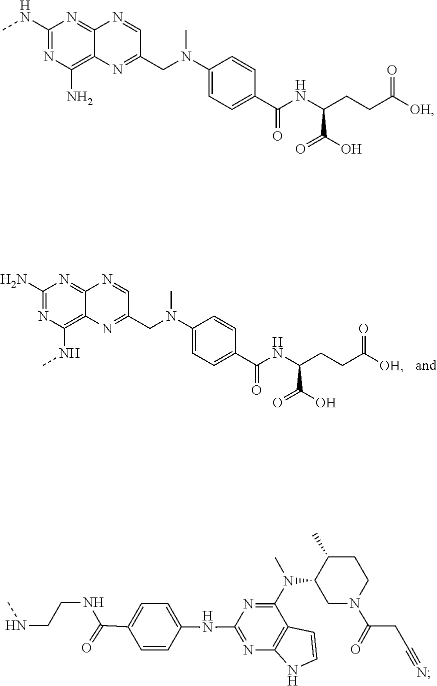
and
(d) L comprises n instances of Z, wherein:
each occurrence of Z is independently selected from the group consisting of —O—, —N(R⁷)—, —S—, —S(═O)—, —S(═O)₂—, —C(═O)—, optionally substituted C₁-C₁₀ alkylenyl, optionally substituted C₁-C₁₀ heteroalkylenyl, optionally substituted C₃-C₈ cycloalkylenyl, optionally substituted C₂-C₈ heterocycloalkylenyl, optionally substituted C₁-C₁₀ alkenylenyl, optionally substituted C₃-C₈ cycloalkenylenyl, optionally substituted C₂-C₈ heterocycloalkenylenyl, optionally substituted C₆-C₁₀ arylenyl, and optionally substituted C₂-C₁₀ heteroarylenyl;
R⁷ is selected from the group consisting of H and C₁-C₆ alkyl; and
n is an integer ranging from 0 to 500.

3-5. (canceled)

6. The method of claim 1, wherein the compound of formula (I) is selected from the group consisting of: 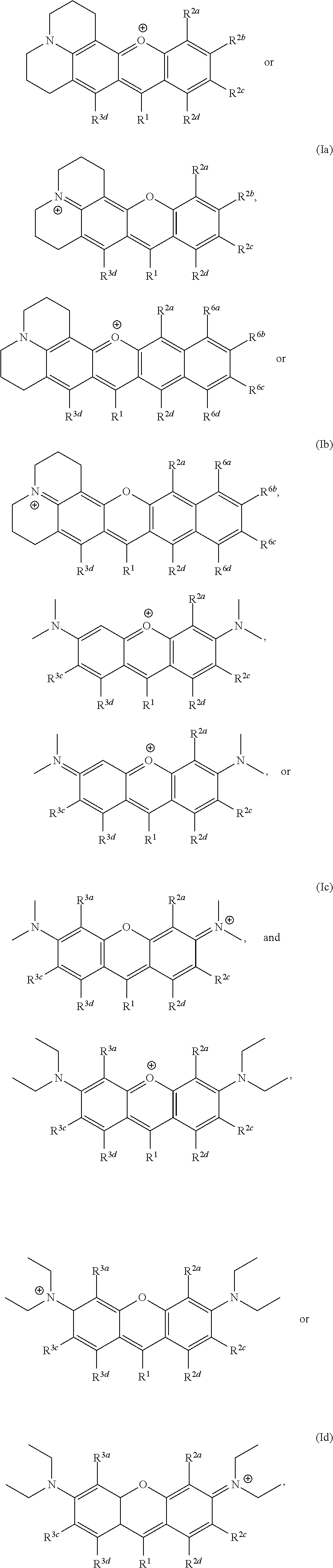
wherein R^{6a}, R^{6b}, R^{6c}, and R^{6d}, if present, are each independently selected from the group consisting of H, C₁-C₆ alkyl, C₁-C₆ haloalkyl, halogen, CN, and NO₂.

7. (canceled)

8. The method of claim 6, wherein one of the following applies:
(a) R^{2a} is H, R^{2b} is Br, R^{2c} is H, and R^{2d} is H;
(b) R^{2a} is H, R^{2b} is F, R^{2c} is H, and R^{2d} is H;
(c) R^{2a} is H, R^{2b} is Cl, R^{2c} is H, and R^{2d} is H;
(d) R^{2a} is H, R^{2b} is I, R^{2c} is H, and R^{2d} is H;
(e) R^{2a} is Br, R^{2b} is H, R^{2c} is H, and R^{2d} is H;
(f) R^{2a} is H, R^{2b} is H, R^{2c} is Br, and R^{2d} is H;
(g) R^{2a} is Br, R^{2b} is H, R^{2c} is Br, and R^{2d} is H;
(h) R^{2a} is H, R^{2b} is F, R^{2c} is F, and R^{2d} is H;
(i) R^{2a} is H, R^{2b} is F, R^{2c} is Cl, and R^{2d} is H;
(j) R^{2a} is H, R^{2b} is F, R^{2c} is Br, and R^{2d} is H;
(k) R^{2a} is H, R^{2b} is Cl, R^{2c} is F, and R^{2d} is H;
(l) R^{2a} is H, R^{2b} is Cl, R^{2c} is Cl, and R^{2d} is H;
(m) R^{2a} is H, R^{2b} is Cl, R^{2c} is Br, and R^{2d} is H;
(n) R^{2a} is H, R^{2b} is Br, R^{2c} is F, and R^{2d} is H;
(o) R^{2a} is H, R^{2b} is Br, R^{2c} is Cl, and R^{2d} is H;
(p) R^{2a} is H, R^{2b} is I, R^{2c} is Cl, and R^{2d} is H;
(q) R^{2a} is H, R^{2b} is CF₃, R^{2c} is Br, and R^{2d} is H;
(r) R^{2a} is F, R^{2b} is F, R^{2c} is H, and R^{2d} is H;
(s) R^{2a} is F, R^{2b} is Cl, R^{2c} is H, and R^{2d} is H;
(t) R^{2a} is F, R^{2b} is Br, R^{2c} is H, and R^{2d} is H;
(u) R^{2a} is Cl, R^{2b} is F, R^{2c} is H, and R^{2d} is H;
(v) R^{2a} is Cl, R^{2b} is Cl, R^{2c} is H, and R^{2d} is H;
(w) R^{2a} is H, R^{2b} is NO₂, R^{2c} is H, and R^{2d} is H;
(x) R^{2a} is H, R^{2b} is N(CH₃)₂, R^{2c} is H, and R^{2d} is H;
(y) R^{2a} is H, R^{2b} is —(CH₂)₂C(═O)OH, R^{2c} is H, and R^{2d} is H;
(z) R^{2a} is H, R^{2b} is CH₃, R^{2c} is H, and R^{2d} is H;
(aa) R^{2a} is H, R^{2b} is B(OH)₂, R^{2c} is H, and R^{2d} is H;
(ab) R^{2a} is H, R⁶a is H, R⁶b is H, R⁶c is Br, R⁶d is H, and R^{2d} is H;
(ac) R^{2a} is Br, R⁶a is H, R⁶b is H, R⁶c is H, R⁶d is H, and R^{2d} is H;
(ad) at least one of R^{4a}, R^{4b}, R^{4c}, R^{4d}, and R^{4e} is H:
(ae) at least two of R^{4a}, R^{4b}, R^{4c}, R^{4d}, and R^{4e} are H:
(af) at least three of R^{4a}, R^{4b}, R^{4c}, R^{4d}, and R^{4e} are H; and
(ag) four of R^{4a}, R^{4b}, R^{4c}, R^{4d}, and R^{4e} are H.

9-13. (canceled)

14. The method of claim 1, wherein at least one of the following applies:
(a) the therapeutic agent is selected from the group consisting of a small molecule, polypeptide, protein, and aptamer, optionally wherein the small molecule is a compound useful for the treatment of cancer;
(b) the therapeutic agent is selected from the group consisting of colchicine, deferoxamine, paclitaxel (taxol), tofacitinib, methotrexate, hydrocortisone, prednisone, triiodothyronine, cyclophosphamide, amphotericin B, vancomycin, doxorubicin, mitoxantrone, imatinib, darunavir, and fosamprenavir;
(c) the therapeutic agent comprises at least one modification and/or derivatization; and
(d) wherein the therapeutic modification and/or derivatization comprises bond

[symbol]

15-18. (canceled)

19. The method of claim 1, wherein A is selected from the group consisting of:

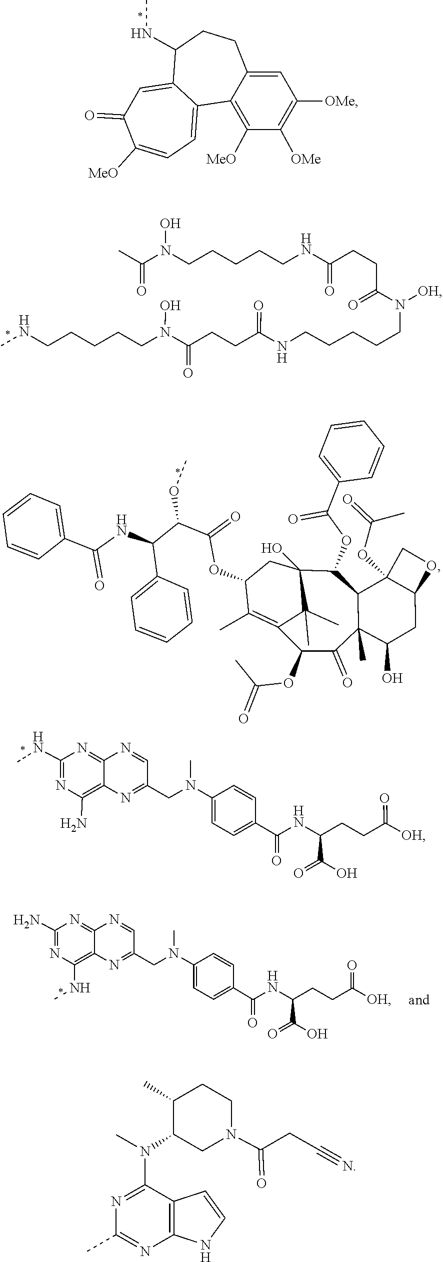

20. The method of claim 1, wherein A is a compound of formula (III): 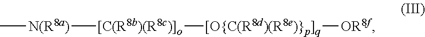
wherein:
R^{8a} and R^{8f} are each independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
each occurrence of R^{8b}, R^{8c}, R^{8d}, and R^{8e} is independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
o is 1, 2, 3, 4, or 5;
p is 1, 2, 3, 4, or 5; and
q is an integer ranging from 1 to 500.

21-22. (canceled)

23. The method of claim 1, wherein R⁵ is selected from the group consisting of:

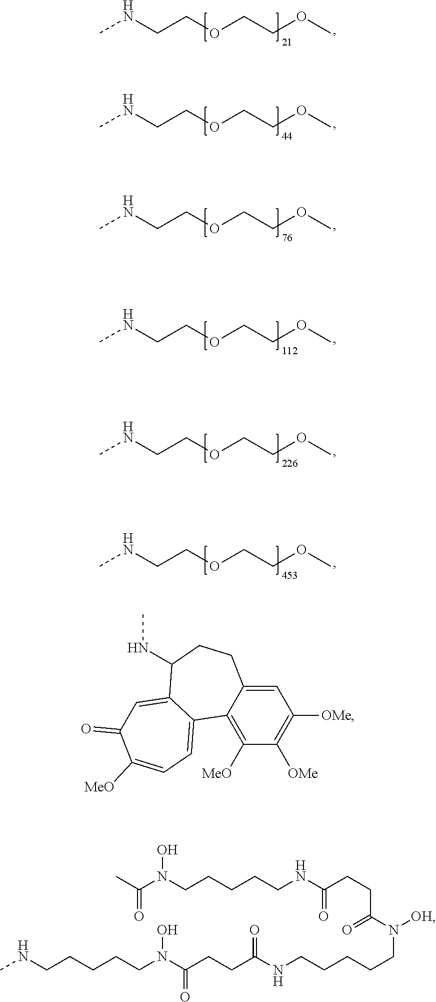

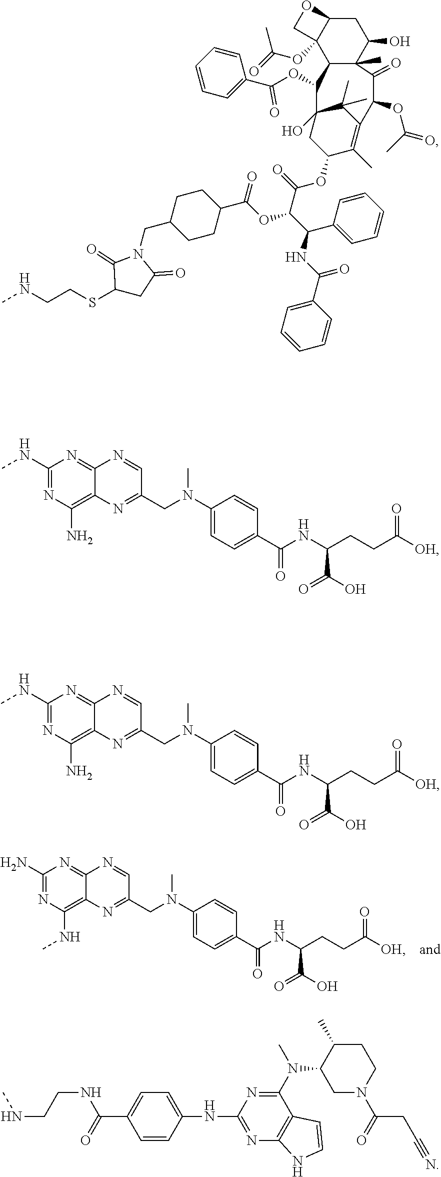

24. (canceled)

25. The method of claim 1, wherein the isotopologue thereof comprises at least one radioactive tracer selected from the group consisting of ¹¹C, ¹³N, ¹⁵O, ¹⁸F, ¹²⁴I, ¹³¹I and ¹³⁵I.

26. The method of claim 1, which is selected from the group consisting of: 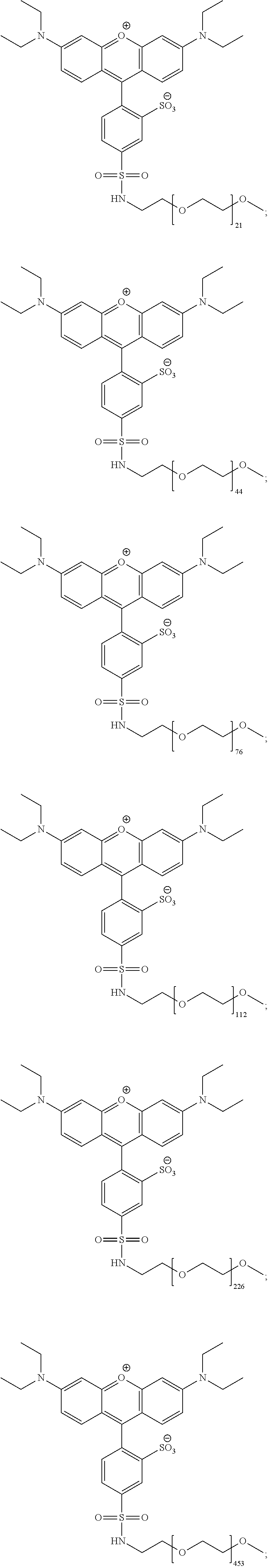
2-(3,6-bis(dimethylamino)xanthylium-9-yl)-5-(N-(1,2,3,10-tetramethoxy-9-oxo-5,6,7,9-tetrahydrobenzo[a]heptalen-7-yl)sulfamoyl)benzenesulfonate;
2-(3,6-bis(diethylamino)xanthylium-9-yl)-5-(N-(1,2,3,10-tetramethoxy-9-oxo-5,6,7,9-tetrahydrobenzo[a]heptalen-7-yl)sulfamoyl)benzenesulfonate;
2-(3,6-bis(dimethylamino)xanthylium-9-yl)-5-(N-(3,14,25-trihydroxy-2,10,13,21,24-pentaoxo-3,9,14,20,25-pentaazatriacontan-30-yl)sulfamoyl)benzenesulfonate;
5-(N-(2-((1-((4-((((1R,2S)-1-benzamido-3-(((2aS,4R,4aR,6S,9R,11R,12R,12aS,12bR)-6,12b-diacetoxy-12-(benzoyloxy)-4,11-dihydroxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-9-yl)oxy)-3-oxo-1-phenylpropan-2-yl)oxy)carbonyl)cyclohexyl)methyl)-2,5-dioxopyrrolidin-3-yl)thio)ethyl)sulfamoyl)-2-(3,6-bis(dimethylamino)xanthylium-9-yl)benzenesulfonate;
(S)-5-(N-(4-amino-6-(((4-((1,3-dicarboxypropyl)carbamoyl)phenyl)(methyl)amino)methyl)pteridin-2-yl)sulfamoyl)-2-(12-bromo-1,2,3,5,6,7-hexahydrochromeno[2,3-f]pyrido[3,2,1-ij]quinolin-4-ium-9-yl)benzenesulfonate;
(R)-5-(N-(2-amino-6-(((4-((1,3-dicarboxypropyl)carbamoyl)phenyl)(methyl)amino)methyl)pteridin-4-yl)sulfamoyl)-2-(12-bromo-1,2,3,5,6,7-hexahydrochromeno[2,3-f]pyrido[3,2,1-ij]quinolin-4-ium-9-yl)benzenesulfonate; and
5-(N-(2-(4-((4-(((3R,4R)-1-(2-cyanoacetyl)-4-methylpiperidin-3-yl)(methyl)amino)-7H-pyrrolo[2,3-d]pyrimidin-2-yl)amino)benzamido)ethyl)sulfamoyl)-2-(6-(diethylamino)-3-(diethyliminio)-3H-xanthen-9-yl)benzenesulfonate.

27. The method of claim 1, wherein expressing Slco1a4 or SLCO1A2 in the cell comprises:
delivering a Slco1a4 protein or a SLCO1A2 protein into the cell;
delivering an mRNA encoding Slco1a4 or a SLCO1A2 into the cell, wherein the mRNA comprises a constitutively active promoter or a promoter active in the cell;
delivering a DNA encoding Slco1a4 or a SLCO1A2 into the cell, wherein the DNA comprises a constitutively active promoter or a promoter active in the cell;
introducing a DNA encoding Slco1a4 or a SLCO1A2 into the genome of the cell, wherein the DNA comprises a constitutively active promoter or a promoter active in the cell that drives the expression of the Slco1a4 or SLCO1A2, or wherein the DNA is inserted downstream of a promoter active in the cell; or
introducing a constitutively active promoter or a promoter active in the cell to drive the expression of a Slco1a4 gene or a SLCO1A2 gene in the cell.

28. The method of claim 1, wherein at least one of the following applies:
(a) the compound is co-formulated with a pharmaceutically acceptable carrier; and
(b) the cell is in a subject, and wherein the method comprises administering to the subject an effective amount of the compound, optionally wherein the compound is administered topically, intravenously, orally, intramuscularly, intrathecally, or intraperitoneally.

29-32. (canceled)

33. A kit for delivering a compound into a cell, comprising:
a Slco1a4 protein, a SLCO1A2 protein, or a nucleic acid encoding the same; and
a compound of formula (I), or a salt, solvate, tautomer, geometric isomer, or isotopologue thereof, and any combinations thereof: 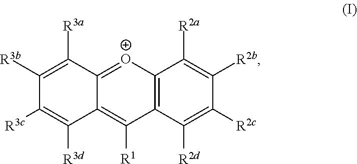
wherein:
R¹ is 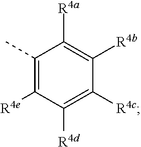
R^{2a}, R^{2b}, R^{2c}, and R^{2d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B},
wherein two vicinal substituents selected from the group consisting of R^{2a}, R^{2b}, R^{2c}, and R^{2d} can combine with the atoms to which they are bound to form an optionally substituted C₆-C₁₀ aryl or optionally substituted C₂-C₁₀ heteroaryl, and
wherein no more than three of R^{2a}, R^{2b}, R^{2c}, and R^{2d} are H;
R^{3a}, R^{3b}, R^{3c}, R^{3d} are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D},
wherein two vicinal substituents selected from the group consisting of R^{3a}, R^{3b}, R^{3c}, and R^{3d} can combine with the atoms to which they are bound to form an optionally substituted C₆-C₁₀ aryl or optionally substituted C₂-C₁₀ heteroaryl, and
wherein no more than three of R^{3a}, R^{3b}, R^{3c}, and R^{3d} are H;
R^{4a}, R^{4b}, R^{4c}, R^{4d}, and R^{4e} are each independently selected from the group consisting of H, optionally substituted C₁-C₆ alkyl, CN, NO₂, S(═O)₂OH, and S(═O)₂R⁵,
wherein at least one of R^{4a}, R^{4b}, R^{4c}, R^{4d}, and R^{4e} is S(═O)₂OH, and
wherein at least one of R^{4a}, R^{4b}, R^{4c}, R^{4d}, and R^{4e} is S(═O)₂R⁵;
R⁵ is at least one selected from the group consisting of H, OH, halogen, and 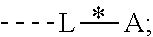
each occurrence of L is independently selected from the group consisting of a bond and a linker;
each occurrence of A is H, optionally substituted C₁-C₆ alkyl, an imaging agent, a polymeric macromolecule, or a therapeutic agent;
R^{A} and R^{B}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
wherein each of R^{A} and R^{B} can independently with one of R^{2a}, R^{2b}, R^{2c}, and R^{2d} to form an optionally substituted C₅-C₈ heterocycloalkyl or C₅-C₈ heterocycloalkenyl; and
R^{C} and R^{D}, if present, are each independently selected from the group consisting of H, optionally substituted C₁-C₆ alkyl, optionally substituted C₁-C₆ haloalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl,
wherein each of R^{C} and R^{D} can independently with one of R^{3a}, R^{3b}, R^{3c}, and R^{3d} to form an optionally substituted C₅-C₈ heterocycloalkyl or C₅-C₈ heterocycloalkenyl.

34-37. (canceled)

38. The kit of claim 33, wherein the compound of formula (I) is selected from the group consisting of: 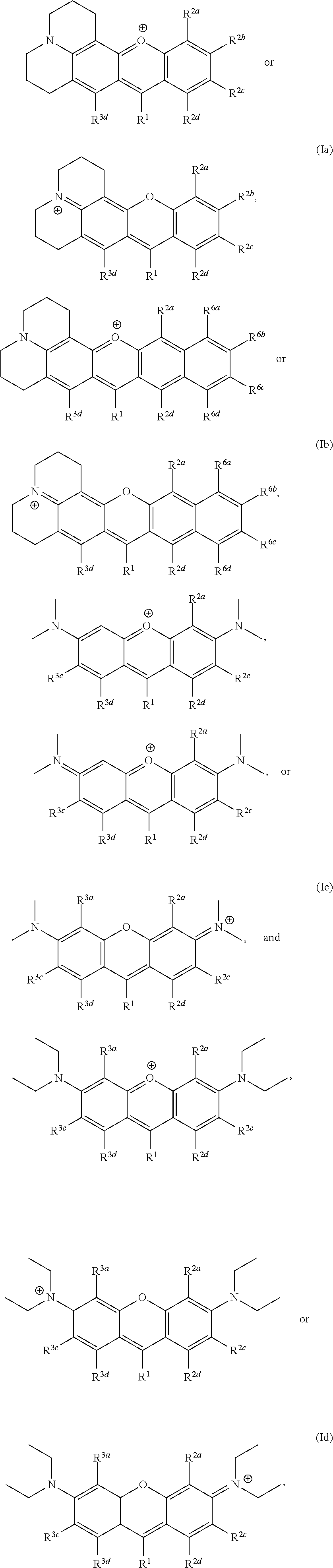
wherein R^{6a}, R^{6b}, R^{6c}, and R^{6d}, if present, are each independently selected from the group consisting of H, C₁-C₆ alkyl, C₁-C₆ haloalkyl, halogen, CN, and NO₂.

39-45. (canceled)

46. The kit of claim 33, wherein at least one of the following applies:
(a) the therapeutic agent is selected from the group consisting of a small molecule, polypeptide, protein, and aptamer, optionally wherein the small molecule is a compound useful for the treatment of cancer; and
(b) the therapeutic agent is selected from the group consisting of colchicine, deferoxamine, paclitaxel (taxol), tofacitinib, methotrexate, hydrocortisone, prednisone, triiodothyronine, cyclophosphamide, amphotericin B, vancomycin, doxorubicin, mitoxantrone, imatinib, darunavir, and fosamprenavir.

47-57. (canceled)

58. The kit of claim 33, which is selected from the group consisting of: 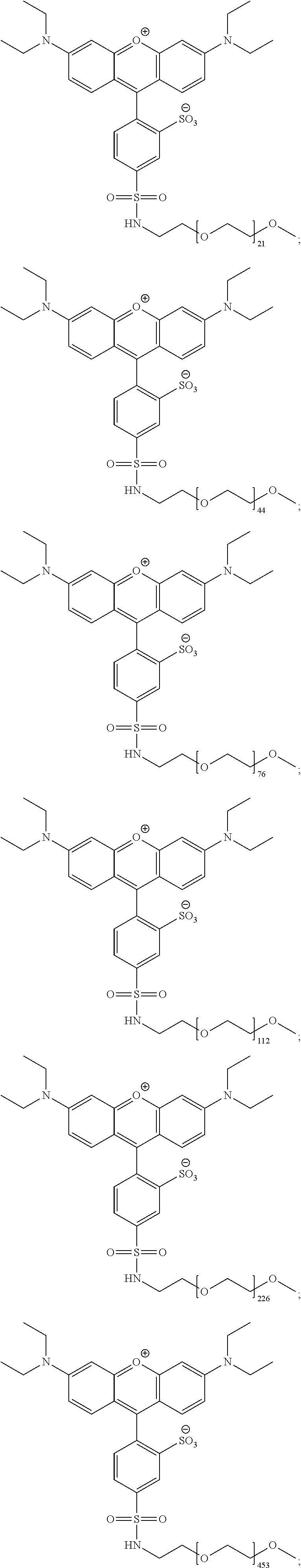
2-(3,6-bis(dimethylamino)xanthylium-9-yl)-5-(N-(1,2,3,10-tetramethoxy-9-oxo-5,6,7,9-tetrahydrobenzo[a]heptalen-7-yl)sulfamoyl)benzenesulfonate;
2-(3,6-bis(diethylamino)xanthylium-9-yl)-5-(N-(1,2,3,10-tetramethoxy-9-oxo-5,6,7,9-tetrahydrobenzo[a]heptalen-7-yl)sulfamoyl)benzenesulfonate;
2-(3,6-bis(dimethylamino)xanthylium-9-yl)-5-(N-(3,14,25-trihydroxy-2,10,13,21,24-pentaoxo-3,9,14,20,25-pentaazatriacontan-30-yl)sulfamoyl)benzenesulfonate;
5-(N-(2-((1-((4-((((1R,2S)-1-benzamido-3-(((2aS,4R,4aR,6S,9R,11R,12R,12aS,12bR)-6,12b-diacetoxy-12-(benzoyloxy)-4,11-dihydroxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-9-yl)oxy)-3-oxo-1-phenylpropan-2-yl)oxy)carbonyl)cyclohexyl)methyl)-2,5-dioxopyrrolidin-3-yl)thio)ethyl)sulfamoyl)-2-(3,6-bis(dimethylamino)xanthylium-9-yl)benzenesulfonate;
(S)-5-(N-(4-amino-6-(((4-((1,3-dicarboxypropyl)carbamoyl)phenyl)(methyl)amino)methyl)pteridin-2-yl)sulfamoyl)-2-(12-bromo-1,2,3,5,6,7-hexahydrochromeno[2,3-f]pyrido[3,2,1-ij]quinolin-4-ium-9-yl)benzenesulfonate;
(R)-5-(N-(2-amino-6-(((4-((1,3-dicarboxypropyl)carbamoyl)phenyl)(methyl)amino)methyl)pteridin-4-yl)sulfamoyl)-2-(12-bromo-1,2,3,5,6,7-hexahydrochromeno[2,3-f]pyrido[3,2,1-ij]quinolin-4-ium-9-yl)benzenesulfonate; and
5-(N-(2-(4-((4-(((3R,4R)-1-(2-cyanoacetyl)-4-methylpiperidin-3-yl)(methyl)amino)-7H-pyrrolo[2,3-d]pyrimidin-2-yl)amino)benzamido)ethyl)sulfamoyl)-2-(6-(diethylamino)-3-(diethyliminio)-3H-xanthen-9-yl)benzenesulfonate.

59. The kit of claim 33, wherein at least one of the following applies:
the kit comprises the Slco1a4 protein or the SLCO1A2 protein;
the kit comprises an mRNA encoding Slco1a4 or a SLCO1A2, wherein the mRNA comprises a constitutively active promoter or a promoter active in the cell;
the kit comprises a DNA encoding Slco1a4 or a SLCO1A2, wherein the DNA comprises a constitutively active promoter or a promoter active in the cell;
the kit comprises a DNA encoding Slco1a4 or a SLCO1A2 for being inserted into the genome of the cell, wherein the DNA comprises a constitutively active promoter or a promoter active in the cell that drives the expression of the Slco1a4 or SLCO1A2;
the kit comprises a DNA encoding Slco1a4 or a SLCO1A2 for being inserted into the genome of the cell, wherein the DNA is inserted downstream of a promoter active in the cell; or
the kit comprises a DNA encoding a constitutively active promoter or a promoter active in the cell, wherein the DNA is inserted upstream of a Slco1a4 gene or a SLCO1A2 gene to drive the expression of the Slco1a4 gene or the SLCO1A2 gene in the cell.

60. The kit of claim 33, wherein the compound is co-formulated with a pharmaceutically acceptable carrier.

61. The kit of claim 33, further comprising a component for delivering the Slco1a4 protein, the SLCO1A2 protein, or the nucleic acid encoding the same into the cell.

62. The kit of claim 61, wherein the component Slco1a4 protein, the SLCO1A2 protein, or the nucleic acid encoding the same into the cell comprises a liposome, a metal nanoparticle, a micelle, a viral vector, an electroporation cuvette, or a microinjector.
